(19) United States
(12) Patent Application Publication
(10) Pub. No.: US 2023/0057130 A1
(43) Pub. Date: Feb. 23, 2023

(54) FORMULATIONS OF 19-NOR C3,3-DISUBSTITUTED C21-N-PYRAZOLYL STEROID AND METHODS OF USE THEREOF

(71) Applicant: Sage Therapeutics, Inc., Cambridge, MA (US)

(72) Inventors: Paul Steven Watson, Carrboro, NC (US); Bret Berner, Seattle, WA (US); Padma Narayan, Arlington, MA (US); Xiaoxia Chen, Acton, MA (US); Todd Anthony Stutzman, Overland Park, KS (US); Jianing Meng, Kansas City, MO (US); Carolyn Wilkerson, Somerset, NJ (US); Raj Ramnik Jain, Overland Park, KS (US)

(21) Appl. No.: 17/579,541

(22) Filed: Jan. 19, 2022

Related U.S. Application Data
(63) Continuation of application No. PCT/US2021/042394, filed on Jul. 20, 2021.
(60) Provisional application No. 63/054,070, filed on Jul. 20, 2020.

(51) Int. Cl. A61K 31/58; A61K 9/20; A61K 9/48

(52) U.S. Cl. CPC A61K 31/58; A61K 9/2018; A61K 9/2027; A61K 9/2054; A61K 9/2009; A61K 9/2013; A61K 9/4858; A61K 9/4866; A61K 9/485

(57) ABSTRACT

This invention relates to a 19-nor C3,3-disubstituted C21-pyrazolyl steroid of formula (I) and pharmaceutical compositions thereof. Also disclosed herein are methods of making the pharmaceutical compositions of the 19-nor C3,3-disubstituted C21-pyrazolyl steroid of formula (I) and methods of using the 19-nor C3,3-disubstituted C21-pyrazolyl steroid of formula (I) or crystalline solid forms, pharmaceutically acceptable salts, and pharmaceutically acceptable compositions thereof.

### CROSS-REFERENCE TO RELATED APPLICATIONS

This application is a continuation application under 35 U.S.C. 111(a) of International Application PCT/US2021/042394, filed Jul. 20, 2021, which claims priority to U.S. Provisional Application No. 63/054,070, filed Jul. 20, 2020, of which the contents of each are incorporated herein in its entirety.

### BACKGROUND OF THE INVENTION

Brain excitability is defined as the level of arousal of an animal, a continuum that ranges from coma to convulsions, and is regulated by various neurotransmitters. In general, neurotransmitters are responsible for regulating the conductance of ions across neuronal membranes. At rest, the neuronal membrane possesses a potential (or membrane voltage) of approximately −70 mV, the cell interior being negative with respect to the cell exterior. The potential (voltage) is the result of ion (K+, Na+, Cl−, organic anions) balance across the neuronal semipermeable membrane. Neurotransmitters are stored in presynaptic vesicles and are released under the influence of neuronal action potentials. When released into the synaptic cleft, an excitatory chemical transmitter such as acetylcholine will cause membrane depolarization (change of potential occurs from −70 mV to −50 mV). This effect is mediated by postsynaptic nicotinic receptors which are stimulated by acetylcholine to increase membrane permeability to Na+ ions. The reduced membrane potential stimulates neuronal excitability in the form of a postsynaptic action potential.

In the case of the GABA receptor complex (GRC), the effect on brain excitability is mediated by γ-aminobutyric acid (GABA), a neurotransmitter. GABA has a profound influence on overall brain excitability because up to 40% of the neurons in the brain utilize GABA as a neurotransmitter. GABA regulates the excitability of individual neurons by regulating the conductance of chloride ions across the neuronal membrane. GABA interacts with its recognition site on the GRC to facilitate the flow of chloride ions down an electrochemical gradient of the GRC into the cell. An intracellular increase in the levels of this anion causes hyperpolarization of the transmembrane potential, rendering the neuron less susceptible to excitatory inputs, i.e., reduced neuron excitability. In other words, the higher the chloride ion concentration in the neuron, the lower the brain excitability and level of arousal.

It is well-documented that the GRC is responsible for the mediation of anxiety, seizure activity, and sedation. Thus, GABA and drugs that act like GABA or facilitate the effects of GABA (e.g., the therapeutically useful barbiturates and benzodiazepines (BZs), such as Valium®) produce their therapeutically useful effects by interacting with specific regulatory sites on the GRC. Accumulated evidence has now indicated that in addition to the benzodiazepine and barbiturate binding site, the GRC contains a distinct site for neuroactive steroids. See, e.g., Lan, N. C. et al., Neurochem. Res. (1991) 16:347-356.

Neuroactive steroids can occur endogenously. The most potent endogenous neuroactive steroids are 3α-hydroxy-5-reduced pregnan-20-one and 3α-21-dihydroxy-5-reduced pregnan-20-one, metabolites of hormonal steroids progesterone and deoxycorticosterone, respectively. The ability of these steroid metabolites to alter brain excitability was recognized in 1986 (Majewska, M. D. et al., Science 232:1004-1007 (1986); Harrison, N. L. et al., J. Pharmacol. Exp. Ther. 241:346-353 (1987)).

Compound of Formula (I), a neuroactive steroid described herein, has been shown to be a positive allosteric modulator of GABA_{A} receptors that targets synaptic and extrasynaptic GABA_{A} receptors. As a positive allosteric modulator of GABA_{A} receptors, Compound 1 serves as a therapeutic agent to treat CNS related disorders, e.g., depression (e.g., postpartum depression and major depressive disorder) and anxiety.

There is a need for neuroactive steroids, in particular 19-NOR C3,3-disubstituted C21-N-pyrazolyl steroids, that display desirable physicochemical properties (e.g., polymorphism, melting point, solubility), are convenient to manufacture and exhibit bioavailability profiles that provide sufficient drug exposures to treat diseases.

As such, the development and manufacture of pharmaceutical formulations with the necessary bioavailability, chemical stability and physical stability and material properties required to produce safe and efficacious oral drug products for the treatment of brain and/or CNS-related disease and disorders remains a significant challenge.

Accordingly, there remains an unmet need to develop pharmaceutical compositions comprising neuroactive steroids and methods of manufacturing said pharmaceutical compositions, whereby the pharmaceutical compositions have the efficacy and safety profiles required by regulatory agencies to produce a commercializable drug product.

### SUMMARY OF THE INVENTION

In an aspect, provided herein are pharmaceutical compositions of the compound of formula (I)

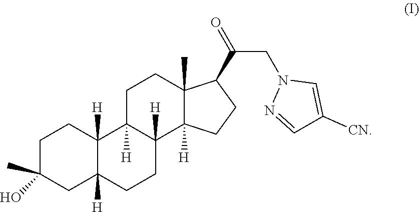

In various embodiments, provided herein are pharmaceutical compositions comprising a plurality of particles of a crystalline form of the compound of formula (I)

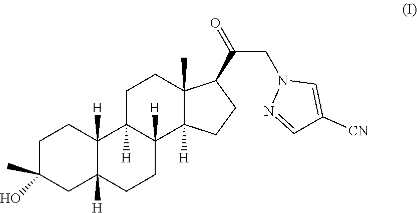

wherein the plurality of particles of the crystalline form of the compound of formula (I) comprises at least one of the following features:
(i) a particle size distribution which is defined by a D₉₀ of about 1 μm to about 100 μm;
(ii) a yield pressure of about 40 MPa to about 200 MPa;
(iii) a strain rate sensitivity of less than about 10%; and
(iv) a contact angle of about 60 degrees to about 110 degrees, wherein the contact angle is measured using a sessile drop technique.

In various embodiments, provided herein are pharmaceutical compositions comprising a crystalline form of the compound of formula (I)

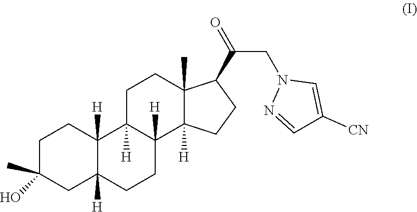

wherein the pharmaceutical composition comprises at least one of the following features:
(i) a true density of about 1.0 g/cc to about 2.5 g/cc;
(ii) a bulk density of about 0.2 g/cc to about 0.8 g/cc and a tapped density of about 0.3 g/cc to about 1.1 g/cc, wherein the tapped density of the pharmaceutical composition is higher than the bulk density;
(iii) a Carr Index of about 10 to about 38;
(iv) about 0.2% to about 90% of the pharmaceutical composition is retained when passed through a 710/25 (microns/mesh) sieve, about 0.2% to about 75% of the material is retained when the pharmaceutical composition is passed through a 425/40 (microns/mesh) sieve, and about 0.1% to about 55% of the pharmaceutical composition is retained when passed through a 63/230 (microns/mesh) sieve;
(v) a solid fraction of about 0.5 to about 0.95;
(vi) a flow rate index (FRI) of about 0.1 kg/sec to about 4 kg/sec; and
(vii) the pharmaceutical composition releases at least about 50% of the compound of formula (I) after about 20 minutes, when tested using a USP 1 or a USP 2 apparatus.

In various embodiments, provided herein are pharmaceutical compositions comprising:
(i) a crystalline form of the compound of formula (I)

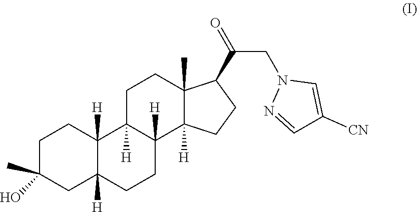

(ii) a filler;
(iii) a lubricant; and
(iv) a glidant.

In various embodiments, provided herein are pharmaceutical compositions comprising:
(i) about 0.4% (w/w) to about 60% (w/w) of a crystalline form of the compound of formula (I)

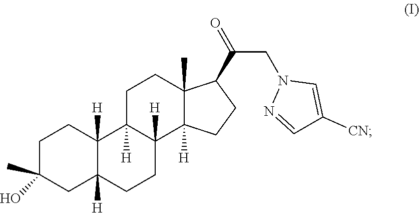

(ii) about 0% (w/w) to about 90% (w/w) of a brittle filler;
(iii) about 0% (w/w) to about 90% (w/w) of a ductile filler;
(iv) about 0% (w/w) to about 15% (w/w) of a disintegrant;
(v) about 0.1% (w/w) to about 5% (w/w) of a lubricant; and
(vi) about 0.1% (w/w) to about 5% (w/w) of a glidant.

In various embodiments, provided herein are pharmaceutical compositions comprising:
(i) about 0.4% (w/w) to about 36% (w/w) of a crystalline form of the compound of formula (I)

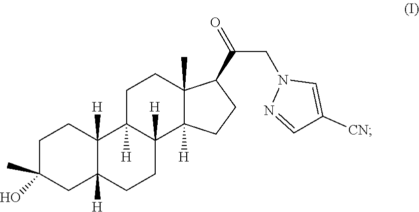

(ii) about 15% (w/w) to about 75% (w/w) of a brittle filler;
(iii) about 10% (w/w) to about 60% (w/w) of a ductile filler;
(iv) about 3% (w/w) to about 12% (w/w) of a disintegrant;
(v) about 0.25% (w/w) to about 5% (w/w) of a glidant; and
(vi) about 0.5% (w/w) to about 3% (w/w) of a lubricant.

In various embodiments, provided herein are pharmaceutical compositions comprising:
(i) about 10% (w/w) to about 15% (w/w) of a crystalline form of the compound of formula (I)

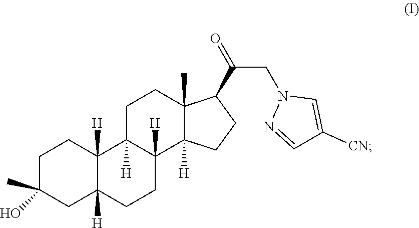

(ii) about 60% (w/w) to about 70% (w/w) of a brittle filler;
(iii) about 10% (w/w) to about 20% (w/w) of a ductile filler;
(iv) about 4% (w/w) to about 8% (w/w) of a disintegrant;
(v) about 0.5% (w/w) to about 2% (w/w) of a glidant; and
(vi) about 1% (w/w) to about 2% (w/w) of a lubricant.

In various embodiments, provided herein are pharmaceutical compositions comprising: (i) about 20 mg of a crystalline form of the compound of formula (I)

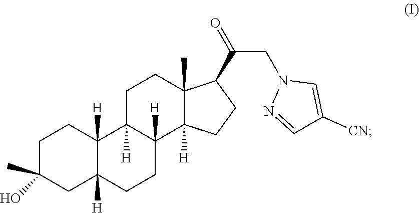

(ii) about 105.9 mg of mannitol;
(iii) about 26.2 mg of silicified microcrystalline cellulose;
(iv) about 10 mg of croscarmellose sodium;
(v) about 1.7 mg of colloidal silicon dioxide; and
(vi) about 2.9 mg of sodium stearyl fumarate.

In various embodiments, provided herein are pharmaceutical compositions comprising:
(i) about 25 mg of a crystalline form of the compound of formula (I)

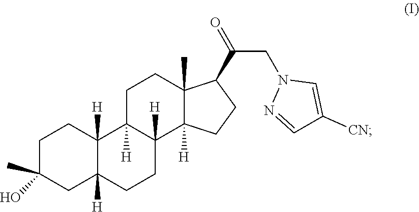

(ii) about 132 mg of mannitol;
(vii) about 32.7 mg of the silicified microcrystalline cellulose;
(viii) about 12.5 mg of croscarmellose sodium;
(ix) about 2.1 mg of colloidal silicon dioxide; and
(x) about 3.6 mg of sodium stearyl fumarate.

In various embodiments, provided herein are pharmaceutical compositions comprising:
(i) about 30 mg of a crystalline form of the compound of formula (I)

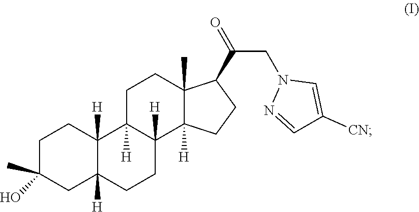

(ii) about 159 mg of mannitol;
(iii) about 39.3 mg of the silicified microcrystalline cellulose;
(iv) about 15 mg of croscarmellose sodium;
(v) about 2.5 mg of colloidal silicon dioxide; and
(vi) about 4.4 mg of sodium stearyl fumarate.

In various embodiments, provided herein are pharmaceutical compositions comprising:
(i) about 40 mg of a crystalline form of the compound of formula (I)

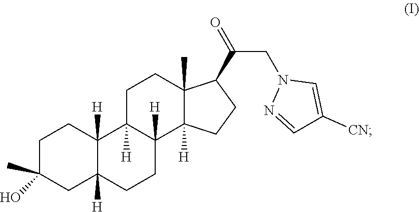

(ii) about 212 mg of mannitol;
(vii) about 52.4 mg of the silicified microcrystalline cellulose;
(viii) about 20 mg of croscarmellose sodium;
(ix) about 3.3 mg of colloidal silicon dioxide; and
(x) about 5.8 mg of sodium stearyl fumarate.

In various embodiments, provided herein are pharmaceutical compositions comprising:
(i) about 50 mg of a crystalline form of the compound of formula (I)

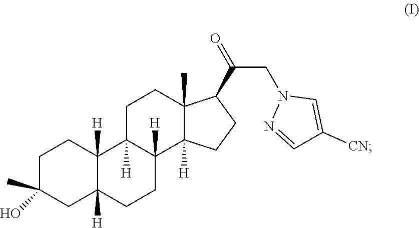

(ii) about 265 mg of mannitol;
(iii) about 65.5 mg of the silicified microcrystalline cellulose;
(iv) about 25 mg of croscarmellose sodium;
(v) about 4.2 mg of colloidal silicon dioxide; and
(vi) about 7.3 mg of sodium stearyl fumarate.

In various embodiments, provided herein are pharmaceutical compositions comprising: (i) about 60 mg of a crystalline form of the compound of formula (I)

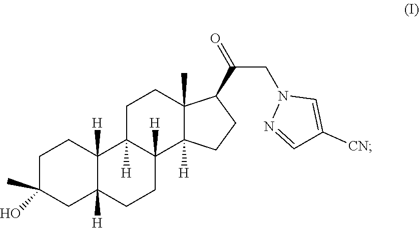

(ii) about 317.7 mg of mannitol;
(iii) about 78.6 mg of the silicified microcrystalline cellulose;
(iv) about 30 mg of croscarmellose sodium;
(v) about 5 mg of colloidal silicon dioxide; and
(vi) about 8.8 mg of sodium stearyl fumarate.

In various embodiments, provided herein are pharmaceutical compositions comprising a plurality of particles of a crystalline form of the compound of formula (I)

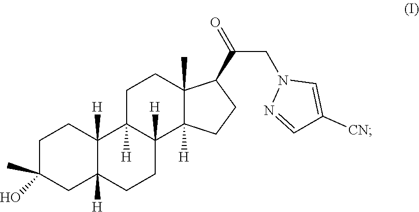

wherein the plurality of particles of the crystalline form of the compound of formula (I) have a particle size distribution which is defined by a D₉₀ of about 1 μm to about 100 μm.

In various embodiments, provided herein are pharmaceutical compositions comprising:
(i) a plurality of particles of a crystalline form of the compound of formula (I)

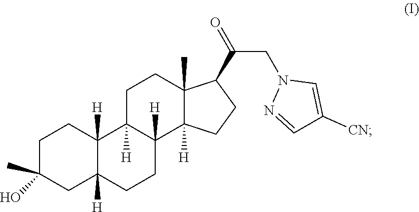

(ii) a filler; and
(vii) one or more pharmaceutically acceptable excipients selected from the group consisting of a disintegrant, a binder, a wetting agent, a lubricant, a glidant, and combinations thereof, wherein the plurality of particles of the crystalline form of the compound of formula (I) has a particle size distribution which is defined by a D₉₀ of about 1 μm to about 20 μm.

In various embodiments, provided herein are pharmaceutical compositions comprising:
(i) a crystalline form of the compound of formula (I)

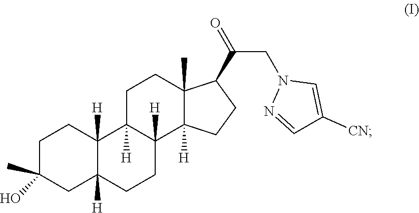

(ii) a filler; and
(vii) one or more pharmaceutically acceptable excipients selected from the group consisting of a disintegrant, a binder, a wetting agent, a lubricant, a glidant, and combinations thereof,
   wherein the pharmaceutical composition has a bulk density of about 0.2 g/cc to about 0.8 g/cc and a tapped density of about 0.3 g/cc to about 1.1 g/cc and wherein the tapped density of the pharmaceutical composition is higher than the bulk density.

In various embodiments, provided herein are pharmaceutical compositions comprising:
(i) a crystalline form of the compound of formula (I)

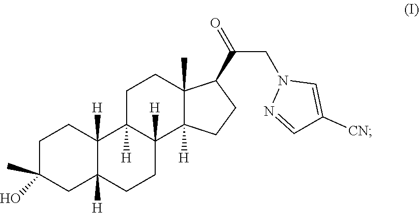

(ii) a filler; and
(iii) one or more pharmaceutically acceptable excipients selected from the group consisting of a disintegrant, a binder, a wetting agent, a lubricant, a glidant, and combinations thereof,
   wherein the pharmaceutical composition has an average flow rate index (FRI) of about 0.05 to about 3.1 kg/sec.

In various embodiments, provided herein are pharmaceutical compositions comprising:
(i) a crystalline form of the compound of formula (I)

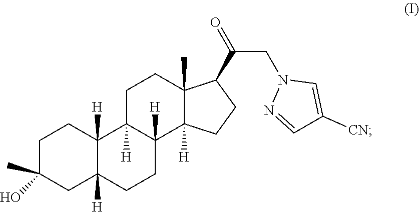

(ii) a filler; and
(iii) one or more pharmaceutically acceptable excipients selected from the group consisting of a disintegrant, a binder, a wetting agent, a lubricant, a glidant, and combinations thereof,
   wherein the pharmaceutical composition releases at least about 50% of the compound of formula (I) after about 20 minutes, when tested using a USP 1 or a USP 2 apparatus.

In various embodiments, provided herein are pharmaceutical compositions comprising:
(i) a crystalline form of the compound of formula (I)

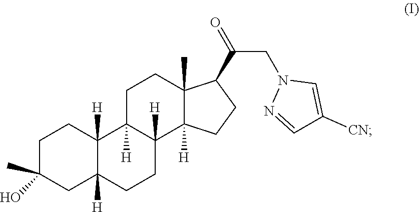

(ii) a filler; and
(iii) one or more pharmaceutically acceptable excipients selected from the group consisting of a disintegrant, a binder, a wetting agent, a lubricant, a glidant, and combinations thereof;
   wherein the pharmaceutical composition exhibits the following dissolution profile:
at least about 70% of the compound of formula (I) is released after about 20 minutes; and
at least about 80% of the compound of formula (I) is released after about 30 minutes, when tested in about 500 mL to about 900 mL of 50 mM sodium phosphate buffer, pH 6.8 with about 0.2% to about 0.6% SDS in a USP 2 apparatus at about 37° C.

In another aspect, provided herein are dosage forms intended for oral administration comprising a pharmaceutical composition described herein.

In another aspect, provided herein are methods of making a pharmaceutical composition described herein.

In various embodiments, provided herein is a process for making a pharmaceutical composition comprising:
(a) micronizing a crystalline form of the compound of formula (I) to obtain a micronized crystalline form of the compound of formula (I)

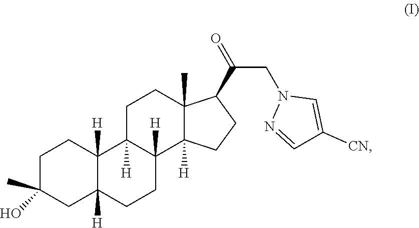

wherein the micronized crystalline form of the compound of formula (I) has a particle size distribution which is defined by a D₉₀ of about 1 μm to about 100 μm;
(b) blending the micronized crystalline form of the compound of formula (I) with one or more pharmaceutically acceptable excipients to obtain a blend;
(c) granulating the blend to obtain granules;
(d) milling the granules to obtain an intragranular phase; and
(e) blending the intragranular phase with one or more extragranular pharmaceutical excipients to obtain the pharmaceutical composition.

In another aspect, provided herein are pharmaceutical compositions comprising the compound of formula (I) useful for the treatment of the various conditions, diseases, and disorders described herein. In various embodiments, the condition, disease, or disorder is insomnia. In various embodiments, the condition, disease, or disorder is major depressive disorder. In various embodiments, the condition, disease, or disorder is bipolar disorder. In various embodiments, the condition, disease, or disorder is insomnia. In various embodiments, the condition, disease, or disorder is postpartum depression. In various embodiments, the condition, disease, or disorder is anxiety. In various embodiments, the condition, disease, or disorder is treatment-resistant depression.

### BRIEF DESCRIPTION OF THE DRAWINGS

FIG. 1A is an exemplary X-ray powder diffraction pattern of crystalline Form A of Compound 1.

FIG. 1B shows exemplary thermogravimetric analysis (upper) and differential scanning calorimetry (lower) curves for crystalline Form A of Compound 1.

FIG. 2A is an exemplary X-ray powder diffraction pattern of crystalline Form C of Compound 1.

FIG. 2B shows exemplary thermogravimetric analysis (upper) and differential scanning calorimetry (lower) curves for crystalline Form C of Compound 1.

FIG. 3 depicts a Jet Mill clogged with particles of crystalline Form A of Compound 1.

FIG. 4A is an exemplary particle size distribution for particles of micronized crystalline Form C of Compound 1.

FIG. 4B is an exemplary particle size distribution for particles of micronized crystalline Form C of Compound 1.

FIG. 4C is an exemplary particle size distribution for particles of micronized crystalline Form C of Compound 1.

FIG. 5 is an exemplary particle size distribution for particles of micronized crystalline Form A of Compound 1.

FIG. 6 is an overlay of the pharmacokinetic profiles of un-micronized particles of crystalline Form C of Compound 1 and micronized particles of crystalline Form C of Compound 1 dosed orally to Male Sprague Dawley Rats.

FIG. 7 is an exemplary flow diagram of the invention for the preparation of capsules described herein using the direct blend process.

FIG. 8 is an overlay of the pharmacokinetic profiles of three direct blend hand-filled 5 mg capsule formulations, as described in Example 21, dosed orally to dogs.

FIG. 9 is an exemplary flow diagram of the invention for the preparation of capsules described herein using the dry granulation process.

FIG. 10 is an overlay of dissolution data collected for exemplary hand-filled capsules (30 mg dose strength) comprising varying amounts of crystalline Form A of Compound 1 to crystalline Form C of Compound 1, as further described in Example 33.

FIG. 11 is an overlay of dissolution data collected for exemplary hand-filled capsules, each comprising 30 mg of crystalline Form C of Compound 1 with varying particle size distributions, as further described in Example 34.

FIG. 12A is an exemplary DSC thermogram for micronized Form C of Compound 1a.

FIG. 12B is an exemplary DSC thermogram for micronized Form C of Compound 1b.

FIG. 12C is an exemplary DSC thermogram for micronized Form C of Compound 1c.

FIG. 12D is an exemplary DSC thermogram for micronized Form C of Compound 1d.

FIG. 13A is an exemplary particle size distribution for particles of micronized crystalline Form C of Compound 1a.

FIG. 13B is an exemplary particle size distribution for particles of un-micronized crystalline Form C of Compound 1a.

FIG. 14A is an exemplary particle size distribution for particles of micronized crystalline Form C of Compound 1b.

FIG. 14B is an exemplary particle size distribution for particles of un-micronized crystalline Form C of Compound 1b.

FIG. 15A is an exemplary particle size distribution for particles of micronized crystalline Form C of Compound 1c.

FIG. 15B is an exemplary particle size distribution for particles of un-micronized crystalline Form C of Compound 1c.

FIG. 16A is an exemplary particle size distribution for particles of micronized crystalline Form C of Compound 1d.

FIG. 16B is an exemplary particle size distribution for particles of un-micronized crystalline Form C of Compound 1d.

### DETAILED DESCRIPTION

As generally described herein, the present disclosure provides pharmaceutical compositions containing a neuroactive steroid e.g., a compound of formula (I):

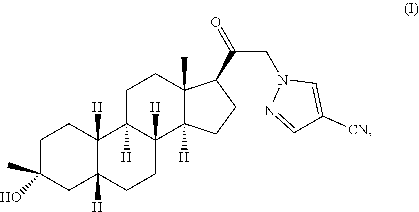

methods of making the pharmaceutical compositions, and methods of using the pharmaceutical compositions to treat medical conditions, diseases, and disorders e.g., a central nervous system-related disorder.

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred from the context.

At various places in the present specification, variables or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

As used herein, “XRPD” refers to X-ray powder diffraction. An XRPD pattern is an x-y graph with 2θ (diffraction angle) plotted on the x-axis and intensity plotted on the y-axis. These are the diffraction peaks which may be used to characterize a crystalline material. The diffraction peaks are usually represented and referred to by their position on the x-axis rather than the intensity of the diffraction peaks on the y-axis because diffraction peak intensity can be particularly sensitive to sample orientation (see Pharmaceutical Analysis, Lee & Web, pp. 255-257 (2003)). Thus, intensity is not typically used by those of skill in the art to characterize a crystalline material. As with any data measurement, there may be variability in XRPD data. In addition to the variability in diffraction peak intensity, there may also be variability in the position of the diffraction peaks on the x-axis. This variability can, however, typically be accounted for when reporting the positions of diffraction peaks for purposes of characterization. Such variability in the position of diffraction peaks along the x-axis may be derived from several sources. One such source can be sample preparation. Samples of the same crystalline material prepared under different conditions may yield slightly different diffractograms. Factors such as particle size, moisture content, solvent content, temperature, and orientation may all affect how a sample diffracts X-rays. Another source of variability comes from instrument parameters. Different X-ray powder diffractometers operate using different parameters and may lead to slightly different diffraction patterns from the same crystalline material. Likewise, different software packages process XRPD data differently and this may also lead to variability. These and other sources of variability are known to those of ordinary skill in the art. Due to such sources of variability, the values of each X-ray diffraction peak may be preceded with the term “about” or proceeded with an appropriate range defining the experimental variability (e.g., ±0.1°, ±0.2°, ±0.3°, ±0.4°, ±0.5°, etc.).

The term “characteristic peaks” when referring to the peaks in an XRPD pattern of a crystalline form of a given chemical entity (e.g., a crystalline form of a compound of formula (I)) refers to a collection of specific diffraction peaks whose values span a range of 2θ values (e.g., 0°-40°) that are, as a whole, unique to that specific crystalline form.

As used herein, “crystalline” refers to a solid phase of a given chemical entity having well-defined 3-dimensional structural order. The atoms, ions, and/or molecules are arranged in a regular, periodic manner within a repeating 3-dimensional lattice. In various embodiments, a crystalline material may comprise one or more discreet crystalline forms.

As used herein, the terms “crystalline form”, “crystalline solid form,” “crystal form,” “solid form,” and related terms herein refer to crystalline modifications comprising a given substance (e.g., the compound of formula (I)), including single-component crystal forms and multiple-component crystal forms, and including, but not limited to, polymorphs, solvates, hydrates, and salts.

The term “substantially crystalline” refers to solid forms that may be at least a particular weight percent crystalline. Particular weight percentages may include 70%, 75%, 80%, 85%, 87%, 88%, 89%, 90%, 91%, 92%, 93%, 94%, 95%, 96%, 97%, 98%, 99%, 99.5%, 99.9%, or any percentage between 70% and 100%. In certain embodiments, the particular weight percent of crystallinity is at least 90%. In certain other embodiments, the particular weight percent of crystallinity is at least 95%. In some embodiments, the compound of formula (I) can be a substantially crystalline sample of any of the crystalline solid forms described herein (e.g., crystalline Forms A and C).

The term “substantially pure” relates to the composition of a specific crystalline solid form (e.g., a crystalline form of the compound of formula (I)) that may be at least a particular weight percent free of impurities and/or other solid forms. Particular weight percentages may include 70%, 75%, 80%, 85%, 90%, 95%, 99%, or any percentage between 70% and 100%. In certain embodiments, the compound of formula (I) can be a substantially pure sample of any of the crystalline solid forms described herein, (e.g., crystalline Forms A and C). In certain embodiments, the compound of formula (I) can be substantially pure Form A. In certain embodiments, the compound of formula (I) can be substantially pure Form C.

As used herein, the term “anhydrous” or “anhydrate” when referring to a crystalline form (e.g., a crystalline form of the compound of formula (I)) means that no water molecules form a portion of the unit cell of the crystalline form. An anhydrous crystalline form may nonetheless contain water molecules that do not form part of the unit cell of the anhydrous crystalline form (e.g., as residual solvent molecule left behind from the production of the crystalline form). In a preferred embodiment, water can make up about 0.5% by weight of the total composition of a sample of an anhydrous form. In a more preferred embodiment, water can make up about 0.2% by weight of the total composition of a sample of an anhydrous form. In some embodiments, a sample of an anhydrous crystalline form of the compound of formula (I) contains no water molecules, e.g., no detectable amount of water.

As used herein, the term “desolvated” or “unsolvated” when referring to a crystalline form (e.g., a crystalline form of the compound of formula (I)) means that no solvent molecules form a portion of the unit cell of the crystalline form. An unsolvated crystalline form may nonetheless contain solvent molecules that do not form part of the unit cell of the unsolvated crystalline form (e.g., as residual solvent molecule left behind from the production of the crystalline form). In a preferred embodiment, the solvent can make up 0.5% by weight of the total composition of a sample of an unsolvated form. In a more preferred embodiment, solvent can make up 0.2% by weight of the total composition of a sample of an unsolvated form. In some embodiments, a sample of an unsolvated crystalline form of the compound of formula (I) contains no solvent molecules, e.g., no detectable amount of solvent.

As used herein, the terms “polymorph,” “polymorphic form,” “polymorphs,” “polymorphic forms” and related terms herein refer to two or more crystal forms that consist essentially of the same molecule, molecules, or ions (e.g., the compound of formula (I)). Different polymorphs may exhibit different physicochemical properties including, but not limited to, melting temperatures, solubilities, dissolution rates, and physical stabilities as a result of differences in the arrangement or conformation of the molecules or ions in the crystal lattice.

The term “solvate” when referring to a crystalline form of the compound of formula (I) means that solvent molecules (e.g., organic solvents and water), form a portion of the unit cell of the crystalline form. Solvates that contain water as the solvent are also referred to herein as “hydrates.”

As used herein, the term “particle size” or “particle size distribution (PSD)” when referring to the compound of formula (I) is a list of values that defines the relative amount, typically by mass or volume, of particles present according to size. The distribution data may be reported as cumulative distributions and/or as density distributions by volume or mass such as D(v, 0.1), D(v, 0.5) and D(v, 0.9). In various embodiments, the particle size of a compound of formula (I) may be measured using laser diffraction techniques (e.g., dispersing particles in a sample and illuminating the sample in a collimated laser-beam by scattering light over a range of angles). The large particles generate a high scattering intensity at relatively narrow angles to the incident beam, while the smaller particles produce a lower intensity signal but at much wider angles. Using an array of detectors, laser-diffraction analyzers record the pattern of scattered light produced by the sample. With the application of an appropriate model of light behavior the particle-size distribution of the sample can be determined from the scattering data, via a deconvolution step.

As used herein, “yield pressure” may refer to, for example, the pressure at which a material (e.g., the compound of formula (I)) begins to deform irreversibly (plastically or by brittle fracture) or the pressure at which the material exhibits nonlinear (the sum of elastic plus irreversible) deformation. Yield pressure is the reciprocal of the slope of a Heckel plot of the ln(1/(1−D)) vs P where D is the tablet relative compact density, P is the applied compression pressure, and the slope is taken at the pressure where plastic flow first occurs. A very low yield pressure of 50 MPa is typical for Avicel PH 101 (MCC), which is ductile, and a really high yield pressure of over 959 MPa is typical of dicalcium phosphate, DCP, which is extremely brittle (Pharmaceutical Powder Compaction Technology, 2nd Edition, Volume 197, Drugs and the Pharmaceutical Sciences, Edited by Metin Çelik, ©2011 by CRC Press).

As used herein, “strain rate” refers to the change in strain of a material (i.e., the rate at which a material expands or shrinks and also the rate at which it deforms with progressive shearing without a change in the volume of the material), for example the compound of formula (I), as a function of time.

As used herein, “contact angle” refers to the angle formed by a liquid at the three-phase boundary where a solid (e.g., the compound of formula (I)), a liquid and a vapor intersect. Contact angle measurements may be used to quantify the wettability of a solid surface (e.g., the smooth surfaces of a compressed tablet prepared using a crystalline form of the compound of formula (I)) by a given liquid or solution. The contact angle may be measured, for example, using a sessile drop technique. Other methods are routinely used and found in textbooks on surface chemistry (J T Davies and E K Rideal, “Interfacial Phenomena”, Academic Press, 1963 or A. W. Adamson and A P Gast, “The Physical Chemistry of Surfaces,” Wiley, 1997) such as the ring method, the drop-weight method, the Wilhemy plate method, the pendant drop method, the ripple method, the capillary rise method, and the maximum bubble pressure method. The measurements reported in this application use the sessile drop method.

As used herein, “true density” or “particle density” refers to the measurement of volume excluding the porous area both within and among particles (e.g., a powder, a particulate solid). It is typically measured with a gas pycnometer, such as the Accupyc, using helium as the gas to allow permeation of the smallest pores.

As used herein, “bulk density” refers to the mass of particles per unit of total volume that the particles occupy. The total volume includes, for example, particle volume, inter-particle void volume, and internal pore volume.

As used herein, “tapped density”, “volumetric density” or “apparent density” refers to the increased bulk density of a powdered or particulate material (e.g., a pharmaceutical composition described herein) after mechanically “tapping” the container in which the powdered or particulate material resides.

As used herein, “Can Index” or “Carr Compressibility Index” is a measure of the propensity of a powder or particulate material (e.g., a pharmaceutical composition described herein) to be compressed and is a function of both the bulk and tapped densities of the material.

As used herein, “Flow Rate Index (FRI)” may be defined as the rate at which a solid will flow through a given hopper outlet diameter when totally de-aerated. Low values of FRI usually indicate fine, highly compressible powders. Particles of sizes in excess of 400 μm are usually incompressible, very permeable, and have a high FRI. Variations in the value of the index may be a signal of segregation or changes in composition of powder mixtures during processing. FRI can be measured with a Johanson Flow Rate Indicizer system.

As used herein, “envelope density” refers to the volume of the particles including the pores or spaces between the particles and is measured by flowing particles, such as the Geopyc, with rigid spheres around the sample establishing a controlled dry flow.

As used herein, “solid fraction” refers to the ratio of envelope density to true density, Solid fraction is critical to understanding and controlling roller compaction or compression processes, and is typically measured for the ribbons, granules and/or final powder blend prior to encapsulation or tableting. It is also measured on the uncoated tablets.

As used herein, “dissolution profile” refers to dissolution testing of a drug substance or drug product at multiple time points. Dissolution profiles for drug substances (e.g., the compound of formula (I)) or drug products (e.g., the pharmaceutical compositions described herein) may be performed for characterization and quality control to ensure the drug is released at a defined range of rates in a well-defined dissolution aqueous media that is at least sink conditions for that drug, or in biorelevant media such as simulated gastric or intestinal fluids representing either the fasted or fed states. In certain cases, but not others, dissolution testing may be predictive of or give insight into in vivo bioavailability of the drug substance. Dissolution testing may be performed using USP testing protocols and dissolution apparatus.

As used herein, “granulation” refers to a process of forming granules from a powdered or particulate material. As used herein, “Dry granulation” refers to a process in which granules are formed without the presence of a liquid solution and may be useful in the preparation of granules of materials sensitive to heat, moisture, or solvents. For example, roller compaction is a dry granulation process. As used herein, “Wet granulation” refers to the formation of granules wherein the particles are bound together using a binder or a liquid solution. Examples of wet granulation are high shear granulation and fluid bed granulation.

As used herein, “pharmaceutical composition” or “pharmaceutical formulation” refer to the combination of a therapeutically active agent with a pharmaceutically acceptable excipient, inert or active, making the composition especially suitable for diagnostic or therapeutic use in vivo or ex vivo.

“Pharmaceutically acceptable” refers to compounds, molecular entities, compositions, materials and/or dosage forms that do not produce an adverse, allergic or other untoward reaction when administered to an animal, or human, as appropriate; or means approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that may be present in a compound of the present invention (e.g., the compound of formula (I)), which salt is compatible with pharmaceutical administration.

As is known to those of skill in the art, “salts” of compounds may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW₄⁺, wherein W is C₁₋₄ alkyl, and the like.

Examples of salts include, but are not limited, to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, K⁺, Ca²⁺, NH₄⁺, and NW₄⁺ (where W can be a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions of the present invention without causing a significant adverse toxicological effect on the patient. Non-limiting examples of pharmaceutically acceptable excipients include, binders, diluents or fillers (e.g., brittle diluents or fillers and ductile diluents or fillers), disintegrants, lubricants, coatings, sweeteners, flavors, gelatins, carbohydrates such as lactose, amylose or starch, fatty acid esters, hydroxypropylmethylcellulose, polyvinyl pyrrolidine, and colors, and the like. For examples of excipients, see Martin, Remington's Pharmaceutical Sciences, 15th Ed., Mack Publ. Co., Easton, Pa. (1975) or Rowe, Shesky, and Quinn, Handbook of Pharmaceutical Excipients, 6^{th} Ed. Pharmaceutical Press, London, UK (2009).

Examples of diluents or fillers include, but are not limited to, a sugar (e.g., mannitol, lactose, sorbitol, lactitol, erythritol, sucrose, fructose, glucose, agarose, maltose, isomalt, polydextrose, and combinations thereof), an inorganic material (e.g., dibasic calcium phosphate, hydroxyapatite, sodium carbonate, sodium bicarbonate, calcium carbonate, calcium sulfate, magnesium carbonate, magnesium oxide, bentonite, kaolin), calcium lactate, a starch (e.g., a pregelatinized starch), a microcrystalline cellulose, a silicified microcrystalline cellulose, a polysaccharide, a cellulose (e.g., a hydroxypropylcellulose, a hypromellose, a carboxymethylcellulose, a methylcellulose, a hydroxypropylmethylcellulose, a hydroxyethylcellulose), a dextrin, a maltodextrin, an alginate, a collagen, a polyvinylpyrrolidone, a polyvinylacrylate, polyethylene oxide, and polyethylene glycol. Sugar is defined herein to include sugar alcohols.

Examples of disintegrants include, but are not limited to, alginic acid, an alginate, primogel, a cellulose (e.g., hydroxypropylcellulose), polacrillin potassium, sodium starch glycolate, sodium croscarmellose, a polyplasdone (e.g., a crospovidone), and a starch (e.g., corn starch, pregelatinized starch, hydroxypropyl starch, and carboxymethyl starch).

Examples of binders include, but are not limited to, a hydroxypropylcellulose, hydroxyethylcellulose, a hydroxypropylmethylcellulose (e.g., a low viscosity hydroxypropylmethylcellulose), a sugar, a polyvinylpyrrolidone, a polyvinyl alcohol, a polyvinyl acetate, a polydextrose, a chitosan, a carrageenan, carbophil, a microcrystalline cellulose, gum tragacanth, guar gum, gellan gum, gelatin, and a starch (e.g., corn starch).

Examples of wetting agents include, but are not limited to, a poloxamer (e.g., poloxamer 407), sodium dodecyl sulfate, sodium lauryl sulfate (SLS), sodium stearyl fumarate (SSF), a polydimethylsiloxane, a polysorbate (e.g., polyoxyethylene 20 sorbitan mono-oleate (Tween® 20)), sorbitan monooleate, sorbitan trioleate, sorbitan laurate, sorbitan stearate, sorbitan monopalmitate, lecithin, sodium taurocholate, ursodeoxycholate, polyethoxylated castor oil, cetyl trimethylammonium bromide, nonoxynol, \hat{α}-tocopherol polyethylene glycol 1000 succinate, and docusate sodium.

Examples of lubricants and glidants include, but are not limited to, a wax, a glyceride, a light mineral oil, a polyethylene glycol, sodium stearyl fumarate, magnesium stearate, stearic acid, hydrogenated oil (e.g., hydrogenated vegetable oil), an alkyl sulfate, sodium benzoate, sodium acetate, glyceryl behenate, palmitic acid, and coconut oil.

Examples of glidants include, but are not limited to, colloidal silicon dioxide, talc, kaolin, bentonite, and activated carbon/charcoal.

Examples of colorants include, but are not limited to, titanium dioxide, aluminum lakes, iron oxides and carbon black.

Examples of coatings, include but are not limited to, a film forming polymer (e.g., a hypromellose, a methyl cellulose, an ethylcellulose, cellulose acetate, a hydroxypropylmethyl cellulose, a hydroxypropyl cellulose, hydroxypropylmethyl cellulose acetate succinate, cellulose acetate phthalate, a polyvinylpyrrolidone, polyvinyl alcohol, a Eudragit/acrylate) and a plasticizer (e.g., triacetin, polyethylene glycol, propylene glycol).

Pharmaceutical compositions for oral administration (e.g., pharmaceutical compositions of the compound of formula (I) described herein) can take the form of bulk liquid solutions or suspensions or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pills, tablets, capsules or the like in the case of solid compositions.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a nonhuman animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

As used herein, “solid dosage form” means a pharmaceutical dose(s) in solid form, e.g., tablets, capsules, granules, powders, minitabs, sachets, stickpacks, reconstitutable powders, dry powder inhalers, lozenges, and chewables.

As used herein, “administering” means oral administration, administration as a pulmonary, suppository, intramuscular administration, intrathecal administration, intranasal administration or subcutaneous administration, or the implantation of a slow-release device, e.g., a mini-osmotic pump, to a subject. Administration is by any route, including transmucosal (e.g., buccal, sublingual, palatal, gingival, nasal, vaginal, rectal, or). Parenteral administration includes, e.g., intramuscular and subcutaneous. Other modes of delivery include, but are not limited to, the use of liposomal formulations, etc. By “co-administer” it is meant that a composition described herein is administered at the same time, just prior to, or just after the administration of one or more additional therapies (e.g., anti-cancer agent, chemotherapeutic, or treatment for a neurodegenerative disease). The compound of formula (I), can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (e.g., “therapeutic treatment”).

In general, an “effective amount” of a compound refers to an amount sufficient to elicit the desired biological response, e.g., to treat a disease or disorder of the brain and/or central nervous system. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In an alternate embodiment, the present invention contemplates administration of the compounds of the present invention or a pharmaceutically acceptable salt or a pharmaceutically acceptable composition thereof, as a prophylactic before a subject begins to suffer from the specified disease, disorder or condition. As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, an “episodic dosing regimen” is a dosing regimen wherein a compound of formula (I) or a composition comprising a compound of formula (I) is administered to a subject for a finite period of time in response to the diagnosis of a disorder or symptom thereof, e.g., a diagnosis or symptom of depression, an episode of major depressive disorder, bipolar depression, anxiety, or postpartum depression. In some embodiments, the major depressive disorder is moderate major depressive disorder. In some embodiments, the major depressive disorder is severe major depressive disorder. In some embodiments, the compound is formulated as individual dosage units, each unit comprising a compound of formula (I) and one or more suitable pharmaceutical excipients. In some embodiments, the episodic dosing regimen has a duration of a plurality of weeks, e.g. about 8 weeks. In contrast with chronic administration as defined herein, episodic dosing of a compound occurs over a finite period of time, e.g., from about 2 weeks to about 8 weeks, in response to a diagnosis of a disorder, e.g., depression, or a symptom thereof. In some embodiments, episodic dosing occurs once per day across a plurality of weeks, e.g., from about 2 weeks to about 6 weeks. In one embodiment, the episodic dosing has a duration of two weeks. In some embodiments, more than one episodic dosing regimen is administered to the subject, e.g., two or more episodic regimens throughout the subject's life.

### Compound

The present invention describes a compound of formula (I)

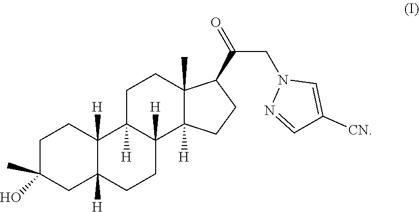

A method of chemically synthesizing the compound of formula (I) (including Example 1 provided herein), which may also be referred to herein as Compound 1, was described in U.S. Pat. No. 9,725,481 and PCT Application Publication No. WO 2014169831, which are incorporated by reference in their entirety herein. Several crystalline forms of the compound of formula (I) (including the crystalline forms A and C described herein) and methods of preparing said forms were described in U.S. Patent Application Publication No. US 20190177359 and PCT Application Publication No. WO 2018039378, which are incorporated by reference in their entirety herein.

The compound of formula (I) has a solubility in water of approximately 0.8-3 μg/mL and has similar solubility in pH 1.2 simulated gastric fluid. The solubility in fasted simulated intestinal fluid (FaSSIF) mirrors the solubility in these media and the solubility of the compound of formula (I) is considered to be practically insoluble in aqueous media. The compound of formula (I) belongs in the high permeability, poor solubility drug classification (BCS 2) described in the Biopharmaceutics Classification System (BCS). In the improved widely accepted Developability Classification System (DCS) discussed by Butler and Dressman, the high permeability low solubility category is further divided into two classifications. Specifically, the ratio of the dose to the solubility of the drug in FaSSIF can be used to predict the extent of human absorption. Class 2a describes compounds that have good permeability and poor solubility with a smaller dose to solubility ratio. Class 2b describes compounds that have a higher dose to solubility ratio and likely will be incompletely absorbed unless the drug is formulated in an already solubilized form, and therefore might present a major challenge to the formulator. This ratio of the dose to solubility in FaSSIF is greater than about 10,000 for a 10 mg dose, 20,000 for a 20 mg dose, 30,000 for a 30 mg dose, and similarly through 90,000 for a 90 mg dose and 100,000 for a 100 mg dose. That is, even for a dose of 10 mg about only 1.2% of the dose of the drug may dissolve in a glass of water or FaSSIF. For Class 2b compounds the system predicts that particle reduction should have no impact on bioavailability. Martinez and Amidon offer: “In the case of solubility-limited absorption particle size exerts minimal effect on the fraction of drug absorbed. In this situation, fraction of drug absorbed can only be improved by enhancing drug solubility (e.g., via inclusion of surfactants in the drug formulation.) Conversely particle size exerts its greatest effect when solubility is not a problem.” (Martinez and Amidon; J. Clin. Pharmacol.; 2002; 42; 620-643). In light of the very large solubility to dose ratio for the compound of formula (I), oral absorption of the compound of formula (I) is solubility limited, and reduction of particle size should not affect its bioavailability. Surprisingly though, as described herein, a reduction in the particle size of the compound of formula (I) results in an increase in bioavailability.

In an aspect, provided herein is a compound of formula (I), or a pharmaceutically acceptable salt thereof, for the preparation of pharmaceutical compositions for use in the treatment of a variety of diseases and disorders in the brain and/or central nervous system in a patient in need thereof.

In certain embodiments, the compound of formula (I), or pharmaceutically acceptable salt thereof, is a crystalline form of the compound of formula (I), or pharmaceutically acceptable salt thereof. In certain embodiments, the crystalline form of the compound of formula (I), or pharmaceutically acceptable salt thereof is any crystalline form disclosed in PCT Application Publication No. WO 2018039378.

In certain embodiments, the crystalline form of the compound of formula (I) is anhydrous crystalline Form A. In certain embodiments, Form A has an XRPD pattern with characteristic peaks between and including the following values of 2θ in degrees: 9.3 to 9.7 (e.g., 9.5±0.2), 10.6 to 11.0 (e.g., 10.8±0.2), 13.0 to 13.4 (e.g., 13.2±0.2), 14.7 to 15.1 (e.g., 14.9±0.2), 15.8 to 16.2 (e.g., 16.0±0.2), 18.1 to 18.5 (e.g., 18.3±0.2), 18.7 to 19.1 (e.g., 18.9±0.2), 20.9 to 21.3 (e.g., 21.1±0.2), 21.4 to 21.8 (e.g., 21.6±0.2), and 23.3 to 23.7 (e.g., 23.5±0.2). In certain embodiments, Form A has an XRPD pattern with characteristic peaks between and including the following values of 2θ in degrees: 9.3 to 9.7 (e.g., 9.5±0.2), 10.6 to 11.0 (e.g., 10.8±0.2), 13.0 to 13.4 (e.g., 13.2±0.2), 18.7 to 19.1 (e.g., 18.9±0.2), and 21.4 to 21.8 (e.g., 21.6±0.2). In certain embodiments, Form A has an XRPD pattern with characteristic peaks at the following values of 2θ in degrees: 9.5±0.2, 10.8±0.2, 13.2±0.2, 14.9±0.2, 16.0±0.2, 18.3±0.2, 18.9±0.2, 21.1±0.2, 21.6±0.2, and 23.5±0.2. In certain embodiments, the X-ray powder diffraction pattern for Form A may comprise one, two, three, four, five, six, seven, eight, nine, or ten characteristic peaks, in terms of 20, selected from the peaks at 9.5±0.2, 10.8±0.2, 13.2±0.2, 14.9±0.2, 16.0±0.2, 18.3±0.2, 18.9±0.2, 21.1±0.2, 21.6±0.2, and 23.5±0.2. In certain embodiments, Form A has an XRPD pattern with characteristic peaks at the following values of 2θ in degrees: 9.5±0.2, 10.8±0.2, 13.2±0.2, 18.9±0.2, and 21.6±0.2.

In certain embodiments, the crystalline form of the compound of formula (I) is anhydrous crystalline Form C. In certain embodiments, Form C can have an XRPD pattern with characteristic peaks between and including the following values of 2θ in degrees: 9.7 to 10.1 (e.g., 9.9±0.2), 11.6 to 12.0 (e.g., 11.8±0.2), 13.2 to 13.6 (e.g., 13.4±0.2), 14.2 to 14.6 (e.g., 14.4±0.2), 14.6 to 15.0 (e.g., 14.8±0.2), 16.8 to 17.2 (e.g., 17.0±0.2), 20.5 to 20.9 (e.g., 20.7±0.2), 21.3 to 21.7 (e.g., 21.5±0.2), 21.4 to 21.8 (e.g., 21.6±0.2), and 22.4 to 22.8 (e.g., 22.6±0.2). In certain embodiments, Form C can have an XRPD pattern with characteristic peaks between and including the following values of 2θ in degrees: 9.7 to 10.1 (e.g., 9.9±0.2), 14.6 to 15.0 (e.g., 14.8±0.2), 16.8 to 17.2 (e.g., 17.0±0.2), 20.5 to 20.9 (e.g., 20.7±0.2), and 21.3 to 21.7 (e.g., 21.5±0.2). In certain embodiments, Form C can have an XRPD pattern with characteristic peaks at the following values of 2θ in degrees: 9.9±0.2, 11.8±0.2, 13.4±0.2, 14.4±0.2, 14.8±0.2, 17.0±0.2, 20.7±0.2, 21.5±0.2, 21.6±0.2, and 22.6±0.2. In certain embodiments, the X-ray powder diffraction pattern for Form C may comprise one, two, three, four, five, six, seven, eight, nine, or ten characteristic peaks, in terms of 20, selected from the peaks at 9.9±0.2, 11.8±0.2, 13.4±0.2, 14.4±0.2, 14.8±0.2, 17.0±0.2, 20.7±0.2, 21.5±0.2, 21.6±0.2, and 22.6±0.2. In certain embodiments, Form C can have an XRPD pattern with characteristic peaks at the following values of 2θ in degrees: 9.9±0.2, 14.8±0.2, 17.0±0.2, 20.7±0.2, and 21.5±0.2.

In certain embodiments, the crystalline form of the compound of formula (I) comprises a mixture of two or more crystalline forms. In certain embodiments, the crystalline form of the compound of formula (I) comprises anhydrous crystalline Form A and anhydrous crystalline Form C.

### Pharmaceutical Compositions

As described herein, in an aspect, the invention provides pharmaceutical compositions of the compound of formula (I), or a pharmaceutically acceptable salt thereof.

In various embodiments, the pharmaceutical composition comprises a plurality of particles of a crystalline form of the compound of formula (I)

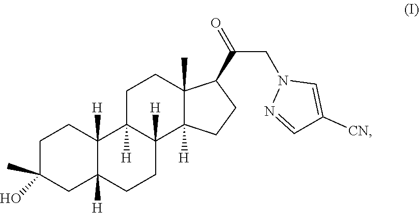

wherein the plurality of particles of the crystalline form of the compound of formula (I) comprises at least one of the following features:
(i) a particle size distribution which is defined by a D₉₀ of about 1 μm to about 100 μm;
(ii) a yield pressure of about 40 MPa to about 200 MPa;
(iii) a strain rate sensitivity of less than about 10%; and
(iv) a contact angle of about 60 degrees to about 110 degrees, wherein the contact angle is measured using a sessile drop technique.

In certain embodiments, the plurality of particles of the crystalline form of the compound of formula (I) has a particle size distribution which is defined by a D₉₀ of about 1 μm to about 100 μm, about 5 μm to about 100 μm, about 10 μm to about 100 μm, about 15 μm to about 100 μm, about 20 μm to about 100 μm, about 25 μm to about 100 μm, about 30 μm to about 100 μm, about 35 μm to about 100 μm, about 40 μm to about 100 μm, about 45 μm to about 100 μm, about 50 μm to about 100 μm, about 60 μm to about 100 μm, about 70 μm to about 100 μm, about 80 μm to about 100 μm, about 90 μm to about 100 μm, about 1 μm to about 90 μm, about 1 μm to about 80 μm, about 1 μm to about 70 μm, about 1 μm to about 60 μm, about 1 μm to about 50 μm, about 1 μm to about 45 μm, about 1 μm to about 40 μm, about 1 μm to about 35 μm, about 1 μm to about 30 μm, about 1 μm to about 25 μm, about 1 μm to about 20 μm, about 1 μm to about 15 μm, about 1 μm to about 10 μm, about 1 μm to about 5 μm, about 5 μm to about 90 μm, about 5 μm to about 80 μm, about 5 μm to about 70 μm, about 5 μm to about 60 μm, about 5 μm to about 50 μm, about 5 μm to about 45 μm, about 5 μm to about 40 μm, about 5 μm to about 35 μm, about 5 μm to about 30 μm, about 5 μm to about 25 μm, about 5 μm to about 20 μm, about 5 μm to about 15 μm, about 5 μm to about 10 μm, about 10 μm to about 90 μm, about 10 μm to about 80 μm, about 10 μm to about 70 μm, about 10 μm to about 60 μm, about 10 μm to about 50 μm, about 10 μm to about 45 μm, about 10 μm to about 40 μm, about 10 μm to about 35 μm, about 10 μm to about 30 μm, about 10 μm to about 25 μm, about 10 μm to about 20 μm, about 10 μm to about 15 μm, about 15 μm to about 90 μm, about 15 μm to about 80 μm, about 15 μm to about 70 μm, about 15 μm to about 60 μm, about 15 μm to about 50 μm, about 15 μm to about 45 μm, about 15 μm to about 40 μm, about 15 μm to about 35 μm, about 15 μm to about 30 μm, about 15 μm to about 25 μm, about 15 μm to about 20 μm, about 20 μm to about 90 μm, about 20 μm to about 80 μm, about 20 μm to about 70 μm, about 20 μm to about 60 μm, about 20 μm to about 50 μm, about 20 μm to about 45 μm, about 20 μm to about 40 μm, about 20 μm to about 35 μm, about 20 μm to about 30 μm, about 20 μm to about 25 μm, about 30 μm to about 90 μm, about 30 μm to about 80 μm, about 30 μm to about 70 μm, about 30 μm to about 60 μm, about 30 μm to about 50 μm, about 30 μm to about 45 μm, about 30 μm to about 40 μm, about 30 μm to about 35 μm, about 35 μm to about 90 μm, about 35 μm to about 80 μm, about 35 μm to about 70 μm, about 35 μm to about 60 μm, about 35 μm to about 50 μm, about 35 μm to about 45 μm, about 35 μm to about 40 μm, about 40 μm to about 90 μm, about 40 μm to about 80 μm, about 40 μm to about 70 μm, about 40 μm to about 50 μm, about 40 μm to about 45 μm, about 45 μm to about 90 μm, about 45 μm to about 80 μm, about 45 μm to about 70 μm, about 45 μm to about 60 μm, about 45 μm to about 50 μm, about 50 μm to about 90 μm, about 50 μm to about 80 μm, about 50 μm to about 70 μm, about 50 μm to about 60 μm, about 60 μm to about 90 μm, about 60 μm to about 80 μm, about 60 μm to about 70 μm, about 70 μm to about 90 μm, about 70 μm to about 80 μm, or about 80 μm to about 90 μm. In certain embodiments, the plurality of particles of the crystalline form of the compound of formula (I) has a particle size distribution which is defined by a D₉₀ of about 1 μm to about 20 μm.

In certain embodiments, the plurality of particles of the crystalline form of the compound of formula (I) has a particle size distribution which is defined by a D₉₀ of about 1 μm to about 20 μm, about 2 μm to about 20 μm, about 3 μm to about 20 μm, about 4 μm to about 20 μm, about 5 μm to about 20 μm, about 6 μm to about 20 μm, about 7 μm to about 20 μm, about 8 μm to about 20 μm, about 9 μm to about 20 μm, about 10 μm to about 20 μm, about 11 μm to about 20 μm, about 12 μm to about 20 μm, about 13 μm to about 20 μm, about 14 μm to about 20 μm, about 15 μm to about 20 μm, about 16 μm to about 20 μm, about 17 μm to about 20 μm, about 18 μm to about 20 μm, about 2 μm to about 19 μm, about 1 μm to about 19 μm, about 1 μm to about 18 μm, about 1 μm to about 17 μm, about 1 μm to about 16 μm, about 1 μm to about 15 μm, about 1 μm to about 14 μm, about 1 μm to about 13 μm, about 1 μm to about 12 μm, about 1 μm to about 11 μm, about 1 μm to about 10 μm, about 1 μm to about 9 μm, about 1 μm to about 8 μm, about 1 μm to about 7 μm, about 1 μm to about 6 μm, about 1 μm to about 5 μm, about 1 μm to about 4 μm, about 1 μm to about 3 μm, about 1 μm to about 2 μm, about 2 μm to about 19 μm, about 2 μm to about 18 μm, about 2 μm to about 17 μm, about 2 μm to about 16 μm, about 2 μm to about 15 μm, about 2 μm to about 14 μm, about 2 μm to about 13 μm, about 2 μm to about 12 μm, about 2 μm to about 11 μm, about 2 μm to about 10 μm, about 2 μm to about 9 μm, about 2 μm to about 8 μm, about 2 μm to about 7 μm, about 2 μm to about 6 μm, about 2 μm to about 5 μm, about 2 μm to about 4 μm, about 2 μm to about 3 μm, about 3 μm to about 19 μm, about 3 μm to about 18 μm, about 3 μm to about 17 μm, about 3 μm to about 16 μm, about 3 μm to about 15 μm, about 3 μm to about 14 μm, about 3 μm to about 13 μm, about 3 μm to about 12 μm, about 3 μm to about 11 μm, about 3 μm to about 10 μm, about 3 μm to about 9 μm, about 3 μm to about 8 μm, about 3 μm to about 7 μm, about 3 μm to about 6 μm, about 3 μm to about 5 μm, about 3 μm to about 4 μm, about 4 μm to about 19 μm, about 4 μm to about 18 μm, about 4 μm to about 17 μm, about 4 μm to about 16 μm, about 4 μm to about 15 μm, about 4 μm to about 14 μm, about 4 μm to about 13 μm, about 4 μm to about 12 μm, about 4 μm to about 11 μm, about 4 μm to about 10 μm, about 4 μm to about 9 μm, about 4 μm to about 8 μm, about 4 μm to about 7 μm, about 4 μm to about 6 μm, about 4 μm to about 5 μm, about 5 μm to about 19 μm, about 5 μm to about 18 μm, about 5 μm to about 17 μm, about 5 μm to about 16 μm, about 5 μm to about 15 μm, about 5 μm to about 14 μm, about 5 μm to about 13 μm, about 5 μm to about 12 μm, about 5 μm to about 11 μm, about 5 μm to about 10 μm, about 5 μm to about 9 μm, about 5 μm to about 8 μm, about 5 μm to about 7 μm, about 5 μm to about 6 μm, about 6 μm to about 19 μm, about 6 μm to about 18 μm, about 6 μm to about 17 μm, about 6 μm to about 16 μm, about 6 μm to about 15 μm, about 6 μm to about 14 μm, about 6 μm to about 13 μm, about 6 μm to about 12 μm, about 6 μm to about 11 μm, about 6 μm to about 10 μm, about 6 μm to about 9 μm, about 6 μm to about 8 μm, about 6 μm to about 7 μm, about 7 μm to about 19 μm, about 7 μm to about 18 μm, about 7 μm to about 17 μm, about 7 μm to about 16 μm, about 7 μm to about 15 μm, about 7 μm to about 14 μm, about 7 μm to about 13 μm, about 7 μm to about 12 μm, about 7 μm to about 11 μm, about 7 μm to about 10 μm, about 7 μm to about 9 μm, about 7 μm to about 8 μm, about 8 μm to about 19 μm, about 8 μm to about 18 μm, about 8 μm to about 17 μm, about 8 μm to about 16 μm, about 8 μm to about 15 μm, about 8 μm to about 14 μm, about 8 μm to about 13 μm, about 8 μm to about 12 μm, about 8 μm to about 11 μm, about 8 μm to about 10 μm, about 8 μm to about 9 μm, about 9 μm to about 19 μm, about 9 μm to about 18 μm, about 9 μm to about 17 μm, about 9 μm to about 16 μm, about 9 μm to about 15 μm, about 9 μm to about 14 μm, about 9 μm to about 13 μm, about 9 μm to about 12 μm, about 9 μm to about 11 μm, about 9 μm to about 10 μm, about 10 μm to about 19 μm, about 10 μm to about 18 μm, about 10 μm to about 17 μm, about 10 μm to about 16 μm, about 10 μm to about 15 μm, about 10 μm to about 14 μm, about 10 μm to about 13 μm, about 10 μm to about 12 μm, about 10 μm to about 11 μm, about 11 μm to about 19 μm, about 11 μm to about 18 μm, about 11 μm to about 17 μm, about 11 μm to about 16 μm, about 11 μm to about 15 μm, about 11 μm to about 14 μm, about 11 μm to about 13 μm, about 11 μm to about 12 μm, about 12 μm to about 19 μm, about 12 μm to about 18 μm, about 12 μm to about 17 μm, about 12 μm to about 16 μm, about 12 μm to about 15 μm, about 12 μm to about 14 μm, about 12 μm to about 13 μm, about 13 μm to about 19 μm, about 13 μm to about 18 μm, about 13 μm to about 17 μm, about 13 μm to about 16 μm, about 13 μm to about 15 μm, about 13 μm to about 14 μm, about 14 μm to about 19 μm, about 14 μm to about 18 μm, about 14 μm to about 17 μm, about 14 μm to about 16 μm, about 14 μm to about 15 μm, about 15 μm to about 19 μm, about 15 μm to about 18 μm, about 15 μm to about 17 μm, about 15 μm to about 16 μm, about 16 μm to about 19 μm, about 16 μm to about 18 μm, about 16 μm to about 17 μm, about 17 μm to about 19 μm, about 17 μm to about 18 μm, or about 18 μm to about 19 μm.

In certain embodiments, the plurality of particles of the crystalline form of the compound of formula (I) has a particle size distribution which is defined by a D₉₀ of about 5 μm to about 10 μm, about 5.5 μm to about 10 μm, about 6 μm to about 10 μm, about 6.5 μm to about 10 μm, about 7 μm to about 10 μm, about 7.5 μm to about 10 μm, about 8 μm to about 10 μm, about 8.5 μm to about 10 μm, about 9 μm to about 10 μm, about 9.5 μm to about 10 μm, about 5 μm to about 9.5 μm, about 5 μm to about 9 μm, about 5 μm to about 8.5 μm, about 5 μm to about 8 μm, about 5 μm to about 7.5 μm, about 5 μm to about 7 μm, about 5 μm to about 6.5 μm, about 5 μm to about 6 μm, about 5 μm to about 5.5 μm, about 5.5 μm to about 9.5 μm, about 5.5 μm to about 9 μm, about 5.5 μm to about 8.5 μm, about 5.5 μm to about 8 μm, about 5.5 μm to about 7.5 μm, about 5.5 μm to about 7 μm, about 5.5 μm to about 6.5 μm, about 5.5 μm to about 6 μm, about 6 μm to about 9.5 μm, about 6 μm to about 9 μm, about 6 μm to about 8.5 μm, about 6 μm to about 8 μm, about 6 μm to about 7.5 μm, about 6 μm to about 7 μm, about 6 μm to about 6.5 μm, about 6.5 μm to about 9.5 μm, about 6.5 μm to about 9 μm, about 6.5 μm to about 8.5 μm, about 6.5 μm to about 8 μm, about 6.5 μm to about 7.5 μm, about 6.5 μm to about 7 μm, about 7 μm to about 9.5 μm, about 7 μm to about 9 μm, about 7 μm to about 8.5 μm, about 7 μm to about 8 μm, about 7 μm to about 7.5 μm, about 7.5 μm to about 9.5 μm, about 7.5 μm to about 9 μm, about 7.5 μm to about 8.5 μm, about 7.5 μm to about 8 μm, about 8 μm to about 9.5 μm, about 8 μm to about 9 μm, about 8 μm to about 8.5 μm, about 8.5 μm to about 9.5 μm, about 8.5 μm to about 9 μm, or about 9 μm to about 9.5 μm.

In certain embodiments, the plurality of particles of the crystalline form of the compound of formula (I) has a particle size distribution which is defined by a D₉₀ of about 6.1 μm to about 7.7 μm, about 6.1 μm to about 8.0 μm, about 4.1 μm to about 10.0 μm, about 5.6 μm to about 10.6 μm, about 4.9 μm to about 12.4 μm, about 3.9 μm to about 11.0 μm, or about 4.2 μm to about 11.6 μm.

In certain embodiments, the plurality of particles of the crystalline form of the compound of formula (I) has a particle size distribution which is defined by a D₉₀ of about 6.1 μm to about 7.7 μm.

In certain embodiments, the plurality of particles of the crystalline form of the compound of formula (I) has a particle size distribution which is defined by a D₉₀ of about 6.1 μm to about 8.0 μm.

In certain embodiments, the plurality of particles of the crystalline form of the compound of formula (I) has a particle size distribution which is defined by a D₉₀ of about 4.1 μm to about 10.0 μm.

In certain embodiments, the plurality of particles of the crystalline form of the compound of formula (I) has a particle size distribution which is defined by a D₉₀ of about 5.6 μm to about 10.6 μm.

In certain embodiments, the plurality of particles of the crystalline form of the compound of formula (I) has a particle size distribution which is defined by a D₉₀ of about 4.9 μm to about 12.4 μm.

In certain embodiments, the plurality of particles of the crystalline form of the compound of formula (I) has a particle size distribution which is defined by a D₉₀ of about 3.9 μm to about 11.0 μm.

In certain embodiments, the plurality of particles of the crystalline form of the compound of formula (I) has a particle size distribution which is defined by a D₉₀ of about 4.2 μm to about 11.6 μm.

In certain embodiments, the plurality of particles of the crystalline form of the compound of formula (I) has an average particle size which is defined by a D₉₀ of about 11 μm.

In certain embodiments, the plurality of particles of the crystalline form of the compound of formula (I) have a yield pressure of about 40 MPa to about 200 MPa, about 50 MPa to about 200 MPa, about 60 MPa to about 200 MPa, about 70 MPa to about 200 MPa, about 80 MPa to about 200 MPa, about 90 MPa to about 200 MPa, about 95 MPa to about 200 MPa, about 100 MPa to about 200 MPa, about 150 MPa to about 200 MPa, about 40 MPa to about 150 MPa, about 40 MPa to about 100 MPa, about 40 MPa to about 95 MPa, about 40 MPa to about 90 MPa, about 40 MPa to about 80 MPa, about 40 MPa to about 70 MPa, about 40 MPa to about 60 MPa, about 40 MPa to about 50 MPa, about 50 MPa to about 150 MPa, about 50 MPa to about 100 MPa, about 50 MPa to about 95 MPa, about 50 MPa to about 90 MPa, about 50 MPa to about 80 MPa, about 50 MPa to about 70 MPa, about 50 MPa to about 60 MPa, about 60 MPa to about 150 MPa, about 60 MPa to about 100 MPa, about 60 MPa to about 95 MPa, about 60 MPa to about 90 MPa, about 60 MPa to about 80 MPa, about 60 MPa to about 70 MPa, about 70 MPa to about 150 MPa, about 70 MPa to about 100 MPa, about 70 MPa to about 95 MPa, about 70 MPa to about 90 MPa, about 70 MPa to about 80 MPa, about 80 MPa to about 150 MPa, about 80 MPa to about 100 MPa, about 80 MPa to about 95 MPa, about 80 MPa to about 90 MPa, about 90 MPa to about 150 MPa, about 90 MPa to about 100 MPa, about 90 MPa to about 95 MPa, about 95 MPa to about 150 MPa, about 95 MPa to about 100 MPa, or about 100 MPa to about 150 MPa.

In certain embodiments, the plurality of particles of the crystalline form of the compound of formula (I) have a yield pressure of about 60 MPa to about 100 MPa. In certain embodiments, the plurality of particles of the crystalline form of the compound of formula (I) have a yield pressure of about 70 MPa to about 95 MPa. In certain embodiments, the plurality of particles of the crystalline form of the compound of formula (I) have a yield pressure of about 80 MPa to about 90 MPa.

In certain embodiments, the plurality of particles of the crystalline form of the compound of formula (I) have a strain rate sensitivity of less than about 5%, less than about 6%, less than about 7%, less than about 8%, less than about 9%, less than about 10%, less than about 15%, or less than about 20%. In certain embodiments, the plurality of particles of the crystalline form of the compound of formula (I) have a strain rate sensitivity of less than about 10%.

In certain embodiments, the plurality of particles of the crystalline form of the compound of formula (I) have a contact angle of about 60 degrees to about 110 degrees, about 65 degrees to about 110 degrees, about 70 degrees to about 110 degrees, about 75 degrees to about 110 degrees, about 80 degrees to about 110 degrees, about 90 degrees to about 110 degrees, about 100 degrees to about 110 degrees, about 60 degrees to about 100 degrees, about 60 degrees to about 90 degrees, about 60 degrees to about 80 degrees, about 60 degrees to about 75 degrees, about 60 degrees to about 70 degrees, about 60 degrees to about 65 degrees, about 65 degrees to about 100 degrees, about 65 degrees to about 90 degrees, about 65 degrees to about 80 degrees, about 65 degrees to about 75 degrees, about 65 degrees to about 70 degrees, about 70 degrees to about 100 degrees, about 70 degrees to about 90 degrees, about 70 degrees to about 80 degrees, about 70 degrees to about 75 degrees, about 75 degrees to about 100 degrees, about 75 degrees to about 90 degrees, about 75 degrees to about 80 degrees, about 80 degrees to about 100 degrees, about 80 degrees to about 90 degrees, or about 90 degrees to about 100 degrees, wherein the contact angle is measured using a sessile drop technique. In certain embodiments, the plurality of particles of the crystalline form of the compound of formula (I) have a contact angle of about 70 degrees to about 80 degrees. In certain embodiments, the plurality of particles of the crystalline form of the compound of formula (I) have a contact angle of about 70 degrees to about 75 degrees.

In various embodiments, the pharmaceutical composition comprises a plurality of particles of a crystalline form of the compound of formula (I)

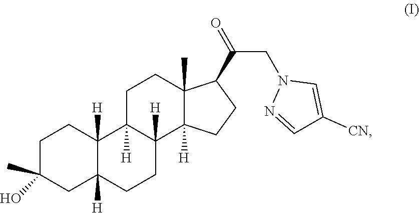

wherein the plurality of particles of the crystalline form of the compound of formula (I) comprises:
(v) a particle size distribution which is defined by a D₉₀ of about 1 μm to about 100 μm;
(vi) a yield pressure of about 80 MPa to about 90 MPa;
(vii) a strain rate sensitivity of less than about 10%; and
(viii) a contact angle of about 70 degrees to about 75 degrees, wherein the contact angle is measured using a sessile drop technique.

In various embodiments, the pharmaceutical composition comprises a plurality of particles of a crystalline form of the compound of formula (I)

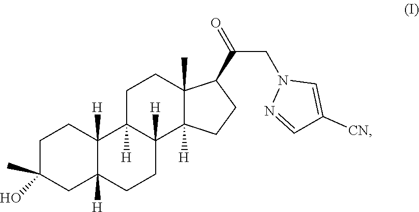

wherein the plurality of particles of the crystalline form of the compound of formula (I) comprises:
(i) a particle size distribution which is defined by a D₉₀ of about 6.1 μm to about 7.7 μm;
(ii) a yield pressure of about 80 MPa to about 90 MPa;
(iii) a strain rate sensitivity of less than about 10%; and
(iv) a contact angle of about 70 degrees to about 75 degrees, wherein the contact angle is measured using a sessile drop technique.

In various embodiments, the pharmaceutical composition comprises a plurality of particles of a crystalline form of the compound of formula (I)

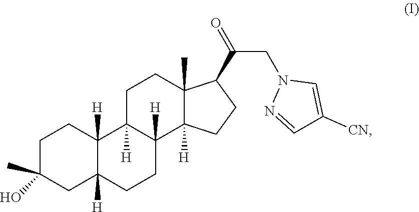

wherein the plurality of particles of the crystalline form of the compound of formula (I) comprises:
(i) a particle size distribution which is defined by a D₉₀ of about 4.2 μm to about 11.6 μm;
(ii) a yield pressure of about 80 MPa to about 90 MPa;
(iii) a strain rate sensitivity of less than about 10%; and
(iv) a contact angle of about 70 degrees to about 75 degrees, wherein the contact angle is measured using a sessile drop technique.

In various embodiments, the pharmaceutical composition comprises a crystalline form of the compound of formula (I)

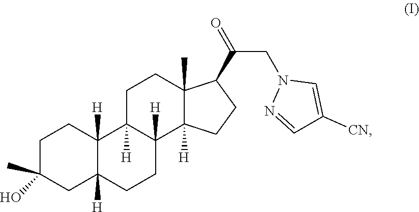

wherein the pharmaceutical composition comprises at least one of the following features:
(i) a true density of about 1.0 g/cc to about 2.5 g/cc;
(ii) a bulk density of about 0.2 g/cc to about 0.8 g/cc and a tapped density of about 0.3 g/cc to about 1.1 g/cc, wherein the tapped density of the pharmaceutical composition is higher than the bulk density;
(iii) a Carr Index of about 10 to about 38;
(iv) about 0.2% to about 90% of the pharmaceutical composition is retained when passed through a 710/25 (microns/mesh) sieve, about 0.2% to about 75% of the material is retained when the pharmaceutical composition is passed through a 425/40 (microns/mesh) sieve, and about 0.1% to about 55% of the pharmaceutical composition is retained when passed through a 63/230 (microns/mesh) sieve;
(v) a solid fraction of about 0.5 to about 0.95;
(vi) a flow rate index (FRI) of about 0.05 kg/sec to about 4 kg/sec; and
(vii) the pharmaceutical composition releases at least about 50% of the compound of formula (I) after about 20 minutes, when tested using a USP 1 or a USP 2 apparatus.

In certain embodiments, the pharmaceutical composition has a true density of about 1.0 g/cc to about 2.5 g/cc, about 1.1 g/cc to about 2.5 g/cc, about 1.2 g/cc to about 2.5 g/cc, about 1.3 g/cc to about 2.5 g/cc, about 1.4 g/cc to about 2.5 g/cc, about 1.5 g/cc to about 2.5 g/cc, about 1.6 g/cc to about 2.5 g/cc, about 1.7 g/cc to about 2.5 g/cc, about 1.8 g/cc to about 2.5 g/cc, about 1.9 g/cc to about 2.5 g/cc, about 2.0 g/cc to about 2.5 g/cc, about 2.2 g/cc to about 2.5 g/cc, about 1.0 g/cc to about 2.2 g/cc, about 1.0 g/cc to about 2.0 g/cc, about 1.0 g/cc to about 1.9 g/cc, about 1.0 g/cc to about 1.8 g/cc, about 1.0 g/cc to about 1.7 g/cc, about 1.0 g/cc to about 1.6 g/cc, about 1.0 g/cc to about 1.5 g/cc, about 1.0 g/cc to about 1.4 g/cc, about 1.0 g/cc to about 1.3 g/cc, about 1.0 g/cc to about 1.2 g/cc, about 1.0 g/cc to about 1.1 g/cc, about 1.1 g/cc to about 2.2 g/cc, about 1.1 g/cc to about 2.0 g/cc, about 1.1 g/cc to about 1.9 g/cc, about 1.1 g/cc to about 1.8 g/cc, about 1.1 g/cc to about 1.7 g/cc, about 1.1 g/cc to about 1.6 g/cc, about 1.1 g/cc to about 1.5 g/cc, about 1.1 g/cc to about 1.4 g/cc, about 1.1 g/cc to about 1.3 g/cc, about 1.1 g/cc to about 1.2 g/cc, about 1.2 g/cc to about 2.2 g/cc, about 1.2 g/cc to about 2.0 g/cc, about 1.2 g/cc to about 1.9 g/cc, about 1.2 g/cc to about 1.8 g/cc, about 1.2 g/cc to about 1.7 g/cc, about 1.2 g/cc to about 1.6 g/cc, about 1.2 g/cc to about 1.5 g/cc, about 1.2 g/cc to about 1.4 g/cc, about 1.2 g/cc to about 1.3 g/cc, about 1.3 g/cc to about 2.2 g/cc, about 1.3 g/cc to about 2.0 g/cc, about 1.3 g/cc to about 1.9 g/cc, about 1.3 g/cc to about 1.8 g/cc, about 1.3 g/cc to about 1.7 g/cc, about 1.3 g/cc to about 1.6 g/cc, about 1.3 g/cc to about 1.5 g/cc, about 1.3 g/cc to about 1.4 g/cc, about 1.4 g/cc to about 2.2 g/cc, about 1.4 g/cc to about 2.0 g/cc, about 1.4 g/cc to about 1.9 g/cc, about 1.4 g/cc to about 1.8 g/cc, about 1.4 g/cc to about 1.7 g/cc, about 1.4 g/cc to about 1.6 g/cc, about 1.4 g/cc to about 1.5 g/cc, about 1.5 g/cc to about 2.2 g/cc, about 1.5 g/cc to about 2.0 g/cc, about 1.5 g/cc to about 1.9 g/cc, about 1.5 g/cc to about 1.8 g/cc, about 1.5 g/cc to about 1.7 g/cc, about 1.5 g/cc to about 1.6 g/cc, about 1.6 g/cc to about 2.2 g/cc, about 1.6 g/cc to about 2.0 g/cc, about 1.6 g/cc to about 1.9 g/cc, about 1.6 g/cc to about 1.8 g/cc, about 1.6 g/cc to about 1.7 g/cc, about 1.7 g/cc to about 2.2 g/cc, about 1.7 g/cc to about 2.0 g/cc, about 1.7 g/cc to about 1.9 g/cc, about 1.7 g/cc to about 1.8 g/cc, about 1.8 g/cc to about 2.2 g/cc, about 1.8 g/cc to about 2.0 g/cc, about 1.8 g/cc to about 1.9 g/cc, about 1.9 g/cc to about 2.2 g/cc, about 1.9 g/cc to about 2.0 g/cc, or about 2.0 g/cc to about 2.2 g/cc.

In certain embodiments, the pharmaceutical composition has a true density of about 1.1 g/cc to about 2.0 g/cc. In certain embodiments, the pharmaceutical composition has a true density of about 1.2 g/cc to about 1.6 g/cc.

In certain embodiments, the pharmaceutical composition has a bulk density of about 0.2 g/cc to about 0.8 g/cc, about 0.3 g/cc to about 0.8 g/cc, about 0.4 g/cc to about 0.8 g/cc, about 0.5 g/cc to about 0.8 g/cc, about 0.6 g/cc to about 0.8 g/cc, about 0.65 g/cc to about 0.8 g/cc, about 0.7 g/cc to about 0.8 g/cc, about 0.2 g/cc to about 0.7 g/cc, about 0.2 g/cc to about 0.65 g/cc, about 0.2 g/cc to about 0.6 g/cc, about 0.2 g/cc to about 0.5 g/cc, about 0.2 g/cc to about 0.4 g/cc, about 0.2 g/cc to about 0.3 g/cc, about 0.3 g/cc to about 0.7 g/cc, about 0.3 g/cc to about 0.65 g/cc, about 0.3 g/cc to about 0.6 g/cc, about 0.3 g/cc to about 0.5 g/cc, about 0.3 g/cc to about 0.4 g/cc, about 0.4 g/cc to about 0.7 g/cc, about 0.4 g/cc to about 0.65 g/cc, about 0.4 g/cc to about 0.6 g/cc, about 0.4 g/cc to about 0.5 g/cc, about 0.5 g/cc to about 0.7 g/cc, about 0.5 g/cc to about 0.65 g/cc, about 0.5 g/cc to about 0.6 g/cc, about 0.6 g/cc to about 0.7 g/cc, about 0.6 g/cc to about 0.65 g/cc, or about 0.65 g/cc to about 0.8 g/cc.

In certain embodiments, the pharmaceutical composition has a bulk density of about 0.2 g/cc to about 0.7 g/cc. In certain embodiments, the pharmaceutical composition has a bulk density of about 0.3 g/cc to about 0.65 g/cc. In certain embodiments, the pharmaceutical composition has a bulk density of about 0.4 g/cc to about 0.7 g/cc. In certain embodiments, the pharmaceutical composition has a bulk density of about 0.5 g/cc to about 0.65 g/cc.

In certain embodiments, the pharmaceutical composition has a tapped density of about 0.3 g/cc to about 1.1 g/cc, about 0.35 g/cc to about 1.1 g/cc, about 0.4 g/cc to about 1.1 g/cc, about 0.5 g/cc to about 1.1 g/cc, about 0.6 g/cc to about 1.1 g/cc, about 0.7 g/cc to about 1.1 g/cc, about 0.8 g/cc to about 1.1 g/cc, about 0.85 g/cc to about 1.1 g/cc, about 0.9 g/cc to about 1.1 g/cc, about 1.0 g/cc to about 1.1 g/cc, about 0.3 g/cc to about 1.0 g/cc, about 0.3 g/cc to about 0.9 g/cc, about 0.3 g/cc to about 0.85 g/cc, about 0.3 g/cc to about 0.8 g/cc, about 0.3 g/cc to about 0.7 g/cc, about 0.3 g/cc to about 0.6 g/cc, about 0.3 g/cc to about 0.5 g/cc, about 0.3 g/cc to about 0.4 g/cc, about 0.3 g/cc to about 0.35 g/cc, about 0.35 g/cc to about 1.0 g/cc, about 0.35 g/cc to about 0.9 g/cc, about 0.35 g/cc to about 0.85 g/cc, about 0.35 g/cc to about 0.8 g/cc, about 0.35 g/cc to about 0.7 g/cc, about 0.35 g/cc to about 0.6 g/cc, about 0.35 g/cc to about 0.5 g/cc, about 0.35 g/cc to about 0.4 g/cc, about 0.4 g/cc to about 1.0 g/cc, about 0.4 g/cc to about 0.9 g/cc, about 0.4 g/cc to about 0.85 g/cc, about 0.4 g/cc to about 0.8 g/cc, about 0.4 g/cc to about 0.7 g/cc, about 0.4 g/cc to about 0.6 g/cc, about 0.4 g/cc to about 0.5 g/cc, about 0.5 g/cc to about 1.0 g/cc, about 0.5 g/cc to about 0.9 g/cc, about 0.5 g/cc to about 0.85 g/cc, about 0.5 g/cc to about 0.8 g/cc, about 0.5 g/cc to about 0.7 g/cc, about 0.5 g/cc to about 0.6 g/cc, about 0.6 g/cc to about 0.85 g/cc, about 0.6 g/cc to about 1.0 g/cc, about 0.6 g/cc to about 0.9 g/cc, about 0.6 g/cc to about 0.8 g/cc, about 0.6 g/cc to about 0.7 g/cc, about 0.7 g/cc to about 1.0 g/cc, about 0.7 g/cc to about 0.9 g/cc, about 0.7 g/cc to about 0.85 g/cc, about 0.7 g/cc to about 0.8 g/cc, about 0.8 g/cc to about 1.0 g/cc, about 0.8 g/cc to about 0.9 g/cc, about 0.8 g/cc to about 0.85 g/cc, about 0.85 g/cc to about 1.0 g/cc, about 0.85 g/cc to about 0.9 g/cc, or about 0.9 g/cc to about 1.0 g/cc.

In certain embodiments, the pharmaceutical composition has a tapped density of about 0.3 g/cc to about 0.9 g/cc. In certain embodiments, the pharmaceutical composition has a tapped density of about 0.35 g/cc to about 0.85 g/cc. In certain embodiments, the pharmaceutical composition has a tapped density of about 0.6 g/cc to about 0.85 g/cc. In certain embodiments, the pharmaceutical composition has a tapped density of about 0.7 g/cc to about 0.8 g/cc.

In certain embodiments, about 0.2% to about 90%, about 0.5% to about 90%, about 1% to about 90%, about 2% to about 90%, about 5% to about 90%, about 10% to about 90%, about 15% to about 90%, about 20% to about 90%, about 30% to about 90%, about 40% to about 90%, about 50% to about 90%, about 60% to about 90%, about 70% to about 90%, about 75% to about 90%, about 80% to about 90%, about 85% to about 90%, about 0.5% to about 85%, about 0.5% to about 80%, about 0.5% to about 75%, about 0.5% to about 70%, about 0.5% to about 60%, about 0.5% to about 50%, about 0.5% to about 40%, about 0.5% to about 30%, about 0.5% to about 20%, about 0.5% to about 15%, about 0.5% to about 10%, about 0.5% to about 5%, about 0.5% to about 2%, about 0.5% to about 1%, about 1% to about 85%, about 1% to about 80%, about 1% to about 75%, about 1% to about 70%, about 1% to about 60%, about 1% to about 50%, about 1% to about 40%, about 1% to about 30%, about 1% to about 20%, about 1% to about 15%, about 1% to about 10%, about 1% to about 5%, about 1% to about 2%, about 2% to about 85%, about 2% to about 80%, about 2% to about 75%, about 2% to about 70%, about 2% to about 60%, about 2% to about 50%, about 2% to about 40%, about 2% to about 30%, about 2% to about 20%, about 2% to about 15%, about 2% to about 10%, about 2% to about 5%, about 5% to about 85%, about 5% to about 80%, about 5% to about 80%, about 5% to about 75%, about 5% to about 70%, about 5% to about 60%, about 5% to about 50%, about 5% to about 40%, about 5% to about 30%, about 5% to about 20%, about 5% to about 15%, about 5% to about 10%, about 10% to about 85%, about 10% to about 80%, about 10% to about 75%, about 10% to about 70%, about 10% to about 60%, about 10% to about 50%, about 10% to about 40%, about 10% to about 30%, about 10% to about 20%, about 10% to about 15%, about 15% to about 85%, about 15% to about 80%, about 15% to about 75%, about 15% to about 70%, about 15% to about 60%, about 15% to about 50%, about 15% to about 40%, about 15% to about 30%, about 15% to about 20%, about 20% to about 85%, about 20% to about 80%, about 20% to about 75%, about 20% to about 70%, about 20% to about 60%, about 20% to about 50%, about 20% to about 40%, about 20% to about 30%, about 30% to about 85%, about 30% to about 80%, about 30% to about 75%, about 30% to about 70%, about 30% to about 60%, about 30% to about 50%, about 30% to about 40%, about 40% to about 85%, about 40% to about 80%, about 40% to about 75%, about 40% to about 70%, about 40% to about 60%, about 40% to about 50%, about 50% to about 85%, about 50% to about 80%, about 50% to about 75%, about 50% to about 70%, about 50% to about 60%, about 60% to about 85%, about 60% to about 80%, about 60% to about 75%, about 60% to about 70%, about 70% to about 85%, about 70% to about 80%, about 70% to about 75%, about 75% to about 85%, about 75% to about 80%, or about 80% to about 85% of the pharmaceutical composition is retained when passed through a 710/25 (microns/mesh) sieve.

In certain embodiments, about 0.5% to about 75% of the pharmaceutical composition is retained when passed through a 710/25 (microns/mesh) sieve. In certain embodiments, about 0.5% to about 60% of the pharmaceutical composition is retained when passed through a 710/25 (microns/mesh) sieve.

In certain embodiments, about 0.2% to about 75%, about 0.5% to about 75%, about 1% to about 75%, about 2% to about 75%, about 5% to about 75%, about 15% to about 75%, about 25% to about 75%, about 35% to about 75%, about 50% to about 75%, about 0.2% to about 50%, about 0.2% to about 35%, about 0.2% to about 25%, about 0.2% to about 15%, about 0.2% to about 5%, about 0.2% to about 2%, about 0.2% to about 0.5%, about 0.5% to about 50%, about 0.5% to about 35%, about 0.5% to about 25%, about 0.5% to about 15%, about 0.5% to about 5%, about 0.5% to about 2%, about 0.5% to about 1%, about 1% to about 50%, about 1% to about 35%, about 1% to about 25%, about 1% to about 15%, about 1% to about 5%, about 1% to about 2%, about 2% to about 50%, about 2% to about 35%, about 2% to about 25%, about 2% to about 15%, about 2% to about 5%, about 5% to about 50%, about 5% to about 35%, about 5% to about 25%, about 5% to about 15%, about 15% to about 50%, about 15% to about 35%, about 15% to about 25%, about 25% to about 50%, about 25% to about 35%, or about 35% to about 50% of the material is retained when the pharmaceutical composition is passed through a 425/40 (microns/mesh) sieve.

In certain embodiments, about 2% to about 50% of the material is retained when the pharmaceutical composition is passed through a 425/40 (microns/mesh) sieve. In certain embodiments, about 5% to about 35% of the material is retained when the pharmaceutical composition is passed through a 425/40 (microns/mesh) sieve.

In certain embodiments, about 0.1% to about 55%, about 0.5% to about 55%, about 1% to about 55%, about 5% to about 55%, about 10% to about 55%, about 15% to about 55%, about 20% to about 55%, about 25% to about 55%, about 30% to about 55%, about 40% to about 55%, about 0.1% to about 40%, about 0.1% to about 30%, about 0.1% to about 25%, about 0.1% to about 20%, about 0.1% to about 15%, about 0.1% to about 10%, about 0.1% to about 5%, about 0.1% to about 1%, about 0.1% to about 0.5%, about 0.5% to about 40%, about 0.5% to about 30%, about 0.5% to about 25%, about 0.5% to about 20%, about 0.5% to about 15%, about 0.5% to about 10%, about 0.5% to about 5%, about 0.5% to about 1%, about 1% to about 40%, about 1% to about 30%, about 1% to about 25%, about 1% to about 20%, about 1% to about 15%, about 1% to about 10%, about 1% to about 5%, about 5% to about 40%, about 5% to about 30%, about 5% to about 25%, about 5% to about 20%, about 5% to about 15%, about 5% to about 10%, about 10% to about 40%, about 10% to about 30%, about 10% to about 25%, about 10% to about 20%, about 10% to about 15%, about 15% to about 40%, about 15% to about 30%, about 15% to about 25%, about 15% to about 20%, about 20% to about 40%, about 20% to about 30%, about 20% to about 25%, about 25% to about 40%, about 25% to about 30%, or about 30% to about 40% of the pharmaceutical composition is retained when passed through a 63/230 (microns/mesh) sieve.

In certain embodiments, about 0.5% to about 30% of the pharmaceutical composition is retained when passed through a 63/230 (microns/mesh) sieve. In certain embodiments, about 1% to about 25% of the pharmaceutical composition is retained when passed through a 63/230 (microns/mesh) sieve.

In certain embodiments, the pharmaceutical composition has a solid fraction of about 0.5 to about 0.95, about 0.55 to about 0.95, about 0.6 to about 0.95, about 0.7 to about 0.95, about 0.8 to about 0.95, about 0.85 to about 0.95, about 0.9 to about 0.95, about 0.5 to about 0.9, about 0.5 to about 0.85, about 0.5 to about 0.8, about 0.5 to about 0.7, about 0.5 to about 0.6, about 0.5 to about 0.55, about 0.55 to about 0.9, about 0.55 to about 0.85, about 0.55 to about 0.8, about 0.55 to about 0.7, about 0.55 to about 0.6, about 0.6 to about 0.9, about 0.6 to about 0.85, about 0.6 to about 0.8, about 0.6 to about 0.7, about 0.7 to about 0.9, about 0.7 to about 0.85, about 0.7 to about 0.8, about 0.8 to about 0.9, about 0.8 to about 0.85, or about 0.85 to about 0.9. In certain embodiments, the pharmaceutical composition has a solid fraction of about 0.55 to about 0.9. In certain embodiments, the pharmaceutical composition has a solid fraction of about 0.6 to about 0.85.

In certain embodiments, the pharmaceutical composition has a flow rate index (FRI) of about 0.05 kg/sec to about 4 kg/sec, about 0.1 kg/sec to about 4 kg/sec, about 0.5 kg/sec to about 4 kg/sec, about 1 kg/sec to about 4 kg/sec, about 2 kg/sec to about 4 kg/sec, about 3 kg/sec to about 4 kg/sec, about 0.05 kg/sec to about 3 kg/sec, about 0.05 kg/sec to about 2 kg/sec, about 0.05 kg/sec to about 1 kg/sec, about 0.05 kg/sec to about 0.5 kg/sec, about 0.05 kg/sec to about 0.1 kg/sec, about 0.1 kg/sec to about 3 kg/sec, about 0.1 kg/sec to about 2 kg/sec, about 0.1 kg/sec to about 1 kg/sec, about 0.1 kg/sec to about 0.5 kg/sec, about 0.5 kg/sec to about 3 kg/sec, about 0.5 kg/sec to about 2 kg/sec, about 0.5 kg/sec to about 1 kg/sec, about 1 kg/sec to about 3 kg/sec, about 1 kg/sec to about 2 kg/sec, or about 2 kg/sec to about 3 kg/sec.

In certain embodiments, a pharmaceutical composition described herein releases at least about 50%, at least about 55%, at least about 60%, at least about 65%, at least about 70%, at least about 75%, at least about 80%, at least about 85%, at least about 90%, or at least about 95% of the compound of formula (I) after about 20 minutes, when tested using a USP 1 or a USP 2 apparatus. In certain embodiments, a pharmaceutical composition described herein releases at least about 55% of the compound of formula (I) after about 20 minutes, when tested using a USP 1 or a USP 2 apparatus. In certain embodiments, a pharmaceutical composition described herein releases at least about 60% of the compound of formula (I) after about 20 minutes, when tested using a USP 1 or a USP 2 apparatus. In certain embodiments, a pharmaceutical composition described herein releases at least about 65% of the compound of formula (I) after about 20 minutes, when tested using a USP 1 or a USP 2 apparatus. In certain embodiments, a pharmaceutical composition described herein releases at least about 70% of the compound of formula (I) after about 20 minutes, when tested using a USP 1 or a USP 2 apparatus. In certain embodiments, a pharmaceutical composition described herein releases at least about 75% of the compound of formula (I) after about 20 minutes, when tested using a USP 1 or a USP 2 apparatus. In certain embodiments, a pharmaceutical composition described herein releases at least about 80% of the compound of formula (I) after about 20 minutes, when tested using a USP 1 or a USP 2 apparatus. In certain embodiments, a pharmaceutical composition described herein releases at least about 85% of the compound of formula (I) after about 20 minutes, when tested using a USP 1 or a USP 2 apparatus. In certain embodiments, a pharmaceutical composition described herein releases at least about 90% of the compound of formula (I) after about 20 minutes, when tested using a USP 1 or a USP 2 apparatus. In certain embodiments, a pharmaceutical composition described herein releases at least about 95% of the compound of formula (I) after about 20 minutes, when tested using a USP 1 or a USP 2 apparatus.

In certain embodiments, a pharmaceutical composition described herein releases at least about 50%, at least about 55%, at least about 60%, at least about 65%, at least about 70%, at least about 75%, at least about 80%, at least about 85%, at least about 90%, or at least about 95% of the compound of formula (I) after about 30 minutes, when tested using a USP 1 or a USP 2 apparatus. In certain embodiments, a pharmaceutical composition described herein releases at least about 55% of the compound of formula (I) after about 30 minutes, when tested using a USP 1 or a USP 2 apparatus. In certain embodiments, a pharmaceutical composition described herein releases at least about 60% of the compound of formula (I) after about 30 minutes, when tested using a USP 1 or a USP 2 apparatus. In certain embodiments, a pharmaceutical composition described herein releases at least about 65% of the compound of formula (I) after about 30 minutes, when tested using a USP 1 or a USP 2 apparatus. In certain embodiments, a pharmaceutical composition described herein releases at least about 70% of the compound of formula (I) after about 30 minutes, when tested using a USP 1 or a USP 2 apparatus. In certain embodiments, a pharmaceutical composition described herein releases at least about 75% of the compound of formula (I) after about 30 minutes, when tested using a USP 1 or a USP 2 apparatus. In certain embodiments, a pharmaceutical composition described herein releases at least about 80% of the compound of formula (I) after about 30 minutes, when tested using a USP 1 or a USP 2 apparatus. In certain embodiments, a pharmaceutical composition described herein releases at least about 85% of the compound of formula (I) after about 30 minutes, when tested using a USP 1 or a USP 2 apparatus. In certain embodiments, a pharmaceutical composition described herein releases at least about 90% of the compound of formula (I) after about 30 minutes, when tested using a USP 1 or a USP 2 apparatus. In certain embodiments, a pharmaceutical composition described herein releases at least about 95% of the compound of formula (I) after about 30 minutes, when tested using a USP 1 or a USP 2 apparatus.

In certain embodiments, a pharmaceutical composition described herein releases at least about 50%, at least about 55%, at least about 60%, at least about 65%, at least about 70%, at least about 75%, at least about 80%, at least about 85%, at least about 90%, or at least about 95% of the compound of formula (I) after about 45 minutes, when tested using a USP 1 or a USP 2 apparatus. In certain embodiments, a pharmaceutical composition described herein releases at least about 55% of the compound of formula (I) after about 45 minutes, when tested using a USP 1 or a USP 2 apparatus. In certain embodiments, a pharmaceutical composition described herein releases at least about 60% of the compound of formula (I) after about 45 minutes, when tested using a USP 1 or a USP 2 apparatus. In certain embodiments, a pharmaceutical composition described herein releases at least about 65% of the compound of formula (I) after about 45 minutes, when tested using a USP 1 or a USP 2 apparatus. In certain embodiments, a pharmaceutical composition described herein releases at least about 70% of the compound of formula (I) after about 45 minutes, when tested using a USP 1 or a USP 2 apparatus. In certain embodiments, a pharmaceutical composition described herein releases at least about 75% of the compound of formula (I) after about 45 minutes, when tested using a USP 1 or a USP 2 apparatus. In certain embodiments, a pharmaceutical composition described herein releases at least about 80% of the compound of formula (I) after about 45 minutes, when tested using a USP 1 or a USP 2 apparatus. In certain embodiments, a pharmaceutical composition described herein releases at least about 85% of the compound of formula (I) after about 45 minutes, when tested using a USP 1 or a USP 2 apparatus. In certain embodiments, a pharmaceutical composition described herein releases at least about 90% of the compound of formula (I) after about 45 minutes, when tested using a USP 1 or a USP 2 apparatus. In certain embodiments, a pharmaceutical composition described herein releases at least about 95% of the compound of formula (I) after about 45 minutes, when tested using a USP 1 or a USP 2 apparatus.

In certain embodiments, a pharmaceutical composition described herein releases at least about 50%, at least about 55%, at least about 60%, at least about 65%, at least about 70%, at least about 75%, at least about 80%, at least about 85%, at least about 90%, or at least about 95% of the compound of formula (I) after about 60 minutes, when tested using a USP 1 or a USP 2 apparatus. In certain embodiments, a pharmaceutical composition described herein releases at least about 55% of the compound of formula (I) after about 60 minutes, when tested using a USP 1 or a USP 2 apparatus. In certain embodiments, a pharmaceutical composition described herein releases at least about 60% of the compound of formula (I) after about 60 minutes, when tested using a USP 1 or a USP 2 apparatus. In certain embodiments, a pharmaceutical composition described herein releases at least about 60% of the compound of formula (I) after about 60 minutes, when tested using a USP 1 or a USP 2 apparatus. In certain embodiments, a pharmaceutical composition described herein releases at least about 70% of the compound of formula (I) after about 60 minutes, when tested using a USP 1 or a USP 2 apparatus. In certain embodiments, a pharmaceutical composition described herein releases at least about 75% of the compound of formula (I) after about 60 minutes, when tested using a USP 1 or a USP 2 apparatus. In certain embodiments, a pharmaceutical composition described herein releases at least about 80% of the compound of formula (I) after about 60 minutes, when tested using a USP 1 or a USP 2 apparatus. In certain embodiments, a pharmaceutical composition described herein releases at least about 85% of the compound of formula (I) after about 60 minutes, when tested using a USP 1 or a USP 2 apparatus. In certain embodiments, a pharmaceutical composition described herein releases at least about 90% of the compound of formula (I) after about 60 minutes, when tested using a USP 1 or a USP 2 apparatus. In certain embodiments, a pharmaceutical composition described herein releases at least about 95% of the compound of formula (I) after about 60 minutes, when tested using a USP 1 or a USP 2 apparatus.

In certain embodiments, a pharmaceutical composition described herein releases at least about 50%, at least about 55%, at least about 60%, at least about 65%, at least about 70%, at least about 75%, at least about 80%, at least about 85%, at least about 90%, or at least about 95% of the compound of formula (I) after about 75 minutes, when tested using a USP 1 or a USP 2 apparatus. In certain embodiments, a pharmaceutical composition described herein releases at least about 55% of the compound of formula (I) after about 75 minutes, when tested using a USP 1 or a USP 2 apparatus. In certain embodiments, a pharmaceutical composition described herein releases at least about 60% of the compound of formula (I) after about 75 minutes, when tested using a USP 1 or a USP 2 apparatus. In certain embodiments, a pharmaceutical composition described herein releases at least about 65% of the compound of formula (I) after about 75 minutes, when tested using a USP 1 or a USP 2 apparatus. In certain embodiments, a pharmaceutical composition described herein releases at least about 70% of the compound of formula (I) after about 75 minutes, when tested using a USP 1 or a USP 2 apparatus. In certain embodiments, a pharmaceutical composition described herein releases at least about 75% of the compound of formula (I) after about 75 minutes, when tested using a USP 1 or a USP 2 apparatus. In certain embodiments, a pharmaceutical composition described herein releases at least about 80% of the compound of formula (I) after about 75 minutes, when tested using a USP 1 or a USP 2 apparatus. In certain embodiments, a pharmaceutical composition described herein releases at least about 85% of the compound of formula (I) after about 75 minutes, when tested using a USP 1 or a USP 2 apparatus. In certain embodiments, a pharmaceutical composition described herein releases at least about 90% of the compound of formula (I) after about 75 minutes, when tested using a USP 1 or a USP 2 apparatus. In certain embodiments, a pharmaceutical composition described herein releases at least about 95% of the compound of formula (I) after about 75 minutes, when tested using a USP 1 or a USP 2 apparatus.

In certain embodiments, the release profile of a pharmaceutical composition described herein is tested using a USP 1 apparatus. In certain embodiments, the release profile of a pharmaceutical composition described herein is tested using a USP 2 apparatus.

In various embodiments, the pharmaceutical composition comprises:
(i) a crystalline form of the compound of formula (I)

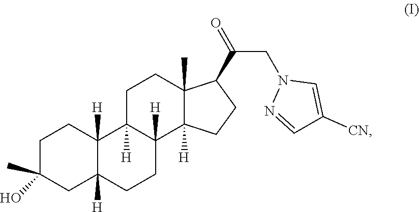

(ii) a filler;
(iii) a lubricant; and
(iv) a glidant.

In certain embodiments, the filler comprises a brittle filler, a ductile filler, or combinations thereof. In certain embodiments, the filler comprises a brittle filler and a ductile filler.

In certain embodiments, the mass ratio of the brittle filler to the ductile filler is about 1 to 9 to about 9 to 1, about 1 to 8 to about 8 to 1, about 1 to 7 to about 7 to 1, about 1 to 6 to about 6 to 1, about 1 to 5 to about 5 to 1, about 1 to 4 to about 4 to 1, about 1 to 3 to about 3 to 1, or about 1 to 2 to about 2 to 1. In certain embodiments, the mass ratio of the brittle filler to the ductile filler is about 1 to 9 to about 9 to 1. In certain embodiments, the mass ratio of the brittle filler to the ductile filler is about 1 to 5 to about 5 to 1. In certain embodiments, the mass ratio of the brittle filler to the ductile filler is about 1 to 4 to about 4 to 1.

In certain embodiments, the mass ratio of the brittle filler to the ductile filler is about 1 to 9, about 1 to 8, about 1 to 7, about 1 to 6, about 1 to 5, about 1 to 4, about 1 to 3, about 1 to 2, about 1 to 1, about 2 to 1, about 3 to 1, about 4 to 1, about 5 to 1, about 6 to 1, about 7 to 1, about 8 to 1, or about 9 to 1.

In certain embodiments, the amount of crystalline form of the compound of formula (I) in the pharmaceutical composition is about 0.4% (w/w) to about 60% (w/w).

In certain embodiments, the amount of the brittle filler in the pharmaceutical composition is about 0% (w/w) to about 90% (w/w).

In certain embodiments, the amount of the ductile filler in the pharmaceutical composition is about 0% (w/w) to about 90% (w/w).

In certain embodiments, the amount of the disintegrant in the pharmaceutical composition is about 0% (w/w) to about 10% (w/w).

In certain embodiments, the amount of the lubricant in the pharmaceutical composition is about 0.1% (w/w) to about 5% (w/w).

In certain embodiments, the amount of the glidant in the pharmaceutical composition is about 0.1% (w/w) to about 3% (w/w).

In certain embodiments, the pharmaceutical composition further comprises a binder. In certain embodiments, the amount of the binder in the pharmaceutical composition is about 0% (w/w) to about 10% (w/w).

In certain embodiments, the pharmaceutical composition further comprises a wetting agent. In certain embodiments, the amount of the wetting agent in the pharmaceutical composition is about 0% (w/w) to about 3% (w/w).

In certain embodiments, the pharmaceutical composition comprises:
(i) a crystalline form of the compound of formula (I)

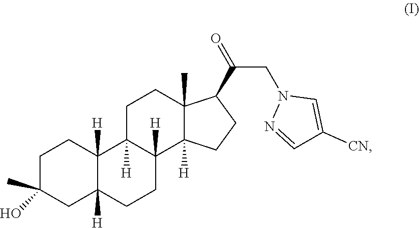

(ii) a brittle filler;
(iii) a ductile filler;
(iv) a lubricant; and
(v) a glidant.

In certain embodiments, the pharmaceutical composition comprises:
(i) about 0.4% (w/w) to about 60% (w/w) of a crystalline form of the compound of formula (I)

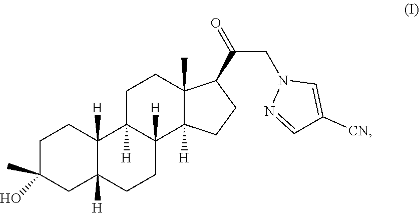

(ii) a brittle filler;
(iii) a ductile filler;
(iv) about 0.1% (w/w) to about 5% (w/w) of a lubricant; and
(v) about 0.1% (w/w) to about 3% (w/w) of a glidant.

In various embodiments, the pharmaceutical composition comprises:
(i) about 0.4% (w/w) to about 60% (w/w) of a crystalline form of the compound of formula (I)

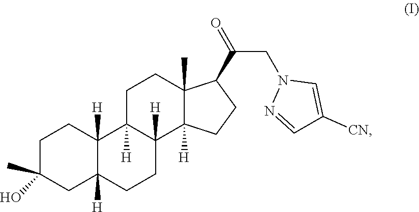

(ii) about 0% (w/w) to about 90% (w/w) of a brittle filler;
(iii) about 0% (w/w) to about 90% (w/w) of a ductile filler;
(iv) about 0% (w/w) to about 15% (w/w) of a disintegrant;
(v) about 0.1% (w/w) to about 5% (w/w) of a lubricant; and
(vi) about 0.1% (w/w) to about 5% (w/w) of a glidant.

In certain embodiments, the amount of crystalline form of the compound of formula (I) in the pharmaceutical composition is about 0.4% (w/w) to about 60% (w/w), about 5% (w/w) to about 60% (w/w), about 10% (w/w) to about 60% (w/w), about 15% (w/w) to about 60% (w/w), about 20% (w/w) to about 60% (w/w), about 30% (w/w) to about 60% (w/w), about 40% (w/w) to about 60% (w/w), about 50% (w/w) to about 60% (w/w), about 0.4% (w/w) to about 50% (w/w), about 0.4% (w/w) to about 40% (w/w), about 0.4% (w/w) to about 30% (w/w), about 0.4% (w/w) to about 20% (w/w), about 0.4% (w/w) to about 15% (w/w), about 0.4% (w/w) to about 10% (w/w), about 0.4% (w/w) to about 5% (w/w), about 5% (w/w) to about 50% (w/w), about 5% (w/w) to about 40% (w/w), about 5% (w/w) to about 30% (w/w), about 5% (w/w) to about 20% (w/w), about 5% (w/w) to about 15% (w/w), about 5% (w/w) to about 10% (w/w), about 10% (w/w) to about 50% (w/w), about 10% (w/w) to about 40% (w/w), about 10% (w/w) to about 30% (w/w), about 10% (w/w) to about 20% (w/w), about 10% (w/w) to about 15% (w/w), about 15% (w/w) to about 50% (w/w), about 15% (w/w) to about 40% (w/w), about 15% (w/w) to about 30% (w/w), about 15% (w/w) to about 20% (w/w), about 20% (w/w) to about 50% (w/w), about 20% (w/w) to about 40% (w/w), about 20% (w/w) to about 30% (w/w), about 30% (w/w) to about 50% (w/w), about 30% (w/w) to about 40% (w/w), or about 40% (w/w) to about 50% (w/w). In certain embodiments, the amount of crystalline form of the compound of formula (I) in the pharmaceutical composition is about 0.4% (w/w) to about 40% (w/w). In certain embodiments, the amount of crystalline form of the compound of formula (I) in the pharmaceutical composition is about 10% (w/w) to about 40% (w/w). In certain embodiments, the amount of crystalline form of the compound of formula (I) in the pharmaceutical composition is about 10% (w/w) to about 15% (w/w).

In certain embodiments, the amount of crystalline form of the compound of formula (I) in the pharmaceutical composition is about 0.4% (w/w), about 0.5% (w/w), about 0.6% (w/w), about 0.7% (w/w), about 0.8% (w/w), about 0.9% (w/w), about 1% (w/w), about 2% (w/w), about 3% (w/w), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 21% (w/w), about 22% (w/w), about 23% (w/w), about 24% (w/w), about 25% (w/w), about 26% (w/w), about 27% (w/w), about 28% (w/w), about 29% (w/w), about 30% (w/w), about 31% (w/w), about 32% (w/w), about 33% (w/w), about 34% (w/w), about 35% (w/w), about 36% (w/w), about 37% (w/w), about 38% (w/w), about 39% (w/w), about 40% (w/w), about 41% (w/w), about 42% (w/w), about 43% (w/w), about 44% (w/w), about 45% (w/w), about 46% (w/w), about 47% (w/w), about 48% (w/w), about 49% (w/w), about 50% (w/w), about 51% (w/w), about 52% (w/w), about 53% (w/w), about 54% (w/w), about 55% (w/w), about 56% (w/w), about 57% (w/w), about 58% (w/w), about 59% (w/w), or about 60% (w/w). In certain embodiments, the amount of crystalline form of the compound of formula (I) in the pharmaceutical composition is about 12% (w/w). In certain embodiments, the amount of crystalline form of the compound of formula (I) in the pharmaceutical composition is about 24% (w/w). In certain embodiments, the amount of crystalline form of the compound of formula (I) in the pharmaceutical composition is about 36% (w/w).

In certain embodiments, the amount of crystalline form of the compound of formula (I) in the pharmaceutical composition is about 0.1 mg to about 100 mg, about 1 mg to about 100 mg, about 5 mg to about 100 mg, about 10 mg to about 100 mg, about 15 mg to about 100 mg, about 20 mg to about 100 mg, about 30 mg to about 100 mg, about 40 mg to about 100 mg, about 50 mg to about 100 mg, about 60 mg to about 100 mg, about 70 mg to about 100 mg, about 80 mg to about 100 mg, about 90 mg to about 100 mg, about 0.1 mg to about 90 mg, about 0.1 mg to about 80 mg, about 0.1 mg to about 70 mg, about 0.1 mg to about 60 mg, about 0.1 mg to about 50 mg, about 0.1 mg to about 40 mg, about 0.1 mg to about 30 mg, about 0.1 mg to about 20 mg, about 0.1 mg to about 15 mg, about 0.1 mg to about 10 mg, about 0.1 mg to about 5 mg, about 0.1 mg to about 1 mg, about 1 mg to about 90 mg, about 1 mg to about 80 mg, about 1 mg to about 70 mg, about 1 mg to about 60 mg, about 1 mg to about 50 mg, about 1 mg to about 40 mg, about 1 mg to about 30 mg, about 1 mg to about 20 mg, about 1 mg to about 15 mg, about 1 mg to about 10 mg, about 1 mg to about 5 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 15 mg, about 5 mg to about 10 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 40 mg, about 10 mg to about 30 mg, about 10 mg to about 20 mg, about 10 mg to about 15 mg, about 15 mg to about 90 mg, about 15 mg to about 80 mg, about 15 mg to about 70 mg, about 15 mg to about 60 mg, about 15 mg to about 50 mg, about 15 mg to about 40 mg, about 15 mg to about 30 mg, about 15 mg to about 20 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 90 mg, about 40 mg to about 80 mg, about 40 mg to about 70 mg, about 40 mg to about 60 mg, about 40 mg to about 50 mg, about 50 mg to about 90 mg, about 50 mg to about 80 mg, about 50 mg to about 70 mg, about 50 mg to about 60 mg, about 60 mg to about 90 mg, about 60 mg to about 80 mg, about 60 mg to about 70 mg, about 70 mg to about 90 mg, about 70 mg to about 80 mg, or about 80 mg to about 90 mg.

In certain embodiments, the amount of crystalline form of the compound of formula (I) in the pharmaceutical composition is about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, or about 100 mg. In certain embodiments, the amount of crystalline form of the compound of formula (I) in the pharmaceutical composition is about 20 mg. In certain embodiments, the amount of crystalline form of the compound of formula (I) in the pharmaceutical composition is about 25 mg. In certain embodiments, the amount of crystalline form of the compound of formula (I) in the pharmaceutical composition is about 30 mg. In certain embodiments, the amount of crystalline form of the compound of formula (I) in the pharmaceutical composition is about 40 mg. In certain embodiments, the amount of crystalline form of the compound of formula (I) in the pharmaceutical composition is about 50 mg. In certain embodiments, the amount of crystalline form of the compound of formula (I) in the pharmaceutical composition is about 60 mg.

In certain embodiments, the amount of the brittle filler in the pharmaceutical composition is about 0% (w/w) to about 90% (w/w), about 10% (w/w) to about 90% (w/w), about 20% (w/w) to about 90% (w/w), about 30% (w/w) to about 90% (w/w), about 40% (w/w) to about 90% (w/w), about 50% (w/w) to about 90% (w/w), about 60% (w/w) to about 90% (w/w), about 70% (w/w) to about 90% (w/w), about 75% (w/w) to about 90% (w/w), about 80% (w/w) to about 90% (w/w), about 85% (w/w) to about 90% (w/w), about 0% (w/w) to about 85% (w/w), about 0% (w/w) to about 80% (w/w), about 0% (w/w) to about 75% (w/w), about 0% (w/w) to about 70% (w/w), about 0% (w/w) to about 60% (w/w), about 0% (w/w) to about 50% (w/w), about 0% (w/w) to about 40% (w/w), about 0% (w/w) to about 30% (w/w), about 0% (w/w) to about 20% (w/w), about 0% (w/w) to about 10% (w/w), about 10% (w/w) to about 85% (w/w), about 10% (w/w) to about 80% (w/w), about 10% (w/w) to about 75% (w/w), about 10% (w/w) to about 70% (w/w), about 10% (w/w) to about 60% (w/w), about 10% (w/w) to about 50% (w/w), about 10% (w/w) to about 40% (w/w), about 10% (w/w) to about 30% (w/w), about 10% (w/w) to about 20% (w/w), about 20% (w/w) to about 85% (w/w), about 20% (w/w) to about 80% (w/w), about 20% (w/w) to about 75% (w/w), about 20% (w/w) to about 70% (w/w), about 20% (w/w) to about 60% (w/w), about 20% (w/w) to about 50% (w/w), about 20% (w/w) to about 40% (w/w), about 20% (w/w) to about 30% (w/w), about 30% (w/w) to about 85% (w/w), about 30% (w/w) to about 80% (w/w), about 30% (w/w) to about 75% (w/w), about 30% (w/w) to about 70% (w/w), about 30% (w/w) to about 60% (w/w), about 30% (w/w) to about 50% (w/w), about 30% (w/w) to about 40% (w/w), about 40% (w/w) to about 80% (w/w), about 40% (w/w) to about 70% (w/w), about 40% (w/w) to about 60% (w/w), about 40% (w/w) to about 50% (w/w), about 50% (w/w) to about 85% (w/w), about 50% (w/w) to about 80% (w/w), about 50% (w/w) to about 75% (w/w), about 50% (w/w) to about 70% (w/w), about 50% (w/w) to about 60% (w/w), about 60% (w/w) to about 85% (w/w), about 60% (w/w) to about 80% (w/w), about 60% (w/w) to about 75% (w/w), about 60% (w/w) to about 70% (w/w), about 70% (w/w) to about 85% (w/w), about 70% (w/w) to about 80% (w/w), about 70% (w/w) to about 75% (w/w), about 75% (w/w) to about 85% (w/w), about 75% (w/w) to about 80% (w/w), or about 80% (w/w) to about 85% (w/w). In certain embodiments, the amount of the brittle filler in the pharmaceutical composition is about 30% (w/w) to about 75% (w/w). In certain embodiments, the amount of the brittle filler in the pharmaceutical composition is about 60% (w/w) to about 70% (w/w).

In certain embodiments, the amount of the brittle filler in the pharmaceutical composition is about 35% (w/w) to about 45% (w/w), about 37% (w/w) to about 45% (w/w), about 39% (w/w) to about 45% (w/w), about 41% (w/w) to about 45% (w/w), about 43% (w/w) to about 45% (w/w), about 35% (w/w) to about 43% (w/w), about 35% (w/w) to about 41% (w/w), about 35% (w/w) to about 39% (w/w), about 35% (w/w) to about 37% (w/w), about 37% (w/w) to about 43% (w/w), about 37% (w/w) to about 41% (w/w), about 37% (w/w) to about 39% (w/w), about 39% (w/w) to about 43% (w/w), about 39% (w/w) to about 41% (w/w), or about 41% (w/w) to about 43% (w/w). In certain embodiments, the amount of the brittle filler in the pharmaceutical composition is about 41% (w/w) to about 45% (w/w).

In certain embodiments, the amount of the brittle filler in the pharmaceutical composition is about 100 mg to about 400 mg, about 125 mg to about 400 mg, about 150 mg to about 400 mg, about 175 mg to about 400 mg, about 200 mg to about 400 mg, about 225 mg to about 400 mg, about 250 mg to about 400 mg, about 275 mg to about 400 mg, about 300 mg to about 400 mg, about 325 mg to about 400 mg, about 350 mg to about 400 mg, about 375 mg to about 400 mg, about 100 mg to about 375 mg, about 100 mg to about 350 mg, about 100 mg to about 325 mg, about 100 mg to about 300 mg, about 100 mg to about 275 mg, about 100 mg to about 250 mg, about 100 mg to about 225 mg, about 100 mg to about 200 mg, about 100 mg to about 175 mg, about 100 mg to about 150 mg, about 100 mg to about 125 mg, about 125 mg to about 375 mg, about 125 mg to about 350 mg, about 125 mg to about 325 mg, about 125 mg to about 300 mg, about 125 mg to about 275 mg, about 125 mg to about 250 mg, about 125 mg to about 225 mg, about 125 mg to about 200 mg, about 125 mg to about 175 mg, about 125 mg to about 150 mg, about 150 mg to about 375 mg, about 150 mg to about 350 mg, about 150 mg to about 325 mg, about 150 mg to about 300 mg, about 150 mg to about 275 mg, about 150 mg to about 250 mg, about 150 mg to about 225 mg, about 150 mg to about 200 mg, about 150 mg to about 175 mg, about 175 mg to about 375 mg, about 175 mg to about 350 mg, about 175 mg to about 325 mg, about 175 mg to about 300 mg, about 175 mg to about 275 mg, about 175 mg to about 250 mg, about 175 mg to about 225 mg, about 175 mg to about 200 mg, about 200 mg to about 400 mg, about 200 mg to about 375 mg, about 200 mg to about 350 mg, about 200 mg to about 325 mg, about 200 mg to about 300 mg, about 200 mg to about 275 mg, about 200 mg to about 250 mg, about 200 mg to about 225 mg, about 225 mg to about 375 mg, about 225 mg to about 350 mg, about 225 mg to about 325 mg, about 225 mg to about 300 mg, about 225 mg to about 275 mg, about 225 mg to about 250 mg, about 250 mg to about 375 mg, about 250 mg to about 350 mg, about 250 mg to about 325 mg, about 250 mg to about 300 mg, about 250 mg to about 275 mg, about 275 mg to about 375 mg, about 275 mg to about 350 mg, about 275 mg to about 325 mg, about 275 mg to about 300 mg, about 300 mg to about 375 mg, about 300 mg to about 350 mg, about 300 mg to about 325 mg, about 325 mg to about 375 mg, about 325 mg to about 350 mg, or about 350 mg to about 400 mg.

In certain embodiments, the amount of the brittle filler in the pharmaceutical composition is about 100 mg, about 125 mg, about 150 mg, about 175 mg, about 200 mg, about 225 mg, about 250 mg, about 275 mg, about 300 mg, about 325 mg, about 350 mg, about 375 mg, or about 400 mg. In certain embodiments, the amount of the brittle filler in the pharmaceutical composition is about 105.9 mg. In certain embodiments, the amount of the brittle filler in the pharmaceutical composition is about 132 mg. In certain embodiments, the amount of the brittle filler in the pharmaceutical composition is about 159 mg. In certain embodiments, the amount of the brittle filler in the pharmaceutical composition is about 212 mg. In certain embodiments, the amount of the brittle filler in the pharmaceutical composition is about 265 mg. In certain embodiments, the amount of the brittle filler in the pharmaceutical composition is about 317.7 mg.

In certain embodiments, the amount of the ductile filler in the pharmaceutical composition is about 0% (w/w) to about 90% (w/w), about 5% (w/w) to about 90% (w/w), about 10% (w/w) to about 90% (w/w), about 15% (w/w) to about 90% (w/w), about 20% (w/w) to about 90% (w/w), about 25% (w/w) to about 90% (w/w), about 35% (w/w) to about 90% (w/w), about 45% (w/w) to about 90% (w/w), about 55% (w/w) to about 90% (w/w), about 65% (w/w) to about 90% (w/w), about 75% (w/w) to about 90% (w/w), about 0% (w/w) to about 75% (w/w), about 0% (w/w) to about 65% (w/w), about 0% (w/w) to about 55% (w/w), about 0% (w/w) to about 45% (w/w), about 0% (w/w) to about 35% (w/w), about 0% (w/w) to about 25% (w/w), about 0% (w/w) to about 20% (w/w), about 0% (w/w) to about 15% (w/w), about 0% (w/w) to about 10% (w/w), about 0% (w/w) to about 5% (w/w), about 5% (w/w) to about 75% (w/w), about 5% (w/w) to about 65% (w/w), about 5% (w/w) to about 55% (w/w), about 5% (w/w) to about 45% (w/w), about 5% (w/w) to about 35% (w/w), about 5% (w/w) to about 25% (w/w), about 5% (w/w) to about 20% (w/w), about 5% (w/w) to about 15% (w/w), about 5% (w/w) to about 10% (w/w), about 10% (w/w) to about 75% (w/w), about 10% (w/w) to about 65% (w/w), about 10% (w/w) to about 55% (w/w), about 10% (w/w) to about 45% (w/w), about 10% (w/w) to about 35% (w/w), about 10% (w/w) to about 25% (w/w), about 10% (w/w) to about 20% (w/w), about 10% (w/w) to about 15% (w/w), about 15% (w/w) to about 75% (w/w), about 15% (w/w) to about 65% (w/w), about 15% (w/w) to about 55% (w/w), about 15% (w/w) to about 45% (w/w), about 15% (w/w) to about 35% (w/w), about 15% (w/w) to about 25% (w/w), about 15% (w/w) to about 20% (w/w), about 20% (w/w) to about 75% (w/w), about 20% (w/w) to about 65% (w/w), about 20% (w/w) to about 55% (w/w), about 20% (w/w) to about 45% (w/w), about 20% (w/w) to about 35% (w/w), about 20% (w/w) to about 25% (w/w), about 25% (w/w) to about 75% (w/w), about 25% (w/w) to about 65% (w/w), about 25% (w/w) to about 55% (w/w), about 25% (w/w) to about 45% (w/w), about 25% (w/w) to about 35% (w/w), about 35% (w/w) to about 75% (w/w), about 35% (w/w) to about 65% (w/w), about 35% (w/w) to about 55% (w/w), about 35% (w/w) to about 45% (w/w), about 45% (w/w) to about 75% (w/w), about 45% (w/w) to about 65% (w/w), about 45% (w/w) to about 55% (w/w), about 55% (w/w) to about 75% (w/w), about 55% (w/w) to about 65% (w/w), or about 65% (w/w) to about 75% (w/w). In certain embodiments, the amount of the ductile filler in the pharmaceutical composition is about 5% (w/w) to about 25% (w/w). In certain embodiments, the amount of the ductile filler in the pharmaceutical composition is about 10% (w/w) to about 20% (w/w).

In certain embodiments, the amount of the ductile filler in the pharmaceutical composition is about 35% (w/w) to about 45% (w/w), about 37% (w/w) to about 45% (w/w), about 39% (w/w) to about 45% (w/w), about 41% (w/w) to about 45% (w/w), about 43% (w/w) to about 45% (w/w), about 35% (w/w) to about 43% (w/w), about 35% (w/w) to about 41% (w/w), about 35% (w/w) to about 39% (w/w), about 35% (w/w) to about 37% (w/w), about 37% (w/w) to about 43% (w/w), about 37% (w/w) to about 41% (w/w), about 37% (w/w) to about 39% (w/w), about 39% (w/w) to about 43% (w/w), about 39% (w/w) to about 41% (w/w), or about 41% (w/w) to about 43% (w/w). In certain embodiments, the amount of the ductile filler in the pharmaceutical composition is about 41% (w/w) to about 45% (w/w).

In certain embodiments, the amount of the ductile filler in the pharmaceutical composition is about 10 mg to about 100 mg, about 20 mg to about 100 mg, about 30 mg to about 100 mg, about 40 mg to about 100 mg, about 50 mg to about 100 mg, about 60 mg to about 100 mg, about 70 mg to about 100 mg, about 80 mg to about 100 mg, about 90 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 40 mg, about 10 mg to about 30 mg, about 10 mg to about 20 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 90 mg, about 40 mg to about 80 mg, about 40 mg to about 70 mg, about 40 mg to about 60 mg, about 40 mg to about 50 mg, about 50 mg to about 90 mg, about 50 mg to about 80 mg, about 50 mg to about 70 mg, about 50 mg to about 60 mg, about 60 mg to about 90 mg, about 60 mg to about 80 mg, about 60 mg to about 70 mg, about 70 mg to about 90 mg, about 70 mg to about 80 mg, or about 80 mg to about 90 mg.

In certain embodiments, the amount of the ductile filler in the pharmaceutical composition is about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In certain embodiments, the amount of the ductile filler in the pharmaceutical composition is about 26.2 mg. In certain embodiments, the amount of the ductile filler in the pharmaceutical composition is about 32.7 mg. In certain embodiments, the amount of the ductile filler in the pharmaceutical composition is about 39.3 mg. In certain embodiments, the amount of the ductile filler in the pharmaceutical composition is about 52.4 mg. In certain embodiments, the amount of the ductile filler in the pharmaceutical composition is about 65.5 mg. In certain embodiments, the amount of the ductile filler in the pharmaceutical composition is about 78.6 mg.

In certain embodiments, the amount of the disintegrant in the pharmaceutical composition is about 0% (w/w) to about 15% (w/w), about 0.5% (w/w) to about 15% (w/w), about 1% (w/w) to about 15% (w/w), about 1.5% (w/w) to about 15% (w/w), about 2% (w/w) to about 15% (w/w), about 2.5% (w/w) to about 15% (w/w), about 4% (w/w) to about 15% (w/w), about 6% (w/w) to about 15% (w/w), about 8% (w/w) to about 15% (w/w), about 10% (w/w) to about 15% (w/w), about 12% (w/w) to about 15% (w/w), about 0% (w/w) to about 12% (w/w), about 0% (w/w) to about 10% (w/w), about 0% (w/w) to about 8% (w/w), about 0% (w/w) to about 6% (w/w), about 0% (w/w) to about 4% (w/w), about 0% (w/w) to about 2.5% (w/w), about 0% (w/w) to about 2% (w/w), about 0% (w/w) to about 1.5% (w/w), about 0% (w/w) to about 1% (w/w), about 0% (w/w) to about 0.5% (w/w), about 0.5% (w/w) to about 12% (w/w), about 0.5% (w/w) to about 10% (w/w), about 0.5% (w/w) to about 8% (w/w), about 0.5% (w/w) to about 6% (w/w), about 0.5% (w/w) to about 4% (w/w), about 0.5% (w/w) to about 2.5% (w/w), about 0.5% (w/w) to about 2% (w/w), about 0.5% (w/w) to about 1.5% (w/w), about 0.5% (w/w) to about 1% (w/w), about 1% (w/w) to about 12% (w/w), about 1% (w/w) to about 10% (w/w), about 1% (w/w) to about 8% (w/w), about 1% (w/w) to about 6% (w/w), about 1% (w/w) to about 4% (w/w), about 1% (w/w) to about 2.5% (w/w), about 1% (w/w) to about 2% (w/w), about 1% (w/w) to about 1.5% (w/w), about 1.5% (w/w) to about 12% (w/w), about 1.5% (w/w) to about 10% (w/w), about 1.5% (w/w) to about 8% (w/w), about 1.5% (w/w) to about 6% (w/w), about 1.5% (w/w) to about 4% (w/w), about 1.5% (w/w) to about 2.5% (w/w), about 1.5% (w/w) to about 2% (w/w), about 2% (w/w) to about 12% (w/w), about 2% (w/w) to about 10% (w/w), about 2% (w/w) to about 8% (w/w), about 2% (w/w) to about 6% (w/w), about 2% (w/w) to about 4% (w/w), about 2% (w/w) to about 2.5% (w/w), about 2.5% (w/w) to about 12% (w/w), about 2.5% (w/w) to about 10% (w/w), about 2.5% (w/w) to about 8% (w/w), about 2.5% (w/w) to about 6% (w/w), about 2.5% (w/w) to about 4% (w/w), about 4% (w/w) to about 12% (w/w), about 4% (w/w) to about 10% (w/w), about 4% (w/w) to about 8% (w/w), about 4% (w/w) to about 6% (w/w), about 6% (w/w) to about 12% (w/w), about 6% (w/w) to about 10% (w/w), about 6% (w/w) to about 8% (w/w), about 8% (w/w) to about 12% (w/w), about 8% (w/w) to about 10% (w/w), or about 10% (w/w) to about 12% (w/w). In certain embodiments, the amount of the disintegrant in the pharmaceutical composition is about 2.5% (w/w) to about 10% (w/w). In certain embodiments, the amount of the disintegrant in the pharmaceutical composition is about 4% (w/w) to about 8% (w/w).

In certain embodiments, the amount of the disintegrant in the pharmaceutical composition is about 5 mg to about 40 mg, about 10 mg to about 40 mg, about 15 mg to about 40 mg, about 20 mg to about 40 mg, about 25 mg to about 40 mg, about 30 mg to about 40 mg, about 35 mg to about 40 mg, about 5 mg to about 35 mg, about 5 mg to about 30 mg, about 5 mg to about 25 mg, about 5 mg to about 20 mg, about 5 mg to about 15 mg, about 5 mg to about 10 mg, about 10 mg to about 35 mg, about 10 mg to about 30 mg, about 10 mg to about 25 mg, about 10 mg to about 20 mg, about 10 mg to about 15 mg, about 15 mg to about 35 mg, about 15 mg to about 30 mg, about 15 mg to about 25 mg, about 15 mg to about 20 mg, about 20 mg to about 35 mg, about 15 mg to about 30 mg, about 15 mg to about 25 mg, about 15 mg to about 20 mg, about 20 mg to about 35 mg, about 20 mg to about 30 mg, about 20 mg to about 25 mg, about 25 mg to about 35 mg, about 25 mg to about 30 mg, or about 30 mg to about 35 mg.

In certain embodiments, the amount of the disintegrant in the pharmaceutical composition is about 5 mg, about 7.5 mg, about 10 mg, about 12.5 mg, about 15 mg, about 17.5 mg, about 20 mg, about 22.5 mg, about 25 mg, about 27.5 mg, about 30 mg, about 32.5 mg, about 35 mg, about 37.5 mg, or about 40 mg. In certain embodiments, the amount of the disintegrant in the pharmaceutical composition is about 10 mg. In certain embodiments, the amount of the disintegrant in the pharmaceutical composition is about 12.5 mg. In certain embodiments, the amount of the disintegrant in the pharmaceutical composition is about 15 mg. In certain embodiments, the amount of the disintegrant in the pharmaceutical composition is about 20 mg. In certain embodiments, the amount of the disintegrant in the pharmaceutical composition is about 25 mg. In certain embodiments, the amount of the disintegrant in the pharmaceutical composition is about 30 mg.

In certain embodiments, the amount of the lubricant in the pharmaceutical composition is about 0.1% (w/w) to about 5% (w/w), about 0.25% (w/w) to about 5% (w/w), about 0.5% (w/w) to about 5% (w/w), about 1% (w/w) to about 5% (w/w), about 2% (w/w) to about 5% (w/w), about 3% (w/w) to about 5% (w/w), about 4% (w/w) to about 5% (w/w), about 0.1% (w/w) to about 4% (w/w), about 0.1% (w/w) to about 3% (w/w), about 0.1% (w/w) to about 2% (w/w), about 0.1% (w/w) to about 1% (w/w), about 0.1% (w/w) to about 0.5% (w/w), about 0.1% (w/w) to about 0.25% (w/w), about 0.25% (w/w) to about 4% (w/w), about 0.25% (w/w) to about 3% (w/w), about 0.25% (w/w) to about 2% (w/w), about 0.25% (w/w) to about 1% (w/w), about 0.25% (w/w) to about 0.5% (w/w), about 0.5% (w/w) to about 4% (w/w), about 0.5% (w/w) to about 3% (w/w), about 0.5% (w/w) to about 2% (w/w), about 0.5% (w/w) to about 1% (w/w), about 1% (w/w) to about 4% (w/w), about 1% (w/w) to about 3% (w/w), about 1% (w/w) to about 2% (w/w), about 2% (w/w) to about 4% (w/w), about 2% (w/w) to about 3% (w/w), or about 3% (w/w) to about 4% (w/w). In certain embodiments, the amount of the lubricant in the pharmaceutical composition is about 0.25% (w/w) to about 3% (w/w). In certain embodiments, the amount of the lubricant in the pharmaceutical composition is about 1% (w/w) to about 2% (w/w).

In certain embodiments, the amount of the lubricant in the pharmaceutical composition is about 1 mg to about 10 mg, about 1.2 mg to about 10 mg, about 1.4 mg to about 10 mg, about 1.6 mg to about 10 mg, about 1.8 mg to about 10 mg, about 2 mg to about 10 mg, about 2.5 mg to about 10 mg, about 3 mg to about 10 mg, about 4 mg to about 10 mg, about 5 mg to about 10 mg, about 6 mg to about 10 mg, about 8 mg to about 10 mg, about 1 mg to about 8 mg, about 1 mg to about 6 mg, about 1 mg to about 5 mg, about 1 mg to about 4 mg, about 1 mg to about 3 mg, about 1 mg to about 2.5 mg, about 1 mg to about 2 mg, about 1 mg to about 1.8 mg, about 1 mg to about 1.6 mg, about 1 mg to about 1.4 mg, about 1 mg to about 1.2 mg, about 1.2 mg to about 8 mg, about 1.2 mg to about 6 mg, about 1.2 mg to about 5 mg, about 1.2 mg to about 4 mg, about 1.2 mg to about 3 mg, about 1.2 mg to about 2.5 mg, about 1.2 mg to about 2 mg, about 1.2 mg to about 1.8 mg, about 1.2 mg to about 1.6 mg, about 1.2 mg to about 1.4 mg, about 1.4 mg to about 8 mg, about 1.4 mg to about 6 mg, about 1.4 mg to about 5 mg, about 1.4 mg to about 4 mg, about 1.4 mg to about 3 mg, about 1.4 mg to about 2.5 mg, about 1.4 mg to about 2 mg, about 1.4 mg to about 1.8 mg, about 1.4 mg to about 1.6 mg, about 1.6 mg to about 8 mg, about 1.6 mg to about 6 mg, about 1.6 mg to about 5 mg, about 1.6 mg to about 4 mg, about 1.6 mg to about 3 mg, about 1.6 mg to about 2.5 mg, about 1.6 mg to about 2 mg, about 1.6 mg to about 1.8 mg, about 1.8 mg to about 8 mg, about 1.8 mg to about 6 mg, about 1.8 mg to about 5 mg, about 1.8 mg to about 4 mg, about 1.8 mg to about 3 mg, about 1.8 mg to about 2.5 mg, about 1.8 mg to about 2 mg, about 2 mg to about 8 mg, about 2 mg to about 6 mg, about 2 mg to about 5 mg, about 2 mg to about 4 mg, about 2 mg to about 3 mg, about 2 mg to about 2.5 mg, about 2.5 mg to about 8 mg, about 2.5 mg to about 6 mg, about 2.5 mg to about 5 mg, about 2.5 mg to about 4 mg, about 2.5 mg to about 3 mg, about 3 mg to about 8 mg, about 3 mg to about 6 mg, about 3 mg to about 5 mg, about 3 mg to about 4 mg, about 4 mg to about 8 mg, about 4 mg to about 6 mg, about 4 mg to about 5 mg, about 5 mg to about 8 mg, about 5 mg to about 6 mg, or about 6 mg to about 8 mg.

In certain embodiments, the amount of the lubricant in the pharmaceutical composition is about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg. In certain embodiments, the amount of the lubricant in the pharmaceutical composition is about 1.7 mg. In certain embodiments, the amount of the lubricant in the pharmaceutical composition is about 2.1 mg. In certain embodiments, the amount of the lubricant in the pharmaceutical composition is about 2.5 mg. In certain embodiments, the amount of the lubricant in the pharmaceutical composition is about 3.3 mg. In certain embodiments, the amount of the lubricant in the pharmaceutical composition is about 4.2 mg. In certain embodiments, the amount of the lubricant in the pharmaceutical composition is about 5 mg.

In certain embodiments, the amount of the glidant in the pharmaceutical composition is about 0.1% (w/w) to about 5% (w/w), about 0.25% (w/w) to about 5% (w/w), about 0.5% (w/w) to about 5% (w/w), about 1% (w/w) to about 5% (w/w), about 1.5% (w/w) to about 5% (w/w), about 2% (w/w) to about 5% (w/w), about 2.5% (w/w) to about 5% (w/w), about 3% (w/w) to about 5% (w/w), about 4% (w/w) to about 5% (w/w), about 0.1% (w/w) to about 4% (w/w), about 0.1% (w/w) to about 3% (w/w), about 0.1% (w/w) to about 2.5% (w/w), about 0.1% (w/w) to about 2% (w/w), about 0.1% (w/w) to about 1.5% (w/w), about 0.1% (w/w) to about 1% (w/w), about 0.1% (w/w) to about 0.5% (w/w), about 0.1% (w/w) to about 0.25% (w/w), about 0.25% (w/w) to about 4% (w/w), about 0.25% (w/w) to about 3% (w/w), about 0.25% (w/w) to about 2.5% (w/w), about 0.25% (w/w) to about 2% (w/w), about 0.25% (w/w) to about 1.5% (w/w), about 0.25% (w/w) to about 1% (w/w), about 0.25% (w/w) to about 0.5% (w/w), about 0.5% (w/w) to about 4% (w/w), about 0.5% (w/w) to about 3% (w/w), about 0.5% (w/w) to about 2.5% (w/w), about 0.5% (w/w) to about 2% (w/w), about 0.5% (w/w) to about 1.5% (w/w), about 0.5% (w/w) to about 1% (w/w), about 1% (w/w) to about 4% (w/w), about 1% (w/w) to about 3% (w/w), about 1% (w/w) to about 2.5% (w/w), about 1% (w/w) to about 2% (w/w), about 1% (w/w) to about 1.5% (w/w), about 1.5% (w/w) to about 4% (w/w), about 1.5% (w/w) to about 3% (w/w), about 1.5% (w/w) to about 2.5% (w/w), about 1.5% (w/w) to about 2% (w/w), about 2% (w/w) to about 4% (w/w), about 2% (w/w) to about 3% (w/w), about 2% (w/w) to about 2.5% (w/w), about 2.5% (w/w) to about 4% (w/w), about 2.5% (w/w) to about 3% (w/w), or about 3% (w/w) to about 4% (w/w). In certain embodiments, the amount of the glidant in the pharmaceutical composition is about 0.25% (w/w) to about 2.5% (w/w). In certain embodiments, the amount of the glidant in the pharmaceutical composition is about 0.5% (w/w) to about 2% (w/w).

In certain embodiments, the amount of the glidant in the pharmaceutical composition is about 1 mg to about 10 mg, about 1.2 mg to about 10 mg, about 1.4 mg to about 10 mg, about 1.6 mg to about 10 mg, about 1.8 mg to about 10 mg, about 2 mg to about 10 mg, about 2.5 mg to about 10 mg, about 3 mg to about 10 mg, about 3.5 mg to about 10 mg, about 4 mg to about 10 mg, about 4.5 mg to about 10 mg, about 5 mg to about 10 mg, about 6 mg to about 10 mg, about 8 mg to about 10 mg, about 1 mg to about 8 mg, about 1 mg to about 6 mg, about 1 mg to about 5 mg, about 1 mg to about 4.5 mg, about 1 mg to about 4 mg, about 1 mg to about 3.5 mg, about 1 mg to about 3 mg, about 1 mg to about 2.5 mg, about 1 mg to about 2 mg, about 1 mg to about 1.8 mg, about 1 mg to about 1.6 mg, about 1 mg to about 1.4 mg, about 1 mg to about 1.2 mg, about 1.2 mg to about 8 mg, about 1.2 mg to about 6 mg, about 1.2 mg to about 5 mg, about 1.2 mg to about 4.5 mg, about 1.2 mg to about 4 mg, about 1.2 mg to about 3.5 mg, about 1.2 mg to about 3 mg, about 1.2 mg to about 2.5 mg, about 1.2 mg to about 2 mg, about 1.2 mg to about 1.8 mg, about 1.2 mg to about 1.6 mg, about 1.2 mg to about 1.4 mg, about 1.4 mg to about 8 mg, about 1.4 mg to about 6 mg, about 1.4 mg to about 5 mg, about 1.4 mg to about 4.5 mg, about 1.4 mg to about 4 mg, about 1.4 mg to about 3.5 mg, about 1.4 mg to about 3 mg, about 1.4 mg to about 2.5 mg, about 1.4 mg to about 2 mg, about 1.4 mg to about 1.8 mg, about 1.4 mg to about 1.6 mg, about 1.6 mg to about 8 mg, about 1.6 mg to about 6 mg, about 1.6 mg to about 5 mg, about 1.6 mg to about 4.5 mg, about 1.6 mg to about 4 mg, about 1.6 mg to about 3.5 mg, about 1.6 mg to about 3 mg, about 1.6 mg to about 2.5 mg, about 1.6 mg to about 2 mg, about 1.6 mg to about 1.8 mg, about 1.8 mg to about 8 mg, about 1.8 mg to about 6 mg, about 1.8 mg to about 5 mg, about 1.8 mg to about 4.5 mg, about 1.8 mg to about 4 mg, about 1.8 mg to about 3.5 mg, about 1.8 mg to about 3 mg, about 1.8 mg to about 2.5 mg, about 1.8 mg to about 2 mg, about 2 mg to about 8 mg, about 2 mg to about 6 mg, about 2 mg to about 5 mg, about 2 mg to about 4 mg, about 2 mg to about 4 mg, about 2 mg to about 3.5 mg, about 2 mg to about 3 mg, about 2 mg to about 2.5 mg, about 2.5 mg to about 8 mg, about 2.5 mg to about 6 mg, about 2.5 mg to about 5 mg, about 2.5 mg to about 4.5 mg, about 2.5 mg to about 4 mg, about 2.5 mg to about 3.5 mg, about 2.5 mg to about 3 mg, about 3 mg to about 8 mg, about 3 mg to about 6 mg, about 3 mg to about 5 mg, about 3 mg to about 4.5 mg, about 3 mg to about 4 mg, about 3 mg to about 3.5 mg, about 3.5 mg to about 8 mg, about 3.5 mg to about 6 mg, about 3.5 mg to about 5 mg, about 3.5 mg to about 4.5 mg, about 3.5 mg to about 4 mg, about 4 mg to about 8 mg, about 4 mg to about 6 mg, about 4 mg to about 5 mg, about 4 mg to about 4.5 mg, about 4.5 mg to about 8 mg, about 4.5 mg to about 6 mg, about 4.5 mg to about 5 mg, about 5 mg to about 8 mg, about 5 mg to about 6 mg, or about 6 mg to about 8 mg.

In certain embodiments, the amount of the glidant in the pharmaceutical composition is about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg. In certain embodiments, the amount of the glidant in the pharmaceutical composition is about 2.9 mg. In certain embodiments, the amount of the glidant in the pharmaceutical composition is about 3.6 mg. In certain embodiments, the amount of the glidant in the pharmaceutical composition is about 4.4 mg. In certain embodiments, the amount of the glidant in the pharmaceutical composition is about 5.8 mg. In certain embodiments, the amount of the glidant in the pharmaceutical composition is about 7.3 mg. In certain embodiments, the amount of the glidant in the pharmaceutical composition is about 8.8 mg.

In certain embodiments, the amount of the binder in the pharmaceutical composition is about 0% (w/w) to about 10% (w/w), about 2% (w/w) to about 10% (w/w), about 4% (w/w) to about 10% (w/w), about 6% (w/w) to about 10% (w/w), about 8% (w/w) to about 10% (w/w), about 0% (w/w) to about 8% (w/w), about 0% (w/w) to about 6% (w/w), about 0% (w/w) to about 4% (w/w), about 0% (w/w) to about 2% (w/w), about 2% (w/w) to about 8% (w/w), about 2% (w/w) to about 6% (w/w), about 2% (w/w) to about 4% (w/w), about 4% (w/w) to about 8% (w/w), about 4% (w/w) to about 6% (w/w), or about 6% (w/w) to about 8% (w/w).

In certain embodiments, the amount of the binder in the pharmaceutical composition is about 1 mg to about 20 mg, about 2 mg to about 20 mg, about 4 mg to about 20 mg, about 6 mg to about 20 mg, about 8 mg to about 20 mg, about 10 mg to about 20 mg, about 12 mg to about 20 mg, about 15 mg to about 20 mg, about 1 mg to about 15 mg, about 1 mg to about 12 mg, about 1 mg to about 10 mg, about 1 mg to about 8 mg, about 1 mg to about 6 mg, about 1 mg to about 4 mg, about 1 mg to about 2 mg, about 2 mg to about 15 mg, about 2 mg to about 12 mg, about 2 mg to about 10 mg, about 2 mg to about 8 mg, about 2 mg to about 6 mg, about 2 mg to about 4 mg, about 4 mg to about 15 mg, about 4 mg to about 12 mg, about 4 mg to about 10 mg, about 4 mg to about 8 mg, about 4 mg to about 6 mg, about 6 mg to about 15 mg, about 6 mg to about 12 mg, about 6 mg to about 10 mg, about 6 mg to about 8 mg, about 8 mg to about 15 mg, about 8 mg to about 12 mg, about 8 mg to about 10 mg, about 10 mg to about 15 mg, about 10 mg to about 12 mg, or about 12 mg to about 15 mg.

In certain embodiments, the amount of the binder in the pharmaceutical composition is about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg.

In certain embodiments, the amount of the wetting agent in the pharmaceutical composition is about 0% (w/w) to about 3% (w/w), about 0.5% (w/w) to about 3% (w/w), about 1% (w/w) to about 3% (w/w), about 1.5% (w/w) to about 3% (w/w), about 2% (w/w) to about 3% (w/w), about 2.5% (w/w) to about 3% (w/w), about 0% (w/w) to about 2.5% (w/w), about 0% (w/w) to about 2% (w/w), about 0% (w/w) to about 1.5% (w/w), about 0% (w/w) to about 1% (w/w), about 0% (w/w) to about 0.5% (w/w), about 0.5% (w/w) to about 2.5% (w/w), about 0.5% (w/w) to about 2% (w/w), about 0.5% (w/w) to about 1.5% (w/w), about 0.5% (w/w) to about 1% (w/w), about 1% (w/w) to about 2.5% (w/w), about 1% (w/w) to about 2% (w/w), about 1% (w/w) to about 1.5% (w/w), about 1.5% (w/w) to about 2.5% (w/w), about 1.5% (w/w) to about 2% (w/w), or about 2% (w/w) to about 2.5% (w/w).

In certain embodiments, the amount of the wetting agent in the pharmaceutical composition is about 1 mg to about 20 mg, about 2 mg to about 20 mg, about 4 mg to about 20 mg, about 6 mg to about 20 mg, about 8 mg to about 20 mg, about 10 mg to about 20 mg, about 12 mg to about 20 mg, about 15 mg to about 20 mg, about 1 mg to about 15 mg, about 1 mg to about 12 mg, about 1 mg to about 10 mg, about 1 mg to about 8 mg, about 1 mg to about 6 mg, about 1 mg to about 4 mg, about 1 mg to about 2 mg, about 2 mg to about 15 mg, about 2 mg to about 12 mg, about 2 mg to about 10 mg, about 2 mg to about 8 mg, about 2 mg to about 6 mg, about 2 mg to about 4 mg, about 4 mg to about 15 mg, about 4 mg to about 12 mg, about 4 mg to about 10 mg, about 4 mg to about 8 mg, about 4 mg to about 6 mg, about 6 mg to about 15 mg, about 6 mg to about 12 mg, about 6 mg to about 10 mg, about 6 mg to about 8 mg, about 8 mg to about 15 mg, about 8 mg to about 12 mg, about 8 mg to about 10 mg, about 10 mg to about 15 mg, about 10 mg to about 12 mg, or about 12 mg to about 15 mg.

In certain embodiments, the amount of the wetting agent in the pharmaceutical composition is about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg.

In various embodiments, the pharmaceutical composition comprises:
(i) about 0.4% (w/w) to 36% (w/w) of a crystalline form of the compound of formula (I)

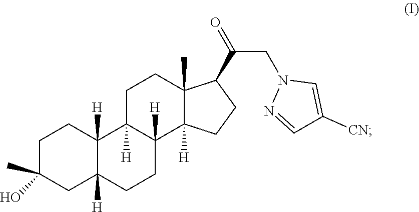

(ii) about 15% (w/w) to about 75% (w/w) of a brittle filler;
(iii) about 10% (w/w) to about 60% (w/w) of a ductile filler;
(iv) about 3% (w/w) to about 12% (w/w) of a disintegrant;
(v) about 0.25% (w/w) to about 2.5% (w/w) of a glidant; and
(vi) about 0.5% (w/w) to about 3% (w/w) of a lubricant.

In various embodiments, the pharmaceutical composition comprises:
(i) about 10% (w/w) to about 15% (w/w) of a crystalline form of the compound of formula (I)

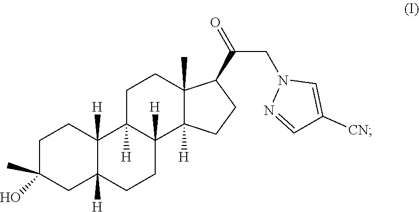

(ii) about 60% (w/w) to about 70% (w/w) of a brittle filler;
(iii) about 10% (w/w) to about 20% (w/w) of a ductile filler;
(iv) about 4% (w/w) to about 8% (w/w) of a disintegrant;
(v) about 0.5% (w/w) to about 2% (w/w) of a glidant; and about 1% (w/w) to about 2% (w/w) of a lubricant.

In certain embodiments, the brittle filler is selected from the group consisting of a sugar, an inorganic material, and combinations thereof. In certain embodiments, the sugar is selected from the group consisting of mannitol, lactose, sucrose, fructose, glucose, maltose, and combinations thereof. In certain embodiments, the inorganic material is selected from the group consisting of dibasic calcium phosphate, hydroxyapatite, sodium carbonate, sodium bicarbonate, calcium carbonate, bentonite, kaolin, and combinations thereof.

In certain embodiments, the brittle filler is selected from the group consisting of mannitol, lactose, dibasic calcium phosphate, and combinations thereof. In certain embodiments, the brittle filler is mannitol. In certain embodiments, the brittle filler is lactose. In certain embodiments, the brittle filler is dibasic calcium phosphate.

In certain embodiments, the ductile filler is selected from the group consisting of a microcrystalline cellulose, a starch, a polysaccharide, a cellulose, a polyvinylpyrrolidone, a polyvinyl acrylate, and combinations thereof. In certain embodiments, the cellulose is selected from the group consisting of a hydroxypropylcellulose, a hypromellose, a carboxymethylcellulose, a methylcellulose, a hydroxypropylmethylcellulose, and combinations thereof.

In certain embodiments, the ductile filler is a microcrystalline cellulose. In certain embodiments, the ductile filler is a starch.

In certain embodiments, the disintegrant is selected from the group consisting of sodium starch glycolate, a crospovidone, croscarmellose sodium, and combinations thereof. In certain embodiments, the disintegrant is croscarmellose sodium.

In certain embodiments, the lubricant is selected from the group consisting of sodium stearyl fumarate, magnesium stearate, stearic acid, glyceryl behenate, and combinations thereof. In certain embodiments, the lubricant is sodium stearyl fumarate.

In certain embodiments, the glidant is selected from the group consisting of colloidal silicon dioxide, talc, kaolin, bentonite, or combinations thereof. In certain embodiments, the glidant is colloidal silicon dioxide.

In certain embodiments, the binder is selected from the group consisting of a hydroxypropylcellulose, a hydroxypropylmethylcellulose, a polyvinylpyrrolidone, polyvinyl alcohol, polyvinyl acetate, a starch, and combinations thereof.

In certain embodiments, the wetting agent is selected from the group consisting of a poloxamer, sodium dodecyl sulfate, docusate sodium, and combinations thereof.

In certain embodiments, the filler comprises mannitol and microcrystalline cellulose and the mass ratio of mannitol to the microcrystalline cellulose in the pharmaceutical composition is about 1:4 to about 4:1. In certain embodiments, the filler comprises lactose and microcrystalline cellulose and the mass ratio of lactose to the microcrystalline cellulose in the pharmaceutical composition is about 1:4 to about 4:1. In certain embodiments, the filler comprises dibasic calcium phosphate and microcrystalline cellulose and the mass ratio of dibasic calcium phosphate to the microcrystalline cellulose in the pharmaceutical composition is about 1:4 to about 4:1. In certain embodiments, the filler comprises mannitol and a starch and the mass ratio of mannitol to the starch in the pharmaceutical composition is about 1:4 to about 4:1. In certain embodiments, the filler comprises dibasic calcium phosphate and a starch and the mass ratio of dibasic calcium phosphate to the starch is about 1:4 to about 4:1.

In various embodiments, the pharmaceutical composition comprises:
(i) about 10 mg of a crystalline form of the compound of formula (I)

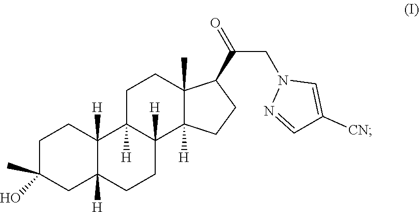

(ii) about 53 mg of mannitol;
(iii) about 13 mg of silicified microcrystalline cellulose;
(iv) about 5 mg of croscarmellose sodium;
(v) about 1 mg of colloidal silicon dioxide; and
(vi) about 1 mg of sodium stearyl fumarate.

In various embodiments, the pharmaceutical composition comprises:
(i) about 20 mg of a crystalline form of the compound of Formula (I)

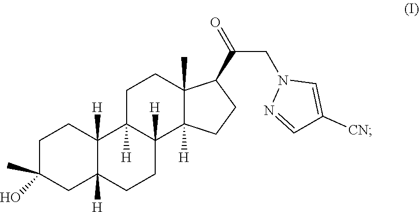

(ii) about 105.9 mg of mannitol;
(iii) about 26.2 mg of silicified microcrystalline cellulose;
(iv) about 10 mg of croscarmellose sodium;
(v) about 1.7 mg of colloidal silicon dioxide; and
(vi) about 2.9 mg of sodium stearyl fumarate.

In various embodiments, the pharmaceutical composition comprises:
(i) about 25 mg of a crystalline form of the compound of formula (I)

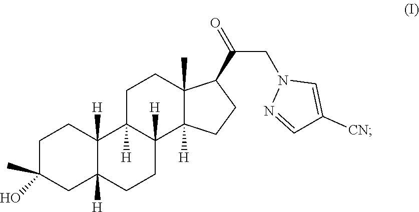

(ii) about 132 mg of mannitol;
(iii) about 32.7 mg of the silicified microcrystalline cellulose;
(iv) about 12.5 mg of croscarmellose sodium;
(v) about 2.1 mg of colloidal silicon dioxide; and
(vi) about 3.6 mg of sodium stearyl fumarate.

In various embodiments, the pharmaceutical composition comprises:
(i) about 30 mg of the crystalline form of the compound of formula (I)

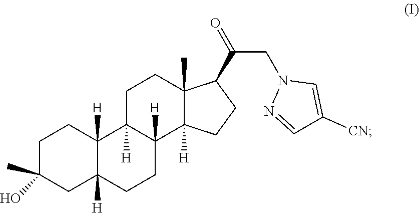

(ii) about 159 mg of mannitol;
(iii) about 39.3 mg of the silicified microcrystalline cellulose;
(iv) about 15 mg of croscarmellose sodium;
(v) about 2.5 mg of colloidal silicon dioxide; and
(vi) about 4.4 mg of sodium stearyl fumarate.

In various embodiments, the pharmaceutical composition comprises:
(i) about 40 mg of a crystalline form of the compound of formula (I)

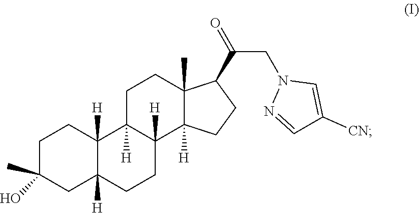

(ii) about 212 mg of mannitol;
(iii) about 52.4 mg of the silicified microcrystalline cellulose;
(iv) about 20 mg of croscarmellose sodium;
(v) about 3.3 mg of colloidal silicon dioxide; and
(vi) about 5.8 mg of sodium stearyl fumarate.

In various embodiments, the pharmaceutical composition comprises:
(i) about 50 mg of a crystalline form of the compound of formula (I)

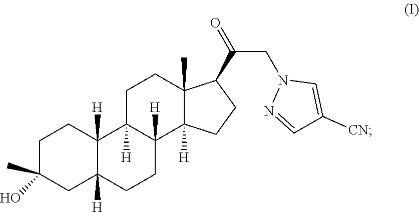

(ii) about 265 mg of mannitol;
(iii) about 65.5 mg of the silicified microcrystalline cellulose;
(iv) about 25 mg of croscarmellose sodium;
(v) about 4.2 mg of colloidal silicon dioxide; and
(vi) about 7.3 mg of sodium stearyl fumarate.

In various embodiments, the pharmaceutical composition comprises:
(i) about 60 mg of a crystalline form of the compound of formula (I)
(ii) about 317.7 mg of mannitol;
(iii) about 78.6 mg of the silicified microcrystalline cellulose;
(iv) about 30 mg of croscarmellose sodium;
(v) about 5 mg of colloidal silicon dioxide; and
(vi) about 8.8 mg of sodium stearyl fumarate.

In various embodiments, the pharmaceutical composition comprises a plurality of particles of a crystalline form of the compound of formula (I)

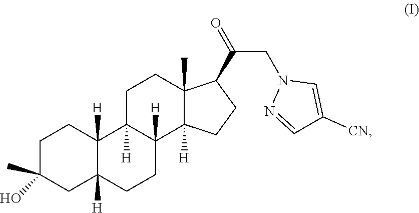

wherein the plurality of particles of the crystalline form of the compound of formula (I) have a particle size distribution which is defined by a D₉₀ of about 1 μm to about 100 μm.

In certain embodiments, the plurality of particles of the crystalline form of the compound of formula (I) has a particle size distribution which is defined by a D₉₀ of about 1 μm to about 20 μm. In certain embodiments, the plurality of particles of the crystalline form of the compound of formula (I) has a particle size distribution which is defined by a D₉₀ of about 1 μm to about 13 μm.

In various embodiments, the pharmaceutical composition comprises:
(i) a plurality of particles of a crystalline form of the compound of formula (I)

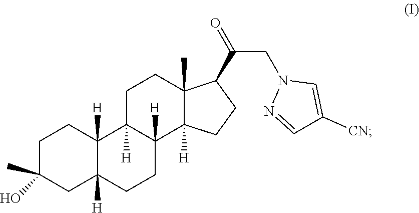

(ii) a filler; and
(iii) one or more pharmaceutically acceptable excipients selected from the group consisting of a disintegrant, a binder, a wetting agent, a lubricant, a glidant, and combinations thereof,
wherein the plurality of particles of the crystalline form of the compound of formula (I) have a particle size distribution which is defined by a D₉₀ of about 1 μm to about 20 μm.

In certain embodiments, the plurality of particles of the crystalline form of the compound of formula (I) have a particle size distribution which is defined by a D₉₀ of about 1 μm to about 13 μm.

In certain embodiments, the plurality of particles of the crystalline form of the compound of formula (I) have a particle size distribution which is defined by a D₉₀ of about 5 μm to about 10 μm, about 5.5 μm to about 10 μm, about 6 μm to about 10 μm, about 6.5 μm to about 10 μm, about 7 μm to about 10 μm, about 7.5 μm to about 10 μm, about 8 μm to about 10 μm, about 8.5 μm to about 10 μm, about 9 μm to about 10 μm, about 9.5 μm to about 10 μm, about 5 μm to about 9.5 μm, about 5 μm to about 9 μm, about 5 μm to about 8.5 μm, about 5 μm to about 8 μm, about 5 μm to about 7.5 μm, about 5 μm to about 7 μm, about 5 μm to about 6.5 μm, about 5 μm to about 6 μm, about 5 μm to about 5.5 μm, about 5.5 μm to about 9.5 μm, about 5.5 μm to about 9 μm, about 5.5 μm to about 8.5 μm, about 5.5 μm to about 8 μm, about 5.5 μm to about 7.5 μm, about 5.5 μm to about 7 μm, about 5.5 μm to about 6.5 μm, about 5.5 μm to about 6 μm, about 6 μm to about 9.5 μm, about 6 μm to about 9 μm, about 6 μm to about 8.5 μm, about 6 μm to about 8 μm, about 6 μm to about 7.5 μm, about 6 μm to about 7 μm, about 6 μm to about 6.5 μm, about 6.5 μm to about 9.5 μm, about 6.5 μm to about 9 μm, about 6.5 μm to about 8.5 μm, about 6.5 μm to about 8 μm, about 6.5 μm to about 7.5 μm, about 6.5 μm to about 7 μm, about 7 μm to about 9.5 μm, about 7 μm to about 9 μm, about 7 μm to about 8.5 μm, about 7 μm to about 8 μm, about 7 μm to about 7.5 μm, about 7.5 μm to about 9.5 μm, about 7.5 μm to about 9 μm, about 7.5 μm to about 8.5 μm, about 7.5 μm to about 8 μm, about 8 μm to about 9.5 μm, about 8 μm to about 9 μm, about 8 μm to about 8.5 μm, about 8.5 μm to about 9.5 μm, about 8.5 μm to about 9 μm, or about 9 μm to about 9.5 μm. In various embodiments, the pharmaceutical composition comprises:
(i) a crystalline form or forms of the compound of formula (I)

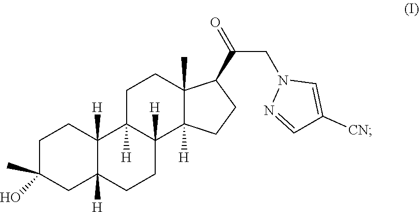

(ii) a filler; and
(iii) one or more pharmaceutically acceptable excipients selected from the group consisting of a disintegrant, a binder, a wetting agent, a lubricant, a glidant, and combinations thereof,
wherein the pharmaceutical composition has a bulk density from about 0.2 g/cc to about 0.8 g/cc and a tapped density of about 0.3 g/cc to about 1.1 g/cc and wherein the tapped density of the pharmaceutical composition is higher than the bulk density.

In certain embodiments, the pharmaceutical composition has a bulk density from about 0.3 g/cc to about 0.7 g/cc and a tapped density of about 0.5 g/cc to about 0.9 g/cc, wherein the tapped density of the pharmaceutical composition is higher than the bulk density. In certain embodiments, the pharmaceutical composition has a bulk density from about 0.4 g/cc to about 0.7 g/cc and a tapped density of about 0.5 g/cc to about 0.9 g/cc and wherein the tapped density of the pharmaceutical composition is higher than the bulk density.

In various embodiments, the pharmaceutical composition comprises:
(i) a crystalline form of the compound of formula (I)

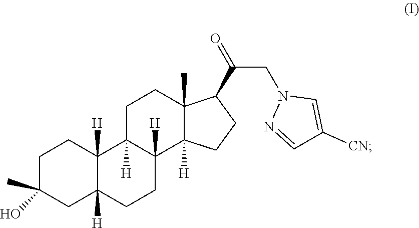

(ii) a filler; and
(iii) one or more pharmaceutically acceptable excipients selected from the group consisting of a disintegrant, a binder, a wetting agent, a lubricant, a glidant, and combinations thereof,
wherein the pharmaceutical composition has an average flow rate index (FRI) from about 0.05 to about 3.1 kg/sec.

In certain embodiments, the pharmaceutical composition has an average (FRI) from about 0.2 to about 1.5 kg/sec. In certain embodiments, the pharmaceutical composition has an average (FRI) from about 0.4 to about 0.9 kg/sec.

In various embodiments, the pharmaceutical composition comprises:
(i) a crystalline form of the compound of formula (I)

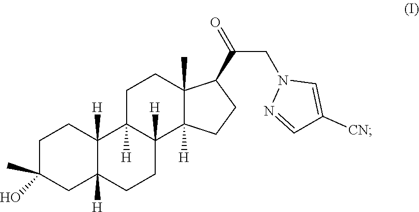

(ii) a filler; and
(iii) one or more pharmaceutically acceptable excipients selected from the group consisting of a disintegrant, a binder, a wetting agent, a lubricant, a glidant, a capsule, and combinations thereof,
   wherein the pharmaceutical composition releases at least about 50% of the compound of formula (I) after about 20 minutes, when tested using a USP 1 or a USP 2 apparatus.

In certain embodiments, the pharmaceutical composition releases at least about 65% of the compound of formula (I) after about 30 minutes, when tested using a USP 1 or a USP 2 apparatus.

In various embodiments, the pharmaceutical composition comprises:
(i) a crystalline form of the compound of formula (I)

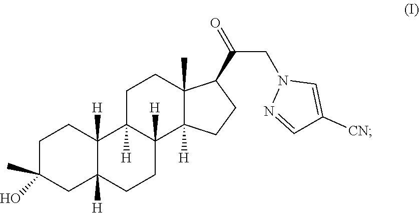

(ii) a filler; and
(iii) one or more pharmaceutically acceptable excipients selected from the group consisting of a disintegrant, a binder, a wetting agent, a lubricant, a glidant, a capsule, and combinations thereof;
   wherein the pharmaceutical composition exhibits the following dissolution profile:
at least about 70% of the compound of formula (I) is released after about 20 minutes; and
at least about 80% of the compound of formula (I) is released after about 30 minutes, when tested in 500 mL to 900 mL of 50 mM sodium phosphate buffer, pH 6.8 with 0.2% to 0.6% SDS in a USP 2 apparatus at about 37° C.

In certain embodiments, a pharmaceutical composition described herein further comprises a coating. In certain embodiments, the coating is selected from the group consisting of a film forming polymer, a plasticizer, and combinations thereof. In certain embodiments, the film forming polymer is selected from the group consisting of a hypromellose, a ethylcellulose, cellulose acetate, a polyvinylpyrrolidone, a polyvinyl alcohol, a polyacrylate, and combinations thereof. In certain embodiments, the plasticizer is selected from the group consisting of triacetin, polyethylene glycol, propylene glycol, and combinations thereof.

In certain embodiments, a pharmaceutical composition described herein further comprises a capsule. In certain embodiments, the capsule is a gelatin capsule.

### Dosage Forms

In an aspect, the invention provides dosage forms comprising a pharmaceutical composition described herein.

In another aspect, the invention provides dosage forms intended for oral administration comprising a pharmaceutical composition described herein.

In certain embodiments, the dosage form is selected from the group consisting of a powder, a sachet, a stickpack, a capsule, a minitab, and a tablet.

In certain embodiments, the dosage form is a capsule. In certain embodiments, the size of the capsule is selected from the group consisting of 000, 00, 0, 1, 2, 3, 4, and 5.

In certain embodiments, the total weight of the pharmaceutical composition in the capsule is about 25 mg to about 1000 mg, about 50 mg to about 1000 mg, about 75 mg to about 1000 mg, about 100 mg to about 1000 mg, about 150 mg to about 1000 mg, about 200 mg to about 1000 mg, about 250 mg to about 1000 mg, about 300 mg to about 1000 mg, about 400 mg to about 1000 mg, about 500 mg to about 1000 mg, about 600 mg to about 1000 mg, about 700 mg to about 1000 mg, about 800 mg to about 1000 mg, about 900 mg to about 1000 mg, about 25 mg to about 900 mg, about 25 mg to about 800 mg, about 25 mg to about 700 mg, about 25 mg to about 600 mg, about 25 mg to about 500 mg, about 25 mg to about 400 mg, about 25 mg to about 300 mg, about 25 mg to about 250 mg, about 25 mg to about 200 mg, about 25 mg to about 150 mg, about 25 mg to about 100 mg, about 25 mg to about 75 mg, about 25 mg to about 50 mg, about 50 mg to about 900 mg, about 50 mg to about 800 mg, about 50 mg to about 700 mg, about 50 mg to about 600 mg, about 50 mg to about 500 mg, about 50 mg to about 400 mg, about 50 mg to about 300 mg, about 50 mg to about 250 mg, about 50 mg to about 200 mg, about 50 mg to about 150 mg, about 50 mg to about 100 mg, about 50 mg to about 75 mg, about 75 mg to about 900 mg, about 75 mg to about 800 mg, about 75 mg to about 700 mg, about 75 mg to about 600 mg, about 75 mg to about 500 mg, about 75 mg to about 400 mg, about 75 mg to about 300 mg, about 75 mg to about 200 mg, about 75 mg to about 100 mg, about 100 mg to about 900 mg, about 100 mg to about 800 mg, about 100 mg to about 700 mg, about 100 mg to about 600 mg, about 100 mg to about 500 mg, about 100 mg to about 400 mg, about 100 mg to about 300 mg, about 100 mg to about 250 mg, about 100 mg to about 200 mg, about 100 mg to about 150 mg, about 150 mg to about 900 mg, about 150 mg to about 800 mg, about 150 mg to about 700 mg, about 150 mg to about 600 mg, about 150 mg to about 500 mg, about 150 mg to about 400 mg, about 150 mg to about 300 mg, about 150 mg to about 250 mg, about 150 mg to about 200 mg, about 200 mg to about 900 mg, about 200 mg to about 800 mg, about 200 mg to about 700 mg, about 200 mg to about 600 mg, about 200 mg to about 500 mg, about 200 mg to about 400 mg, about 200 mg to about 300 mg, about 200 mg to about 250 mg, about 250 mg to about 900 mg, about 250 mg to about 800 mg, about 250 mg to about 700 mg, about 250 mg to about 600 mg, about 250 mg to about 500 mg, about 250 mg to about 400 mg, about 250 mg to about 300 mg, about 300 mg to about 900 mg, about 300 mg to about 800 mg, about 300 mg to about 700 mg, about 300 mg to about 600 mg, about 300 mg to about 500 mg, about 300 mg to about 400 mg, about 400 mg to about 900 mg, about 400 mg to about 800 mg, about 400 mg to about 700 mg, about 400 mg to about 600 mg, about 400 mg to about 500 mg, about 500 mg to about 900 mg, about 500 mg to about 800 mg, about 500 mg to about 700 mg, about 500 mg to about 600 mg, about 600 mg to about 900 mg, about 600 mg to about 800 mg, about 600 mg to about 700 mg, about 700 mg to about 900 mg, about 700 mg to about 800 mg, or about 800 mg to about 900 mg.

In certain embodiments, the dosage form is a tablet.

In certain embodiments, the total weight of the pharmaceutical composition in the tablet is about 20 mg to about 1000 mg, about 50 mg to about 1000 mg, about 75 mg to about 1000 mg, about 100 mg to about 1000 mg, about 150 mg to about 1000 mg, about 200 mg to about 1000 mg, about 250 mg to about 1000 mg, about 300 mg to about 1000 mg, about 400 mg to about 1000 mg, about 500 mg to about 1000 mg, about 600 mg to about 1000 mg, about 700 mg to about 1000 mg, about 800 mg to about 1000 mg, about 900 mg to about 1000 mg, about 20 mg to about 900 mg, about 20 mg to about 800 mg, about 20 mg to about 700 mg, about 20 mg to about 600 mg, about 20 mg to about 500 mg, about 20 mg to about 400 mg, about 20 mg to about 300 mg, about 20 mg to about 250 mg, about 20 mg to about 200 mg, about 20 mg to about 150 mg, about 20 mg to about 100 mg, about 20 mg to about 75 mg, about 20 mg to about 50 mg, about 50 mg to about 900 mg, about 50 mg to about 800 mg, about 50 mg to about 700 mg, about 50 mg to about 600 mg, about 50 mg to about 500 mg, about 50 mg to about 400 mg, about 50 mg to about 300 mg, about 50 mg to about 250 mg, about 50 mg to about 200 mg, about 50 mg to about 150 mg, about 50 mg to about 100 mg, about 50 mg to about 75 mg, about 75 mg to about 900 mg, about 75 mg to about 800 mg, about 75 mg to about 700 mg, about 75 mg to about 600 mg, about 75 mg to about 500 mg, about 75 mg to about 400 mg, about 75 mg to about 300 mg, about 75 mg to about 200 mg, about 75 mg to about 100 mg, about 100 mg to about 900 mg, about 100 mg to about 800 mg, about 100 mg to about 700 mg, about 100 mg to about 600 mg, about 100 mg to about 500 mg, about 100 mg to about 400 mg, about 100 mg to about 300 mg, about 100 mg to about 250 mg, about 100 mg to about 200 mg, about 100 mg to about 150 mg, about 150 mg to about 900 mg, about 150 mg to about 800 mg, about 150 mg to about 700 mg, about 150 mg to about 600 mg, about 150 mg to about 500 mg, about 150 mg to about 400 mg, about 150 mg to about 300 mg, about 150 mg to about 250 mg, about 150 mg to about 200 mg, about 200 mg to about 900 mg, about 200 mg to about 800 mg, about 200 mg to about 700 mg, about 200 mg to about 600 mg, about 200 mg to about 500 mg, about 200 mg to about 400 mg, about 200 mg to about 300 mg, about 200 mg to about 250 mg, about 250 mg to about 900 mg, about 250 mg to about 800 mg, about 250 mg to about 700 mg, about 250 mg to about 600 mg, about 250 mg to about 500 mg, about 250 mg to about 400 mg, about 250 mg to about 300 mg, about 300 mg to about 900 mg, about 300 mg to about 800 mg, about 300 mg to about 700 mg, about 300 mg to about 600 mg, about 300 mg to about 500 mg, about 300 mg to about 400 mg, about 400 mg to about 900 mg, about 400 mg to about 800 mg, about 400 mg to about 700 mg, about 400 mg to about 600 mg, about 400 mg to about 500 mg, about 500 mg to about 900 mg, about 500 mg to about 800 mg, about 500 mg to about 700 mg, about 500 mg to about 600 mg, about 600 mg to about 900 mg, about 600 mg to about 800 mg, about 600 mg to about 700 mg, about 700 mg to about 900 mg, about 700 mg to about 800 mg, or about 800 mg to about 900 mg.

In certain embodiments, wherein the tablet further comprises a coating. In certain embodiments, the coating is selected from the group consisting of a film forming polymer, a plasticizer, and combinations thereof. In certain embodiments, the film forming polymer is selected from the group consisting of a hypromellose, a ethylcellulose, cellulose acetate, a polyvinylpyrrolidone, a polyvinyl alcohol, a polyacrylate, and combinations thereof. In certain embodiments, the plasticizer is selected from the group consisting of triacetin, polyethylene glycol, propylene glycol, and combinations thereof.

### Methods of Making

In an aspect, the invention provides processes for preparing the pharmaceutical compositions described herein, for example, comprising:
(a) micronizing a crystalline form of the compound of formula (I) to obtain a micronized crystalline form of the compound of formula (I)

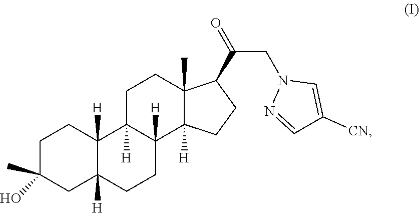

wherein the micronized crystalline form of the compound of formula (I) has a particle size distribution which is defined by a D₉₀ of about 1 μm to about 100 μm;
(b) blending the micronized crystalline form of the compound of formula (I) with one or more pharmaceutically acceptable excipients to obtain a blend;
(c) granulating the blend to obtain granules;
(d) milling the granules to obtain an intragranular phase; and
(e) blending the intragranular phase with one or more extragranular pharmaceutical excipients to obtain the pharmaceutical composition.

In certain embodiments, in step (a), the micronized crystalline form of the compound of formula (I) has a particle size distribution which is defined by a D₉₀ of about 1 μm to about 100 μm, about 5 μm to about 100 μm, about 10 μm to about 100 μm, about 15 μm to about 100 μm, about 20 μm to about 100 μm, about 25 μm to about 100 μm, about 30 μm to about 100 μm, about 35 μm to about 100 μm, about 40 μm to about 100 μm, about 45 μm to about 100 μm, about 50 μm to about 100 μm, about 60 μm to about 100 μm, about 70 μm to about 100 μm, about 80 μm to about 100 μm, about 90 μm to about 100 μm, about 1 μm to about 90 μm, about 1 μm to about 80 μm, about 1 μm to about 70 μm, about 1 μm to about 60 μm, about 1 μm to about 50 μm, about 1 μm to about 45 μm, about 1 μm to about 40 μm, about 1 μm to about 35 μm, about 1 μm to about 30 μm, about 1 μm to about 25 μm, about 1 μm to about 20 μm, about 1 μm to about 15 μm, about 1 μm to about 10 μm, about 1 μm to about 5 μm, about 5 μm to about 90 μm, about 5 μm to about 80 μm, about 5 μm to about 70 μm, about 5 μm to about 60 μm, about 5 μm to about 50 μm, about 5 μm to about 45 μm, about 5 μm to about 40 μm, about 5 μm to about 35 μm, about 5 μm to about 30 μm, about 5 μm to about 25 μm, about 5 μm to about 20 μm, about 5 μm to about 15 μm, about 5 μm to about 10 μm, about 10 μm to about 90 μm, about 10 μm to about 80 μm, about 10 μm to about 70 μm, about 10 μm to about 60 μm, about 10 μm to about 50 μm, about 10 μm to about 45 μm, about 10 μm to about 40 μm, about 10 μm to about 35 μm, about 10 μm to about 30 μm, about 10 μm to about 25 μm, about 10 μm to about 20 μm, about 10 μm to about 15 μm, about 15 μm to about 90 μm, about 15 μm to about 80 μm, about 15 μm to about 70 μm, about 15 μm to about 60 μm, about 15 μm to about 50 μm, about 15 μm to about 45 μm, about 15 μm to about 40 μm, about 15 μm to about 35 μm, about 15 μm to about 30 μm, about 15 μm to about 25 μm, about 15 μm to about 20 μm, about 20 μm to about 90 μm, about 20 μm to about 80 μm, about 20 μm to about 70 μm, about 20 μm to about 60 μm, about 20 μm to about 50 μm, about 20 μm to about 45 μm, about 20 μm to about 40 μm, about 20 μm to about 35 μm, about 20 μm to about 30 μm, about 20 μm to about 25 μm, about 30 μm to about 90 μm, about 30 μm to about 80 μm, about 30 μm to about 70 μm, about 30 μm to about 60 μm, about 30 μm to about 50 μm, about 30 μm to about 45 μm, about 30 μm to about 40 μm, about 30 μm to about 35 μm, about 35 μm to about 90 μm, about 35 μm to about 80 μm, about 35 μm to about 70 μm, about 35 μm to about 60 μm, about 35 μm to about 50 μm, about 35 μm to about 45 μm, about 35 μm to about 40 μm, about 40 μm to about 90 μm, about 40 μm to about 80 μm, about 40 μm to about 70 μm, about 40 μm to about 50 μm, about 40 μm to about 45 μm, about 45 μm to about 90 μm, about 45 μm to about 80 μm, about 45 μm to about 70 μm, about 45 μm to about 60 μm, about 45 μm to about 50 μm, about 50 μm to about 90 μm, about 50 μm to about 80 μm, about 50 μm to about 70 μm, about 50 μm to about 60 μm, about 60 μm to about 90 μm, about 60 μm to about 80 μm, about 60 μm to about 70 μm, about 70 μm to about 90 μm, about 70 μm to about 80 μm, or about 80 μm to about 90 μm.

In certain embodiments, in step (a), the micronized crystalline form of the compound of formula (I) has a particle size distribution which is defined by a D₉₀ of about 1 μm to about 20 μm, about 2 μm to about 20 μm, about 4 μm to about 20 μm, about 6 μm to about 20 μm, about 8 μm to about 20 μm, about 10 μm to about 20 μm, about 12 μm to about 20 μm, about 14 μm to about 20 μm, about 16 μm to about 20 μm, about 18 μm to about 20 μm, about 1 μm to about 18 μm, about 1 μm to about 16 μm, about 1 μm to about 14 μm, about 1 μm to about 12 μm, about 1 μm to about 10 μm, about 1 μm to about 8 μm, about 1 μm to about 6 μm, about 1 μm to about 4 μm, about 1 μm to about 2 μm, about 2 μm to about 18 μm, about 2 μm to about 16 μm, about 2 μm to about 14 μm, about 2 μm to about 12 μm, about 2 μm to about 10 μm, about 2 μm to about 8 μm, about 2 μm to about 6 μm, about 2 μm to about 4 μm, about 4 μm to about 18 μm, about 4 μm to about 16 μm, about 4 μm to about 14 μm, about 4 μm to about 12 μm, about 4 μm to about 10 μm, about 4 μm to about 8 μm, about 4 μm to about 6 μm, about 6 μm to about 18 μm, about 6 μm to about 16 μm, about 6 μm to about 14 μm, about 6 μm to about 12 μm, about 6 μm to about 10 μm, about 6 μm to about 8 μm, about 8 μm to about 18 μm, about 8 μm to about 16 μm, about 8 μm to about 14 μm, about 8 μm to about 12 μm, about 8 μm to about 10 μm, about 10 μm to about 18 μm, about 10 μm to about 16 μm, about 10 μm to about 14 μm, about 10 μm to about 12 μm, about 12 μm to about 18 μm, about 12 μm to about 16 μm, about 12 μm to about 14 μm, about 14 μm to about 18 μm, about 14 μm to about 16 μm, or about 16 μm to about 18 μm. In certain embodiments, in step (a), the micronized crystalline form of the compound of formula (I) has a particle size distribution which is defined by a D₉₀ of about 1 μm to about 20 μm.

In certain embodiments, in step (a), the micronized crystalline form of the compound of formula (I) has a particle size distribution which is defined by a D₉₀ of about 1 μm to about 15 μm, about 3 μm to about 15 μm, about 5 μm to about 15 μm, about 7 μm to about 15 μm, about 9 μm to about 15 μm, about 11 μm to about 15 μm, about 13 μm to about 15 μm, about 1 μm to about 13 μm, about 1 μm to about 11 μm, about 1 μm to about 9 μm, about 1 μm to about 7 μm, about 1 μm to about 5 μm, about 1 μm to about 3 μm, about 3 μm to about 13 μm, about 3 μm to about 11 μm, about 3 μm to about 9 μm, about 3 μm to about 7 μm, about 3 μm to about 5 μm, about 5 μm to about 13 μm, about 5 μm to about 11 μm, about 5 μm to about 9 μm, about 5 μm to about 7 μm, about 9 μm to about 13 μm, about 9 μm to about 11 μm, or about 11 μm to about 13 μm. In certain embodiments, in step (a), the micronized crystalline form of the compound of formula (I) has a particle size distribution which is defined by a D₉₀ of about 1 μm to about 13 μm.

In certain embodiments, in step (a), the micronized crystalline form of the compound of formula (I) has a particle size distribution which is defined by a D₉₀ of about 5 μm to about 10 μm, about 5.5 μm to about 10 μm, about 6 μm to about 10 μm, about 6.5 μm to about 10 μm, about 7 μm to about 10 μm, about 7.5 μm to about 10 μm, about 8 μm to about 10 μm, about 8.5 μm to about 10 μm, about 9 μm to about 10 μm, about 9.5 μm to about 10 μm, about 5 μm to about 9.5 μm, about 5 μm to about 9 μm, about 5 μm to about 8.5 μm, about 5 μm to about 8 μm, about 5 μm to about 7.5 μm, about 5 μm to about 7 μm, about 5 μm to about 6.5 μm, about 5 μm to about 6 μm, about 5 μm to about 5.5 μm, about 5.5 μm to about 9.5 μm, about 5.5 μm to about 9 μm, about 5.5 μm to about 8.5 μm, about 5.5 μm to about 8 μm, about 5.5 μm to about 7.5 μm, about 5.5 μm to about 7 μm, about 5.5 μm to about 6.5 μm, about 5.5 μm to about 6 μm, about 6 μm to about 9.5 μm, about 6 μm to about 9 μm, about 6 μm to about 8.5 μm, about 6 μm to about 8 μm, about 6 μm to about 7.5 μm, about 6 μm to about 7 μm, about 6 μm to about 6.5 μm, about 6.5 μm to about 9.5 μm, about 6.5 μm to about 9 μm, about 6.5 μm to about 8.5 μm, about 6.5 μm to about 8 μm, about 6.5 μm to about 7.5 μm, about 6.5 μm to about 7 μm, about 7 μm to about 9.5 μm, about 7 μm to about 9 μm, about 7 μm to about 8.5 μm, about 7 μm to about 8 μm, about 7 μm to about 7.5 μm, about 7.5 μm to about 9.5 μm, about 7.5 μm to about 9 μm, about 7.5 μm to about 8.5 μm, about 7.5 μm to about 8 μm, about 8 μm to about 9.5 μm, about 8 μm to about 9 μm, about 8 μm to about 8.5 μm, about 8.5 μm to about 9.5 μm, about 8.5 μm to about 9 μm, or about 9 μm to about 9.5 μm.

In certain embodiments, in step (a), the micronized crystalline form of the compound of formula (I) has a particle size distribution which is defined by a D₉₀ of about 6.1 μm to about 7.7 μm, about 6.1 μm to about 8.0 μm, about 4.1 μm to about 10.0 μm, about 5.6 μm to about 10.6 μm, about 4.9 μm to about 12.4 μm, about 3.9 μm to about 11.0 μm, or about 4.2 μm to about 11.6 μm.

In certain embodiments, in step (a), the micronized crystalline form of the compound of formula (I) has a particle size distribution which is defined by a D₉₀ of about 6.1 μm to about 7.7 μm.

In certain embodiments, in step (a), the micronized crystalline form of the compound of formula (I) has a particle size distribution which is defined by a D₉₀ of about 6.1 μm to about 8.0 μm.

In certain embodiments, in step (a), the micronized crystalline form of the compound of formula (I) has a particle size distribution which is defined by a D₉₀ of about 4.1 μm to about 10.0 μm.

In certain embodiments, in step (a), the micronized crystalline form of the compound of formula (I) has a particle size distribution which is defined by a D₉₀ of about 5.6 μm to about 10.6 μm.

In certain embodiments, in step (a), the micronized crystalline form of the compound of formula (I) has a particle size distribution which is defined by a D₉₀ of about 4.9 μm to about 12.4 μm.

In certain embodiments, in step (a), the micronized crystalline form of the compound of formula (I) has a particle size distribution which is defined by a D₉₀ of about 3.9 μm to about 11.0 μm.

In certain embodiments, in step (a), the micronized crystalline form of the compound of formula (I) has a particle size distribution which is defined by a D₉₀ of about 4.2 μm to about 11.6 μm.

In certain embodiments, in step (b), the one or more pharmaceutically acceptable excipients is selected from the group consisting of a filler, a disintegrant, a binder, a wetting agent, a lubricant, a glidant, and combinations thereof. In certain embodiments, in step (b), the micronized crystalline compound of formula (I) is blended with a filler, a disintegrant, a lubricant, and a glidant.

In certain embodiments, in step (b), the filler is selected from the group consisting of a brittle filler, a ductile filler, and combinations thereof. In certain embodiments, in step (b), the filler comprises a brittle filler and a ductile filler.

In certain embodiments, in step (b), the brittle filler is selected from the group consisting of mannitol, lactose, dibasic calcium phosphate, and combinations thereof.

In certain embodiments, in step (b), the ductile filler is selected from the group consisting of a microcrystalline cellulose, a starch, a polysaccharide, a cellulose, a polyvinylpyrrolidone, a polyvinyl acrylate, and combinations thereof.

In certain embodiments, in step (b), the disintegrant is selected from the group consisting of sodium starch glycolate, a crosslinked polyvinylpyrrolidone, croscarmellose sodium, and combinations thereof.

In certain embodiments, in step (b), the glidant is selected from colloidal silicon dioxide, talc, and combinations thereof.

In certain embodiments, in step (b), the lubricant is selected from the group consisting of magnesium stearate, sodium stearyl fumarate, glyceryl behenate, stearic acid, and combinations thereof.

In certain embodiments, in step (c), granulating the blend to obtain granules comprises a dry granulation process step. In certain embodiments, in step (c), granulating the blend to obtain granules comprises a wet granulation process step.

In certain embodiments, in step (c), the granules have a solid fraction of about 0.5 to about 0.95, about 0.55 to about 0.95, about 0.6 to about 0.95, about 0.7 to about 0.95, about 0.8 to about 0.95, about 0.85 to about 0.95, about 0.9 to about 0.95, about 0.5 to about 0.9, about 0.5 to about 0.85, about 0.5 to about 0.8, about 0.5 to about 0.7, about 0.5 to about 0.6, about 0.5 to about 0.55, about 0.55 to about 0.9, about 0.55 to about 0.85, about 0.55 to about 0.8, about 0.55 to about 0.7, about 0.55 to about 0.6, about 0.6 to about 0.9, about 0.6 to about 0.85, about 0.6 to about 0.8, about 0.6 to about 0.7, about 0.7 to about 0.9, about 0.7 to about 0.85, about 0.7 to about 0.8, about 0.8 to about 0.9, about 0.8 to about 0.85, or about 0.85 to about 0.9. In certain embodiments, the granules have a solid fraction of about 0.55 to about 0.9. In certain embodiments, the granules have a solid fraction of about 0.6 to about 0.85.

In certain embodiments, in step (e), the one or more extragranular excipients is selected from the group consisting of a disintegrant, a lubricant, a glidant, and combinations thereof. In certain embodiments, in step (e), the intragranular phase is blended with a disintegrant, a lubricant, and a glidant.

In certain embodiments, in step (e), the disintegrant is selected from the group consisting of sodium starch glycolate, a crosslinked polyvinylpyrrolidone, croscarmellose sodium, and combinations thereof.

In certain embodiments, in step (e), the glidant is selected from colloidal silicon dioxide, talc, and combinations thereof.

In certain embodiments, in step (e), the lubricant is selected from the group consisting of magnesium stearate, sodium stearyl fumarate, glyceryl behenate, stearic acid, and combinations thereof.

In certain embodiments, the process further comprises compressing the pharmaceutical composition into a tablet.

In certain embodiments, the tablet comprises a coating. In certain embodiments, the coating comprises one or more film-forming polymers selected from the group consisting of a hypromellose, an ethylcellulose, a polyvinylpyrrolidone, a polyacrylate, a plasticizer, and combinations thereof. In certain embodiments, the coating comprises a colorant selected from the group consisting of titanium dioxide, an aluminum lake, an iron oxide, carbon black, and combinations thereof.

In certain embodiments, the process further comprises filling a capsule with the pharmaceutical composition.

In certain embodiments, the capsule size is 000, 00, 0, 1, 2, 3, 4, and 5. In certain embodiments, the capsule comprises a gelatin, a polysaccharide, a starch, a hypromellose, or combinations thereof. In certain embodiments, the capsules comprise a colorant. In certain embodiments, the colorant is selected from the group consisting of titanium dioxide, an aluminum lake, an iron oxide, carbon black, and combinations thereof.

In another aspect, the invention provides a pharmaceutical composition as described herein (e.g., pharmaceutical compositions of the compound of formula (I), or a pharmaceutically acceptable salt thereof), wherein the process for making the pharmaceutical composition comprises a micronization step. In certain embodiments, the micronization step comprises micronizing a crystalline form of the compound of formula (I) to obtain a micronized crystalline form of the compound of formula (I). In certain embodiments, the micronized crystalline form of the compound of formula (I) has a particle size distribution as described herein.

In various embodiments, the invention provides a pharmaceutical composition comprising
(i) a crystalline form of the compound of formula (I)

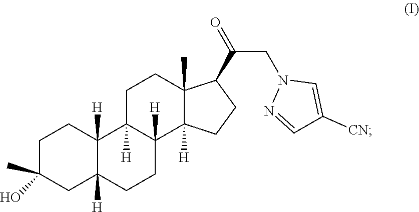

(ii) a filler;
(iii) a lubricant; and
(iv) a glidant,

wherein the process for making the pharmaceutical composition comprises a micronization step that includes micronizing a crystalline form of the compound of formula (I) to obtain a micronized crystalline form of the compound of formula (I). In certain embodiments, the micronized crystalline form of the compound of formula (I) has a particle size distribution which is defined by a D₉₀ of about 6.1 μm to about 7.7 μm, about 6.1 μm to about 8.0 μm, about 4.1 μm to about 10.0 μm, about 5.6 μm to about 10.6 μm, about 4.9 μm to about 12.4 μm, about 3.9 μm to about 11.0 μm, or about 4.2 μm to about 11.6 μm.

In certain embodiments, the micronized crystalline form of the compound of formula (I) has a particle size distribution which is defined by a D₉₀ of about 6.1 μm to about 7.7 μm.

In certain embodiments, the micronized crystalline form of the compound of formula (I) has a particle size distribution which is defined by a D₉₀ of about 6.1 μm to about 8.0 μm.

In certain embodiments, the micronized crystalline form of the compound of formula (I) has a particle size distribution which is defined by a D₉₀ of about 4.1 μm to about 10.0 μm.

In certain embodiments, the micronized crystalline form of the compound of formula (I) has a particle size distribution which is defined by a D₉₀ of about 5.6 μm to about 10.6 μm.

In certain embodiments, the micronized crystalline form of the compound of formula (I) has a particle size distribution which is defined by a D₉₀ of about 4.9 μm to about 12.4 μm.

In certain embodiments, the micronized crystalline form of the compound of formula (I) has a particle size distribution which is defined by a D₉₀ of about 3.9 μm to about 11.0 μm.

In certain embodiments, the micronized crystalline form of the compound of formula (I) has a particle size distribution which is defined by a D₉₀ of about 4.2 μm to about 11.6 μm.

In various embodiments, the invention provides a pharmaceutical composition comprising
(i) about 0.4% (w/w) to about 60% (w/w) of a crystalline form of the compound of formula (I)

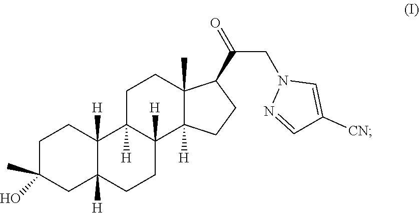

(ii) about 0% (w/w) to about 90% (w/w) of a brittle filler;
(iii) about 0% (w/w) to about 90% (w/w) of a ductile filler;
(iv) about 0% (w/w) to about 15% (w/w) of a disintegrant;
(v) about 0.1% (w/w) to about 5% (w/w) of a lubricant; and
(vi) about 0.1% (w/w) to about 5% (w/w) of a glidant,
   wherein the process for making the pharmaceutical composition comprises a micronization step that includes micronizing a crystalline form of the compound of formula (I) to obtain a micronized crystalline form of the compound of formula (I).

In certain embodiments, the micronized crystalline form of the compound of formula (I) has a particle size distribution which is defined by a D₉₀ of about 6.1 μm to about 7.7 μm, about 6.1 μm to about 8.0 μm, about 4.1 μm to about 10.0 μm, about 5.6 μm to about 10.6 μm, about 4.9 μm to about 12.4 μm, about 3.9 μm to about 11.0 μm, or about 4.2 μm to about 11.6 μm.

In certain embodiments, the micronized crystalline form of the compound of formula (I) has a particle size distribution which is defined by a D₉₀ of about 6.1 μm to about 7.7 μm.

In certain embodiments, the micronized crystalline form of the compound of formula (I) has a particle size distribution which is defined by a D₉₀ of about 6.1 μm to about 8.0 μm.

In certain embodiments, the micronized crystalline form of the compound of formula (I) has a particle size distribution which is defined by a D₉₀ of about 4.1 μm to about 10.0 μm.

In certain embodiments, the micronized crystalline form of the compound of formula (I) has a particle size distribution which is defined by a D₉₀ of about 5.6 μm to about 10.6 μm.

In certain embodiments, the micronized crystalline form of the compound of formula (I) has a particle size distribution which is defined by a D₉₀ of about 4.9 μm to about 12.4 μm.

In certain embodiments, the micronized crystalline form of the compound of formula (I) has a particle size distribution which is defined by a D₉₀ of about 3.9 μm to about 11.0 μm.

In certain embodiments, the micronized crystalline form of the compound of formula (I) has a particle size distribution which is defined by a D₉₀ of about 4.2 μm to about 11.6 μm.

In various embodiments, the invention provides a pharmaceutical composition comprising
(i) about 0.4% (w/w) to about 36% (w/w) of a crystalline form of the compound of formula (I)

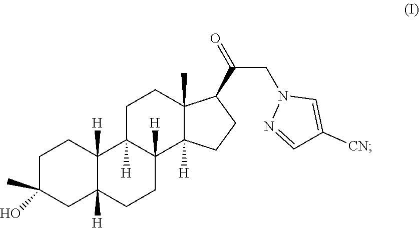

(ii) about 15% (w/w) to about 75% (w/w) of a brittle filler;
(iii) about 10% (w/w) to about 60% (w/w) of a ductile filler;
(iv) about 3% (w/w) to about 12% (w/w) of a disintegrant;
(v) about 0.25% (w/w) to about 5% (w/w) of a glidant; and
(vi) about 0.5% (w/w) to about 3% (w/w) of a lubricant,
   wherein the process for making the pharmaceutical composition comprises a micronization step that includes micronizing a crystalline form of the compound of formula (I) to obtain a micronized crystalline form of the compound of formula (I).

In certain embodiments, the micronized crystalline form of the compound of formula (I) has a particle size distribution which is defined by a D₉₀ of about 6.1 μm to about 7.7 μm, about 6.1 μm to about 8.0 μm, about 4.1 μm to about 10.0 μm, about 5.6 μm to about 10.6 μm, about 4.9 μm to about 12.4 μm, about 3.9 μm to about 11.0 μm, or about 4.2 μm to about 11.6 μm.

In certain embodiments, the micronized crystalline form of the compound of formula (I) has a particle size distribution which is defined by a D₉₀ of about 6.1 μm to about 7.7 μm.

In certain embodiments, the micronized crystalline form of the compound of formula (I) has a particle size distribution which is defined by a D₉₀ of about 6.1 μm to about 8.0 μm.

In certain embodiments, the micronized crystalline form of the compound of formula (I) has a particle size distribution which is defined by a D₉₀ of about 4.1 μm to about 10.0 μm.

In certain embodiments, the micronized crystalline form of the compound of formula (I) has a particle size distribution which is defined by a D₉₀ of about 5.6 μm to about 10.6 μm.

In certain embodiments, the micronized crystalline form of the compound of formula (I) has a particle size distribution which is defined by a D₉₀ of about 4.9 μm to about 12.4 μm.

In certain embodiments, the micronized crystalline form of the compound of formula (I) has a particle size distribution which is defined by a D₉₀ of about 3.9 μm to about 11.0 μm.

In certain embodiments, the micronized crystalline form of the compound of formula (I) has a particle size distribution which is defined by a D₉₀ of about 4.2 μm to about 11.6 μm.

In various embodiments, the invention provides a pharmaceutical composition comprising
(i) about 10% (w/w) to about 15% (w/w) of a crystalline form of the compound of formula (I)

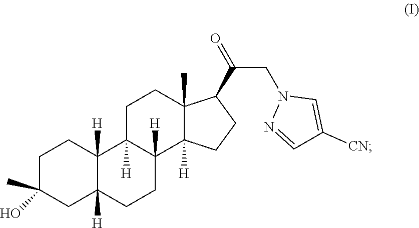

(ii) about 60% (w/w) to about 70% (w/w) of a brittle filler;
(iii) about 10% (w/w) to about 20% (w/w) of a ductile filler;
(iv) about 4% (w/w) to about 8% (w/w) of a disintegrant;
(v) about 0.5% (w/w) to about 2% (w/w) of a glidant; and
(vi) about 1% (w/w) to about 2% (w/w) of a lubricant,
   wherein the process for making the pharmaceutical composition comprises a micronization step that includes micronizing a crystalline form of the compound of formula (I) to obtain a micronized crystalline form of the compound of formula (I).

In certain embodiments, the micronized crystalline form of the compound of formula (I) has a particle size distribution which is defined by a D₉₀ of about 6.1 μm to about 7.7 μm, about 6.1 μm to about 8.0 μm, about 4.1 μm to about 10.0 μm, about 5.6 μm to about 10.6 μm, about 4.9 μm to about 12.4 μm, about 3.9 μm to about 11.0 μm, or about 4.2 μm to about 11.6 μm.

In certain embodiments, the micronized crystalline form of the compound of formula (I) has a particle size distribution which is defined by a D₉₀ of about 6.1 μm to about 7.7 μm.

In certain embodiments, the micronized crystalline form of the compound of formula (I) has a particle size distribution which is defined by a D₉₀ of about 6.1 μm to about 8.0 μm.

In certain embodiments, the micronized crystalline form of the compound of formula (I) has a particle size distribution which is defined by a D₉₀ of about 4.1 μm to about 10.0 μm.

In certain embodiments, the micronized crystalline form of the compound of formula (I) has a particle size distribution which is defined by a D₉₀ of about 5.6 μm to about 10.6 μm.

In certain embodiments, the micronized crystalline form of the compound of formula (I) has a particle size distribution which is defined by a D₉₀ of about 4.9 μm to about 12.4 μm.

In certain embodiments, the micronized crystalline form of the compound of formula (I) has a particle size distribution which is defined by a D₉₀ of about 3.9 μm to about 11.0 μm.

In certain embodiments, the micronized crystalline form of the compound of formula (I) has a particle size distribution which is defined by a D₉₀ of about 4.2 μm to about 11.6 μm.

In various embodiments, the invention provides a pharmaceutical composition comprising
(i) about 20 mg of a crystalline form of the compound of formula (I)

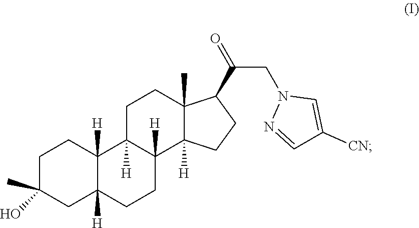

(ii) about 105.9 mg of mannitol;
(iii) about 26.2 mg of silicified microcrystalline cellulose;
(iv) about 10 mg of croscarmellose sodium;
(v) about 1.7 mg of colloidal silicon dioxide; and
(vi) about 2.9 mg of sodium stearyl fumarate,
   wherein the process for making the pharmaceutical composition comprises a micronization step that includes micronizing a crystalline form of the compound of formula (I) to obtain a micronized crystalline form of the compound of formula (I).

In certain embodiments, the micronized crystalline form of the compound of formula (I) has a particle size distribution which is defined by a D₉₀ of about 6.1 μm to about 7.7 μm, about 6.1 μm to about 8.0 μm, about 4.1 μm to about 10.0 μm, about 5.6 μm to about 10.6 μm, about 4.9 μm to about 12.4 μm, about 3.9 μm to about 11.0 μm, or about 4.2 μm to about 11.6 μm.

In certain embodiments, the micronized crystalline form of the compound of formula (I) has a particle size distribution which is defined by a D₉₀ of about 6.1 μm to about 7.7 μm.

In certain embodiments, the micronized crystalline form of the compound of formula (I) has a particle size distribution which is defined by a D₉₀ of about 6.1 μm to about 8.0 μm.

In certain embodiments, the micronized crystalline form of the compound of formula (I) has a particle size distribution which is defined by a D₉₀ of about 4.1 μm to about 10.0 μm.

In certain embodiments, the micronized crystalline form of the compound of formula (I) has a particle size distribution which is defined by a D₉₀ of about 5.6 μm to about 10.6 μm.

In certain embodiments, the micronized crystalline form of the compound of formula (I) has a particle size distribution which is defined by a D₉₀ of about 4.9 μm to about 12.4 μm.

In certain embodiments, the micronized crystalline form of the compound of formula (I) has a particle size distribution which is defined by a D₉₀ of about 3.9 μm to about 11.0 μm.

In certain embodiments, the micronized crystalline form of the compound of formula (I) has a particle size distribution which is defined by a D₉₀ of about 4.2 μm to about 11.6 μm.

In various embodiments, the invention provides a pharmaceutical composition comprising
(i) about 25 mg of a crystalline form of the compound of formula (I)

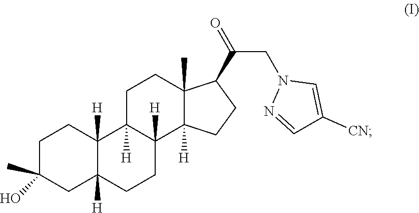

(ii) about 132 mg of mannitol;
(iii) about 32.7 mg of the silicified microcrystalline cellulose;
(iv) about 12.5 mg of croscarmellose sodium;
(v) about 2.1 mg of colloidal silicon dioxide; and
(vi) about 3.6 mg of sodium stearyl fumarate,
   wherein the process for making the pharmaceutical composition comprises a micronization step that includes micronizing a crystalline form of the compound of formula (I) to obtain a micronized crystalline form of the compound of formula (I).

In certain embodiments, the micronized crystalline form of the compound of formula (I) has a particle size distribution which is defined by a D₉₀ of about 6.1 μm to about 7.7 μm, about 6.1 μm to about 8.0 μm, about 4.1 μm to about 10.0 μm, about 5.6 μm to about 10.6 μm, about 4.9 μm to about 12.4 μm, about 3.9 μm to about 11.0 μm, or about 4.2 μm to about 11.6 μm.

In certain embodiments, the micronized crystalline form of the compound of formula (I) has a particle size distribution which is defined by a D₉₀ of about 6.1 μm to about 7.7 μm.

In certain embodiments, the micronized crystalline form of the compound of formula (I) has a particle size distribution which is defined by a D₉₀ of about 6.1 μm to about 8.0 μm.

In certain embodiments, the micronized crystalline form of the compound of formula (I) has a particle size distribution which is defined by a D₉₀ of about 4.1 μm to about 10.0 μm.

In certain embodiments, the micronized crystalline form of the compound of formula (I) has a particle size distribution which is defined by a D₉₀ of about 5.6 μm to about 10.6 μm.

In certain embodiments, the micronized crystalline form of the compound of formula (I) has a particle size distribution which is defined by a D₉₀ of about 4.9 μm to about 12.4 μm.

In certain embodiments, the micronized crystalline form of the compound of formula (I) has a particle size distribution which is defined by a D₉₀ of about 3.9 μm to about 11.0 μm.

In certain embodiments, the micronized crystalline form of the compound of formula (I) has a particle size distribution which is defined by a D₉₀ of about 4.2 μm to about 11.6 μm.

In various embodiments, the invention provides a pharmaceutical composition comprising
(i) about 30 mg of a crystalline form of the compound of formula (I)

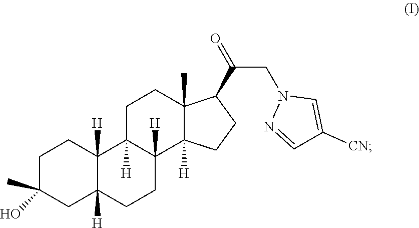

(ii) about 159 mg of mannitol;
(iii) about 39.3 mg of the silicified microcrystalline cellulose;
(iv) about 15 mg of croscarmellose sodium;
(v) about 2.5 mg of colloidal silicon dioxide; and
(vi) about 4.4 mg of sodium stearyl fumarate,
   wherein the process for making the pharmaceutical composition comprises a micronization step that includes micronizing a crystalline form of the compound of formula (I) to obtain a micronized crystalline form of the compound of formula (I).

In certain embodiments, the micronized crystalline form of the compound of formula (I) has a particle size distribution which is defined by a D₉₀ of about 6.1 μm to about 7.7 μm, about 6.1 μm to about 8.0 μm, about 4.1 μm to about 10.0 μm, about 5.6 μm to about 10.6 μm, about 4.9 μm to about 12.4 μm, about 3.9 μm to about 11.0 μm, or about 4.2 μm to about 11.6 μm.

In certain embodiments, the micronized crystalline form of the compound of formula (I) has a particle size distribution which is defined by a D₉₀ of about 6.1 μm to about 7.7 μm.

In certain embodiments, the micronized crystalline form of the compound of formula (I) has a particle size distribution which is defined by a D₉₀ of about 6.1 μm to about 8.0 μm.

In certain embodiments, the micronized crystalline form of the compound of formula (I) has a particle size distribution which is defined by a D₉₀ of about 4.1 μm to about 10.0 μm.

In certain embodiments, the micronized crystalline form of the compound of formula (I) has a particle size distribution which is defined by a D₉₀ of about 5.6 μm to about 10.6 μm.

In certain embodiments, the micronized crystalline form of the compound of formula (I) has a particle size distribution which is defined by a D₉₀ of about 4.9 μm to about 12.4 μm.

In certain embodiments, the micronized crystalline form of the compound of formula (I) has a particle size distribution which is defined by a D₉₀ of about 3.9 μm to about 11.0 μm.

In certain embodiments, the micronized crystalline form of the compound of formula (I) has a particle size distribution which is defined by a D₉₀ of about 4.2 μm to about 11.6 μm.

In various embodiments, the invention provides a pharmaceutical composition comprising
(i) about 40 mg of a crystalline form of the compound of formula (I)

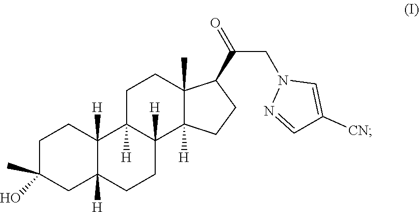

(ii) about 212 mg of mannitol;
(iii) about 52.4 mg of the silicified microcrystalline cellulose;
(iv) about 20 mg of croscarmellose sodium;
(v) about 3.3 mg of colloidal silicon dioxide; and
(vi) about 5.8 mg of sodium stearyl fumarate,
   wherein the process for making the pharmaceutical composition comprises a micronization step that includes micronizing a crystalline form of the compound of formula (I) to obtain a micronized crystalline form of the compound of formula (I).

In certain embodiments, the micronized crystalline form of the compound of formula (I) has a particle size distribution which is defined by a D₉₀ of about 6.1 μm to about 7.7 μm, about 6.1 μm to about 8.0 μm, about 4.1 μm to about 10.0 μm, about 5.6 μm to about 10.6 um, about 4.9 μm to about 12.4 μm, about 3.9 μm to about 11.0 μm, or about 4.2 μm to about 11.6 μm.

In certain embodiments, the micronized crystalline form of the compound of formula (I) has a particle size distribution which is defined by a D₉₀ of about 6.1 μm to about 7.7 μm.

In certain embodiments, the micronized crystalline form of the compound of formula (I) has a particle size distribution which is defined by a D₉₀ of about 6.1 μm to about 8.0 μm.

In certain embodiments, the micronized crystalline form of the compound of formula (I) has a particle size distribution which is defined by a D₉₀ of about 4.1 μm to about 10.0 μm.

In certain embodiments, the micronized crystalline form of the compound of formula (I) has a particle size distribution which is defined by a D₉₀ of about 5.6 μm to about 10.6 μm.

In certain embodiments, the micronized crystalline form of the compound of formula (I) has a particle size distribution which is defined by a D₉₀ of about 4.9 μm to about 12.4 μm.

In certain embodiments, the micronized crystalline form of the compound of formula (I) has a particle size distribution which is defined by a D₉₀ of about 3.9 μm to about 11.0 μm.

In certain embodiments, the micronized crystalline form of the compound of formula (I) has a particle size distribution which is defined by a D₉₀ of about 4.2 μm to about 11.6 μm.

In various embodiments, the invention provides a pharmaceutical composition comprising
(i) about 50 mg of a crystalline form of the compound of formula (I)

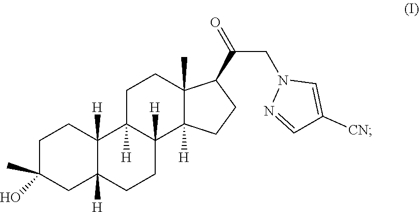

(ii) about 265 mg of mannitol;
(iii) about 65.5 mg of the silicified microcrystalline cellulose;
(iv) about 25 mg of croscarmellose sodium;
(v) about 4.2 mg of colloidal silicon dioxide; and
(vi) about 7.3 mg of sodium stearyl fumarate,
   wherein the process for making the pharmaceutical composition comprises a micronization step that includes micronizing a crystalline form of the compound of formula (I) to obtain a micronized crystalline form of the compound of formula (I).

In certain embodiments, the micronized crystalline form of the compound of formula (I) has a particle size distribution which is defined by a D₉₀ of about 6.1 μm to about 7.7 μm, about 6.1 μm to about 8.0 μm, about 4.1 μm to about 10.0 μm, about 5.6 μm to about 10.6 μm, about 4.9 μm to about 12.4 μm, about 3.9 μm to about 11.0 μm, or about 4.2 μm to about 11.6 μm.

In certain embodiments, the micronized crystalline form of the compound of formula (I) has a particle size distribution which is defined by a D₉₀ of about 6.1 μm to about 7.7 μm.

In certain embodiments, the micronized crystalline form of the compound of formula (I) has a particle size distribution which is defined by a D₉₀ of about 6.1 μm to about 8.0 μm.

In certain embodiments, the micronized crystalline form of the compound of formula (I) has a particle size distribution which is defined by a D₉₀ of about 4.1 μm to about 10.0 μm.

In certain embodiments, the micronized crystalline form of the compound of formula (I) has a particle size distribution which is defined by a D₉₀ of about 5.6 μm to about 10.6 μm.

In certain embodiments, the micronized crystalline form of the compound of formula (I) has a particle size distribution which is defined by a D₉₀ of about 4.9 μm to about 12.4 μm.

In certain embodiments, the micronized crystalline form of the compound of formula (I) has a particle size distribution which is defined by a D₉₀ of about 3.9 μm to about 11.0 μm.

In certain embodiments, the micronized crystalline form of the compound of formula (I) has a particle size distribution which is defined by a D₉₀ of about 4.2 μm to about 11.6 μm.

In various embodiments, the invention provides a pharmaceutical composition comprising
(i) about 60 mg of a crystalline form of the compound of formula (I)

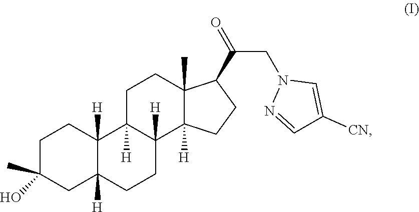

(ii) about 317.7 mg of mannitol;
(iii) about 78.6 mg of the silicified microcrystalline cellulose;
(iv) about 30 mg of croscarmellose sodium;
(v) about 5 mg of colloidal silicon dioxide; and
(vi) about 8.8 mg of sodium stearyl fumarate,
   wherein the process for making the pharmaceutical composition comprises a micronization step that includes micronizing a crystalline form of the compound of formula (I) to obtain a micronized crystalline form of the compound of formula (I).

In certain embodiments, the micronized crystalline form of the compound of formula (I) has a particle size distribution which is defined by a D₉₀ of about 6.1 μm to about 7.7 μm, about 6.1 μm to about 8.0 μm, about 4.1 μm to about 10.0 μm, about 5.6 μm to about 10.6 μm, about 4.9 μm to about 12.4 μm, about 3.9 μm to about 11.0 μm, or about 4.2 μm to about 11.6 μm.

In certain embodiments, the micronized crystalline form of the compound of formula (I) has a particle size distribution which is defined by a D₉₀ of about 6.1 μm to about 7.7 μm.

In certain embodiments, the micronized crystalline form of the compound of formula (I) has a particle size distribution which is defined by a D₉₀ of about 6.1 μm to about 8.0 μm.

In certain embodiments, the micronized crystalline form of the compound of formula (I) has a particle size distribution which is defined by a D₉₀ of about 4.1 μm to about 10.0 μm.

In certain embodiments, the micronized crystalline form of the compound of formula (I) has a particle size distribution which is defined by a D₉₀ of about 5.6 μm to about 10.6 μm.

In certain embodiments, the micronized crystalline form of the compound of formula (I) has a particle size distribution which is defined by a D₉₀ of about 4.9 μm to about 12.4 μm.

In certain embodiments, the micronized crystalline form of the compound of formula (I) has a particle size distribution which is defined by a D₉₀ of about 3.9 μm to about 11.0 μm.

In certain embodiments, the micronized crystalline form of the compound of formula (I) has a particle size distribution which is defined by a D₉₀ of about 4.2 μm to about 11.6 μm.

In certain embodiments, the micronized crystalline form of the compound of formula (I) has a particle size distribution which is defined by a D₉₀ of about 1 μm to about 100 μm.

In certain embodiments, the process for making the pharmaceutical composition further comprises one or more of the following process steps:
blending the micronized crystalline form of the compound of formula (I) with one or more pharmaceutically acceptable excipients described herein to obtain a blend; granulating the blend to obtain granules;
milling the granules to obtain an intragranular phase; and
blending the intragranular phase with one or more extragranular pharmaceutical excipients described herein to obtain the pharmaceutical composition.

In certain embodiments, granulating the blend to obtain granules comprises a dry granulation process step. In certain embodiments, granulating the blend to obtain granules comprises a wet granulation process step.

### Methods of Use and Treatment

In an aspect, pharmaceutical compositions described herein, e.g., pharmaceutical compositions of the compound of formula (I), or a pharmaceutically acceptable salt thereof, are envisioned to be useful as therapeutic compositions for treating a CNS-related disorder (e.g., sleep disorder, a mood disorder such as depression, a schizophrenia spectrum disorder, a convulsive disorder, epileptogenesis, a disorder of memory and/or cognition, a movement disorder, a personality disorder, autism spectrum disorder, pain, traumatic brain injury, a vascular disease, a substance abuse disorder and/or withdrawal syndrome, or tinnitus) in a subject in need (e.g., a subject with Rett syndrome, Fragile X syndrome, or Angelman syndrome). Exemplary CNS conditions related to GABA-modulation include, but are not limited to, sleep disorders (e.g., insomnia), mood disorders (e.g., depression (e.g., major depressive disorder (MDD), treatment-resistant depression (TRD)), dysthymic disorder (e.g., mild depression), bipolar disorder (e.g., I and/or II), anxiety disorders (e.g., generalized anxiety disorder (GAD), social anxiety disorder), stress, post-traumatic stress disorder (PTSD), compulsive disorders (e.g., obsessive compulsive disorder (OCD), schizophrenia spectrum disorders (e.g., schizophrenia, schizoaffective disorder), convulsive disorders (e.g., epilepsy (e.g., status epilepticus (SE)), seizures), disorders of memory and/or cognition (e.g., attention disorders (e.g., attention deficit hyperactivity disorder (ADHD)), dementia (e.g., Alzheimer's type dementia, Lewis body type dementia, vascular type dementia), movement disorders (e.g., Huntington's disease, Parkinson's disease), personality disorders (e.g., anti-social personality disorder, obsessive compulsive personality disorder), autism spectrum disorders (ASD) (e.g., autism, monogenetic causes of autism such as synaptophathy's, e.g., Rett syndrome, Fragile X syndrome, Angelman syndrome), pain (e.g., neuropathic pain, injury related pain syndromes, acute pain, chronic pain), traumatic brain injury (TBI), vascular diseases (e.g., stroke, ischemia, vascular malformations), substance abuse disorders and/or withdrawal syndromes (e.g., addition to opiates, cocaine, and/or alcohol), and tinnitus.

In certain embodiments, CNS-related disorder is a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, or status epilepticus. In certain embodiments, the CNS-related disorder is depression. In certain embodiments, the CNS-related disorder is postpartum depression. In certain embodiments, the CNS-related disorder is major depressive disorder. In certain embodiments, the major depressive disorder is moderate major depressive disorder. In certain embodiments, the major depressive disorder is severe major depressive disorder.

In an aspect, provided is a method of alleviating or preventing seizure activity in a subject, comprising administering to the subject in need of such treatment a pharmaceutical composition of the present invention comprising an effective amount of the compound of formula (I), or a pharmaceutically acceptable salt thereof. In some embodiments, the method alleviates or prevents epileptogenesis.

In yet another aspect, provided is a combination of the compound of formula (I) or a pharmaceutically acceptable salt thereof, and another pharmacologically active agent. The compound of formula (I), or a pharmaceutically acceptable salt thereof, can be administered as the sole active agent or they can be administered in combination with other agents. Administration in combination can proceed by any technique apparent to those of skill in the art including, for example, separate, sequential, concurrent and alternating administration.

In another aspect, provided is a method of treating or preventing brain excitability in a subject susceptible to or afflicted with a condition associated with brain excitability, comprising administering to the subject a pharmaceutical composition of the present invention comprising an effective amount of the compound of formula (I), or a pharmaceutically acceptable salt thereof.

In yet another aspect, provided is a method of treating or preventing stress or anxiety in a subject, comprising administering to the subject in need of such treatment a pharmaceutical composition of the present invention comprising an effective amount of the compound of formula (I), or a pharmaceutically acceptable salt thereof.

In yet another aspect, provided is a method of alleviating or preventing insomnia in a subject, comprising administering to the subject in need of such treatment a pharmaceutical composition of the present invention comprising an effective amount of the compound of formula (I), or a pharmaceutically acceptable salt thereof.

In yet another aspect, provided is a method of inducing sleep and maintaining substantially the level of REM sleep that is found in normal sleep, wherein substantial rebound insomnia is not induced, comprising administering a pharmaceutical composition of the present invention comprising an effective amount of the compound of formula (I), or a pharmaceutically acceptable salt thereof.

In yet another aspect, provided is a method of alleviating or preventing premenstrual syndrome (PMS) or postpartum depression (PPD) in a subject, comprising administering to the subject in need of such treatment a pharmaceutical composition of the present invention comprising an effective amount of the compound of formula (I), or a pharmaceutically acceptable salt thereof.

In yet another aspect, provided is a method of treating or preventing mood disorders in a subject, comprising administering to the subject in need of such treatment a pharmaceutical composition of the present invention comprising an effective amount of the compound of formula (I), or a pharmaceutically acceptable salt thereof. In certain embodiments the mood disorder is depression.

In yet another aspect, provided is a method of cognition enhancement or treating memory disorder by administering to the subject a pharmaceutical composition of the present invention comprising an effective amount of the compound of formula (I), or a pharmaceutically acceptable salt thereof. In certain embodiments, the disorder is Alzheimer's disease. In certain embodiments, the disorder is Rett syndrome.

In yet another aspect, provided is a method of treating attention disorders by administering to the subject a pharmaceutical composition of the present invention comprising an effective amount of the compound of formula (I), or a pharmaceutically acceptable salt thereof. In certain embodiments, the attention disorder is ADHD.

In certain embodiments, the administration of a pharmaceutical composition described herein to the subject is acute administration, chronic administration, or episodic administration. In certain embodiments, the pharmaceutical composition is administered to the subject orally.

### Neuroendocrine Disorders and Dysfunction

Provided herein are methods that can be used for treating neuroendocrine disorders and dysfunction. As used herein, “neuroendocrine disorder” or “neuroendocrine dysfunction” refers to a variety of conditions caused by imbalances in the body's hormone production directly related to the brain. Neuroendocrine disorders involve interactions between the nervous system and the endocrine system. Because the hypothalamus and the pituitary gland are two areas of the brain that regulate the production of hormones, damage to the hypothalamus or pituitary gland, e.g., by traumatic brain injury, may impact the production of hormones and other neuroendocrine functions of the brain. In some embodiments, the neuroendocrine disorder or dysfunction is associated with a women's health disorder or condition (e.g., a women's health disorder or condition described herein). In some embodiments, the neuroendocrine disorder or dysfunction is associated with a women's health disorder or condition is polycystic ovary syndrome.

Symptoms of neuroendocrine disorder include, but are not limited to, behavioral, emotional, and sleep-related symptoms, symptoms related to reproductive function, and somatic symptoms; including but not limited to fatigue, poor memory, anxiety, depression, weight gain or loss, emotional lability, lack of concentration, attention difficulties, loss of libido, infertility, amenorrhea, loss of muscle mass, increased belly body fat, low blood pressure, reduced heart rate, hair loss, anemia, constipation, cold intolerance, and dry skin.

### Neurodegenerative Diseases and Disorders

The methods described herein can be used for treating neurodegenerative diseases and disorders. The term “neurodegenerative disease” includes diseases and disorders that are associated with the progressive loss of structure or function of neurons, or death of neurons. Neurodegenerative diseases and disorders include, but are not limited to, Alzheimer's disease (including the associated symptoms of mild, moderate, or severe cognitive impairment); amyotrophic lateral sclerosis (ALS); anoxic and ischemic injuries; ataxia and convulsion (including for the treatment and prevention and prevention of seizures that are caused by schizoaffective disorder or by drugs used to treat schizophrenia); benign forgetfulness; brain edema; cerebellar ataxia including McLeod neuroacanthocytosis syndrome (MLS); closed head injury; coma; contusive injuries (e.g., spinal cord injury and head injury); dementias including multi-infarct dementia and senile dementia; disturbances of consciousness; Down syndrome; drug-induced or medication-induced Parkinsonism (such as neuroleptic-induced acute akathisia, acute dystonia, Parkinsonism, or tardive dyskinesia, neuroleptic malignant syndrome, or medication-induced postural tremor); epilepsy; fragile X syndrome; Gilles de la Tourette's syndrome; head trauma; hearing impairment and loss; Huntington's disease; Lennox syndrome; levodopa-induced dyskinesia; mental retardation; movement disorders including akinesias and akinetic (rigid) syndromes (including basal ganglia calcification, corticobasal degeneration, multiple system atrophy, Parkinsonism-ALS dementia complex, Parkinson's disease, postencephalitic parkinsonism, and progressively supranuclear palsy); muscular spasms and disorders associated with muscular spasticity or weakness including chorea (such as benign hereditary chorea, drug-induced chorea, hemiballism, Huntington's disease, neuroacanthocytosis, Sydenham's chorea, and symptomatic chorea), dyskinesia (including tics such as complex tics, simple tics, and symptomatic tics), myoclonus (including generalized myoclonus and focal cyloclonus), tremor (such as rest tremor, postural tremor, and intention tremor) and dystonia (including axial dystonia, dystonic writer's cramp, hemiplegic dystonia, paroxysmal dystonia, and focal dystonia such as blepharospasm, oromandibular dystonia, and spasmodic dysphonia and torticollis); neuronal damage including ocular damage, retinopathy or macular degeneration of the eye; neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest; Parkinson's disease; seizure; status epilecticus; stroke; tinnitus; tubular sclerosis, and viral infection induced neurodegeneration (e.g., caused by acquired immunodeficiency syndrome (AIDS) and encephalopathies). Neurodegenerative diseases also include, but are not limited to, neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest. Methods of treating or preventing a neurodegenerative disease also include treating or preventing loss of neuronal function characteristic of neurodegenerative disorder.

### Mood Disorders

Also provided herein are methods for treating a mood disorder, for example clinical depression, postpartum depression, perinatal depression, atypical depression, melancholic depression, psychotic major depression, catatonic depression, seasonal affective disorder, dysthymia, double depression, depressive personality disorder, recurrent brief depression, minor depressive disorder, bipolar disorder or manic depressive disorder, depression caused by chronic medical conditions, treatment-resistant depression, refractory depression, suicidality, suicidal ideation, or suicidal behavior. In some embodiments, the method described herein provides therapeutic effect to a subject suffering from depression (e.g., moderate or severe depression). In some embodiments, the mood disorder is associated with a disease or disorder described herein (e.g., neuroendocrine diseases and disorders, neurodegenerative diseases and disorders (e.g., epilepsy), movement disorders, tremor (e.g., Parkinson's Disease), women's health disorders or conditions).

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

Peripartum depression refers to depression in pregnancy. Symptoms include irritability, crying, feeling restless, trouble sleeping, extreme exhaustion (emotional and/or physical), changes in appetite, difficulty focusing, increased anxiety and/or worry, disconnected feeling from baby and/or fetus, and losing interest in formerly pleasurable activities.

Postpartum depression (PPD), refers to a type of clinical depression that affects women after childbirth. Symptoms can include sadness, fatigue, changes in sleeping and eating habits, reduced sexual desire, crying episodes, anxiety, and irritability. In some embodiments, the PPD is a treatment-resistant depression (e.g., a treatment-resistant depression as described herein). In some embodiments, the PPD is refractory depression (e.g., a refractory depression as described herein).

In some embodiments, a subject having PPD also experienced depression, or a symptom of depression during pregnancy. This depression is referred to herein as perinatal depression. In an embodiment, a subject experiencing perinatal depression is at increased risk of experiencing PPD.

Atypical depression (AD) is characterized by mood reactivity (e.g., paradoxical anhedonia) and positivity, significant weight gain or increased appetite. Patients suffering from AD also may have excessive sleep or somnolence (hypersomnia), a sensation of limb heaviness, and significant social impairment as a consequence of hypersensitivity to perceived interpersonal rejection.

Melancholic depression is characterized by loss of pleasure (anhedonia) in most or all activities, failures to react to pleasurable stimuli, depressed mood more pronounced than that of grief or loss, excessive weight loss, or excessive guilt.

Psychotic major depression (PMD) or psychotic depression refers to a major depressive episode, in particular of melancholic nature, where the individual experiences psychotic symptoms such as delusions and hallucinations.

Catatonic depression refers to major depression involving disturbances of motor behavior and other symptoms. An individual may become mute and stuporose, and either is immobile or exhibits purposeless or bizarre movements.

Seasonal affective disorder (SAD) refers to a type of seasonal depression wherein an individual has seasonal patterns of depressive episodes coming on in the fall or winter.

Dysthymia refers to a condition related to unipolar depression, where the same physical and cognitive problems are evident. They are not as severe and tend to last longer (e.g., at least 2 years).

Double depression refers to fairly depressed mood (dysthymia) that lasts for at least 2 years and is punctuated by periods of major depression.

Depressive Personality Disorder (DPD) refers to a personality disorder with depressive features.

Recurrent Brief Depression (RBD) refers to a condition in which individuals have depressive episodes about once per month, each episode lasting 2 weeks or less and typically less than 2-3 days.

Minor depressive disorder or minor depression refers to a depression in which at least 2 symptoms are present for 2 weeks.

Bipolar disorder or manic depressive disorder causes extreme mood swings that include emotional highs (mania or hypomania) and lows (depression). During periods of mania the individual may feel or act abnormally happy, energetic, or irritable. They often make poorly thought out decisions with little regard to the consequences. The need for sleep is usually reduced. During periods of depression there may be crying, poor eye contact with others, and a negative outlook on life. The risk of suicide among those with the disorder is high at greater than 6% over 20 years, while self-harm occurs in 30-40%. Other mental health issues such as anxiety disorder and substance use disorder are commonly associated with bipolar disorder.

Depression caused by chronic medical conditions refers to depression caused by chronic medical conditions such as cancer or chronic pain, chemotherapy, chronic stress.

Treatment-resistant depression refers to a condition where the individuals have been treated for depression, but the symptoms do not improve. For example, antidepressants or psychological counseling (psychotherapy) do not ease depression symptoms for individuals with treatment-resistant depression. In some cases, individuals with treatment-resistant depression improve symptoms, but come back. Refractory depression occurs in patients suffering from depression who are resistant to standard pharmacological treatments, including tricyclic antidepressants, MAOIs, SSRIs, and double and triple uptake inhibitors and/or anxiolytic drugs, as well as non-pharmacological treatments (e.g., psychotherapy, electroconvulsive therapy, vagus nerve stimulation and/or transcranial magnetic stimulation).

Post-surgical depression refers to feelings of depression that follow a surgical procedure (e.g., as a result of having to confront one's mortality). For example, individuals may feel sadness or empty mood persistently, a loss of pleasure or interest in hobbies and activities normally enjoyed, or a persistent felling of worthlessness or hopelessness.

Mood disorder associated with conditions or disorders of women's health refers to mood disorders (e.g., depression) associated with (e.g., resulting from) a condition or disorder of women's health (e.g., as described herein).

Suicidality, suicidal ideation, suicidal behavior refers to the tendency of an individual to commit suicide. Suicidal ideation concerns thoughts about or an unusual preoccupation with suicide. The range of suicidal ideation varies greatly, from e.g., fleeting thoughts to extensive thoughts, detailed planning, role playing, incomplete attempts. Symptoms include talking about suicide, getting the means to commit suicide, withdrawing from social contact, being preoccupied with death, feeling trapped or hopeless about a situation, increasing use of alcohol or drugs, doing risky or self-destructive things, saying goodbye to people as if they won't be seen again.

Symptoms of depression include persistent anxious or sad feelings, feelings of helplessness, hopelessness, pessimism, worthlessness, low energy, restlessness, difficulty sleeping, sleeplessness, irritability, fatigue, motor challenges, loss of interest in pleasurable activities or hobbies, loss of concentration, loss of energy, poor self-esteem, absence of positive thoughts or plans, excessive sleeping, overeating, appetite loss, insomnia, self-harm, thoughts of suicide, and suicide attempts. The presence, severity, frequency, and duration of symptoms may vary on a case to case basis. Symptoms of depression, and relief of the same, may be ascertained by a physician or psychologist (e.g., by a mental state examination).

In some embodiments, the method comprises monitoring a subject with a known depression scale, e.g., the Hamilton Depression (HAM-D) scale, the Clinical Global Impression-Improvement Scale (CGI), and the Montgomery-Åsberg Depression Rating Scale (MADRS). In some embodiments, a therapeutic effect can be determined by reduction in Hamilton Depression (HAM-D) total score exhibited by the subject. Reduction in the HAM-D total score can happen within 4, 3, 2, or 1 days; or 96, 84, 72, 60, 48, 24, 20, 16, 12, 10, 8 hours or less. The therapeutic effect can be assessed across a specified treatment period. For example, the therapeutic effect can be determined by a decrease from baseline in HAM-D total score after administering a compound described herein, e.g., a compound of Formula (I) (e.g., 12, 24, or 48 hours after administration; or 24, 48, 72, or 96 hours or more; or 1 day, 2 days, 14 days, 21 days, or 28 days; or 1 week, 2 weeks, 3 weeks, or 4 weeks; or 1 month, 2 months, 6 months, or 10 months; or 1 year, 2 years, or for life).

In some embodiments, the subject has a mild depressive disorder, e.g., mild major depressive disorder. In some embodiments, the subject has a moderate depressive disorder, e.g., moderate major depressive disorder. In some embodiments, the subject has a severe depressive disorder, e.g., severe major depressive disorder. In some embodiments, the subject has a very severe depressive disorder, e.g., very severe major depressive disorder. In some embodiments, the baseline HAM-D total score of the subject (i.e., prior to treatment with a compound described herein, e.g., a compound of formula (I)) is at least 24. In some embodiments, the baseline HAM-D total score of the subject is at least 18. In some embodiments, the baseline HAM-D total score of the subject is between and including 14 and 18. In some embodiments, the baseline HAM-D total score of the subject is between and including 19 and 22. In some embodiments, the HAM-D total score of the subject before treatment with a compound described herein, e.g., a compound of formula (I), is greater than or equal to 23. In some embodiments, the baseline score is at least 10, 15, or 20. In some embodiments, the HAM-D total score of the subject after treatment with a compound described herein, e.g., a compound of formula (I), is about 0 to 10 (e.g., less than 10; 0 to 10, 0 to 6, 0 to 4, 0 to 3, 0 to 2, or 1.8). In some embodiments, the HAM-D total score after treatment with a compound described herein, e.g., a compound of formula (I), is less than 10, 7, 5, or 3. In some embodiments, the decrease in HAM-D total score is from a baseline score of about 20 to 30 (e.g., 22 to 28, 23 to 27, 24 to 27, 25 to 27, 26 to 27) to a HAM-D total score at about 0 to 10 (e.g., less than 10; 0 to 10, 0 to 6, 0 to 4, 0 to 3, 0 to 2, or 1.8) after treatment with a compound described herein, e.g., a compound of formula (I). In some embodiments, the decrease in the baseline HAM-D total score to HAM-D total score after treatment with a compound described herein, e.g., a compound of formula (I), is at least 1, 2, 3, 4, 5, 7, 10, 25, 40, 50, or 100 fold). In some embodiments, the percentage decrease in the baseline HAM-D total score to HAM-D total score after treatment with a compound described herein, e.g., a compound of formula (I), is at least 50% (e.g., 60%, 70%, 80%, or 90%). In some embodiments, the therapeutic effect is measured as a decrease in the HAM-D total score after treatment with a compound described herein, e.g., a compound of formula (I), relative to the baseline HAM-D total score (e.g., 12, 24, 48 hours after administration; or 24, 48, 72, 96 hours or more; or 1 day, 2 days, 14 days, or more) is at least 10, 15, or 20 points.

In some embodiments, the method of treating a depressive disorder, e.g., major depressive disorder provides a therapeutic effect (e.g., as measured by reduction in Hamilton Depression Score (HAM-D)) within 14, 10, 4, 3, 2, or 1 days, or 24, 20, 16, 12, 10, or 8 hours or less. In some embodiments, the method of treating the depressive disorder, e.g., major depressive disorder, provides a therapeutic effect (e.g., as determined by a statistically significant reduction in HAM-D total score) within the first or second day of the treatment with a compound described herein, e.g., a compound of formula (I). In some embodiments, the method of treating the depressive disorder, e.g., major depressive disorder, provides a therapeutic effect (e.g., as determined by a statistically significant reduction in HAM-D total score) within less than or equal to 14 days since the beginning of the treatment with a compound described herein, e.g., a compound of formula (I). In some embodiments, the method of treating the depressive disorder, e.g., major depressive disorder, provides a therapeutic effect (e.g., as determined by a statistically significant reduction in HAM-D total score) within less than or equal to 21 days since the beginning of the treatment with a compound described herein, e.g., a compound of formula (I). In some embodiments, the method of treating the depressive disorder, e.g., major depressive disorder, provides a therapeutic effect (e.g., as determined by a statistically significant reduction in HAM-D total score) within less than or equal to 28 days since the beginning of the treatment with a compound described herein, e.g., a compound of formula (I). In some embodiments, the therapeutic effect is a decrease from baseline in HAM-D total score after treatment with a compound described herein, e.g., a compound of formula (I) (e.g., treatment with a compound described herein, e.g., a compound of formula (I), once a day for 14 days). In some embodiments, the HAM-D total score of the subject before treatment with a compound described herein, e.g., a compound of formula (I), is at least 24. In some embodiments, the HAM-D total score of the subject before treatment with a compound described herein, e.g., a compound of formula (I), is at least 18. In some embodiments, the HAM-D total score of the subject before treatment with a compound described herein, e.g., a compound of formula (I), is between and including 14 and 18. In some embodiments, the decrease in HAM-D total score after treating the subject with a compound described herein, e.g., a compound of formula (I), relative to the baseline HAM-D total score is at least 10. In some embodiments, the decrease in HAM-D total score after treating the subject with a compound described herein, e.g., a compound of formula (I), relative to the baseline HAM-D total score is at least 15 (e.g., at least 17). In some embodiments, the HAM-D total score associated with treating the subject with a compound described herein, e.g., a compound of formula (I), is no more than a number ranging from 6 to 8. In some embodiments, the HAM-D total score associated with treating the subject with a compound described herein, e.g., a compound of formula (I), is no more than 7.

In some embodiments, the method provides therapeutic effect (e.g., as measured by reduction in Clinical Global Impression-Improvement Scale (CGI)) within 14, 10, 4, 3, 2, or 1 days, or 24, 20, 16, 12, 10, or 8 hours or less. In some embodiments, the CNS-disorder is a depressive disorder, e.g., major depressive disorder. In some embodiments, the method of treating the depressive disorder, e.g., major depressive disorder provides a therapeutic effect within the second day of the treatment period. In some embodiments, the therapeutic effect is a decrease from baseline in CGI score at the end of a treatment period (e.g., 14 days after administration).

In some embodiments, the method provides therapeutic effect (e.g., as measured by reduction in Montgomery-Åsberg Depression Rating Scale (MADRS)) within 14, 10, 4, 3, 2, or 1 days, or 24, 20, 16, 12, 10, or 8 hours or less. In some embodiments, the CNS-disorder is a depressive disorder, e.g., major depressive disorder. In some embodiments, the method of treating the depressive disorder, e.g., major depressive disorder provides a therapeutic effect within the second day of the treatment period. In some embodiments, the therapeutic effect is a decrease from baseline in MADRS score at the end of a treatment period (e.g., 14 days after administration).

A therapeutic effect for major depressive disorder can be determined by a reduction in Montgomery-Åsberg Depression Rating Scale (MADRS) score exhibited by the subject. For example, the MADRS score can be reduced within 4, 3, 2, or 1 days; or 96, 84, 72, 60, 48, 24, 20, 16, 12, 10, 8 hours or less. The Montgomery-Åsberg Depression Rating Scale (MADRS) is a ten-item diagnostic questionnaire (regarding apparent sadness, reported sadness, inner tension, reduced sleep, reduced appetite, concentration difficulties, lassitude, inability to feel, pessimistic thoughts, and suicidal thoughts) which psychiatrists use to measure the severity of depressive episodes in patients with mood disorders.

In some embodiments, the method provides therapeutic effect (e.g., as measured by reduction in Edinburgh Postnatal Depression Scale (EPDS)) within 4, 3, 2, 1 days; 24, 20, 16, 12, 10, 8 hours or less. In some embodiments, the therapeutic effect is an improvement measured by the EPDS.

In some embodiments, the method provides therapeutic effect (e.g., as measured by reduction in Generalized Anxiety Disorder 7-Item Scale (GAD-7)) within 4, 3, 2, 1 days; 24, 20, 16, 12, 10, 8 hours or less.

### Anxiety Disorders

Provided herein are methods for treating anxiety disorders (e.g., generalized anxiety disorder, panic disorder, obsessive compulsive disorder, phobia, post-traumatic stress disorder). Anxiety disorder is a blanket term covering several different forms of abnormal and pathological fear and anxiety. Current psychiatric diagnostic criteria recognize a wide variety of anxiety disorders.

Generalized anxiety disorder is a common chronic disorder characterized by long-lasting anxiety that is not focused on any one object or situation. Those suffering from generalized anxiety experience non-specific persistent fear and worry and become overly concerned with everyday matters. Generalized anxiety disorder is the most common anxiety disorder to affect older adults.

In panic disorder, a person suffers from brief attacks of intense terror and apprehension, often marked by trembling, shaking, confusion, dizziness, nausea, difficulty breathing. These panic attacks, defined by the APA as fear or discomfort that abruptly arises and peaks in less than ten minutes, can last for several hours and can be triggered by stress, fear, or even exercise; although the specific cause is not always apparent. In addition to recurrent unexpected panic attacks, a diagnosis of panic disorder also requires that said attacks have chronic consequences: either worry over the attacks' potential implications, persistent fear of future attacks, or significant changes in behavior related to the attacks. Accordingly, those suffering from panic disorder experience symptoms even outside of specific panic episodes. Often, normal changes in heartbeat are noticed by a panic sufferer, leading them to think something is wrong with their heart or they are about to have another panic attack. In some cases, a heightened awareness (hypervigilance) of body functioning occurs during panic attacks, wherein any perceived physiological change is interpreted as a possible life threatening illness (i.e. extreme hypochondriasis).

Obsessive compulsive disorder is a type of anxiety disorder primarily characterized by repetitive obsessions (distressing, persistent, and intrusive thoughts or images) and compulsions (urges to perform specific acts or rituals). The OCD thought pattern may be likened to superstitions insofar as it involves a belief in a causative relationship where, in reality, one does not exist. Often the process is entirely illogical; for example, the compulsion of walking in a certain pattern may be employed to alleviate the obsession of impending harm. And in many cases, the compulsion is entirely inexplicable, simply an urge to complete a ritual triggered by nervousness. In a minority of cases, sufferers of OCD may only experience obsessions, with no overt compulsions; a much smaller number of sufferers experience only compulsions.

The single largest category of anxiety disorders is that of phobia, which includes all cases in which fear and anxiety is triggered by a specific stimulus or situation. Sufferers typically anticipate terrifying consequences from encountering the object of their fear, which can be anything from an animal to a location to a bodily fluid.

Post-traumatic stress disorder or PTSD is an anxiety disorder which results from a traumatic experience. Post-traumatic stress can result from an extreme situation, such as combat, rape, hostage situations, or even serious accident. It can also result from long term (chronic) exposure to a severe stressor, for example soldiers who endure individual battles but cannot cope with continuous combat. Common symptoms include flashbacks, avoidant behaviors, and depression.

### Women's Health Disorders

Provided herein are methods for treating conditions or disorders related to women's health. Conditions or disorders related to women's health include, but are not limited to, gynecological health and disorders (e.g., premenstrual syndrome (PMS), premenstrual dysphoric disorder (PMDD)), pregnancy issues (e.g., miscarriage, abortion), infertility and related disorders (e.g., polycystic ovary syndrome (PCOS)), other disorders and conditions, and issues related to women's overall health and wellness (e.g., menopause).

Gynecological health and disorders affecting women include menstruation and menstrual irregularities; urinary tract health, including urinary incontinence and pelvic floor disorders; and such disorders as bacterial vaginosis, vaginitis, uterine fibroids, and vulvodynia.

Premenstrual syndrome (PMS) refers to physical and emotional symptoms that occur in the one to two weeks before a women's period. Symptoms vary but can include bleeding, mood swings, tender breasts, food cravings, fatigue, irritability, acne, and depression.

Premenstrual dysphoric disorder (PMDD) is a severe form of PMS. The symptoms of PMDD are similar to PMS but more severe and may interfere with work, social activity, and relationships. PMDD symptoms include mood swings, depressed mood or feelings of hopelessness, marked anger, increased interpersonal conflicts, tension and anxiety, irritability, decreased interest in usual activities, difficulty concentrating, fatigue, change in appetite, feeling out of control or overwhelmed, sleep problems, physical problems (e.g., bloating, breast tenderness, swelling, headaches, joint or muscle pain).

Pregnancy issues include preconception care and prenatal care, pregnancy loss (miscarriage and stillbirth), preterm labor and premature birth, sudden infant death syndrome (SIDS), breastfeeding, and birth defects.

Miscarriage refers to a pregnancy that ends on its own, within the first 20 weeks of gestation.

Abortion refers to the deliberate termination of a pregnancy, which can be performed during the first 28 weeks of pregnancy.

Infertility and related disorders include uterine fibroids, polycystic ovary syndrome, endometriosis, and primary ovarian insufficiency.

Polycystic ovary syndrome (PCOS) refers to an endocrine system disorder among women of reproductive age. PCOS is a set of symptoms resulting from an elevated male hormone in women. Most women with PCOS grow many small cysts on their ovaries. Symptoms of PCOS include irregular or no menstrual periods, heavy periods, excess body and facial hair, acne, pelvic pain, difficulty getting pregnant, and patches of thick, darker, velvety skin. PCOS may be associated with conditions including type 2 diabetes, obesity, obstructive sleep apnea, heart disease, mood disorders, and endometrial cancer.

Other disorders and conditions that affect only women include Turner syndrome, Rett syndrome, and ovarian and cervical cancers.

Issues related to women's overall health and wellness include violence against women, women with disabilities and their unique challenges, osteoporosis and bone health, and menopause.

Menopause refers to the 12 months after a woman's last menstrual period and marks the end of menstrual cycles. Menopause typically occurs in a woman's 40s or 50s. Physical symptoms such as hot flashes and emotional symptoms of menopause may disrupt sleep, lower energy, or trigger anxiety or feelings of sadness or loss. Menopause includes natural menopause and surgical menopause, which is a type of induced menopause due to an event such as surgery (e.g., hysterectomy, oophorectomy; cancer). It is induced when the ovaries are gravely damaged by, e.g., radiation, chemotherapy, or other medications.

### Epilepsy

The pharmaceutical compositions of the compound of formula (I), or a pharmaceutically acceptable salt thereof, can be used in a method described herein, for example in the treatment of a disorder described herein such as epilepsy, status epilepticus, or seizure.

Epilepsy is a brain disorder characterized by repeated seizures over time. Types of epilepsy can include, but are not limited to generalized epilepsy, e.g. childhood absence epilepsy, juvenile nyoclonic epilepsy, epilepsy with grand-mal seizures on awakening, West syndrome, Lennox-Gastaut syndrome, partial epilepsy, e.g., temporal lobe epilepsy, frontal lobe epilepsy, benign focal epilepsy of childhood.

### Epileptogenesis

The pharmaceutical compositions and methods described herein can be used to treat or prevent epileptogenesis. Epileptogenesis is a gradual process by which a normal brain develops epilepsy (a chronic condition in which seizures occur). Epileptogenesis results from neuronal damage precipitated by the initial insult (e.g., status epilepticus).

### Status Epilepticus (SE)

Status epilepticus (SE) can include, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

The compound of formula (I) or pharmaceutically acceptable salt, or a pharmaceutically acceptable composition thereof, can also be administered as a prophylactic to a subject having a CNS disorder e.g., a traumatic brain injury, status epilepticus, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges; prior to the onset of a seizure.

### Seizure

A seizure is the physical findings or changes in behavior that occur after an episode of abnormal electrical activity in the brain. The term “seizure” is often used interchangeably with “convulsion.” Convulsions are when a person's body shakes rapidly and uncontrollably. During convulsions, the person's muscles contract and relax repeatedly.

Based on the type of behavior and brain activity, seizures are divided into two broad categories: generalized and partial (also called local or focal). Classifying the type of seizure helps doctors diagnose whether or not a patient has epilepsy.

Generalized seizures are produced by electrical impulses from throughout the entire brain, whereas partial seizures are produced (at least initially) by electrical impulses in a relatively small part of the brain. The part of the brain generating the seizures is sometimes called the focus.

There are six types of generalized seizures. The most common and dramatic, and therefore the most well-known, is the generalized convulsion, also called the grand-mal seizure. In this type of seizure, the patient loses consciousness and usually collapses. The loss of consciousness is followed by generalized body stiffening (called the “tonic” phase of the seizure) for 30 to 60 seconds, then by violent jerking (the “clonic” phase) for 30 to 60 seconds, after which the patient goes into a deep sleep (the “postictal” or after-seizure phase). During grand-mal seizures, injuries and accidents may occur, such as tongue biting and urinary incontinence.

Absence seizures cause a short loss of consciousness (just a few seconds) with few or no symptoms. The patient, most often a child, typically interrupts an activity and stares blankly. These seizures begin and end abruptly and may occur several times a day. Patients are usually not aware that they are having a seizure, except that they may be aware of “losing time.”

Myoclonic seizures consist of sporadic jerks, usually on both sides of the body. Patients sometimes describe the jerks as brief electrical shocks. When violent, these seizures may result in dropping or involuntarily throwing objects.

Clonic seizures are repetitive, rhythmic jerks that involve both sides of the body at the same time.

Tonic seizures are characterized by stiffening of the muscles.

Atonic seizures consist of a sudden and general loss of muscle tone, particularly in the arms and legs, which often results in a fall.

Seizures described herein can include epileptic seizures; acute repetitive seizures; cluster seizures; continuous seizures; unremitting seizures; prolonged seizures; recurrent seizures; status epilepticus seizures, e.g., refractory convulsive status epilepticus, non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; or withdrawal seizures. In some embodiments, the seizure is a generalized seizure associated with Dravet Syndrome, Lennox-Gastaut Syndrome, Tuberous Sclerosis Complex, Rett Syndrome or PCDH19 Female Pediatric Epilepsy.

### Movement Disorders

Also described herein are methods for treating a movement disorder. As used herein, “movement disorders” refers to a variety of diseases and disorders that are associated with hyperkinetic movement disorders and related abnormalities in muscle control. Exemplary movement disorders include, but are not limited to, Parkinson's disease and parkinsonism (defined particularly by bradykinesia), dystonia, chorea and Huntington's disease, ataxia, tremor (e.g., essential tremor), myoclonus and startle, tics and Tourette syndrome, Restless legs syndrome, stiff person syndrome, and gait disorders.

### Tremor

The methods described herein can be used to treat tremor, for example the pharmaceutical compositions of the compound of formula (I), or a pharmaceutically acceptable salt thereof, can be used to treat cerebellar tremor or intention tremor, dystonic tremor, essential tremor, orthostatic tremor, parkinsonian tremor, physiological tremor, psychogenic tremor, or rubral tremor. Tremor includes hereditary, degenerative, and idiopathic disorders such as Wilson's disease, Parkinson's disease, and essential tremor, respectively; metabolic diseases (e.g., thyroid-parathyroid-, liver disease and hypoglycemia); peripheral neuropathies (associated with Charcot-Marie-Tooth, Roussy-Levy, diabetes mellitus, complex regional pain syndrome); toxins (nicotine, mercury, lead, CO, Manganese, arsenic, toluene); drug-induced (narcoleptics, tricyclics, lithium, cocaine, alcohol, adrenaline, bronchodilators, theophylline, caffeine, steroids, valproate, amiodarone, thyroid hormones, vincristine); and psychogenic disorders. Clinical tremor can be classified into physiologic tremor, enhanced physiologic tremor, essential tremor syndromes (including classical essential tremor, primary orthostatic tremor, and task- and position-specific tremor), dystonic tremor, parkinsonian tremor, cerebellar tremor, Holmes' tremor (i.e., rubral tremor), palatal tremor, neuropathic tremor, toxic or drug-induced tremor, and psychogenic tremor.

Tremor is an involuntary, at times rhythmic, muscle contraction and relaxation that can involve oscillations or twitching of one or more body parts (e.g., hands, arms, eyes, face, head, vocal folds, trunk, legs).

Cerebellar tremor or intention tremor is a slow, broad tremor of the extremities that occurs after a purposeful movement. Cerebellar tremor is caused by lesions in or damage to the cerebellum resulting from, e.g., tumor, stroke, disease (e.g., multiple sclerosis, an inherited degenerative disorder).

Dystonic tremor occurs in individuals affected by dystonia, a movement disorder in which sustained involuntary muscle contractions cause twisting and repetitive motions and/or painful and abnormal postures or positions. Dystonic tremor may affect any muscle in the body. Dystonic tremors occur irregularly and often can be relieved by complete rest.

Essential tremor or benign essential tremor is the most common type of tremor. Essential tremor may be mild and nonprogressive in some, and may be slowly progressive, starting on one side of the body but affect both sides within 3 years. The hands are most often affected, but the head, voice, tongue, legs, and trunk may also be involved. Tremor frequency may decrease as the person ages, but severity may increase. Heightened emotion, stress, fever, physical exhaustion, or low blood sugar may trigger tremors and/or increase their severity. Symptoms generally evolve over time and can be both visible and persistent following onset.

Orthostatic tremor is characterized by fast (e.g., greater than 12 Hz) rhythmic muscle contractions that occurs in the legs and trunk immediately after standing. Cramps are felt in the thighs and legs and the patient may shake uncontrollably when asked to stand in one spot. Orthostatic tremor may occur in patients with essential tremor.

Parkinsonian tremor is caused by damage to structures within the brain that control movement. Parkinsonian tremor is often a precursor to Parkinson's disease and is typically seen as a “pill-rolling” action of the hands that may also affect the chin, lips, legs, and trunk. Onset of parkinsonian tremor typically begins after age 60. Movement starts in one limb or on one side of the body and can progress to include the other side.

Physiological tremor can occur in normal individuals and have no clinical significance. It can be seen in all voluntary muscle groups. Physiological tremor can be caused by certain drugs, alcohol withdrawal, or medical conditions including an overactive thyroid and hypoglycemia. The tremor classically has a frequency of about 10 Hz.

Psychogenic tremor or hysterical tremor can occur at rest or during postural or kinetic movement. Patient with psychogenic tremor may have a conversion disorder or another psychiatric disease.

Rubral tremor is characterized by coarse slow tremor which can be present at rest, at posture, and with intention. The tremor is associated with conditions that affect the red nucleus in the midbrain, classical unusual strokes.

Parkinson's Disease affects nerve cells in the brain that produce dopamine. Symptoms include muscle rigidity, tremors, and changes in speech and gait. Parkinsonism is characterized by tremor, bradykinesia, rigidity, and postural instability. Parkinsonism shares symptoms found in Parkinson's Disease, but is a symptom complex rather than a progressive neurodegenerative disease.

Dystonia is a movement disorder characterized by sustained or intermittent muscle contractions causing abnormal, often repetitive movements or postures. Dystonic movements can be patterned, twisting, and may be tremulous. Dystonia is often initiated or worsened by voluntary action and associated with overflow muscle activation.

Chorea is a neurological disorder characterized by jerky involuntary movements typically affecting the shoulders, hips, and face. Huntington's Disease is an inherited disease that causes nerve cells in the brain to waste away. Symptoms include uncontrolled movements, clumsiness, and balance problems. Huntington's disease can hinder walk, talk, and swallowing.

Ataxia refers to the loss of full control of bodily movements, and may affect the fingers, hands, arms, legs, body, speech, and eye movements.

Myoclonus and Startle is a response to a sudden and unexpected stimulus, which can be acoustic, tactile, visual, or vestibular.

Tics are an involuntary movement usually onset suddenly, brief, repetitive, but non-rhythmical, typically imitating normal behavior and often occurring out of a background of normal activity. Tics can be classified as motor or vocal, motor tics associated with movements while vocal tics associated with sound. Tics can be characterized as simple or complex. For example simple motor tics involve only a few muscles restricted to a specific body part. Tourette Syndrome is an inherited neuropsychiatric disorder with onset in childhood, characterized by multiple motor tics and at least one vocal tic.

Restless Legs Syndrome is a neurologic sensorimotor disorder characterized by an overwhelming urge to move the legs when at rest.

Stiff Person Syndrome is a progressive movement disorder characterized by involuntary painful spasms and rigidity of muscles, usually involving the lower back and legs. Stiff-legged gait with exaggerated lumbar hyperlordosis typically results. Characteristic abnormality on EMG recordings with continuous motor unit activity of the paraspinal axial muscles is typically observed. Variants include “stiff-limb syndrome” producing focal stiffness typically affecting distal legs and feet.

Gait disorders refer to an abnormality in the manner or style of walking, which results from neuromuscular, arthritic, or other body changes. Gait is classified according to the system responsible for abnormal locomotion, and include hemiplegic gait, diplegic gait, neuropathic gait, myopathic gait, parkinsonian gait, choreiform gait, ataxic gait, and sensory gait.

### Anesthesia/Sedation

Anesthesia is a pharmacologically induced and reversible state of amnesia, analgesia, loss of responsiveness, loss of skeletal muscle reflexes, decreased stress response, or all of these simultaneously. These effects can be obtained from a single drug which alone provides the correct combination of effects, or occasionally with a combination of drugs (e.g., hypnotics, sedatives, paralytics, analgesics) to achieve very specific combinations of results. Anesthesia allows patients to undergo surgery and other procedures without the distress and pain they would otherwise experience.

Sedation is the reduction of irritability or agitation by administration of a pharmacological agent, generally to facilitate a medical procedure or diagnostic procedure.

Sedation and analgesia include a continuum of states of consciousness ranging from minimal sedation (anxiolysis) to general anesthesia.

Minimal sedation is also known as anxiolysis. Minimal sedation is a drug-induced state during which the patient responds normally to verbal commands. Cognitive function and coordination may be impaired. Ventilatory and cardiovascular functions are typically unaffected.

Moderate sedation/analgesia (conscious sedation) is a drug-induced depression of consciousness during which the patient responds purposefully to verbal command, either alone or accompanied by light tactile stimulation. No interventions are usually necessary to maintain a patent airway. Spontaneous ventilation is typically adequate. Cardiovascular function is usually maintained.

Deep sedation/analgesia is a drug-induced depression of consciousness during which the patient cannot be easily aroused, but responds purposefully (not a reflex withdrawal from a painful stimulus) following repeated or painful stimulation. Independent ventilatory function may be impaired and the patient may require assistance to maintain a patent airway. Spontaneous ventilation may be inadequate. Cardiovascular function is usually maintained.

General anesthesia is a drug-induced loss of consciousness during which the patient is not arousable, even to painful stimuli. The ability to maintain independent ventilatory function is often impaired and assistance is often required to maintain a patent airway. Positive pressure ventilation may be required due to depressed spontaneous ventilation or drug-induced depression of neuromuscular function. Cardiovascular function may be impaired.

Sedation in the intensive care unit (ICU) allows the depression of patients' awareness of the environment and reduction of their response to external stimulation. It can play a role in the care of the critically ill patient, and encompasses a wide spectrum of symptom control that will vary between patients, and among individuals throughout the course of their illnesses. Heavy sedation in critical care has been used to facilitate endotracheal tube tolerance and ventilator synchronization, often with neuromuscular blocking agents.

In some embodiments, sedation (e.g., long-term sedation, continuous sedation) is induced and maintained in the ICU for a prolonged period of time (e.g., 1 day, 2 days, 3 days, 5 days, 1 week, 2 week, 3 weeks, 1 month, 2 months). Long-term sedation agents may have long duration of action. Sedation agents in the ICU may have short elimination half-life.

Procedural sedation and analgesia, also referred to as conscious sedation, is a technique of administering sedatives or dissociative agents with or without analgesics to induce a state that allows a subject to tolerate unpleasant procedures while maintaining cardiorespiratory function.

### EXAMPLES

In order that the disclosure described herein may be more fully understood, the following examples are set forth. The synthetic examples described in this application are offered to illustrate the compounds, pharmaceutical compositions, and methods provided herein and are not to be construed in any way as limiting their scope.

In order that the invention described herein may be more fully understood, the following examples are set forth. The examples described in this application are offered to illustrate the crystalline solid forms provided herein and are not to be construed in any way as limiting their scope.

### Abbreviations and Definitions

API active pharmaceutical ingredient
AUC area under the curve
BA bioavailability
BU blend uniformity
Cₘₐₓ maximum observed plasma concentration
CU content uniformity
DP drug product
DS drug substance
DSC differential scanning calorimetry
DVS dynamic vapor sorption
FaSSIF fasted state simulated intestinal fluid
FeSSIF fed state simulated intestinal fluid
FRI Flow Rate Index
GMP Good Manufacturing Practice
HDPE high density polyethylene
HPLC high performance liquid chromatography
kp kilopond(s)
LOD limit of detection
max maximum
min minimum
MCC microcrystalline cellulose
NA not applicable
ND not detected (or <0.05% area on HPLC analysis)
PVA Polyvinyl alcohol
RH relative humidity
RRT relative retention time
RSD relative standard deviation
RT room temperature or real time
SD Standard Deviation
SDS sodium dodecyl sulfate
SLS sodium lauryl sulfate
SMCC silicified microcrystalline cellulose
SSF sodium stearyl fumarate
TGA thermogravimetric analysis
USP United States Pharmacopoeia
w/w weight-to-weight
XRPD X-ray powder diffraction

### Example 1

### Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-1H-pyrazole-4-carbonitrile (Compound 1)

To a suspension of K₂CO₃ (50 mg, 0.36 mmol) in THF (5 mL) was added 1H-pyrazole-4-carbonitrile (100 mg, 0.97 mmol) and 2-bromo-1-((3R,5R,8R,9R,10S,13S,14S,17S)-3-hydroxy-3,13-dimethylhexadecahydro-1H-cyclopenta[_{α}]phenanthren-17-yl)ethan-1-one (50 mg, 0.12 mmol). The mixture was stirred at room temperature for 15 hours. The reaction mixture was poured into 5 mL H₂O and extracted with ethyl acetate (2×10 mL). The combined organic layers were washed with brine, dried over sodium sulfate, filtered and concentrated. The residue mixture was purified by reverse-phase preparative HPLC to afford Compound 1 as a white solid (9 mg, 17.4% yield). ¹H NMR (500 MHZ, CDCl₃) δ (ppm) 7.87 (1H, s), 7.82 (1H, s), 5.02 (1H, AB), 4.2 (1H, AB), 2.61 (1H, t), 2.16-2.24 (1H, m), 2.05 (1H, dxt), 1.70-1.88 (6H, m), 1.61-1.69 (2H, m), 1.38-1.52 (6H, m), 1.23-1.38 (5H, m), 1.28 (3H, s), 1.06-1.17 (3H, m), 0.67 (3H, s). LCMS: rt=2.24 min, m/z=410.1 [M+H]⁺.

### Example 2

### Preparation of Crystalline Form A of Compound 1

Crude Compound 1 was stirred as a slurry in ethyl acetate below 10° C., filtered and dried under vacuum to provide crystalline Form A. Alternatively, crude Compound 1 was dissolved in dichloromethane and then re-concentrated twice with ethyl acetate under vacuum to dryness to provide crystalline Form A.

### Example 3

### Preparation of Crystalline Form C of Compound 1

Approximately 10 to 20 mg of Form A was suspended in 0.5 mL of a mixture of isopropyl alcohol (IPA) and isopropyl acetate (IPAc). After stirring at room temperature or 50° C. for 48 hours, the solids were isolated by centrifugation to provide crystalline Form C of Compound 1. Alternatively, crude Compound 1 is combined with ethyl acetate and the mixture is heated to reflux, causing the solids to dissolve. The solution is polish-filtered and rinsed with ethyl acetate and the filtrate is concentrated by atmospheric distillation. Once the desired volume is reached, the temperature is lowered to 65-75° C. and the slurry is stirred at this temperature for at least 2 hours. Heptane is then added while keeping the temperature at 6575° C. and the mixture is held at this temperature for 6 hours. The temperature is then slowly lowered to 30-35° C. The slurry is held at this temperature for 1-24 hours and filtered. The resulting cake is washed with ethyl acetate and heptane. The final filter cake is dried under vacuum at ≤50° C. which affords Form C of Compound 1 as a white to off-white crystalline solid.

### Example 4

### Characterization of Crystalline Form A and Crystalline Form C of Compound 1 by XRPD

The XRPD pattern for Form A of Compound 1 was collected with a PANalytical Empyrean diffractometer using an incident beam of Cu radiation produced using an Optix long fine-focus source. An elliptically graded multilayer mirror was used to focus the Cu Kα X-rays through the specimen and onto the detector. Prior to the analysis, a silicon specimen (NIST SRM 640e) was analyzed to verify that the observed position of the Si 111 peak was consistent with the NIST-certified position. A specimen of the sample was sandwiched between two 3 μm-thick films and analyzed in transmission geometry. A beam-stop, short anti-scatter extension and anti-scatter knife edge were used to minimize the background generated by air. Soller slits for the incident and diffracted beams were used to minimize broadening from axial divergence. The diffraction pattern was collected using a scanning position-sensitive detector (X'Celerator) located 240 mm from the specimen with Data Collector software. The instrument parameters used are listed in Table 1.

TABLE 1
Instrument Parameters for XRPD Analysis
of Form A of Compound 1

| | Parameters for Transmission Mode |
|---|---|
| X-Ray wavelength | Cu, kα, Kα1 (Å): 1.54059 |
| X-Ray tube setting | 45 kV, 40 mA |
| Divergence slit | Fixed ½° |
| Scan range (degrees 20) | 1.0° to 40.0° |
| Step size (degrees 20) | 0.017° |
| Scan speed (degrees per minute) | 1.2 |
| Collection time (sec.) | 1937 |
| Revolution time (sec.) | 1.0 |

The XRPD pattern for Form C of Compound 1 was collected with a PANalytical X'Pert PRO MPD diffractometer using an incident beam of Cu radiation produced using an Optix long fine-focus source. An elliptically graded multilayer mirror was used to focus the Cu Kα X-rays through the specimen and onto the detector. Prior to the analysis, a silicon specimen (NIST SRM 640e) was analyzed to verify that the observed position of the Si 111 peak was consistent with the NIST-certified position. A specimen of the sample was sandwiched between two 3 μm-thick films and analyzed in transmission geometry. A beam-stop, short anti-scatter extension and an anti-scatter knife edge were used to minimize the background generated by air. Soller slits for the incident and diffracted beams were used to minimize broadening from axial divergence. The diffraction pattern was collected using a scanning position-sensitive detector (X'Celerator) located 240 mm from the specimen using Data Collector software. The instrument parameters used are listed in Table 2.

TABLE 2
Instrument Parameters for XRPD Analysis
of Form C of Compound 1

| | Parameters for Transmission Mode |
|---|---|
| X-Ray wavelength | Cu, kα, Kα1 (Å): 1.54059 |
| X-Ray tube setting | 45 kV, 40 mA |
| Divergence slit | Fixed ½° |
| Scan range (degrees 20) | 1.0° to 40.0° |
| Step size (degrees 20) | 0.017° |
| Scan speed (degrees/min) | 3.2 |
| Collection time (sec.) | 720 |
| Revolution time (sec.) | 1.0 |

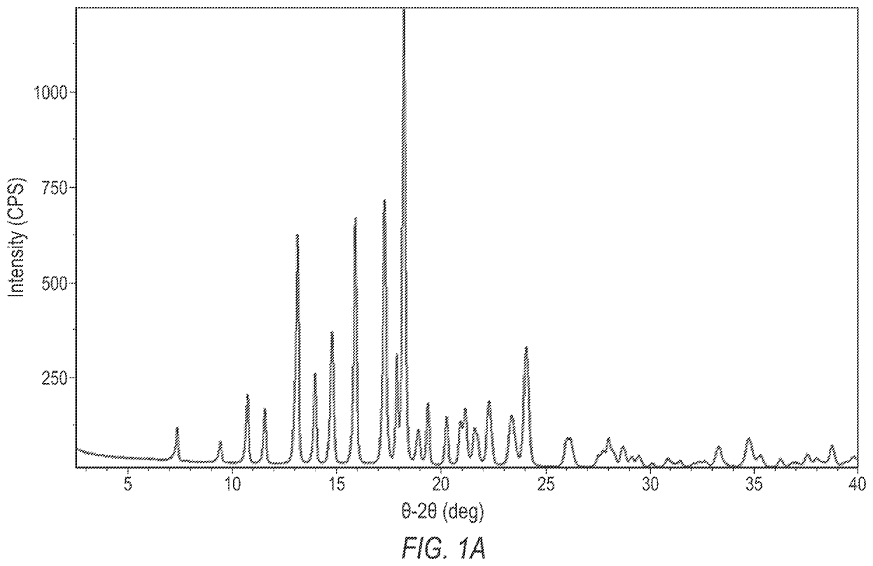

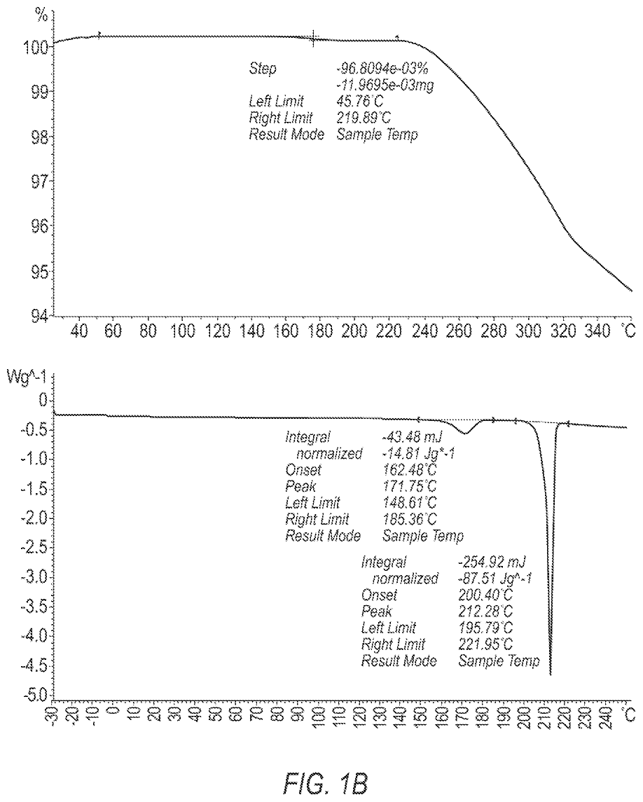

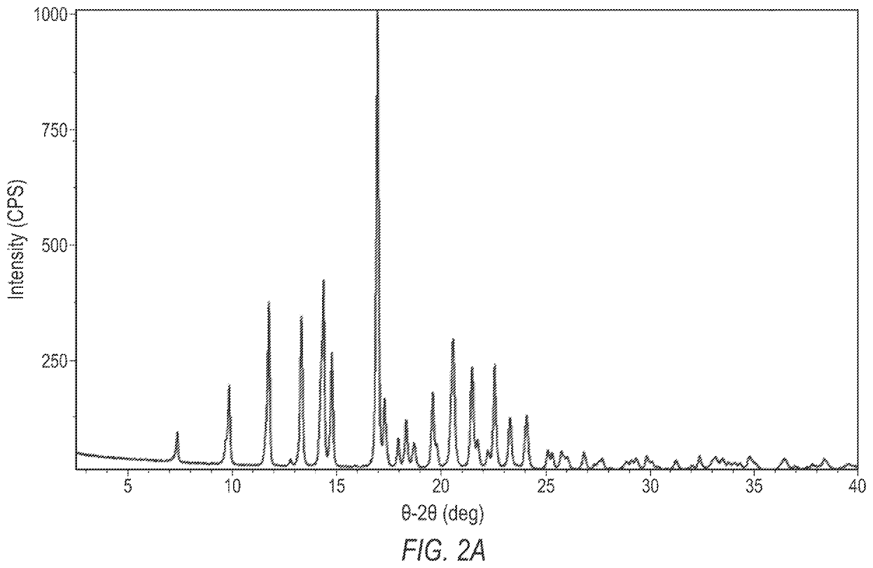

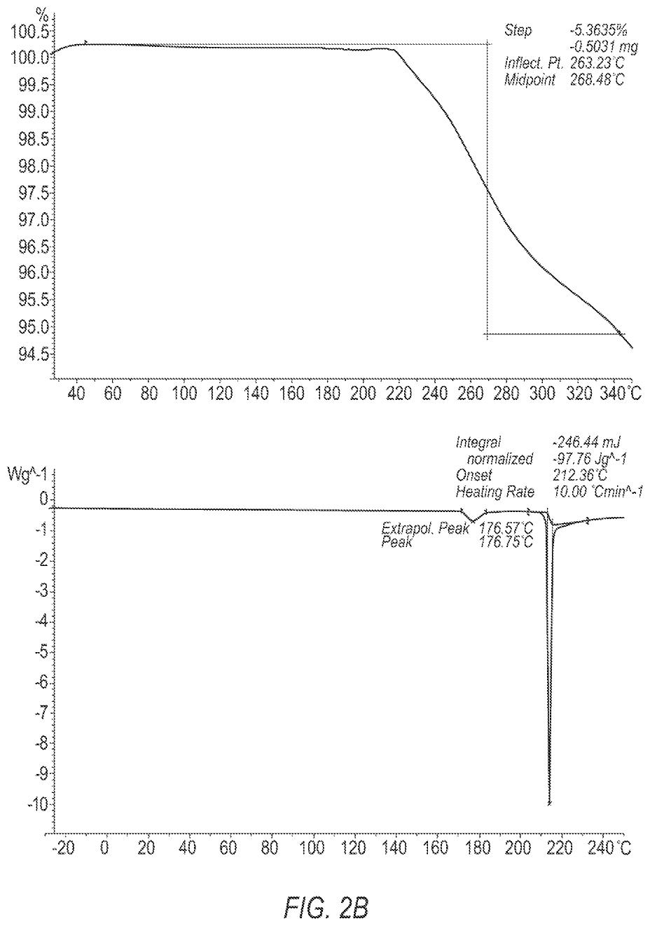

Form A: Form A was observed to be crystalline by XRPD, as shown in FIG. 1A. Form C: Form C was observed to be crystalline by XRPD, as shown in FIG. 2A.

### Example 5

### Preparation of Single Crystals of Form A and Form C of Compound 1

Form A: Single crystals suitable for structure determination were obtained via slow cooling in isopropyl alcohol from 50° C. to 5° C.

Form C: Single crystals suitable for structure determination were obtained via slow cooling at a rate of 0.01° C./min in isopropyl acetate/acetone (6:1, v/v) co-solvents with Form C seeds from 25° C. to 5° C.

### Example 6

### Single Crystal X-ray Diffraction Data for Form A and Form C

X-ray intensity data from the prism-like crystals of Form A (Table 3) and Form C (Table 4) of Compound 1 were collected at 290(2) K using a Bruker D8 Venture diffractometer (Mo Kct radiation, ?=0.71073 Å). The crystal structures of Forms A and C of Compound 1 were solved from the obtained data.

TABLE 3
X-ray Data and Structural Refinement for
a Single Crystal of Form A of Compound 1

| Empirical formula | C₂₅H₃₅N₃O₂ |
|---|---|
| Formula weight | 409.56 |
| Temperature | 100(2) K |
| Wavelength | 0.71073 Å |
| Crystal System, space group | Monoclinic P2₁ |
| Unit cell dimensions | a = 9.379(3) Å, b = 9.922(3) Å, |
| | c = 12.092(4) Å, α = 90⁰, |
| | β = 101.606(9)⁰, γ = 90⁰ |
| Volume | 1102.2(6) A₃ |
| Z, Calculated density | 2, 1.234 mg/m³ |
| Absorption coefficient | 0.079 mm⁻¹ |
| F(000) | 444 |
| Crystal size | 0.30 × 0.20 × 0.10 mm³ |
| Theta range for data collection | 2.22-27.56⁰ |
| Limiting indices | −12 <− h <− 12, −12 <− k <− |
| | 12, −15 <− l <− 15 |
| Reflections collected/unique | 23466/5060 [R(int) = 0.0670] |
| Completeness | 99.9% |
| Refinement method | Full-matrix least-squares on F₂ |
| Data/restraints/parameters | 5060/1/274 |
| Goodness-of-fit on F² | 1.071 |
| Final R indices [I > 2sigma(I)] | R₁ = 0.0425, wR₂ = 0.0989 |
| Largest diff. peak and hole | 0.309 and −0.368e · Å⁻³ |
| Absolute structure parameter | 1.5(11) |

TABLE 4
Crystal Data and Structural Refinement for
a Single Crystal of Form C of Compound 1

| Empirical formula | C₂₅H₃₅N₃O₂ |
|---|---|
| Formula weight | 409.56 |
| Temperature | 290(2) K |
| Wavelength | 0.71073 Å |
| Crystal System, space group | Orthorhombic P2₁2₁2₁ |
| Unit cell dimensions | a = 9.6642(8) Å, b = 9.8676(8) Å, |
| | c = 23.9408(19) Å, α = 90°, β = 90°, |
| | γ = 90° |
| Volume | 2283.1(3) Å₃ |
| Z, Calculated density | 4, 1.192 mg/m³ |
| Absorption coefficient | 0.076 mm⁻¹ |
| F(000) | 888 |
| Crystal size | 0.28 × 0.05 × 0.03 mm³ |
| Theta range for data collection | 2.71-27.61° |
| Limiting indices | −12 ≤ h ≤ 12, −12 ≤ k ≤ |
| | 12, −31 ≤ l ≤ 31 |
| Reflections collected/unique | 33905/5265 [R(int) = 0.0823] |
| Completeness | 99.3% |
| Refinement method | Full-matrix least-squares on F₂ |
| Data/restraints/Parameters | 5265/7/272 |
| Goodness-of-fit on F² | 1.042 |
| Final R indices [I > 2sigma(I)] | R₁ = 0.0647, wR₂ = 0.1128 |
| Largest diff. peak and hole | 0.248 and −0.335 e · Å⁻³ |
| Absolute structure parameter | 0.0(19) |

### Example 7

### Thermogravimetric Analysis and Differential Scanning Calorimetry of Form A and Form C of Compound 1

Thermogravimetric analysis (TGA) data were collected using a TA Q500/Q5000 TGA from TA Instruments. Differential Scanning Calorimetry (DSC) data was collected using a TA Q200/Q2000 DSC from TA Instruments. The instrument parameters used are provided in Table 5.

TABLE 5
Parameters for TGA and DSC Testing

| Parameters | TGA | DSC |
|---|---|---|
| Method | Ramp | Ramp |
| Sample pan | Platinum, open | Aluminum, crimped |
| Temperature | RT to 350° C. | RT to 300° C. |
| Heating rate | 10° C./min | 10° C./min |
| Purge gas | N₂ | N₂ |

For Form A: The TGA and DSC data are provided in FIG. 1C. The TGA analysis of Form A of Compound 1 resulted in negligible weight loss up to 200° C. The onset of an endotherm observed on the DSC curve at approximately 162° C., representing the transformation of Form A to Form K, was followed by an onset of an endotherm at approximately 210° C. for the melting of Form K.

For Form C: The TGA and DSC are provided in FIG. 2C. The TGA analysis of Form C of Compound 1 resulted in negligible weight loss occurring below 100° C. The onset of an endotherm at approximately 170° C., representing the transformation of Form C to Form K, was followed by an onset of an endotherm at approximately 212° C. for the melting of Form K.

### Example 8

### Preparation of Micronized Crystalline Form A of Compound 1

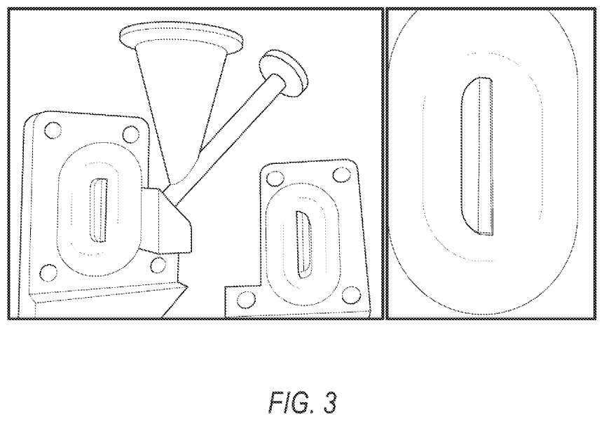

Un-micronized Form A of Compound 1 generates significant back pressure and clogs a standard jet mill (see FIG. 3). Using a Sturtevant model SDM2 Micronizer 2-inch jet mill (a pancake micronizer), Form A of Compound 1 was fed into the mill using a vibratory feeder at approximately 250 g/hr with a Venturi pressure of 80 psi and a mill pressure of 80 psi. Based on the design of the mill the smaller particle sizes move towards the center of the mill and exit at the product outlet and are collected in a filter bag. The yield was 13%. Using a Food Pharma Systems (FPS) model Pilotmill-2 2-inch cryogenic jet mill, at −50° C., un-micronized Form A of Compound 1 was fed into the mill using a vibratory feeder at approximately 200 g/hr with a Venturi pressure of 50-80 psi and a mill pressure of 70-140 psi. Based on the design of the mill the smaller particle sizes move towards the center of the mill and exit at the product outlet and are collected in a filter bag. The contents of the filter bag were assessed for particle size distribution and passed through the mill as needed until the desired particle size distribution is achieved. The yield was improved to 84%.

### Example 9

### Preparation of Micronized Crystalline Form C of Compound 1

Unexpectedly, un-micronized Form C of Compound 1 was more amenable to jet milling to reduce its particle size. Using a Sturtevant model SDM4 Micronizer 4-inch jet mill (a pancake micronizer), Form C of Compound 1 was fed into the mill using a vibratory feeder at 4-6 kg/hr with a Venturi pressure of 80-120 psi and a mill pressure of 80-100 psi. Based on the design of the mill the smaller particle sizes move towards the center of the mill and exit at the product outlet and are collected in a filter bag. A typical yield was >95%. Using a smaller Fluid Energy model 00 Jet-O-Mizer 2-inch loop mill, Form C of Compound 1 was fed into the mill using a vibratory feeder at 240-300 g/hr with a Venturi pressure of 60-120 psi and a mill pressure of 20 psi. A typical yield for this mill was 72%.

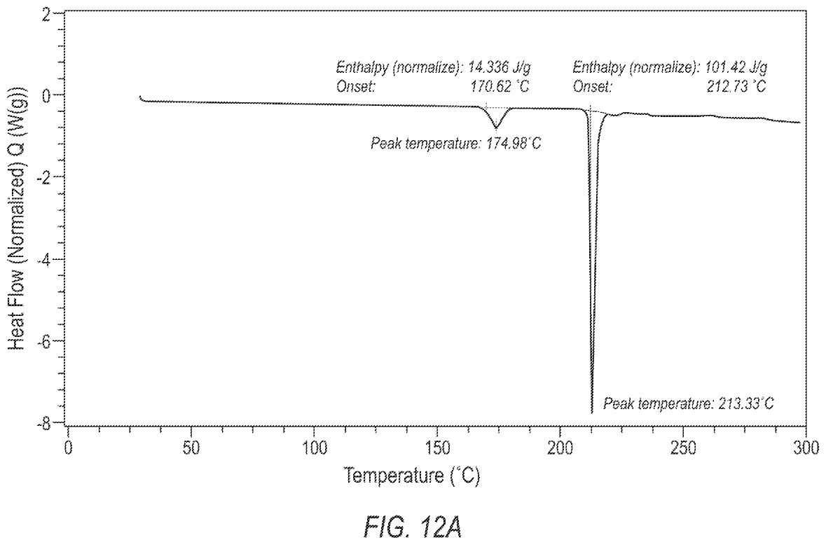

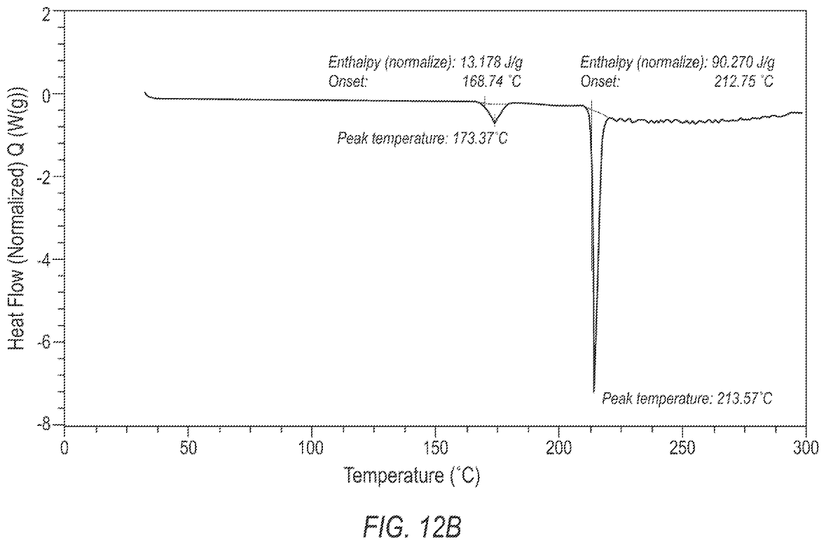

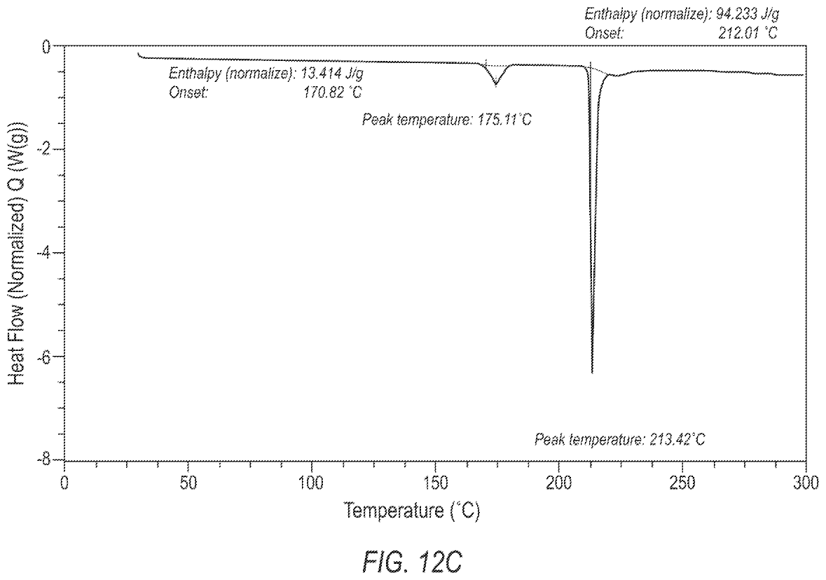

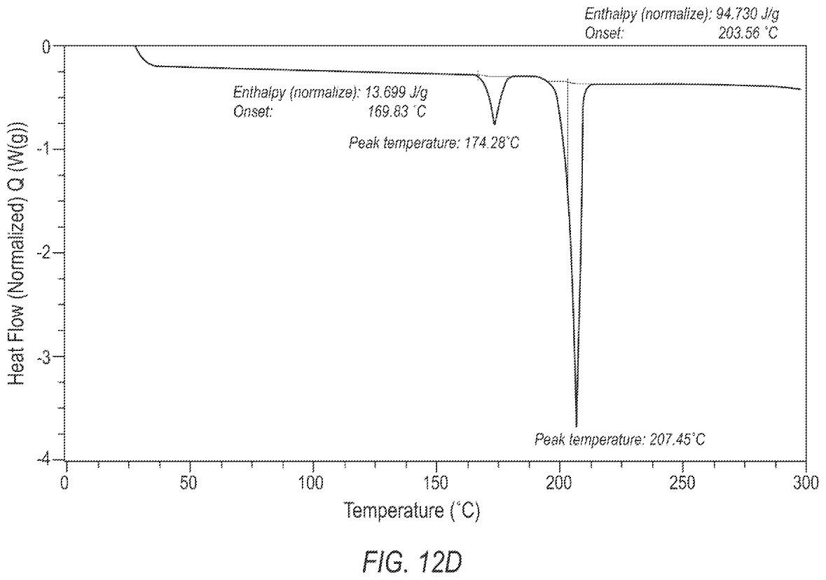

FIG. 12A, FIG. 12B, FIG. 12C, and FIG. 12D contain DSC data collected according to the parameters described in Example 7, including Table 5, for four representative scale up lots of micronized Form C of Compound 1, herein referred to as Compound 1a, Compound 1b, Compound 1c, and Compound 1d. FIG. 12A contains a DSC thermogram for Compound 1a. FIG. 12B contains a DSC thermogram for Compound 1b. FIG. 12C contains a DSC thermogram for Compound 1c. FIG. 12D contains a DSC thermogram for Compound 1d. Onset temperature and enthalpy for Compound 1a-Compound 1d is in Table 6.

TABLE 6
DSC onset temperature and enthalpy
for Compounds 1a, 1b, 1c, and 1d.

| | |
|---|---|
| First Endotherm | Second Endotherm |

| | Onset | | Onset | |
|---|---|---|---|---|
| | Temp. | Enthalpy | Temp. | Enthalpy |
| Compound | (° C.) | (J/g) | (° C.) | (J/g) |
| Compound 1a | 170.6 | 14.3 | 212.7 | 101.4 |
| Compound 1b | 168.7 | 13.2 | 212.8 | 93.3 |
| Compound 1c | 170.6 | 13.4 | 212.6 | 94.2 |
| Compound 1d | 169.8 | 13.7 | 203.6 | 90.7 |

### Example 10

### Determination of Particle Size Distribution for Compound 1

The particle size distribution of Compound 1 was measured using a laser diffraction method. A sample of Compound 1 was either dispersed in water or wetted and dispersed in 0.2% w/v Tween 80 in water to a final concentration spanning approximately 2.5 to 60 mg/mL. Using a Malvern Mastersizer 2000 with a Hydro 2000S sample dispersion unit the obscuration was adjusted to between 10 and 30%. The stir speed was adjusted to 2250-2500 rpm, pre-measurement delay to approximately 30 s, sample measurement time to around 10-15 s, background measurement time to around 10-15 s and the particle shape assumed to be irregular. The samples were at a concentration between 0.0032-0.0044% vol and had specific surface area between 1.65 and 2.10 m²/g, a surface weighted mean between 2.87 and 3.64 μm, and a volume weighed mean between 3.62 and 4.67 μm. The measurements were obtained using the Mie scattering model (material refractive index=1.520, dispersant refractive index=1.330, absorption=0.001 or 0.1) with normal sensitivity.

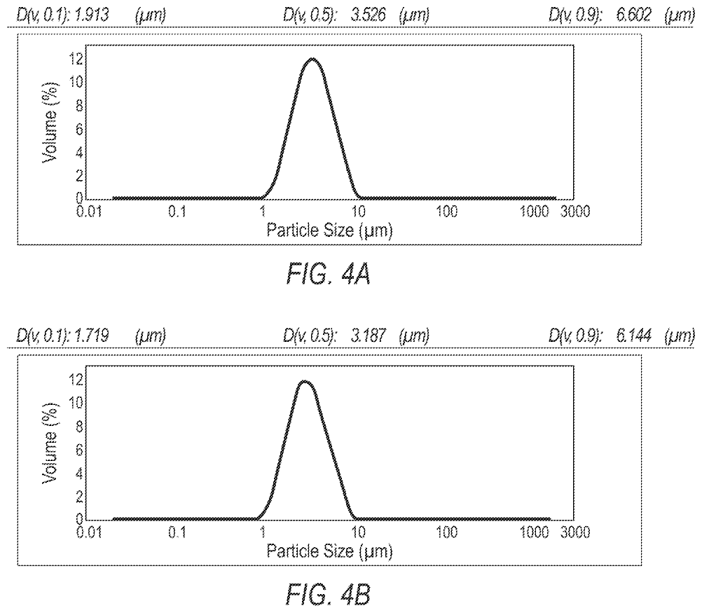

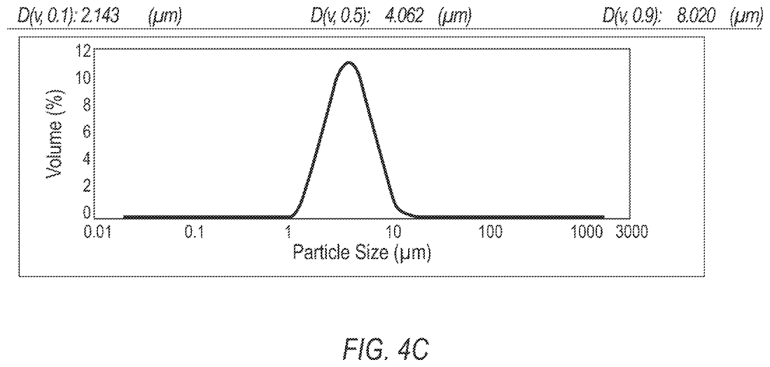

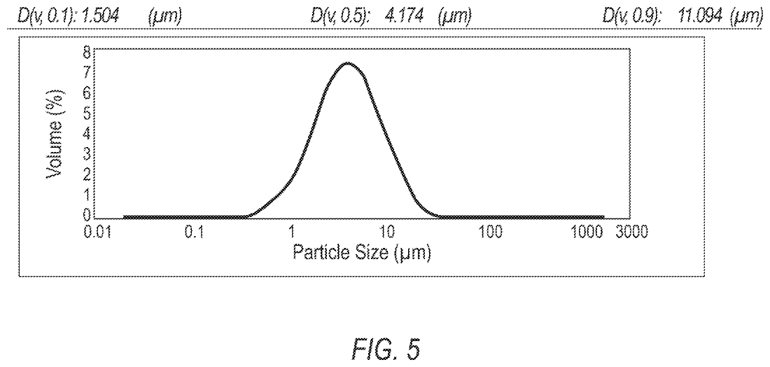

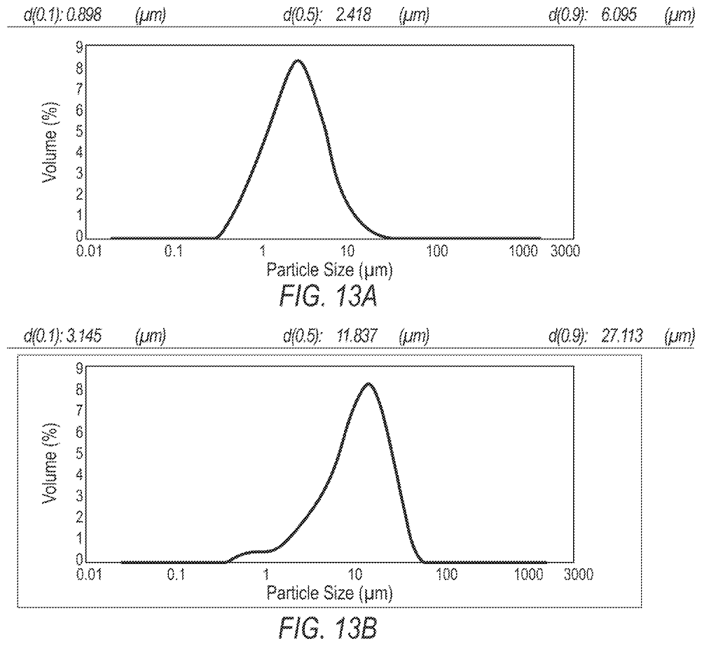

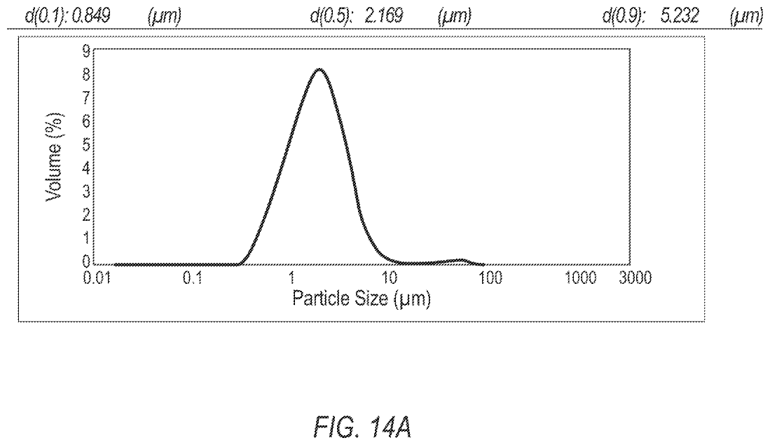

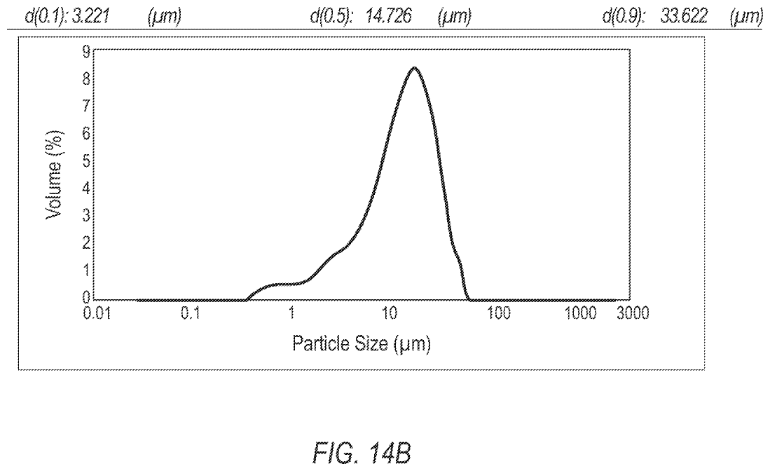

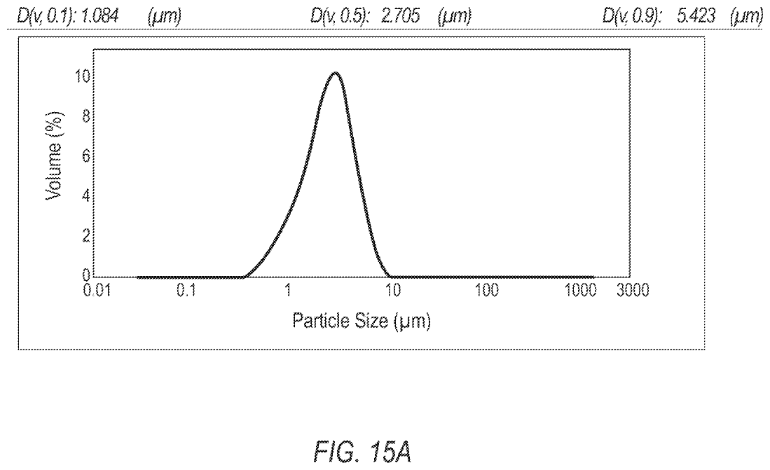

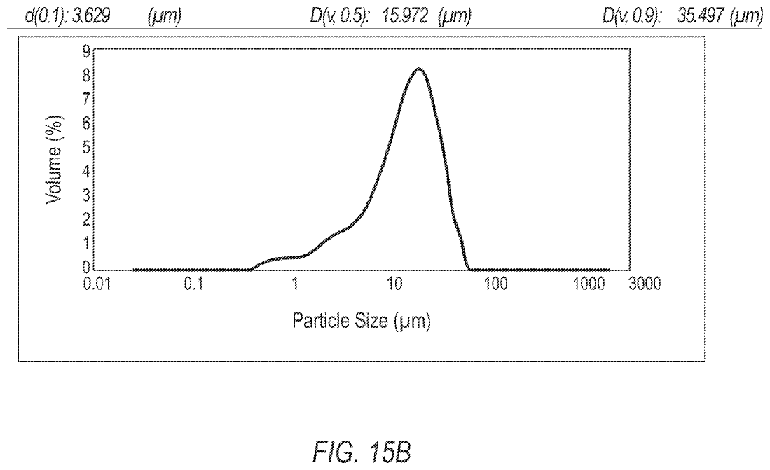

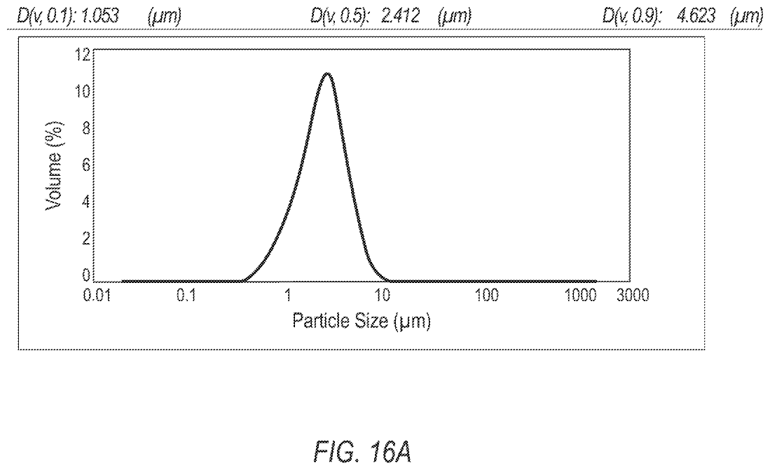

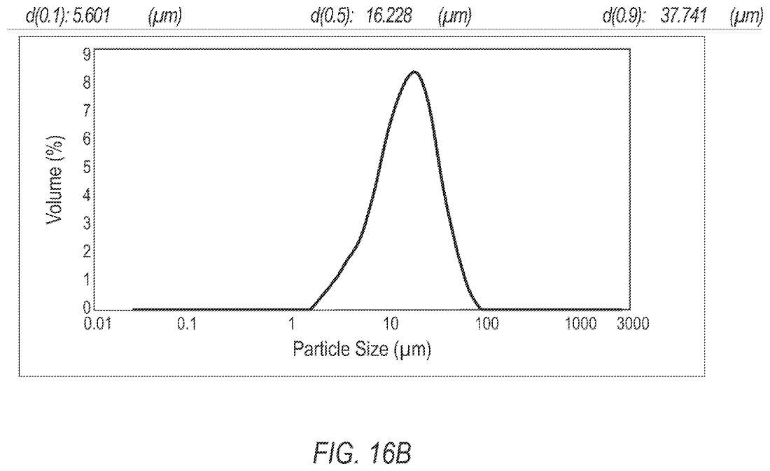

Representative particle size distribution data for lots of un-micronized and micronized Form C of Compound 1 are shown in Table 7. In addition, representative particle size distribution for micronized Form A of Compound 1 is also included in Table 7. FIG. 4A, FIG. 4B and FIG. 4C also contain particle size distribution profiles for three representative lots of micronized Form C of Compound 1. FIG. 13A and FIG. 13B contain particle size distribution profiles for micronized and un-micronized Form C of Compound 1a. FIG. 14A and FIG. 14B contain particle size distribution profiles for micronized and un-micronized Form C of Compound 1b. FIG. 15A and FIG. 15B contain particle size distribution profiles for micronized and un-micronized Form C of Compound 1c. FIG. 16A and FIG. 16B contain particle size distribution profiles for micronized and un-micronized Form C of Compound 1d. A representative particle size distribution for micronized Form A of Compound 1 is shown in FIG. 5.

TABLE 7
Particle Size Distribution of Un-micronized
and Micronized Form C of Compound 1.

| Particle Size | D₁₀ μm | D₅₀ μm | D₉₀ μm |
|---|---|---|---|
| Un-micronized Form C of Compound 1 | 6.5 | 31.6 | 70.5 |
| Un-micronized Form C of Compound 1 | 1.8 | 6.2 | 17.4 |
| Un-micronized Form C of Compound 1 | 4.3 | 11.8 | 26.3 |
| Micronized Form C of Compound 1 | 1.0 | 3.7 | 8.9 |
| Micronized Form C of Compound 1 | 1.9 | 3.5 | 6.6 |
| Micronized Form C of Compound 1 | 1.7 | 3.2 | 6.1 |
| Micronized Form C of Compound 1 | 2.1 | 4.1 | 8.0 |
| Micronized Form A of Compound 1 | 1.5 | 4.2 | 11.1 |
| Un-micronized Form C of Compound 1a | 3.1 | 11.8 | 27.1 |
| Un-micronized Form C of Compound 1b | 3.2 | 14.7 | 33.6 |
| Un-micronized Form C of Compound 1c | 3.6 | 16.0 | 35.5 |
| Un-micronized Form C of Compound 1d | 5.6 | 16.2 | 37.7 |
| Micronized Form C of Compound 1a | 0.9 | 2.4 | 6.1 |
| Micronized Form C of Compound 1b | 0.8 | 2.1 | 5.2 |
| Micronized Form C of Compound 1c | 1.1 | 2.7 | 5.4 |
| Micronized Form C of Compound 1d | 1.0 | 2.4 | 4.6 |

### Example 11

### Pharmacokinetic (PK) Profile of Form C of Compound 1 (Micronized and Un-Micronized) in Rat

Two groups, each with three male Sprague-Dawley rats, received an oral gavage dose of Compound 1 (un-micronized Form C or micronized Form C) suspended in 2% aqueous PVP-VA at a concentration and volume intended to achieve a target dose level of 15 mg/kg.

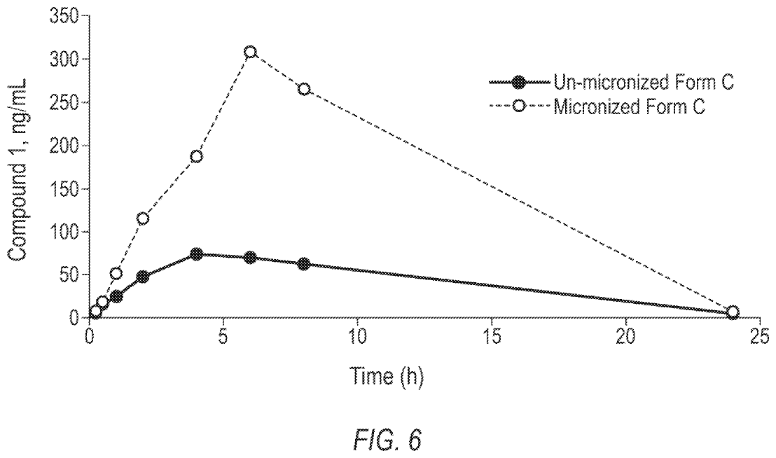

Serial blood samples were collected from each animal at 0.25, 0.5, 1, 2, 4, 6, 8 and 24 hours after dosing. Blood samples, which were collected into tubes containing K₂EDTA, were processed for plasma. Plasma samples were analyzed for Compound 1 by LC-MS/MS. Non-compartmental pharmacokinetic parameter estimates were calculated from the plasma concentration-time data for each animal. During dosing, and at each sample collection, animals were observed for any clinically relevant abnormalities. The PK profiles for the two particle size distributions are shown in FIG. 6 and the parameters summarized in Table 8. In Table 8 the subscripts to AUC last and inf refer to that AUC to the last sampling time point and extrapolated to infinite time, respectively.

TABLE 8
Pharmacokinetic Data in Rats for Un-micronized
and Micronized Form C of Compound 1

| | Tₘₐₓ | Cₘₐₓ | AUCₗₐₛₜ | AUC_{inf} |
|---|---|---|---|---|
| Sample | (hr) | (ng/mL) | (hr*ng/mL) | (hr*ng/mL) |
| Un-micronized | 6.67 | 83.6 | 979 | 854 |
| Micronized | 6.00 | 308 | 3647 | — |

### Example 12

### Excipient Compatibility

Eighteen excipients (including fillers, lubricants, glidants, binders, disintegrants, surfactants and a film coating material) were demonstrated to be compatible with Compound 1. Dry binary mixtures of Compound 1 and an excipient were stored at 40° C./75% RH for four weeks. A sample containing only Compound 1 was prepared as a control for each and stored at the same condition. HPLC analysis was performed comparing the samples to a representative excipient blank. The recovery and purity results are summarized in Table 9.

TABLE 9
Concentration/Purity of Filler Compatibility Samples: 40° C./75% RH (T = 4 weeks)

| | | | % Area of Individual | | | |
|---|---|---|---|---|---|---|
| | | | Impurities by RRT (≥0.05) | | | |
| Sample | % Recovery | % Purity | RRT 0.76 | RRT 1.05 | RRT 1.66 | RRT 2.06 |

Fillers

| | | | | | | |
|---|---|---|---|---|---|---|
| MCC (Avicel PH101) | 104.1 | 99.82 | ND | 0.16 | ND | ND |
| Lactose Monohydrate | 101.5 | 99.71 | ND | 0.17 | 0.12 | ND |
| (FastFlo 316) | | | | | | |
| Mannitol (Parteck M100) | 105.2 | 99.75 | 0.07 | 0.15 | ND | ND |
| Starch (Starch 1500) | 103.9 | 99.76 | ND | 0.15 | 0.10 | ND |
| SMCC (ProSolv HD90) | 102.6 | 99.73 | ND | 0.16 | 0.11 | ND |

Lubricants

| | | | | | | |
|---|---|---|---|---|---|---|
| Magnesium Stearate | 102.0 | 99.55 | 0.29 | 0.17 | ND | ND |
| (Hy-Qual) | | | | | | |
| Sodium Stearyl | 104.5 | 99.84 | ND | 0.16 | ND | ND |
| Fumarate (PRUV) | | | | | | |

Glidants

| | | | | | | |
|---|---|---|---|---|---|---|
| Silicon Dioxide | 103.1 | 52.24 | ND | 0.09 | ND | 47.67 |
| (Aerosil 200) | | | | | | |
| Talc | 106.6 | 99.73 | ND | 0.16 | 0.11 | ND |

Binders

| | | | | | | |
|---|---|---|---|---|---|---|
| PVP (Kollidon K30) | 101.1 | 99.85 | ND | 0.15 | ND | ND |
| HPMC E5 | 102.0 | 99.85 | ND | 0.15 | ND | ND |
| HPC (Klucel) | 100.1 | 99.84 | ND | 0.16 | ND | ND |
| Copovidone | 104.3 | 99.84 | ND | 0.16 | ND | ND |
| (Kollidon VA64) | | | | | | |

Disintegrants

| | | | | | | |
|---|---|---|---|---|---|---|
| Sodium Starch Glycolate | 103.0 | 99.75 | ND | 0.16 | 0.09 | ND |
| (Explotab) | | | | | | |
| Croscarmellose Sodium | 100.6 | 99.84 | ND | 0.16 | ND | ND |
| (Ac-Di-Sol) | | | | | | |
| Crospovidone | 103.0 | 99.78 | ND | 0.15 | 0.07 | ND |
| (Kollidon CL) | | | | | | |

Surfactan

| | | | | | | |
|---|---|---|---|---|---|---|
| Sodium Lauryl Sulfate | 103.7 | 99.87 | ND | 0.13 | ND | ND |

Coating

| | | | | | | |
|---|---|---|---|---|---|---|
| Opadry II While (PVA) | 103.2 | 99.74 | ND | 0.17 | 0.09 | ND |

### Example 13

### Direct Blend Manufacturing Process

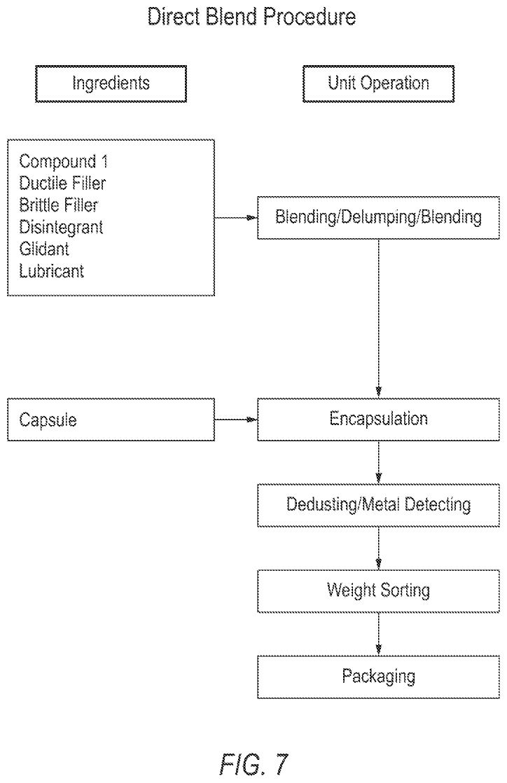

Direct blends were prepared by charging all the materials to a suitably-sized V-blender in the following approximate order: 1) ductile filler, 2) brittle filler, 3) micronized Compound 1, 4) remaining ductile or brittle filler to dry rinse the Compound 1 container and 5) disintegrant, glidant and lubricant. After mixing in the blender, the blend was discharged directly through a sieve. The blend was then charged into the blender a second time and further mixed. Samples for blend uniformity and physical characterization were taken prior to any capsule filling activities. If a surfactant was used, it was added before the disintegrant, glidant and/or lubricant were added and dry rinsed with either the ductile or brittle filler. A general flow diagram for the direct blend manufacturing process is shown in FIG. 7.

### Example 14

### Description of the Capsule Hand Filling Process

Hand-filling of capsules involves the manual filling of powder or blend into individual capsules (ranging from Size 5 up to Size 000) by pouring pre-weighed powder/blend directly into the capsule body and closing it by hand. Only one capsule can be filled at a time with this method.

### Example 15

### Description of a Semi-Automated Capsule Filling Process

A semi-automated capsule filling process involves the manual powder filling of a given number of capsules (typically 100-300) simultaneously with the use of a capsule filling tray system, such as a ProFill unit, that can hold a given number of capsule bodies. A predetermined quantity of powder is manually transferred over the entire template to allow flood filling and/or tamping of powder into all the capsules to ensure even weight distribution into each capsule. Closure of the capsules occurs by using another template housing the same number of corresponding capsule caps and placing them over the capsule bodies. Capsules ranging from Size 5 up to Size 000 can be filled using the semi-automated process by using the appropriately sized change part templates for the unit.

Capsules ranging from 5 mg to 60 mg dose strengths can be semi-automatically filled into capsules using a ProFill unit with change parts allowing the filling of 100 capsules at a time. To account for any losses, an overage of about 1-2% of the powder blend can be distributed onto the ProFill. After manufacture, the capsules can be de-dusted and weight-sorted.

### Example 16

### Use of Different Fillers in Tablets of Compound 1

Blends with different fillers were generated and compressed into 50 mg dose strength tablets. Four fillers were screened in binary mixtures with Compound 1 using variable drug loads (5-10%) for producing a tablet of sufficient hardness. The tablets were compressed on a single station tablet press using 5/16-inch round tooling targeting a 200 mg total tablet weight. Compression forces of 1000, 1500 and 2000 psi were tested. The results of the tableting study are summarized in Table 10.

TABLE 10
Composition and Tablet Hardness vs. Filler
and Drug Load for Tablets of Compound 1

| | Compression | Drug | Weight | Thickness | Hardness |
|---|---|---|---|---|---|
| Filler | Force | Load | (mg) | (mm) | (kp) |
| Starch | 1000 | 10 | 203.5 | 4.34 | 1.5 |
| 1500 | | 5 | 205.3 | 4.44 | 0.9 |
| | 1500 | 10 | 202.2 | 4.23 | 1.9 |
| | | 5 | 206.1 | 4.23 | 2.4 |
| | 2000 | 10 | 204.5 | 4.18 | 2.8 |
| | | 5 | 202.3 | 4.14 | 2.5 |
| Lactose | 1000 | 10 | 203.0 | 4.11 | 2.2 |
| Fast Flo | | 5 | 199.7 | 4.01 | 3.4 |
| 316 | 1500 | 10 | 201.0 | 3.92 | 3.1 |
| | | 5 | 201.4 | 3.86 | 6.8 |
| | 2000 | 10 | 204.7 | 3.91 | 3.7 |
| | | 5 | 204.7 | 3.82 | 6.1 |
| Avicel | 1000 | 10 | 201.6 | 4.07 | 16.5 |
| PH101 | 1500 | 10 | 200.8 | 3.90 | 21.4 |
| | 2000 | 10 | 200.9 | 3.87 | 23.9 |
| Mannitol | 1000 | 10 | 198.0 | 4.35 | 8.0 |
| Parteck | 1500 | 10 | 200.0 | 4.26 | 10.5 |
| M100 | 2000 | 10 | 200.7 | 4.20 | 10.0 |

### Example 17

### Use of Different Disintegrants in Hand-Filled Capsules of Micronized Form C of Compound 1

Four direct blend compositions were prepared from micronized Form C of Compound 1 according to the procedure defined in Example 13. Size 1 capsules were hand-filled to make 50 mg dose strengths. Two fillers, MCC (Avicel PH-101) and Mannitol (Parteck M100), were studied in combination with two disintegrants. The compositions, dissolution conditions and dissolution results for these blends are summarized in Table 11. The dissolution profiles of the resulting capsules were rapid under the stated dissolution conditions (>90% release) after 30 minutes.

TABLE 11
Composition and Dissolution Data for Four Direct Blend Hand-
Filled 50 mg Capsule Formulations Comparing Disintegrants

| Component | Composition 1 | Composition 2 | Composition 3 | Composition 4 |
|---|---|---|---|---|
| Micronized Form C | 33.33% | 33.33% | 18.18% | 18.18% |
| of Compound 1 | | | | |
| Avicel PH101 | 56.67% | 56.67% | — | — |
| Ac-Di-Sol | 10.00% | — | 10.00% | — |
| Explotab | — | 10.00% | — | 10.00% |
| Mannitol Parteck M100 | — | — | 71.82% | 71.82% |

Dissolution Method Conditions

| Parameter | Condition |
|---|---|
| Apparatus | USP Apparatus 2 (paddles with sinkers) |
| Media | 1% SLS in H₂O |
| Media Volume | 900 mL |
| Vessel Size | 1000 mL |
| Temperature | 37.0 ± 0.5° C. |
| Speed | 50 ± 2 RPM |
| Time Points | 10, 20, 30, 45, 60 minutes |
| Filter | 0.45 μm, 25 mm Nylon Syringe Filter |

Dissolution Results

| | Time Point (Minutes) | | | | | |
|---|---|---|---|---|---|---|
| Formulation Composition | | 10 | 20 | 30 | 45 | 60 |
| Composition 1 | Mean | 76 | 89 | 92 | 95 | 96 |
| Composition 2 | Mean | 82 | 90 | 94 | 96 | 97 |
| Composition 3 | Mean | 77 | 90 | 92 | 94 | 95 |
| Composition 4 | Mean | 80 | 93 | 94 | 95 | 96 |

### Example 18

### Use of Different Lubricants and Fillers in Hand-Filled Capsules of Micronized Form C of Compound 1

Four direct blend compositions were prepared from micronized Form C of Compound 1 according to the procedure defined in Example 13. Size 1 capsules were hand-filled to make 50 mg dose strengths. Two lubricants, magnesium stearate and PRUV (sodium stearyl fumarate), were studied in combination with two fillers, MCC and Mannitol and a disintegrant (Ac-Di-Sol). The compositions, dissolution conditions and dissolution results are summarized in Table 12. The dissolution profiles of the resulting capsules were rapid under the stated dissolution conditions (>90% release) after 30 minutes.

TABLE 12
Composition and Dissolution Data for Four Direct Blend Hand-Filled
50 mg Capsule Formulations Comparing Lubricants and Fillers

| Component | Composition 5 | Composition 6 | Composition 7 | Composition 8 |
|---|---|---|---|---|
| Compound 1 | 18.18% | 18.18% | 33.33% | 33.33% |
| Avicel PH101 | — | — | 56.17% | 56.17% |
| Ac-Di-Sol | 10.00% | 10.00% | 10.00% | 10.00% |
| Magnesium stearate | 0.50% | — | 0.50% | — |
| Mannitol Parteck M100 | 71.32% | 71.32% | — | — |
| PRUV | — | 0.50% | — | 0.50% |

Dissolution Method Conditions

| Parameter | Condition |
|---|---|
| Apparatus | USP Apparatus 2 (paddles with sinkers) |
| Media | 1% SLS in H₂O |
| Media Volume | 900 mL |
| Vessel Size | 1000 mL |
| Temperature | 37.0 ± 0.5° C. |
| Speed | 50 ± 2 RPM |
| Time Points | 10, 20, 30, 45, 60 minutes |
| Filter | 0.45 μm, 25 mm Nylon Syringe Filter |

Dissolution Results

| | Time Point (Minutes) | | | | | |
|---|---|---|---|---|---|---|
| Formulation | | 10 | 20 | 30 | 45 | 60 |
| Composition 5 | Mean | 83 | 93 | 95 | 97 | 98 |
| Composition 6 | Mean | 88 | 96 | 98 | 99 | 100 |
| Composition 7 | Mean | 86 | 95 | 97 | 98 | 98 |
| Composition 8 | Mean | 82 | 92 | 94 | 96 | 97 |

### Example 19

### Use of a Silicified Microcrystalline Cellulose, and Effect of Disintegrant and/or Surfactant in Hand-Filled 50 mg Capsules of Micronized Form C of Compound 1

Three direct blend compositions were prepared from micronized Form C of Compound 1 according to the procedure defined in Example 13. Size 1 capsules were hand-filled to make 50 mg dose strengths. A filler without a glidant (Avicel PH101) was replaced with SMCC (Prosolv HD₉₀), which has similar physical characteristics to Avicel PH101 and includes a glidant for improved flow. The three blends each contained a 20% drug load with a 50:50 ratio of SMCC to mannitol, as well as PRUV. Composition 9 included 5% of a disintegrant with no added surfactant. Composition 10 contained neither excipient. Composition 11 contained both excipients. The compositions, dissolution conditions and dissolution results are summarized in Table 13. The dissolution profiles of these resulting capsules were rapid under the stated dissolution conditions (>75% release) after 30 minutes.

TABLE 13
Composition and Dissolution Data for Three Direct Blend Hand-Filled 50 mg
Capsule Formulations Evaluating the Inclusion of Disintegrant or Surfactant

| Component | Composition 9 | Composition 10 | Composition 11 |
|---|---|---|---|
| Compound 1 | 20.00% | 20.00% | 20.00% |
| Prosolv HD90 | 37.25% | 39.75% | 36.75% |
| Mannitol (Parteck M100) | 37.25% | 39.75% | 36.75% |
| Ac-Di-Sol | 5.00% | — | 5.00% |
| PRUV | 0.50% | 0.50% | 0.50% |
| SLS | — | — | 1.00% |

Dissolution Method Conditions

| Parameter | Condition |
|---|---|
| Apparatus | USP Apparatus 2 (paddles with sinkers) |
| Media | 1% SLS in H₂O |
| Media Volume | 900 mL |
| Vessel Size | 1000 mL |
| Temperature | 37.0 ± 0.5° C. |
| Speed | 50 ± 2 RPM |
| Time Points | 10, 20, 30, 45, 60 minutes |
| Filter | 0.45 μm, 25 mm Nylon Syringe Filter |

Dissolution Results

| | Time Point (Minutes) | | | | | |
|---|---|---|---|---|---|---|
| Formulation | | 10 | 20 | 30 | 45 | 60 |
| Composition 9 | Mean | 70 | 77 | 80 | 83 | 85 |
| Composition 10 | Mean | 72 | 86 | 89 | 91 | 93 |
| Composition 11 | Mean | 82 | 90 | 93 | 94 | 84 |

### Example 20

### Effect and Use of Disintegrant and/or Surfactant in Hand-Filled 5 mg Capsules of Micronized Form C of Compound 1

Three direct blends were prepared according to the procedure described in Example 13 using micronized Form C of Compound 1. The compositions of these blends are summarized in Table 14. These blends were hand-filled into Size 1 capsules (250 mg) resulting in 5 mg dose strengths of Compound 1. The dissolution profiles of these resulting capsules were rapid under the stated dissolution conditions (>90% release) at 30 minutes.

TABLE 14
Composition of Three Direct Blend Hand-Filled 5 mg Capsule Formulations

| Ingredient | Composition 12 | Composition 13 | Composition 14 |
|---|---|---|---|
| — | w/w % | w/w % | w/w % |
| Compound 1 | 2.00 | 2.00 | 2.00 |
| SMCC (Prosolv HD90) | 46.25 | 48.75 | 45.75 |
| Mannitol (Parteck M100) | 46.25 | 48.75 | 45.75 |
| Croscarmellose sodium | 5.00 | 0.00 | 5.00 |
| (Ac-Di-Sol SD711) | | | |
| Sodium Lauryl Sulfate | 0.00 | 0.00 | 1.00 |
| Sodium Stearyl Fumarate | 0.50 | 0.50 | 0.50 |
| (PRUV) | | | |

Dissolution Method Conditions

| Parameter | Condition |
|---|---|
| Apparatus | USP Apparatus 2 (paddles with sinkers) |
| Media | 1% SLS in H₂O |
| Media Volume | 500 mL |
| Vessel Size | 1000 mL |
| Temperature | 37.0 ± 0.5° C. |
| Speed | 50 ± 2 RPM |
| Time Points | 10, 20, 30, 45, 60 minutes |
| Filter | 0.45 μm, 25 mm Nylon Syringe Filter |

Dissolution Results

| | Time Point (Minutes) | | | | | |
|---|---|---|---|---|---|---|
| Formulation | | 10 | 20 | 30 | 45 | 60 |
| Composition 12 | Mean | 82 | 90 | 93 | 95 | 96 |
| Composition 13 | Mean | 84 | 91 | 94 | 97 | 98 |
| Composition 14 | Mean | 85 | 92 | 95 | 97 | 98 |

### Example 21

### Pharmacokinetic Profile of Direct Blend 5 mg Hand-Filled Capsule Formulations in Dog

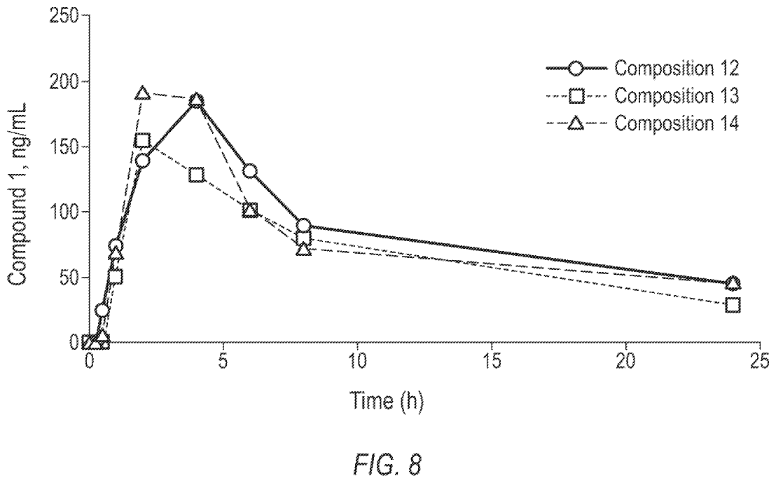

A pharmacokinetic study in dogs was performed with the three 5 mg dose strength capsules described in Example 20. Fed animals, four groups, each with three animals, were dosed with a target dose level of 5 mg/dog. In each dose session, serial blood samples were collected from each animal prior to dosing and at 0.25, 0.5, 1, 2, 4, 6, 8 and 24 hours after dosing. Blood samples, which were collected into tubes containing dipotassium EDTA, were processed for plasma. Plasma samples were analyzed for Compound 1 by LC-MS/MS. Non-compartmental pharmacokinetic parameters were calculated from the plasma concentration-time data for each animal. During dosing and at each sample collection, animals were observed for any clinically relevant signs. The PK profiles for the three compositions are shown in FIG. 8 and the parameters summarized in Table 15.

TABLE 15
Pharmacokinetic Data for Direct Blend 5 mg
Hand-Filled Capsule Formulations in Dogs

| | Tₘₐₓ | Cₘₐₓ | AUCₗₐₛₜ | AUC_{inf} |
|---|---|---|---|---|
| Composition | (hr) | (ng/mL) | (hr*ng/mL) | (hr*ng/mL) |
| 12 | 3.33 | 202 | 2067 | 1659 |
| 13 | 2.67 | 162 | 1680 | 1702 |
| 14 | 2.67 | 206 | 1940 | — |

### Example 22

### Compositions and Dissolution Data for 5, 10, 20, 25 and 30 mg Semi-Automated Fill Capsules

Five direct blends were prepared according to the procedure described in Example 13 and semi-automatically encapsulated as in Example 15 using micronized Form C of Compound 1. The compositions of these blends are summarized in Table 16. These blends were semiautomatically filled into Size 1 capsules resulting in 5, 10, 20, 25 and 30 mg dose strengths of Compound 1. The total fill weight of these dose strengths was 250 mg. The dissolution profiles of the resulting capsules were rapid under the stated dissolution conditions (>90% release) at 30 minutes. The particle size distribution data for the lots of micronized Form C of Compound 1 used in the manufacture are summarized in Table 16.

TABLE 16
Composition and Dissolution Data of Compound 1 Direct Blend
Hand-Filled Capsules for 5 through 30 mg Dose Strengths

| — | Composition 15 | Composition16 | Composition 17 | Composition18 | Composition19 |
|---|---|---|---|---|---|
| Dose Strength (mg) | 5 | 10 | 20 | 25 | 30 |
| Micronized Compound 1, mg | 5.0 | 10.0 | 20.0 | 25 | 30.0 |
| Prosolv SMCC HD90, mg | 115.6 | 113.1 | 108.1 | 105.6 | 103.1 |
| Parteck M100 (Mannitol), mg | 115.6 | 113.1 | 108.1 | 105.6 | 103.1 |
| Croscarmellose Sodium, mg | 12.5 | 12.5 | 12.5 | 12.5 | 12.5 |
| Sodium Stearyl Fumarate, mg | 1.3 | 1.3 | 1.3 | 1.3 | 1.3 |
| Total, mg | 250 | 250 | 250 | 250 | 250 |
| Lot of Micronized Form C | A | A | B | C | A |
| of Compound 1 | | | | | |
| D₁₀ (μm) | 1.7 | 1.7 | 1.9 | 2.1 | 1.7 |
| D₅₀ (μm) | 3.3 | 3.3 | 3.5 | 4.0 | 3.3 |
| D₉₀ (μm) | 6.8 | 6.8 | 6.6 | 7.7 | 6.8 |

Dissolution Method Conditions

| Parameter | Condition |
|---|---|
| Apparatus | USP Apparatus 2 (paddles with sinkers) |
| Media | 1% SLS in H₂O |
| Media Volume | 500 mL for 5 mg |
| | 900 mL for 10-30 mg |
| Vessel Size | 1000 mL |
| Temperature | 37.0 ± 0.5° C. |
| Speed | 50 ± 2 RPM (200 ± 8 RPM from 60 to 75 min) |
| Time Points | 10, 20, 30, 45, 60 and 75 minutes |
| Filter | 0.45 μm, 25 mm Nylon Syringe Filter |

Time Points

| Formulation | | 10 | 20 | 30 | 45 | 60 | 75 |
|---|---|---|---|---|---|---|---|
| Composition 18 (5 mg) | Mean | 86 | 91 | 91 | 92 | 93 | 96 |
| Composition 19 (10 mg) | Mean | 90 | 94 | 96 | 96 | 97 | 9 |
| Composition 20 (20 mg) | Mean | 87 | 91 | 92 | 93 | 94 | 96 |
| Composition 21 (25 mg) | Mean | 89 | 93 | 94 | 94 | 95 | 96 |
| Composition 22 (30 mg) | Mean | 89 | 94 | 95 | 96 | 96 | 98 |

### Example 23

### Pharmacokinetic Profile of Semi-Automated Filled Capsules of Micronized Form C of Compound 1 in Healthy Human Subjects

Twelve (12) subjects completed all four periods of the study; subjects who replaced discontinued subjects were allocated to the same randomization sequence as those discontinued. This study consisted of four periods: Period 1: Subjects (N=20) were randomized on a 1:1 basis to receive a single 30 mg dose of micronized Form C of Compound 1 or a single 30 mg dose of Compound 1 Oral Solution (Compound 1 dissolved in aqueous hydroxypropyl β-cyclodextrin solution) on Day 1. Study drug was administered in the fasting state. Period 2: After a washout period (which ended on Day 7), the same subjects (N=20) as Period 1 were crossed over to the dosage form that they did not receive in Period 1. On Day 8, study drug was administered in the fasting state. Period 3: Food Effect (high fat): All subjects received a single 30 mg dose of Form C of Compound 1 Capsules on Day 15. Study drug was administered after a high-fat meal. Period 4: Food Effect (standard): All subjects received a single 30 mg dose of Form C of Compound 1 Capsules on Day 22. Study drug was administered after a standard meal. The 30 mg capsule dose used in this study consisted of one 5 mg capsule (Composition 15) and one 25 mg capsule (Composition 18) of the compositions described in Table 17. The bioavailability of the capsules is summarized in Table 17. The bioavailability is reduced in the fasted state when compared to the fed state arms of the study. There is approximately a 1.5-fold improvement in AUC and 3-fold improvement in Cₘₐₓ when given with a standard and high-fat meal.

TABLE 1
Relative Bioavailability of Compound
1 as a Semi-Automated Filled Capsule

| Comparison | Cₘₐₓ Ratio | AUC ratio |
|---|---|---|
| High Fat Meal vs. | 2.879 | 1.575 |
| Fasted | (2.56, 3.28) | (1.45, 1.69) |
| Standard Meal vs | 2.894 | 1.581 |
| Fasted | (2.64, 3.25) | (1.43, 1.72) |

Data for Fasted Semi Automated-filled Dose

| | Cₘₐₓ (ng/mL) | AUC (hr*ng/mL) |
|---|---|---|
| Fasted Capsule | 21.81 | 528.62 |
| | (18.81, 25.34) | (474.24, 594.73) |

### Example 24

### Physical Characterization Data for the 5 mg and 30 mg Blends

Two larger blends of 250 g each of a 5 mg composition (Composition 15) and a 30 mg composition (Composition 19) were prepared according to the procedure described in Example 13 using micronized Form C of Compound 1. The physical properties of these two blends are summarized in Table 18.

TABLE 18
Physical Characterization Data for
the Two Compound 1 Direct Blends

| — | 5 mg | 30 mg |
|---|---|---|
| Bulk Density (g/cc) | 0.573 | 0.486 |
| Tapped Density (g/cc) | 0.717 | 0.737 |
| Carr Index (%) | 20 | 34 |

Sieve Analysis (μm/mesh, % Retained)

| | | |
|---|---|---|
| 710/25 | 0.1 | 0.9 |
| 600/30 | 0.5 | 0.5 |
| 425/40 | 0.4 | 0.8 |
| 250/60 | 4.6 | 6.5 |
| 150/100 | 22.9 | 24.0 |
| 63/230 | 46.0 | 31.8 |
| <63pan | 25.1 | 34.8 |

Flow Rate Index (FRI) (kg/sec)

| | | |
|---|---|---|
| Mean | 1.277 | 0.120 |
| Wall Friction Angle (WFA) | 20.97 | 23.12 |
| from shear tester | | |

Blend Uniformity (% assay)

| Sample | 5 mg Blend | 30 mg Blend |
|---|---|---|
| Mean | 97.1 | 98.4 |
| SD | 0.8 | 0.3 |
| RSD | 0.8 | 0.3 |

### Example 25

### Content Uniformity Data for the 5 mg and 30 mg Automated Capsules Manufactured from the Blends Described in Example 24

The two 250 g blends (5 mg and 30 mg) described in Example 24 were encapsulated into Size 1 capsules at a target yield of 1200 capsules on an Incap automated dosing disc encapsulator and the content uniformities (n=10) were determined for both filling runs. The data is summarized in Table 19.

TABLE 19
Content Uniformity (CU) of Automated Filled Capsules
with Composition 15 and Composition 19 Blends

| | Composition 18-5 mg | | | Composition 19-30 mg | | |
|---|---|---|---|---|---|---|
| | Capsule | | Weight | Capsule | | Weight |
| — | Weight | | Corrected | Weight | | Corrected |
| — | [symbol] | % LC | [symbol] | [symbol] | % LC | [symbol] |
| Mean | 329.0 | 97.1 | 95.9 | 329.4 | 99.1 | 97.7 |
| RSD | 1.0 | 1.3 | 0.7 | 2.3 | 2.6 | 1.0 |
| SD | 3.2 | 1.2 | 0.7 | 7.7 | 2.6 | 1.0 |
| Min | 322.8 | 95.0 | 95.1 | 311.9 | 94.7 | 96.5 |
| Max | 332.8 | 99.0 | 97.2 | 337.0 | 102.6 | 99.9 |
| AV | NA | 4.4 | 4.3 | NA | 6.2 | 2.4 |

[symbol]

indicates data missing or illegible when filed

### Example 26

### Composition and Physical Properties of Alternative Improved Flow Direct Blends

Five direct blends with improved flow properties were manufactured on a 300-500 g scale according to the procedure described in Example 13 with micronized Form C of Compound 1. The compositions and physical properties of the blends are summarized in Table 20. All the compositions contained 12% of Compound 1 as the drug load. The blend uniformity results for all five blends showed acceptable variability.

TABLE 20
Composition and Physical Properties of Alternative Improved Flow Direct Blends

| | Composition | Composition | Composition | Composition | Composition |
|---|---|---|---|---|---|
| — | 20 | 21 | 22 | 23 | 24 |

Ingredient (%)

| | | | | | |
|---|---|---|---|---|---|
| Micronized Compound 1 | 12.00 | 12.00 | 12.00 | 12.00 | 12.00 |
| MCC (Avicel PH-200) | 82.0 | 0 | 20.50 | 0 | 20.50 |
| Mannitol (Parteck M100) | 0 | 24.6 | 0 | 0 | 0 |
| Mannitol (Parteck M200) | 0 | 57.4 | 61.49 | 81.98 | 60.50 |
| Croscarmellose sodium | 5.0 | 5.0 | 5.00 | 5.00 | 5.00 |
| (Ac-Di-Sol SD711) | | | | | |
| Colloidal Silica | 0.5 | 0.5 | 0.5 | 0.5 | 0.5 |
| SSF (PRUV) | 0.5 | 0.5 | 0.52 | 0.52 | 1.50 |
| Total | 100 | 100 | 100 | 100 | 100 |

Physical Properties

| | | | | | |
|---|---|---|---|---|---|
| Bulk Density (g/cc) | 0.396 | 0.583 | 0.539 | 0.596 | 0.485 |
| Tapped Density (g/cc) | 0.521 | 0.767 | 0.691 | 0.764 | 0.674 |
| Carr Index (%) | 24 | 24 | 22 | 22 | 28 |

Sieve Analysis (μm/mesh, % Retained)

| | | | | | |
|---|---|---|---|---|---|
| 710/25 | 0.2 | 0.6 | 0.4 | 1.2 | 0.6 |
| 600/30 | 0.2 | 0.8 | 0.3 | 1.1 | 0.7 |
| 425/40 | 0.9 | 3.7 | 5.1 | 6.9 | 6.1 |
| 250/60 | 23.6 | 26.9 | 34.9 | 36.3 | 35.2 |
| 150/100 | 37.1 | 30.6 | 33.1 | 33.1 | 31.4 |
| 63/230 | 25.9 | 26.9 | 16.3 | 16.0 | 16.7 |
| <63pan | 12.0 | 8.2 | 7.4 | 4.2 | 7.8 |

Flow Rate Index (kg/sec)

| | | | | | |
|---|---|---|---|---|---|
| Mean | 0.391 | 0.211 | 0.303 | 0.449 | 0.395 |

Blend Uniformity (% assay)

| | | | | | |
|---|---|---|---|---|---|
| Mean | 96.1 | 97.4 | 94.1 | 90.3 | 99.5 |
| SD | 0.6 | 0.7 | 3.0 | 3.5 | 2.3 |
| RSD | 0.6 | 0.7 | 3.2 | 3.9 | 2.3 |

### Example 27

### Encapsulation of Improved Flow Direct Blends and Dissolution Data

Composition 21 prepared in Example 26 were encapsulated into Size 1 capsules at 30 mg dose strengths using an Incap dosing disc automatic encapsulator. Compositions 22, 23 and 24 prepared in Example 26 were encapsulated into Size 1 capsules at 10 mg and 30 mg dose strengths using an Incap dosing disc automatic encapsulator. All the blends were observed to exhibit good flow during the filling process. The content uniformity and dissolution data are summarized in Table 21. All compositions exhibited rapid release of >80% in 10 minutes into the dissolution media under sink conditions as either 30 mg or 10 and 30 mg dose strengths.

TABLE 21
Content Uniformity and Dissolution Data for Improved
Flow Direct Blend Automatically Encapsulated Capsules
Content Uniformity

| | Composition | | | | | | |
|---|---|---|---|---|---|---|---|
| | 21 | 22 | | 23 | | 24 | |
| | Dose Strength Filled (mg) | | | | | | |
| | 30 | 10 | 30 | 10 | 30 | 10 | 30 |
| Mean | 101.5 | 97.2 | 94.3 | 91.1 | 94.0 | 93.7 | 100.4 |
| RSD (%) | 1.6 | 2.5 | 4.1 | 14.1 | 1.8 | 2.8 | 2.8 |
| Acceptance Value | 3.9 | 7.1 | 13.5 | 38.1 | 8.5 | 11.1 | 6.8 |

Dissolution Method

| Parameter | Condition |
|---|---|
| Apparatus | USP Apparatus 2 (paddles with sinkers) for Compositions 22, 23 and 24 |
| | USP Apparatus 1 for Compositions 20 and 21 |
| Media | 50 mM sodium phosphate buffer, pH 6.8 with 0.4% SDS |
| Media Volume | 900 mL |
| Vessel Size | 1000 mL |
| Temperature | 37.0 ± 0.5° C. |
| Speed | 0-60 minutes: 50 rpm for Compositions 20 and 21 |
| | 0-60 minutes: 75 rpm for Compositions 22, 23 and 24 |
| | 60-75 minutes: 250 rpm (infinity) for all |
| Time Points | 5, 10, 15, 30, 45, 60, 75 minutes |
| Filter | 0.45 μm, 25 mm Nylon Syringe Filter |

Dissolution Results (% dissolved)

| Composition- | Time Points (minutes) | | | | | | | |
|---|---|---|---|---|---|---|---|---|
| Dose Strength | — | 5 | 10 | 15 | 30 | 45 | 60 | 75 |
| 21-30 mg | Mean | 16 | 45 | 68 | 89 | 92 | 94 | 99 |
| | % RSD | 37.9 | 32.1 | 11.3 | 3.4 | 2.8 | 2.3 | 1.7 |
| 22-10 mg | Mean | 40 | 74 | 84 | 93 | 96 | 97 | 98 |
| | % RSD | 51.0 | 11.9 | 6.7 | 4.2 | 3.3 | 2.9 | 2.3 |
| 22-30 mg | Mean | 33 | 71 | 83 | 92 | 93 | 94 | 95 |
| | % RSD | 21.4 | 5.6 | 4.1 | 2.9 | 2.9 | 2.8 | 3.2 |
| 23-10 mg | Mean | 24 | 69 | 87 | 99 | 100 | 101 | 101 |
| | % RSD | 96.8 | 21.4 | 5.9 | 4.1 | 4.1 | 3.7 | 4.0 |
| 23-30 mg | Mean | 34 | 70 | 81 | 91 | 93 | 93 | 93 |
| | % RSD | 17.1 | 9.1 | 6.1 | 1.8 | 1.0 | 0.9 | 1.1 |
| 24-10 mg | Mean | 18 | 60 | 73 | 83 | 86 | 88 | 92 |
| | % RSD | 109.7 | 21.0 | 9.5 | 6.1 | 5.2 | 4.6 | 4.2 |
| 24-30 mg | Mean | 45 | 79 | 88 | 96 | 97 | 98 | 99 |
| | % RSD | 9.5 | 4.6 | 3.2 | 2.8 | 2.8 | 2.8 | 2.6 |

### Example 28

### Preparation and Physical Properties of Improved Flow Roller Compacted Blends

Six roller compacted blends were manufactured with micronized Form C of Compound 1. The compositions and physical properties are summarized in Table 22. These blends were prepared at a target scale of 300 to 1000 g. In these roller compacted blends, the powder blending process, was the same as described for the direct blends described in Example 13. Some ingredients were split between the extragranular and intragranular stages of the process. After the initial sieving step, the roller compacted blends were processed with a Vector TF-156 mini roller compactor. Important roller compactor settings are summarized in Table 22.

TABLE 22
Composition and Physical Properties of Improved Flow Roller Compacted Blends

| | Composi- | Composi- | Composi- | | | Composi- | Composi- |
|---|---|---|---|---|---|---|---|
| Ingredient | tion 25 | tion 26 | tion 27 | Composition 28 | | tion 29 | tion 30 |
| Blend | | | | 30 mg | 10 mg | | |
| Micronized Compound 1 | 12.0 | 12.0 | 12.0 | 12.00 | 12.00 | 12.00 | 12.00 |
| Prosolv SMCC HD90 | 41.24 | 41.12 | 16.36 | 15.71 | 15.71 | 15.71 | 15.71 |
| Mannitol (Parteck M100) | 41.24 | 41.12 | 65.38 | 59.29 | 59.29 | 61.29 | 63.54 |
| Croscarmellose sodium | 2.5 | 5.0 | 5.0 | 5.0 | 5.0 | 5.0 | 5.0 |
| (Ac-Di-Sol SD711) | | | | | | | |
| Colloidal Silica | 0 | 0 | 0.5 | 0.5 | 0.5 | 0.5 | 0.5 |
| SSF (PRUV) | 0.26 | 0.26 | 0.26 | 2.0 | 2.0 | 1.5 | 1.25 |
| Croscarmellose sodium, | 2.5 | 0 | 0 | 4.0 | 4.0 | 2.50 | 1.0 |
| extragranular | | | | | | | |
| SSF, extragranular | 0.26 | 0 | 0 | 1.0 | 1.0 | 1.0 | 0.5 |
| Colloidal Silica, | 0 | 0.5 | 0.5 | 0.5 | 0.5 | 0.5 | 0.5 |
| extragranular | | | | | | | |
| Total (%) | 100 | 100 | 100 | 100 | 100 | 100 | 100 |

Physical Properties

| | | | | | | | |
|---|---|---|---|---|---|---|---|
| Bulk Density (g/cc) | 0.597 | 0.613 | 0.614 | — | 0.601 | 0.587 | 0.615 |
| Tapped Density (g/cc) | 0.786 | 0.806 | 0.807 | — | 0.813 | 0.815 | 0.854 |
| Carr Index (%) | 24 | 24 | 24 | — | 26 | 28 | 28 |

Sieve Analysis (μm/mesh, % Retained)

| | | | | | | | |
|---|---|---|---|---|---|---|---|
| 710/25 | 1.0 | 4.5 | 4.1 | — | 11.6 | 6.9 | 16.0 |
| 600/30 | 3.6 | 5.6 | 5.9 | — | 10.4 | 9.6 | 13.0 |
| 425/40 | 9.5 | 10.3 | 11.6 | — | 15.5 | 14.7 | 15.7 |
| 250/60 | 16.6 | 14.4 | 14.9 | — | 14.9 | 14.5 | 14.6 |
| 150/100 | 20.1 | 16.9 | 13.4 | — | 10.5 | 12.4 | 9.8 |
| 63/230 | 29.6 | 27.1 | 28.9 | — | 18.7 | 22.7 | 15.9 |
| <63pan | 19.4 | 19.6 | 19.5 | — | 16.8 | 19.8 | 13.0 |

Flow Rate Index (kg/sec)

| | | | | | | | |
|---|---|---|---|---|---|---|---|
| Mean | 0.394 | 0.209 | 0.174 | — | 0.218 | 0.191 | 0.627 |

Blend Uniformity (% assay)

| | | | | | | | |
|---|---|---|---|---|---|---|---|
| Mean | 98.2 | 95.6 | 94.3 | — | 98.3 | 98.4 | 99.5 |
| SD | 0.5 | 1.3 | 1.4 | — | 2.3 | 1.3 | 1.4 |
| RSD | 0.5 | 1.4 | 1.5 | — | 2.3 | 1.4 | 1.4 |

Portions of the blend for Composition 28 intended for filling into a 30 mg dose strength were sieved through several different mesh sizes (14, 16, 18 and 20) as well as screened consecutively through a 14 followed by a 20-mesh size. The results of the sieve cut analysis are summarized in Table 23.

TABLE 23
Sieve Cut Distribution for Different Mill Screen
Mesh Sizes for Composition 28 (C19517-25)

| Sieve Analysis | | | | | | |
|---|---|---|---|---|---|---|
| (t/mesh) | | 20 | 14 | 16 | 18 | 14 Mesh/ |
| (% Retained) | Pre-RC | Mesh | Mesh | Mesh | Mesh | 20 Mesh |
| 710/25 | 0.4 | 6.6 | 33.4 | 24.3 | 6.7 | 2.5 |
| 600/30 | 0.4 | 8.5 | 10.1 | 11.5 | 8.3 | 7.4 |
| 425/40 | 1.4 | 14.4 | 11.7 | 14.9 | 15.2 | 17.8 |
| 250/60 | 9.8 | 17.7 | 11.5 | 16.7 | 18.4 | 20.8 |
| 150/100 | 23.4 | 14.0 | 8.6 | 8.7 | 13.7 | 14.8 |
| 63/230 | 42.4 | 23.4 | 15.3 | 15.6 | 22.0 | 23.0 |
| <63t/pan | 20.1 | 14.0 | 8.0 | 10.9 | 15.1 | 14.2 |

The physical properties of the ribbons produced for Compositions 29 and 30 are summarized in Table 24.

TABLE 24
Physical Properties of the Ribbons
from Roller Compacted Compositions

| | |
|---|---|
| — | Roller Compacted Formulations |

| — | Composition 26 | Composition 27 |
|---|---|---|
| Solid Fraction | 0.6915 | 0.6914 |

All five blends were encapsulated into Size 1 capsules at 30 mg dose strengths using an Incap dosing disc automatic encapsulator. Compositions 26-30 were encapsulated in Size 1 capsules at 10 mg dose strengths using an Incap dosing disc automatic encapsulator. Size 1 tamping pins and dosing plates were used for the 30 mg capsules and Size 4 pins and plates for the 10 mg capsules. Weight variation acceptances of 7.5% and 10% were used during weight sorting of the 30 mg and 10 mg capsules, respectively. The content uniformity and dissolution data are summarized in Table 25. All five compositions exhibited rapid release of >80% in 10 minutes into the dissolution media under sink conditions as either 10 or 30 mg dose strengths.

TABLE 25
Content Uniformity and Dissolution Data for Automated Capsules
Made with Improved Flow Roller Compacted Blends
Content Uniformity

| | Composition | | | | | | | | | | |
|---|---|---|---|---|---|---|---|---|---|---|---|
| | 25 | 26 | | 27 | | 28 | | 29 | | 30 | |
| | Dose Strength Filled | | | | | | | | | | |
| | 30 | 10 | 30 | 10 | 30 | 10 | 30 | 10 | 30 | 10 | 30 |
| Mean | 100.3 | 94.7 | 95.6 | 92.8 | 96.7 | 94.3 | 101.5 | 97.9 | 99.1 | 98.1 | 99.1 |
| RSD (%) | 2.2 | 6.5 | 2.9 | 1.5 | 3.6 | 3.2 | 1.1 | 4.3 | 3.9 | 2.5 | 3.7 |
| Acceptance | 5.4 | 18.5 | 9.7 | 9.1 | 10.2 | 11.5 | 2.8 | 10.7 | 9.2 | 6.2 | 8.7 |
| Value | | | | | | | | | | | |

Dissolution Method

| Parameter | Condition |
|---|---|
| Apparatus | USP Apparatus 1 for Composition 25 |
| | USP Apparatus 2 (paddles with sinkers) |
| | for Compositions 26-30 |
| Media | 50 mM sodium phosphate buffer, |
| | pH 6.8 with 0.4% SDS |
| Media Volume | 900 mL for 10 mg Compositions 26 and 27 |
| | 500 mL for 10 mg Compositions 28-30 |
| | 900 mL for 30 mg Compositions |
| Vessel Size | 1000 mL |
| Temperature | 37.0 ± 0.5° C. |
| Speed | 0-60 minutes: 50 rpm for Composition 25 |
| | 0-60 minutes: 75 rpm for Compositions 26-30 |
| | 60-75 minutes: 250 rpm (infinity) for all |
| Time Points | 5, 10, 15, 30, 45, 60, 75 minutes |
| Filter | 0.45 μm, 25 mm Nylon Syringe Filter |

Dissolution Results (% dissolved)

| Composition- | Time Points (minutes) | | | | | | | |
|---|---|---|---|---|---|---|---|---|
| Dose Strength | | 5 | 10 | 15 | 30 | 45 | 60 | 75 |
| 25-30 mg | Mean | 24 | 57 | 70 | 83 | 87 | 88 | 99 |
| | % RSD | 57.0 | 21.8 | 10.6 | 4.0 | 3.4 | 3.8 | 2.7 |
| 26-10 mg | Mean | 26 | 65 | 74 | 82 | 86 | 88 | 93 |
| | % RSD | 70.0 | 12.0 | 8.0 | 6.0 | 5.1 | 3.9 | 1.5 |
| 26-30 mg | Mean | 33 | 70 | 81 | 90 | 93 | 94 | 98 |
| | % RSD | 4.6 | 7.5 | 5.7 | 3.9 | 3.3 | 3.0 | 2.7 |
| 27-10 mg | Mean | 39 | 72 | 81 | 89 | 92 | 93 | 95 |
| | % RSD | 10.7 | 6.8 | 6.1 | 4.7 | 4.3 | 3.6 | 2.7 |
| 27-30 mg | Mean | 32 | 72 | 83 | 92 | 95 | 96 | 98 |
| | % RSD | 17.1 | 9.6 | 7.8 | 5.7 | 4.7 | 4.5 | 3.2 |
| 28-10 mg | Mean | 28 | 66 | 76 | 84 | 87 | 88 | 94 |
| | % RSD | 54.9 | 7.4 | 4.0 | 2.8 | 2.7 | 2.6 | 3.3 |
| 28-30 mg | Mean | 43 | 82 | 92 | 98 | 100 | 101 | 102 |
| | % RSD | 7.0 | 6.7 | 5.1 | 3.0 | 2.4 | 2.4 | 2.3 |
| 29-10 mg | Mean | 36 | 67 | 75 | 82 | 84 | 86 | 93 |
| | % RSD | 41.2 | 15.3 | 12.7 | 9.5 | 8.0 | 7.4 | 5.3 |
| 29-30 mg | Mean | 37 | 73 | 85 | 94 | 96 | 97 | 98 |
| | % RSD | 18.4 | 5.9 | 4.2 | 3.5 | 3.4 | 3.2 | 3.5 |
| 30-10 mg | Mean | 48 | 74 | 82 | 88 | 90 | 91 | 99 |
| | % RSD | 4.7 | 5.3 | 5.2 | 4.4 | 4.1 | 3.7 | 3.4 |
| 30-30 mg | Mean | 39 | 72 | 84 | 92 | 93 | 94 | 96 |
| | % RSD | 15.0 | 5.0 | 5.3 | 5.6 | 5.4 | 5.3 | 5.2 |

### Example 29

### Stability of Improved Flow Direct and Roller-Compacted Blends

Chemical stability data for the capsules (10 mg and 30 mg dose strengths) filled with the improved flow direct blends (Composition 24) and roller-compacted blends (Compositions 28, 29 and 30) are summarized in Table 26. All lots were stable under the tested storage conditions.

TABLE 26
Chemical Stability Data for Improved Flow Direct and Roller-Compacted Blends

| | | Time Point and Storage Conditions | | | | | | | |
|---|---|---|---|---|---|---|---|---|---|
| | | | T = 2 wk | T = 1 M | T = 1 M | | T = 2 wk | T = 1 M | T = 1 M |
| | Dose | | 40° C./75% | 25° C./60% | 40° C./75% | | 40° C./75% | 25° C./60% | 40° C./75% |
| | Strength | T = 0 | RH | RH | RH | T = 0 | RH | RH | RH |
| Composition | (mg) | % Assay | | | | % Assay Weight Corrected | | | |
| 24 | 10 | 94.8 | 93.1 | 94.9 | 94.4 | 96.6 | 95.8 | 94.1 | 97.1 |
| 28 | 10 | 96.1 | 96.8 | 95.5 | 96.5 | 98.3 | 95.7 | 97.1 | 95.2 |
| 29 | 10 | 94.8 | 95.1 | 94.8 | 94.3 | 98.8 | 95.6 | 96.4 | 95.5 |
| 30 | 10 | 98.5 | 96.8 | 97.1 | 96.1 | 98.8 | 94.9 | 97 | 94.6 |
| 24 | 30 | 99.7 | 99.0 | 99.2 | 98.4 | 99.1 | 97.9 | 97.5 | 96 |
| 28 | 30 | 99.8 | 98.4 | 100.5 | 101.1 | 102.1 | 100.7 | 99.2 | 97.6 |
| 29 | 30 | 102.8 | 98.7 | 98.4 | 99.9 | 100.2 | 98.8 | 98.6 | 96.9 |
| 30 | 30 | 99.2 | 98.3 | 100.0 | 98.0 | 101.4 | 99.1 | 98 | 97.1 |

Dissolution data for the capsules (10 mg and 30 mg dose strengths) filled with the improved flow direct blends (Composition 24) and roller-compacted blends (Compositions 28, 29 and 30) are summarized in Table 27. All lots were stable under the tested storage conditions.

TABLE 27
Dissolution Data for Improved Flow Direct and Roller-Compacted Blends
Dissolution Method

| Parameter | Condition |
|---|---|
| Apparatus | USP Apparatus 2 (paddles with sinkers) |
| Media | 50 mM sodium phosphate buffer, pH 6.8 with 0.4% SDS |
| Media Volume | 500 mL for 10 mg |
| | 900 mL for 30 mg |
| Vessel Size | 1000 mL |
| Temperature | 37.0 ± 0.5° C. |
| Speed | 0-60 minutes: 75 rpm, 60-75 minutes: 250 rpm (infinity) |
| Time Points | 5, 10, 15, 30, 45, 60, 75 minutes |
| Filter | 0.45 μm, 25 mm Nylon Syringe Filter |

Dissolution Results (% dissolved)

| Composition- | Time Points (minutes) | | | | | | | |
|---|---|---|---|---|---|---|---|---|
| Dose Strength | | 5 | 10 | 15 | 30 | 45 | 60 | 75 |
| 24 T = 0 10 mg | Mean | 18 | 60 | 73 | 83 | 86 | 88 | 92 |
| | % RSD | 109.7 | 21.0 | 9.5 | 6.1 | 5.2 | 4.6 | 4.2 |
| 28 T = 0 10 mg | Mean | 28 | 66 | 76 | 84 | 87 | 88 | 94 |
| | % RSD | 54.9 | 7.4 | 4.0 | 2.8 | 2.7 | 2.6 | 3.3 |
| 29 T = 0 10 mg | Mean | 36 | 67 | 75 | 82 | 84 | 86 | 93 |
| | % RSD | 41.2 | 15.3 | 12.7 | 9.5 | 8.0 | 7.4 | 5.3 |
| 30 T = 0 10 mg | Mean | 48 | 74 | 82 | 88 | 90 | 91 | 99 |
| | % RSD | 4.7 | 5.3 | 5.2 | 4.4 | 4.1 | 3.7 | 3.4 |
| 24 T = 1 M | Mean | 29 | 65 | 75 | 83 | 86 | 87 | 93 |
| 25° C./60% RH 10 mg | % RSD | 59.3 | 9.8 | 6.5 | 3.9 | 3.0 | 2.7 | 2.5 |
| 28 T = 1 M | Mean | 44 | 71 | 79 | 86 | 88 | 88 | 94 |
| 25° C./60% RH 10 mg | % RSD | 18.0 | 4.0 | 3.2 | 3.3 | 3.6 | 3.9 | 3.8 |
| 29 T = 1 M | Mean | 37 | 71 | 80 | 86 | 88 | 89 | 95 |
| 25° C./60% RH 10 mg | % RSD | 33.2 | 7.2 | 4.8 | 3.5 | 3.2 | 3.2 | 2.8 |
| 30 T = 1 M | Mean | 38 | 69 | 78 | 86 | 88 | 89 | 97 |
| 25° C./60% RH 10 mg | % RSD | 14.1 | 3.0 | 3.4 | 3.7 | 3.7 | 3.7 | 0.8 |
| 24 T = 1 M | Mean | 31 | 64 | 75 | 85 | 88 | 89 | 95 |
| 40° C./75% RH 10 mg | % RSD | 83.4 | 18.3 | 8.2 | 6.1 | 6 | 5.8 | 4.4 |
| 28 T = 1 M | Mean | 34 | 66 | 76 | 85 | 87 | 88 | 95 |
| 40° C./75% RH 10 mg | % RSD | 52.3 | 11.8 | 5.5 | 3.8 | 3.6 | 3.6 | 2.9 |
| 29 T = 1 M | Mean | 29 | 63 | 75 | 84 | 86 | 87 | 93 |
| 40° C./75% RH 10 mg | % RSD | 67.7 | 13.2 | 4.4 | 2.9 | 3.1 | 3.4 | 2.8 |
| 30 T = 1 M | Mean | 33 | 69 | 80 | 89 | 91 | 92 | 98 |
| 40° C./75% RH 10 mg | % RSD | 33.9 | 4.0 | 1.6 | 1.5 | 1.7 | 1.8 | 1.3 |
| 24 T = 0 30 mg | Mean | 45 | 79 | 88 | 96 | 97 | 98 | 99 |
| | % RSD | 9.5 | 4.6 | 3.2 | 2.8 | 2.8 | 2.8 | 2.6 |
| 28 T = 0 30 mg | Mean | 43 | 82 | 92 | 98 | 100 | 101 | 102 |
| | % RSD | 7.0 | 6.7 | 5.1 | 3.0 | 2.4 | 2.4 | 2.3 |
| 29 T = 0 30 mg | Mean | 37 | 73 | 85 | 94 | 96 | 97 | 98 |
| | % RSD | 18.4 | 5.9 | 4.2 | 3.5 | 3.4 | 3.2 | 3.5 |
| 30 T = 0 30 mg | Mean | 39 | 72 | 84 | 92 | 93 | 94 | 96 |
| | % RSD | 15.0 | 5.0 | 5.3 | 5.6 | 5.4 | 5.3 | 5.2 |
| 24 T = 1 M | Mean | 46 | 78 | 88 | 96 | 98 | 98 | 99 |
| 25° C./60% RH 30 mg | % RSD | 13.2 | 7.1 | 4.4 | 3.5 | 3.4 | 3.3 | 3.1 |
| 28 T = 1 M | Mean | 44 | 78 | 87 | 93 | 95 | 95 | 96 |
| 25° C./60% RH 30 mg | % RSD | 11.8 | 3.6 | 2.8 | 2.5 | 2.3 | 2.3 | 2.1 |
| 29 T = 1 M | Mean | 39 | 74 | 85 | 93 | 96 | 97 | 99 |
| 25° C./60% RH 30 mg | % RSD | 5.1 | 2.9 | 2.1 | 2.3 | 2.6 | 2.7 | 3.0 |
| 30 T = 1 M | Mean | 39 | 73 | 85 | 94 | 97 | 98 | 100 |
| 25° C./60% RH 30 mg | % RSD | 11.5 | 3.3 | 4.3 | 4.8 | 4.6 | 4.4 | 4.3 |
| 24 T = 1 M | Mean | 42 | 75 | 84 | 92 | 94 | 95 | 96 |
| 40° C./75% RH 30 mg | % RSD | 14.8 | 3.5 | 2.8 | 2.0 | 1.8 | 1.7 | 1.2 |
| 28 T = 1 M | Mean | 41 | 78 | 88 | 95 | 97 | 98 | 99 |
| 40° C./75% RH 30 mg | % RSD | 10.2 | 3.9 | 3.2 | 3.0 | 3.2 | 3.5 | 3.8 |
| 29 T = 1 M | Mean | 35 | 72 | 85 | 94 | 97 | 98 | 100 |
| 40° C./75% RH 30 mg | % RSD | 15.8 | 4.8 | 2.9 | 2.1 | 1.8 | 1.5 | 1.6 |
| 30 T = 1 M | Mean | 38 | 74 | 86 | 97 | 99 | 100 | 100 |
| 40° C./75% RH 30 mg | % RSD | 9.4 | 2.4 | 2.7 | 2.9 | 3.0 | 3.2 | 3.3 |

### Example 30

### Manufacture of a 15 kg Blend of Composition 30 and Automatically Encapsulating into Size 1 Capsules at 10 mg, 20 mg and 30 mg Dose Strengths

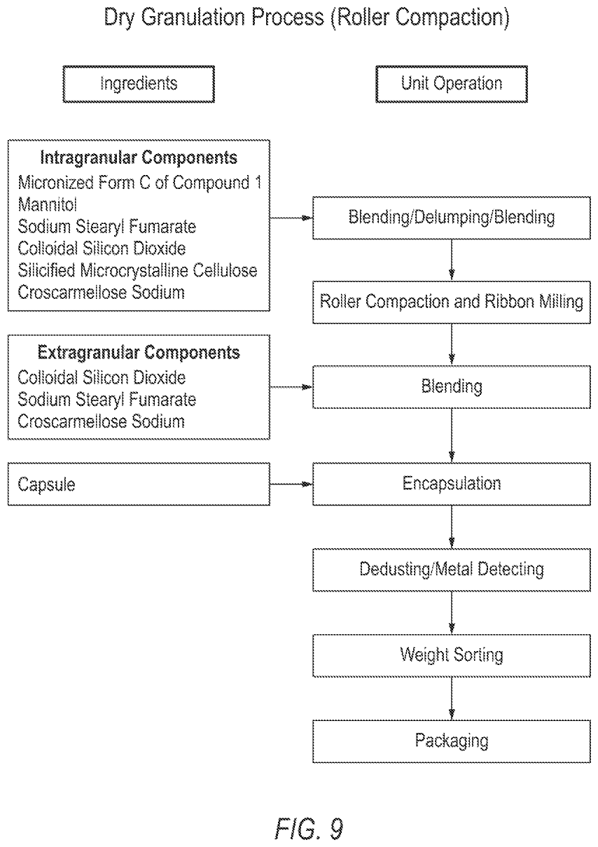

Composition 30 was manufactured on a 15 kg scale. The composition is summarized in Table 28. A general flow diagram for the manufacturing process used for this batch is shown in FIG. 9.

TABLE 28
10 mg, 20 mg and 30 mg Dose Strengths of
Composition 30 of Micronized Form C of Compound
1 Roller Compacted and Encapsulated

| | — | Quantity per capsule (mg) | | |
|---|---|---|---|---|
| Ingredient | % w/w | 10 | 20 | 30 |
| Micronized Form C of | 12.0 | 10 | 20 | 30 |
| Compound 1 | | | | |
| Silicified | 15.71 | 13.1 | 26.2 | 39.3 |
| Microcrystalline | | | | |
| Cellulose (SMCC) | | | | |
| Mannitol | 63.54 | 53.0 | 105.9 | 158.8 |
| Croscarmellose Sodium | 6.0 | 5.0 | 10.0 | 15.0 |
| Colloidal Silicon Dioxide | 1.0 | 0.8 | 1.7 | 2.5 |
| Sodium Stearyl Fumarate | 1.75 | 1.4 | 2.9 | 4.4 |
| (SSF) | | | | |
| Total | 100.0 | 83.3 | 166.7 | 250.0 |

A 50 L Bohle bin blender was used for the lot. The intragranular excipients were mixed and then de-lumped with a conical mill equipped with a 32R screen. The resulting blend was then roller compacted on a Gerteis roller compactor and mill with an initial screen size of 1.5 mm. A series of roll forces from 5.5 to 15 kN/cm and screen mesh sizes of 1.25 and 2.0 were studied. The particle size distributions, ribbon thicknesses and solid fraction measurements for these granulation sublot samples are summarized in Table 29.

TABLE 29
Sieve Cut Fractions for Different Roller Compacted Sub-
Lots with Varying Roller Pressures and Mill Screen Sizes

| Roll Force | | | | | | | | | | |
|---|---|---|---|---|---|---|---|---|---|---|
| (kN/cm)/ | % Granules Retained on Mesh | | | | | | | | | |
| Screen Size | | | | | | | | | | Final |
| (mm) | | | | | | | | | Combined | Blend/ |
| Microns/Mesh | 5.5/1.5 | 7.0/1.5 | 8.0/1.5 | 9.0/1.5 | 12.0/1.25 | 12.0/1.5 | 14.0/1.5 | 15.0/2.0 | Blend/1.0 | 1.0 |
| 710/25 | 50.00 | 59.2 | 58.90 | 61 | 51.4 | 63.2 | 72.2 | 79 | 25 | 23.7 |
| 600/30 | 6.6 | 6.7 | 7.0 | 7.1 | 9.5 | 5.7 | 5.7 | 5.1 | 13.1 | 12.6 |
| 425/40 | 7.3 | 7.1 | 7.8 | 8.6 | 9.6 | 6.9 | 5.6 | 4.1 | 15.9 | 16.9 |
| 250/60 | 8.4 | 7.4 | 7.9 | 8 | 9.1 | 7.3 | 5.6 | 4.2 | 12.5 | 13.6 |
| 150/100 | 6.3 | 5.2 | 5.4 | 4.5 | 6.6 | 5.9 | 3.4 | 3.1 | 8.2 | 9.4 |
| 63/230 | 11.9 | 9.1 | 8.5 | 7.2 | 9.9 | 8.5 | 5.1 | 2.2 | 5.3 | 6.1 |
| <63/Pan | 6.3 | 4.7 | 5.4 | 2.7 | 4 | 2.2 | 1.4 | 0.4 | 17.1 | 16.4 |
| Solid Fraction | 0.688 | 0.709 | 0.716 | 0.737 | 0.756 | 0.759 | 0.761 | 0.807 | NA | NA |

| | | | | | |
|---|---|---|---|---|---|
| Ribbon | 1.83 | 1.87-2.26 | 1.52-2.55 | NA | NA |
| Thickness | | | | | |
| (mm) | | | | | |

A roll force of 9 kN/cm using a smaller 1.0 mm screen size was employed for approximately 5 kg of the blend resulting in 4 kg of material. The 8, 9 and 12 kN/cm roll pressure sub-lots that had been milled with the 1.5 mm screen size were combined with this material and used as a composite for encapsulation. The blend uniformity results for the final blend (% theoretical assay, N=10) were mean=104.2%, SD=0.90 and RSD=0.87. A Planeta MG2 encapsulator with a dosator system that has a maximum production rate of 50,000 capsules/hour was used for the capsule filling. The process parameters used are shown in Table 30. The target fill weights of the blend for the 10, 20 and 30 mg dose strengths were 83, 167 and 250 mg, respectively. The mean empty Size 1 capsule weight was 74.5 mg. A size 3 dosator was used for all three dose strengths. Capsules were dedusted during the run and subsequently weight checked.

TABLE 30
Process Parameters for the Planeta MG2 Encapsulator

| Dose | | Powder Bed Height | Compression |
|---|---|---|---|
| (mg) | Dosator Size | (mm) | (mm) |
| 10 | 3 | 3.05 | 0 |
| 20 | 3 | 8.45 | 0 |
| 30 | 3 | 14.50 | 0 |

The content uniformity and dissolution data for the encapsulated dose strengths are summarized in Table 31.

TABLE 31
Content Uniformity and Dissolution Data for
10, 20 and 30 mg Capsules of Composition 30
Content Uniformity

| | Composition | | |
|---|---|---|---|
| | 30 | | |
| | Dose Strength Filled (mg) | | |
| | 10 | 20 | 30 |
| Mean | 100.5% | 100.9% | 98.1% |
| RSD (%) | 1.3 | 1.7 | 2.6 |
| Acceptance | 3.0 | 4.2 | 6.4 |
| Value | | | |

Dissolution Method

| Parameter | Condition |
|---|---|
| Apparatus | USP Apparatus 2 (paddles with sinkers) |
| Media | 50 mM sodium phosphate buffer, pH 6.8 with 0.3% SDS |
| Media Volume | 500 mL for 10 mg |
| | 900 mL for 20 mg and 30 mg |
| Vessel Size | 1000 mL |
| Temperature | 37.0 ± 0.5° C. |
| Speed | 0-60 minutes: 75 rpm, 60-75 minutes: 250 rpm (infinity) |
| Time Points | 10, 15, 30, 45, 60, 75 minutes |
| Filter | 0.45 μm, 25 mm Nylon Syringe Filter |

Results (% Dissolved)

| Composition- | Time Points (minutes) | | | | | | |
|---|---|---|---|---|---|---|---|
| Dose Strength | | 10 | 15 | 30 | 45 | 60 | 75 |
| 30-10 mg | Mean | 34 | 77 | 85 | 88 | 89 | 98 |
| | % RSD | 62.8 | 5.0 | 2.2 | 1.6 | 1.2 | 1.3 |
| 30-20 mg | Mean | 69 | 88 | 93 | 96 | 98 | 99 |
| | % RSD | 4.2 | 1.0 | 0.7 | 0.6 | 0.6 | 0.8 |
| 30-30 mg | Mean | 67 | 86 | 91 | 95 | 96 | 98 |
| | % RSD | 2.4 | 1.1 | 1.1 | 0.9 | 0.8 | 0.9 |

### Example 31

### Manufacture of Large-Scale Blends of Composition 30 and Encapsulating into Size 1 Capsules at 20 and 30 mg Dose Strengths

Micronized Form C of Compound 1 and mannitol were charged into the appropriately sized bin with a portion of the mannitol used to dry rinse the Compound 1 container. These two materials were then blended using a Bohle PTM 300 mobile blender. Sodium stearyl fumarate (intragranular portion) was charged to the bin with a portion of the silicified microcrystalline cellulose to dry rinse the sodium stearyl fumarate container. Colloidal silicon dioxide was charged to the bin with a portion of the silicified microcrystalline cellulose used to dry rinse the colloidal silicon dioxide container. The remaining silicified microcrystalline cellulose and all the intragranular croscarmellose sodium were charged to the bin and the components blended for approximately 15 minutes at 6 RPM (approximately 90 revolutions). The blended components were processed through a conical mill equipped with a 032R screen (˜812 microns) to de-lump the blend. The resulting material was then blended prior to roller compaction. Roller compaction and subsequent milling was then performed on a Gerteis Mini-Pactor equipped with an inline mill which was equipped with the appropriate screen mesh size. The roller compaction was performed with a roll pressure of approximately 12 kN/cm. The milled granules were transferred to the appropriately sized bin. The amounts for the extra-granular excipients including colloidal silicon dioxide, sodium stearyl fumarate, and croscarmellose sodium were adjusted based on the actual amount of granules after roller compaction and inline milling. Several scoops of milled granules were added to separate containers containing the extragranular colloidal silicon dioxide and extragranular sodium stearyl fumarate and mixed by hand for approximately 30 seconds. Both mixtures were screened through a 20-mesh hand screen directly into the bin. The extragranular croscarmellose sodium was then screened through a 20-mesh hand screen directly into the bin. The bin contents were then blended and the resulting final blend was transferred to a Planeta MG2 or Planeta G140 automatic encapsulator and encapsulated into Size 1 capsules at a target fill weight of 167 mg for the 20 mg capsules and 250 mg for the 30 mg capsules using a compression height of 0 mm and the appropriate dosing chamber depths. The capsule fill weights were maintained using a Statistical Weight Control System. Capsules were passed through a capsule polisher and metal detector. The resulting capsules were weight sorted on an IMA Precisa 150 Capsule Weight Sorter. The composition, actual ingredient amounts, selected manufacturing details, particle size of the Compound 1, physical properties of the blends, blend uniformity results and content uniformity results are summarized in Table 32.

TABLE 32
Selected Information for Large Scale Production of Composition
30 Filled into Size 1 Capsules at 20 and 30 mg Dose Strengths

| | wt/wt | Quantity | Quantity | Quantity | Quantity |
|---|---|---|---|---|---|
| Ingredient | % | (kg) | (kg) | (kg) | (kg) |
| Micronized Form C of Compound 1 | 12.00 | 6.413 | 2.400 | 4.164 | 2.400 |
| Prosolv SMCC HD90 | 15.71 | 8.135 | 3.142 | 5.451 | 3.142 |
| Mannitol (Parteck M100) | 63.54 | 32.902 | 12.708 | 22.048 | 12.708 |
| Croscarmellose sodium (Ac-Di-Sol | 5.000 | 2.600 | 1.000 | 1.735 | 1.000 |
| SD711) | | | | | |
| Aerosil 200 Pharma | 0.500 | 0.260 | 0.1000 | 0.174 | 0.1000 |
| SSF (PRUV) | 1.250 | 0.650 | 0.2500 | 0.434 | 0.2500 |
| Croscarmellose sodium, extragranular | 1.000 | 0.509 | 0.1927 | 0.339 | 0.1927 |
| 1 | | | | | |
| SSF, extragranular 1 | 0.50 | 0.254 | 0.0964 | 0.170 | 0.0964 |
| Aerosil 200 Pharma, extragranular 1 | 0.50 | 0.254 | 0.0964 | 0.170 | 0.0964 |
| Total | 100.0 | 52 | 20.0 | 34.7 | 20.0 |

Manufacturing Details

| | | | | | |
|---|---|---|---|---|---|
| Theoretical Blend Batch Size, kg | — | 52 | 12 | 34.7 | 8 |
| Theoretical Capsule Batch Size | — | 208000 | 48000 | 208158 | 48000 |
| Dose Strength | — | 30 | 30 | 20 | 20 |
| Bin Type | — | Bohle | Bohle | Bohle | Bohle |
| Pre-Blend Bin Size | — | 200 | 100 | 200 | 100 |
| Roller Compactor Mill Mesh Size, mm | — | 0.8 | 1.0 | 0.8 | 1.0 |
| Final Blend Bin Size | | 200 | 50 | 100 | 50 |

Particle Size Distribution of Micronized Form C of Compound 1

| | | | | | |
|---|---|---|---|---|---|
| D₁₀ | — | 2.1 | 1.7 | 1.8 | 1.7 |
| D₅₀ | — | 4.1 | 3.2 | 3.3 | 3.2 |
| D₉₀ | — | 8.0 | 6.1 | 6.5 | 6.1 |

Physical Properties

| | | | | | |
|---|---|---|---|---|---|
| Bulk Density (g/cc) | — | 0.67 | 0.65 | 0.59 | 0.65 |
| Tapped Density (g/cc) | — | 0.84 | 0.85 | 0.79 | 0.85 |
| Carr Index (%) | — | 20.2 | 23.0 | 26.0 | 23.0 |

Sieve Analysis (μm/mesh, % Retained)

| | | | | | |
|---|---|---|---|---|---|
| 710/25 | — | 3.0 | 22.9 | 4.3 | 22.9 |
| 600/30 | — | 11.9 | 10.2 | 12.1 | 10.2 |
| 425/40 | — | 23.5 | 14.0 | 22.4 | 14.0 |
| 250/60 | — | 20.2 | 11.9 | 20.3 | 11.9 |
| 150/100 | — | 11.5 | 8.8 | 12.4 | 8.8 |
| 63/230 | — | 16.2 | 15.6 | 17.6 | 15.6 |
| <63pan | — | 13.0 | 15.4 | 9.8 | 15.4 |
| Mean Flow Rate Index (kg/sec) | — | 3.06 (n = 9) | 1.97 (n = 3) | 0.47 (n = 3) | 1.97 (n = 3) |

Blend Uniformity (% assay)

| | | | | | |
|---|---|---|---|---|---|
| Mean | — | 102.3 | 100.1 | 98.4 | 100.1 |
| SD | — | 0.2 | 0.5 | 0.3 | 0.5 |
| RSD | — | 0.2 | 0.5 | 0.3 | 0.5 |

Content Uniformity (Label of Claim %)

| | | | | | |
|---|---|---|---|---|---|
| Mean | — | 103.1 | 100.0 | 99.6 | 100.4 |
| SD | — | 0.9 | 1.2 | 1.3 | 1.1 |
| Acceptance Value | — | 3.7 | 2.8 | 3.2 | 2.6 |

### Example 32

### Manufacture of a Lab Scale Blend (300 g) of Composition 30 with Different Ratios of Form A to Form C of Compound 1 and Hand Filling into Size 1 Capsules at a 30 mg Dose Strength

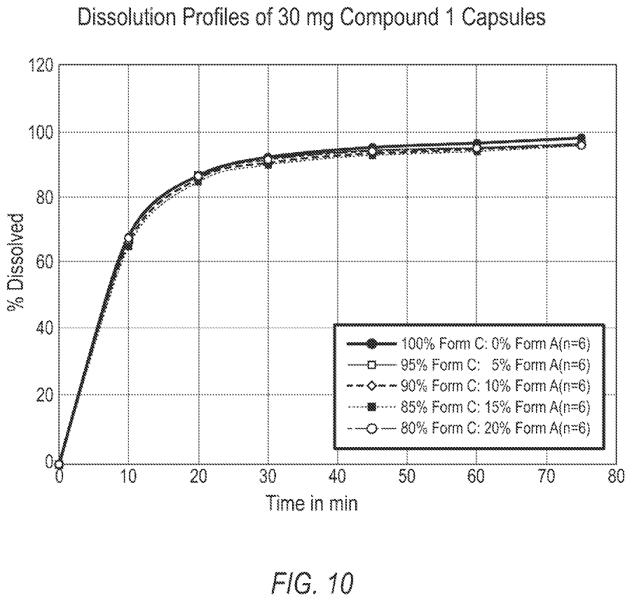

Different ratios of micronized Form A and Form C of similar particle size distribution (5% Form A:95% Form C, 10% Form A:90% Form C, 15% Form A:85% Form C and 20% Form A:80% Form C) of Compound 1 and mannitol were charged into an appropriately sized V-blender and blended. Silicified microcrystalline cellulose, intra-granular sodium stearyl fumarate, intragranular colloidal silica dioxide, and intragranular croscarmellose sodium were charged into the V-blender and the components blended. The blend was then processed through a lab scale conical mill equipped with a 032R screen (˜812 microns) to de-lump the blend. The resulting material was then blended prior to roller compaction. Roller compaction was performed on a TF-Mini roller compactor at a roll pressure of approximately 40 kgf/cm², where the ribbons were passed through an offline oscillating granulator with a 20-mesh screen size. The extragranular excipients were screened through a 20-mesh hand screen into the V-blender and then blended with the. The resulting final blend was hand filled into Size 1 capsules at a target fill weight of 250 mg for 30 mg dose strength capsules. The composition, ratio of Form A to Form C of Compound 1, flowability and particle size distribution of the final blends are summarized in Table 33. A graph of the dissolution profiles of are shown in FIG. 10.

TABLE 33
Physical Properties of Blends with Compound 1 Composition
30 with Different Form A to Form C Ratios

| | | | | | |
|---|---|---|---|---|---|
| | Composition | Composition | Composition | Composition | Composition |
| — | 30a | 30b | 30c | 30d | 30e |

Ingredient (%)

| | | | | | |
|---|---|---|---|---|---|
| Compound 1, Form A | N/A | 0.60 | 1.20 | 1.80 | 2.40 |
| Compound 1, Form C | 12.00 | 11.40 | 10.80 | 10.20 | 9.60 |

| | |
|---|---|
| Prosolv SMCC HD90 | 15.71 |
| Mannitol (Parteck M100) | 63.54 |
| Croscarmellose sodium | 5.00 |
| (Ac-Di-Sol, intra-granular) | |
| Colloidal Silica Dioxide | 1.25 |
| (intra-granular) | |
| Sodium Stearyl Fumarate | 0.50 |
| (intra-granular) | |
| Total | 98.0 |
| Croscarmellose sodium | 1.00 |
| (Ac-Di-Sol, extra-granular) | |
| Colloidal Silica Dioxide | 0.50 |
| (extra-granular) | |
| Sodium Stearyl Fumarate | 0.50 |
| (extra-granular) | |
| Total | 100 |

Physical Properties

| | | | | | |
|---|---|---|---|---|---|
| Bulk Density (g/cc) | 0.63 | 0.57 | 0.57 | 0.57 | 0.57 |
| Tapped Density (g/cc) | 0.80 | 0.79 | 0.78 | 0.80 | 0.78 |
| Carr Index (%) | 22.0 | 27.9 | 27.0 | 27.9 | 26.0 |

Sieve Analysis (μm/mesh, % Retained)

| | | | | | |
|---|---|---|---|---|---|
| 710/25 | 8.2 | 1.0 | 1.3 | 1.2 | 1.1 |
| 600/30 | 9.9 | 2.9 | 1.9 | 1.8 | 2.4 |
| 425/40 | 14.5 | 16.5 | 15.0 | 14.7 | 16.0 |
| 250/60 | 14.1 | 21.4 | 20.8 | 22.7 | 21.4 |
| 150/100 | 11.1 | 15.4 | 15.1 | 16.2 | 15.5 |
| 63/230 | 23.0 | 25.6 | 24.8 | 25.2 | 25.7 |
| <63pan | 18.6 | 15.6 | 15.6 | 15.6 | 15.6 |

Flow Rate Index (kg/sec)

| | | | | | |
|---|---|---|---|---|---|
| Mean (n = 3) | 0.27 | 0.23 | 0.21 | 0.20 | 0.20 |

### Example 33

### Manufacture of a Lab Scale Blend (Batch Size 200-300 g) of Composition 30 with Form C of Compound 1 of Various Particle Size Distributions and Hand Filled into Size 1 Capsules at a 30 mg Dose Strength

Form C of Compound 1 and mannitol were charged into the appropriately sized V-blender and blended. Silicified microcrystalline cellulose, intra-granular sodium stearyl fumarate, intragranular colloidal silica dioxide, and intragranular croscarmellose sodium were charged into the V-blender. The components were blended and then processed through a conical mill equipped with a 032R screen (˜812 microns) to de-lump the blend. The resulting material was then blended prior to roller compaction. Roller compaction was performed on a TF-Mini roller compactor at a roll pressure of approximately 40 kgf/cm², and the resulting ribbons were passed through an offline oscillating granulator with a 20-mesh screen size. The extra-granular excipients were screened through a 20-mesh hand screen into the V-blender and then blended with the granules. The resulting final blend was hand filled into Size 1 capsules at a target fill weight of 250 mg for 30 mg dose strengths capsules. The composition, particle size of the Compound 1, blend flowability and particle size distribution of the final blends are summarized in Table 34.

TABLE 34
Physical Properties of Blends with Form C of Compound 1 Composition 30 with Different Particle Sizes

| | | | | | | |
|---|---|---|---|---|---|---|
| — | Composition 30f | [symbol] omposition30g | [symbol] omposition 30h | [symbol] omposition 30t | [symbol] omposition30j | [symbol] omposition30k |

Ingredient (%)

| | |
|---|---|
| Compound 1 | 12.00 |
| Prosolv SMCC | 15.71 |
| HD90 | |
| Mannitol | 63.54 |
| (Parteck M100) | |
| Croscarmellose | 5.00 |
| sodium (Ac-Di-Sol, | |
| intra-granular) | |
| Colloidal Silica | 1.25 |
| Dioxide (intra- | |
| granular) | |
| Sodium Stearyl | 0.50 |
| Fumarate (intra- | |
| granular) | |
| Total | 98.0 |
| Croscarmellose | 1.00 |
| sodium (Ac-Di-Sol, | |
| extra-granular) | |
| Colloidal Silica | 0.50 |
| Dioxide (extra- | |
| granular) | |
| Sodium Stearyl | 0.50 |
| Fumarate (extra- | |
| granular) | |
| Total | 100 |

Form C of Compound 1 Particle Sizes

| | | | | | | |
|---|---|---|---|---|---|---|
| D₁₀ μm | 1.8 | — | 2.4 | 2.6 | 2.8 | 3.0 |
| D₅₀ μm | 3.2 | — | 4.8 | 5.4 | 6.3 | 7.9 |
| D₉₀ μm | 6.2 | 4.2 | 9.9 | 11.6 | 14.1 | 20.3 |

Physical Properties

| | | | | | | |
|---|---|---|---|---|---|---|
| Bulk Density (g/cc) | 0.63 | 0.51 | 0.55 | 0.55 | 0.54 | 0.58 |
| Tapped Density (g/cc) | 0.80 | 0.66 | 0.73 | 0.77 | 0.77 | 0.79 |
| Carr Index (%) | 22.0 | 22.9 | 25.0 | 29.0 | 30.0 | 26.0 |

Sieve Analysis (μm/mesh, % Retained)

| | | | | | | |
|---|---|---|---|---|---|---|
| 710/25 | 8.2 | 0.8 | 0.9 | 0.7 | 0.7 | 5.3 |
| 600/30 | 9.9 | 3.8 | 3.5 | 3.8 | 5.2 | 9.1 |
| 425/40 | 14.5 | 12.7 | 12.2 | 14.3 | 16.2 | 14.1 |
| 250/60 | 14.1 | 16.2 | 15.2 | 15.0 | 17.3 | 16.7 |
| 150/100 | 11.1 | 14.4 | 14.1 | 13.2 | 14.0 | 13.3 |
| 63/230 | 23.0 | 28.6 | 28.6 | 27.3 | 23.6 | 22.0 |
| <63pan | 18.6 | 22.2 | 24.6 | 25.0 | 21.6 | 17.4 |

Flow Rate Index (kg/sec)

| | | | | | | |
|---|---|---|---|---|---|---|
| Mean (n = 3) | 0.27 | 1.04 | 0.97 | 1.06 | 1.36 | 0.26 |

[symbol]

indicates data missing or illegible when filed

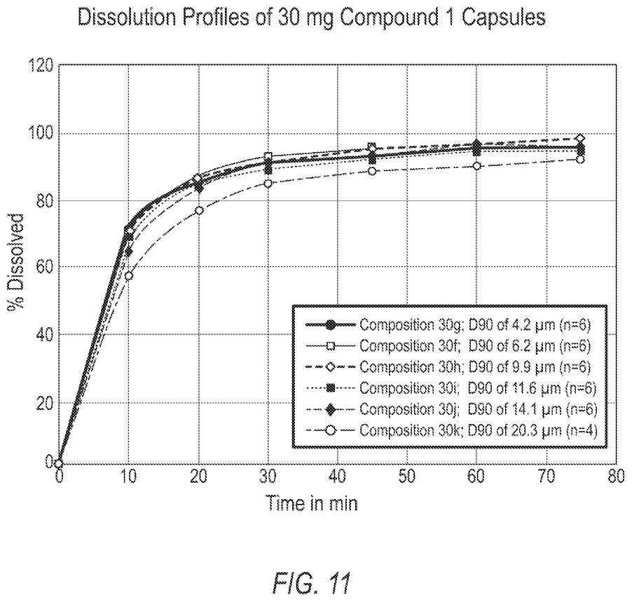

The dissolution results for the 30 mg dose strength capsules prepared from Compositions 30f through 30k are shown in Table 35. The resulting dissolution profiles are shown in FIG. 11.

TABLE 35
Dissolution Results for Blends of Form C of Compound
1 Composition 30 with Different Particle Sizes

| | | | | | | |
|---|---|---|---|---|---|---|
| | Composition | Composition | Composition | Composition | Composition | Composition |
| — | 30f | 30g | 30h | 30i | 30j | 30k |

Dissolution Method Conditions

| Parameter | Condition |
|---|---|
| Apparatus | USP Apparatus 2 (paddles with sinkers) |
| Media | 50 mM sodium phosphate buffer, pH 6.8 with 0.3% SDS |
| Media Volume | 900 mL for 30 mg |
| Vessel Size | 1000 mL |
| Temperature | 37.0 ± 0.5° C. |
| Speed | 0-60 minutes: 75 rpm, 60-75 minutes: 250 rpm (infinity) |
| Time Points | 10, 20, 30, 45, 60 and 75 minutes |
| Filter | 0.45 μm, 25 mm Nylon Syringe Filter |

Dissolution Results (% Dissolved)

Time Points (minutes)

| Composition | | 10 | 20 | 30 | 45 | 60 | 75 |
|---|---|---|---|---|---|---|---|
| Composition 30f | Mean | 68 | 87 | 92 | 95 | 97 | 98 |
| | % RSD | 2.81 | 2.13 | 2.17 | 1.72 | 1.51 | 1.30 |
| Composition 30g | Mean | 71 | 85 | 91 | 92 | 95 | 95 |
| | % RSD | 2.91 | 2.16 | 2.33 | 1.51 | 2.37 | 0.52 |
| Composition 30h | Mean | 68 | 86 | 91 | 95 | 97 | 98 |
| | % RSD | 3.29 | 1.10 | 1.14 | 0.92 | 0.71 | 0.28 |
| Composition 30i | Mean | 67 | 84 | 89 | 92 | 94 | 95 |
| | % RSD | 4.54 | 2.45 | 2.25 | 1.49 | 1.81 | 0.64 |
| Composition 30j | Mean | 64 | 83 | 91 | 93 | 96 | 96 |
| | % RSD | 3.30 | 1.77 | 1.01 | 1.36 | 1.53 | 0.43 |
| Composition 30k | Mean | 57 | 77 | 85 | 88 | 90 | 92 |
| | % RSD | 1.88 | 1.50 | 1.22 | 1.17 | 1.32 | 0.50 |

### Example 34

### Tablets Prepared by Changing the Drug Load of Composition 30 (0.4% and from 33% by Weight) by Reducing or Increasing the Filler Amounts

The amount of micronized Form C of Compound 1 was varied from 12 wt % in Composition 30 to 0.4% and 33% by adjusting the percentages of mannitol (Parteck M100) and SMCC (Prosolv HD90). The compositions of these new blends are described in Table 36. These blends contain the same intragranular and extragranular compositions as Composition 30. Mannitol and micronized Form C of Compound 1 were added to a rotational-blender and mixed before adding the remainder of the intragranular material, which was then mixed, passed through a conical mill, and blended. This blend was then dry granulated with a roller compactor with an inline mill. The final blends were mixed in a V-blender.

TABLE 36
Composition and Physical Properties of Blends Prepared by
Changing the Drug Load of Composition 30 (0.4% and from 33%
by weight) by Reducing or Increasing the Filler Amounts

| | | |
|---|---|---|
| — | Composition 31 | Composition 32 |

Ingredient (%)
Intragranular Components

| | | |
|---|---|---|
| Micronized Compound 1 | 0.40 | 33.00 |
| SMCC (Prosolv HD90) | 18.01 | 11.55 |
| Mannitol (Parteck M100) | 72.84 | 46.70 |

Extragranular Components

| | | |
|---|---|---|
| Croscarmellose sodium | 6.00 | 600 |
| (Ac-Di-Sol SD711) | | |
| Colloidal Silica | 1.00 | 1.00 |
| (Aerosil 200) | | |
| Sodium stearoyl fumarate | 1.75 | 1.75 |
| (PRUV) | | |
| Total | 100.00 | 100.00 |

Physical Properties

| | | |
|---|---|---|
| Ribbon Solid Fraction | 0.862 | 0.778 |
| Bulk Density (g/cc) | 0.638 | 0.484 |
| Tapped Density (g/cc) | 0.788 | 0.711 |
| Carr Index (%) | 19 | 32 |

Sieve Cuts

| | | |
|---|---|---|
| 710/25 | 0.7 | 8.1 |
| 600/30 | 0.9 | 6.6 |
| 425/40 | 1.0 | 13.9 |
| 250/60 | 7.5 | 15.8 |
| 150/100 | 16.5 | 13.4 |
| 63/230 | 51.2 | 24.5 |
| <63pan | 20.8 | 17.9 |
| Mean FRI (kg/s) | 1.882 | 0.097 |

Two dose strengths of tablets were prepared from Composition 31 and Composition 32, respectively, by tableting on a Korsch tablet press. The characterization data for these tablets is shown in Table 37.

TABLE 37
Characterization Data for Tablets Prepared by Changing the Drug Load of Composition
30 (0.4% and from 33% by weight) by Reducing or Increasing the Filler Amounts

| | | Tooling | Target | Hardness | Tablet Solid | Disintegration |
|---|---|---|---|---|---|---|
| Strength | Composition | size | Weight (mg) | (kp) | Fraction | Time (sec) |
| 1 mg | 31 | 5/16″ | 250 | 5 to 12 | 0.761 | 88 to 120 |
| | | standard | | | | |
| | | round | | | | |
| 5 mg | 31 | 0.45″ × | 1250 | 24 to 30 | 0.835 | 56 to 63 |
| | | 0.75″ | | | | |
| | | modified | | | | |
| 20 mg | 32 | 5 mm | 60.6 | 6 to 10 | 0.870 | 49 to 76 |
| | | standard | | | | |
| | | round | | | | |
| 100 mg | 32 | 5/16″ | 303 | 5 to 11 | 0.918 | 41 to 65 |
| | | standard | | | | |
| | | round | | | | |

The dissolution testing results for these tablets (1, 5, 20 and 100 mg) are summarized in Table 38.

TABLE 38
Dissolution Data for Tablets Prepared by Changing the Drug Load of Composition
30 (0.4% and from 33% by weight) by Reducing or Increasing the Filler Amounts
Dissolution Method Conditions

| Parameter | Condition |
|---|---|
| Apparatus | USP Apparatus 2 (paddles) |
| Media | 50 mM sodium phosphate buffer, pH 6.8 with 0.3% SDS (0.6% SDS for |
| | 100 mg) |
| Media Volume | 900 mL for 20 and 100 mg |
| | 500 mL for 1 and 5 mg |
| Vessel Size | 1000 mL |
| Temperature | 37.0 ± 0.5° C. |
| Speed | 0-60 minutes: 75 rpm, 60-75 minutes: 250 rpm (infinity) |
| Time Points | 10, 20, 30, 45, 60 and 75 minutes |
| Filter | 0.45 Jim, 25 mm Nylon Syringe Filter |

Dissolution Results (% Dissolved)

| Composition- | Time Points (minutes) | | | | | | |
|---|---|---|---|---|---|---|---|
| Dose Strength | — | 10 | 20 | 30 | 45 | 60 | 75 |
| 31-1 mg | Mean | 91 | 92 | 93 | 93 | 93 | 93 |
| | % RSD | 2.38 | 2.35 | 1.91 | 1.95 | 1.91 | 1.78 |
| 31-5 mg | Mean | 90 | 91 | 92 | 92 | 92 | 92 |
| | % RSD | 0.74 | 0.56 | 0.55 | 0.54 | 0.51 | 0.49 |
| 32-20 mg | Mean | 37 | 55 | 67 | 78 | 84 | 88 |
| | % RSD | 1.27 | 0.66 | 0.67 | 0.71 | 0.72 | 0.80 |
| 32-100 mg | Mean | 67 | 81 | 86 | 89 | 90 | 90 |
| | % RSD | 4.27 | 1.00 | 0.78 | 1.86 | 2.24 | 2.41 |

### Example 35

### Tablets Prepared by Changing the Nature of the Fillers of Composition 30

Three different blends were made based on Composition 30 by varying the nature of the two fillers. The composition and physical properties of these three blends are summarized in Table 39.

TABLE 39
Summary of Composition and Physical Properties of Blends Prepared
by Changing the Nature of the Fillers of Composition 30

| | | | |
|---|---|---|---|
| — | Composition 33 | Composition 34 | Composition 35 |

Ingredient (wt %)

| | | | |
|---|---|---|---|
| Micronized Compound 1 | 12.00 | 12.00 | 12.00 |
| SMCC (Prosolv HD90) | 0 | 15.71 | 0 |
| Mannitol (Parteck M100) | 0 | 0 | 63.54 |
| Lactose (Fast Flo 316) | 0 | 63.54 | 0 |
| Pregelatinized Starch | 15.71 | 0 | 15.71 |
| Dicalcium phosphate | 63.54 | 0 | 0 |
| (Dical, Encompress) | | | |
| Croscarmellose sodium | 6.00 | 6.00 | 6.00 |
| (Ac-Di-Sol SD711) | | | |
| Colloidal Silica | 1.00 | 1.00 | 1.00 |
| (Aerosil 200) | | | |
| SSF (PRUV) | 1.75 | 1.75 | 1.75 |
| Total | 100.00 | 100.00 | 100.00 |

Roller Compacted Ribbon & Granule Properties

| | | | |
|---|---|---|---|
| Ribbon Solid Fraction | 0.756 | 0.718 | 0.666 |

Physical Properties

| | | | |
|---|---|---|---|
| Bulk Density (g/cc) | 0.747 | 0.562 | 0.540 |
| Tapped Density (g/cc) | 1.067 | 0.815 | 0.772 |
| Carr Index (%) | 30 | 31 | 30 |

Final Blend Sieve Cut (% retained)

| | | | |
|---|---|---|---|
| 710/25 | 0.6 | 1.0 | 0.7 |
| 600/30 | 2.4 | 5.1 | 4.7 |
| 425/40 | 8.7 | 13.8 | 13.0 |
| 250/60 | 26.9 | 15.6 | 17.5 |
| 150/100 | 24.2 | 12.7 | 14.0 |
| 63/230 | 25.6 | 25.0 | 29.1 |
| <63pan | 10.4 | 26.3 | 20.1 |
| Mean FRI (kg/s) | 0.327 | 0.157 | 0.202 |

These blends contain the same intragranular and extragranular compositions as Composition 30. A filler and micronized Form C of Compound 1 were added to a rotational-blender and mixed before adding the remainder of the intragranular material, which was then mixed, passed through a conical mill, and blended. This blend was then dry granulated with a roller compactor with an inline mill. The final blends were mixed in a V-blender.

One dose strength of each composition was pressed into tablets by tableting on a Korsch tablet press. The characterization data for these tablets is shown in Table 40.

TABLE 40
Characterization Data for Tablets Prepared by Changing the Fillers of Composition 30

| Composition- | Tooling | Target Weight | Hardness | Tablet Solid | Disintegration |
|---|---|---|---|---|---|
| Dose Strength | Size | (mg) | (kp) | Fraction | Time (sec) |
| 33-25 mg | 5/16″ standard | 208.3 | 2 to 3 | 0.855 | 34 to 46 |
| | round concave | | | | |
| 34-60 mg | 13/32″ standard | 500 | 8 to 11 | 0.864 | 54 to 68 |
| | round concave | | | | |
| 35-100 mg | ½″ standard | 833.3 | 16 to 24 | 0.880 | 120 to 0140 |
| | round concave | | | | |

The dissolution testing results for these tablets (25, 60 and 100 mg) are summarized in Table 41.

TABLE 41
Dissolution Data for Tablets Prepared by Changing
the Nature of the Fillers for Composition 30
Dissolution Method Conditions

| Parameter | Condition |
|---|---|
| Apparatus | USP Apparatus 2 (paddles) |
| Media | 50 mM sodium phosphate buffer, pH 6.8 with 0.3% SDS for 25 mg, 0.4% |
| | SDS for 60 mg, and 0.6% for 100 mg |
| Media Volume | 900 mL |
| Vessel Size | 1000 mL |
| Temperature | 37.0 ± 0.5° C. |
| Speed | 0-60 minutes: 75 rpm, 60-75 minutes: 250 rpm (infinity) |
| Time Points | 10, 20, 30, 45, 60 and 75 minutes |
| Filter | 0.45 Jim, 25 mm Nylon Syringe Filter |

Dissolution Results (% Dissolved)

| Composition- | Time Points (minutes) | | | | | | |
|---|---|---|---|---|---|---|---|
| Dose Strength | | 10 | 20 | 30 | 45 | 60 | 75 |
| 33-25 mg | Mean | 59 | 74 | 80 | 84 | 86 | 98 |
| | % RSD | 6.19 | 1.96 | 1.28 | 0.98 | 0.81 | 0.86 |
| 34-60 mg | Mean | 85 | 93 | 95 | 96 | 96 | 97 |
| | % RSD | 0.79 | 0.17 | 0.42 | 0.50 | 0.53 | 0.49 |
| 35-100 mg | Mean | 85 | 95 | 96 | 97 | 97 | 98 |
| | % RSD | 0.16 | 0.18 | 0.15 | 0.09 | 0.23 | 0.17 |

### Example 36

### Manufacture of Hand Filled Capsules at 30, 50 and 100 mg Dose Strengths and Tablets at 30 and 100 mg Dose Strengths Using Composition 30 with Crospovidone as a Disintegrant

A roller compacted blend with the same composition as Composition 30 (Composition 36) except replacing croscarmellose sodium with copovidone was prepared according to the procedure described in Example 34 and hand filled into capsules or pressed into tablets of various dose strengths. The composition of the blend for Composition 36 is shown in Table 42.

TABLE 42
Composition of the Roller Compacted Blend based on
Composition 30 using Crospovidone as a Disintegrant

| | Composition 36 |
|---|---|
| Ingredient | wt/wt % |
| Micronized Form C of Compound 1 | 12.00 |
| Silicified Microcrystalline cellulose (Prosolv HD90) | 15.71 |
| Mannitol (Parteck M100) | 63.54 |
| Crospovidone (Polyplasdone XL-10) | 5.000 |
| Colloidal Silica (Aerosil 200 Pharma) | 0.500 |
| Sodium stearoyl fumarate (PRUV) | 1.250 |
| Crospovidone (Polyplasdone XL-10) extragranular | 1.000 |
| Sodium stearoyl fumarate (PRUV), extragranular | 0.500 |
| Colloidal Silica (Aerosil 200 Pharma), extragranular | 0.500 |
| Total | 100.0 |

A description of the dose strengths and formulations made from Composition 36 is shown in Table 43.

TABLE 43
Description of Dose Strengths and Formulations
of the Roller Compacted Blend based on Composition
30 using Crospovidone as a Disintegrant

| Dose | Target Fill | | Capsule |
|---|---|---|---|
| Strength | Weight or | | Shell |
| (mg) | Target weight | Formulation | Size |
| 30 | 250 | capsule | 1 |
| 30 | 250 | tablet | — |
| 100 | 833 | tablet | — |
| 100 | 833 | capsule | 000 |
| 50 | 416 | capsule | 0 |

Two dose strengths of Composition 36 were pressed into tablets by tableting on a Korsch tablet press. The characterization data for these tablets is shown in Table 44.

TABLE 44
Physical Properties of Tablets made from Composition 36

| Dose Strength (mg) | 30 | 100 |
|---|---|---|
| Total tablet weight, mg | 250.0 | 833.3 |
| Tablet Tooling | 5/16″ standard | ½″ standard |
| | round | round |
| Hardness range (kP) | 9-11 | 23-31 |
| Solid Fraction (mean) | 0.693 | 0.820 |
| Disintegration time range (sec) | 27-29 | 50-81 |

The dissolution testing results for a tablet (30 mg) and two capsules (30 and 50 mg) prepared from Composition 36 are summarized in Table 45.

TABLE 45
Dissolution Data for a Tablet and Two Capsules Prepared using Composition 36
Dissolution Method Conditions

| Parameter | Condition |
|---|---|
| Apparatus | USP Apparatus 2 (paddles with size appropriate sinkers/no sinkers for tablets) |
| Media | 50 mM sodium phosphate buffer, pH 6.8 with 0.3% SDS |
| Media Volume | 900 mL |
| Vessel Size | 1000 mL |
| Temperature | 37.0 ± 0.5° C. |
| Speed | 0-60 minutes: 75 rpm, 60-75 minutes: 250 rpm (infinity) |
| Time Points | 10, 20, 30, 45, 60 and 75 minutes |
| Filter | 0.45 μm, 25 mm Nylon Syringe Filter |

Dissolution Results (% Dissolved)

| | Time Points (minutes) | | | | | | |
|---|---|---|---|---|---|---|---|
| Formulation (type) | | 10 | 20 | 30 | 45 | 60 | 75 |
| 30 mg (capsule) | Mean | 67 | 88 | 93 | 96 | 97 | 101 |
| | % RSD | 5.18 | 2.82 | 2.52 | 2.19 | 2.01 | 0.38 |
| 30 mg (tablet) | Mean | 68 | 86 | 93 | 96 | 97 | 98 |
| | % RSD | 0.85 | 0.57 | 0.40 | 0.40 | 0.46 | 0.36 |
| 50 mg (capsule) | Mean | 66 | 85 | 90 | 93 | 95 | 97 |
| | % RSD | 1.55 | 0.87 | 0.63 | 0.61 | 0.55 | 0.27 |

### Example 37

### Tablets Manufactured from Composition 30

Blend from Composition 30 was pressed into tablets (both miniature and conventional sizes) from a dose strength range of 2.5 to 100 mg. The final dry granulated blend of Composition 30 was compressed into tablets using a single station tablet press. The 2.5 mg mini-tabs were compressed using multi-tip tooling. Conventional tablets of 7.5, 25, 30 and 100 mg strengths were compressed with standard tooling. Details of the tablet compositions, parameters, properties and dissolution profiles are presented in Table 46.

TABLE 46
Properties and Dissolution Profiles of Tablets made from Composition 30

| | Composition 30 | | | Composition 30 | | |
|---|---|---|---|---|---|---|
| | Dose Strength (mg) | | | | | |
| — | 7.5 | 30 | 50 | 2.5 | 25 | 100 |
| Total tablet weight, | 62.5 | 250.0 | 416.7 | 20.8 | 208.0 | 833.3 |
| mg | | | | | | |
| Tablet Tooling | 3/16″ | 5/16″ | 13/32″ | 3 mm- | 6/15″ | ½″ |
| | standard | standard | standard | round | standard | standard |
| | round concave | round | round | concave, 8 | round | round |
| Hardness range | 7-11 | 7-11 | 11-17 | 0.5-8.5 | 5-11 | 18-23 |
| (kP) | | | | | | |
| Solid Fraction | 0.953 | 0.841 | 0.838 | N/A | 0.885 | 0.914 |
| (mean) | | | | | | |
| Disintegration time | 112-138 | 47-68 | 68-79 | N/A | 50-69 | 114-160 |
| range (sec) | | | | | | |

—
Dissolution Method Conditions

| Parameter | Condition |
|---|---|
| Apparatus | USP Apparatus 2 (paddles) |
| Media | 50 mM sodium phosphate buffer, pH 6.8, |
| | with 0.3% SDS (0.6% for 100 mg) |
| Media Volume | 900 mL (500 mL for 7.5 mg) |
| Vessel Size | 1000 mL |
| Temperature | 37.0 ± 0.5° C. |
| Speed | 0-60 minutes: 75 rpm, 60-75 minutes: 250 rpm (infinity) |
| Time Points | 10, 20, 30, 45, 60 and 75 minutes |
| Filter | 0.45 μm, 25 mm Nylon Syringe Filter |

Dissolution Results (% Dissolved)

Time Points (minutes)

| Dose Strength | | 10 | 20 | 30 | 45 | 60 | 75 |
|---|---|---|---|---|---|---|---|
| 7.5 mg | Mean | 66 | 82 | 89 | 92 | 94 | 97 |
| | % RSD | 1.30 | 1.56 | 1.96 | 1.86 | 1.82 | 1.02 |
| 25 mg | Mean | 78 | 91 | 94 | 96 | 96 | 96 |
| | % RSD | 0.40 | 0.23 | 0.42 | 0.58 | 0.67 | 0.63 |
| 30 mg | Mean | 80 | 93 | 97 | 98 | 99 | 99 |
| | % RSD | 0.87 | 0.64 | 0.61 | 0.46 | 0.53 | 0.52 |
| 50 mg | Mean | 88 | 97 | 98 | 99 | 99 | 99 |
| | % RSD | 0.83 | 1.18 | 1.26 | 1.30 | 1.28 | 1.30 |
| 100 mg | Mean | 87 | 97 | 98 | 98 | 99 | 99 |
| | % RSD | 0.31 | 0.49 | 0.56 | 0.71 | 0.59 | 0.61 |

Mini-tabs are often used to improve flexibility of dosing by filling into packets or capsules. For example, for a 20 mg dose, about 8 mini tablets can be filled into a size 1 or 2 capsule, for a 25 mg dose, about 10 mini tablets into a size 1 capsule, for a 30 mg dose, about 12 mini tablets into a size 1 capsule, for a 40 mg dose, about 16 mini tablets into a size 0 capsule, for a 50 mg dose, about 20 into size 00 capsule and for a 60 mg dose, about 24 can be filled into a Size 00 gelatin capsule.

### Example 38

### Preparation of Hand Filled Capsules at 25, 40, 50 and 60 mg Dose Strengths with Composition 30

Composition 30 was used to generate capsules of various dose strengths at different target fill weights and different sizes of capsule shells. For a 25 mg dose strength a 208 mg target fill weight with a Size 2 capsule was used. For a 40 mg dose strength a 333 mg target fill weight with a Size 1 capsule was used. For a 50 mg dose strength a 417 mg target fill weight with a Size 0 capsule was used. For a 60 mg dose strength a 500 mg target fill weight with a Size 00 capsule was used. The target fill weights, capsule shell sizes for the different dose strengths and dose strengths prepared are summarized in Table 47.

TABLE 47
Target Fill Weights, Capsule Shell Sizes and Dose
Strengths for Capsules Utilizing Composition 30

| Dose Strength | Target Fill Weight | Capsule Shell |
|---|---|---|
| (mg) | (mg) | Size |
| 25 | 208 | 2 |
| 40 | 333 | 1 |
| 50 | 417 | 0 |
| 60 | 500 | 00 |

The dissolution testing results for these capsules are summarized in Table 48.

TABLE 48
The dissolution testing results for capsules
Dissolution Method Conditions

| Parameter | Condition |
|---|---|
| Apparatus | USP Apparatus 2 (paddles with size appropriate sinkers) |
| Media | 50 mM sodium phosphate buffer, pH 6.8 with 0.3% SDS (0.4% for 60 mg) |
| Media Volume | 900 mL |
| Vessel Size | 1000 mL |
| Temperature | 37.0 ± 0.5° C. |
| Speed | 0-60 minutes: 75 rpm, 60-75 minutes: 250 rpm (infinity) |
| Time Points | 10, 20, 30, 45, 60 and 75 minutes |
| Filter | 0.45 μm, 25 mm Nylon Syringe Filter |

Dissolution Results (% Dissolved)

| Composition- | Time Points (minutes) | | | | | | |
|---|---|---|---|---|---|---|---|
| Dose Strength | | 10 | 20 | 30 | 45 | 60 | 75 |
| 30-25 mg | Mean | 72 | 91 | 96 | 98 | 99 | 99 |
| | % RSD | 0.87 | 0.27 | 0.36 | 0.36 | 0.35 | 0.12 |
| 30-40 mg | Mean | 70 | 88 | 93 | 96 | 97 | 98 |
| | % RSD | 3.19 | 1.90 | 1.80 | 1.70 | 1.50 | 1.97 |
| 30-50 mg | Mean | 72 | 91 | 96 | 98 | 99 | 100 |
| | % RSD | 1.31 | 0.38 | 0.47 | 0.44 | 0.59 | 0.34 |
| 30-60 mg | Mean | 73 | 92 | 96 | 98 | 99 | 100 |
| | % RSD | 1.59 | 1.06 | 1.19 | 1.33 | 1.32 | 0.57 |

### INCORPORATION BY REFERENCE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

We claim:

1-142. (canceled)

143. A pharmaceutical composition comprising:
(i) about 10% (w/w) to about 15% (w/w) of a crystalline form of the compound of formula (I) 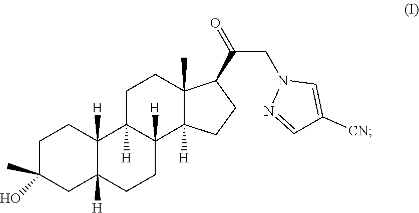
(ii) about 60% (w/w) to about 70% (w/w) of a brittle filler;
(iii) about 10% (w/w) to about 20% (w/w) of a ductile filler;
(iv) about 4% (w/w) to about 8% (w/w) of a disintegrant;
(v) about 0.5% (w/w) to about 2% (w/w) of a glidant; and
(vi) about 1% (w/w) to about 2% (w/w) of a lubricant.

144. The pharmaceutical composition of claim 143, wherein the brittle filler comprises mannitol, lactose, sucrose, fructose, glucose, maltose, dibasic calcium phosphate, hydroxyapatite, sodium carbonate, sodium bicarbonate, calcium carbonate, bentonite, kaolin, or a combination thereof.

145. The pharmaceutical composition of claim 143, wherein the brittle filler comprises mannitol.

146. The pharmaceutical composition of claim 143, wherein the ductile filler comprises a microcrystalline cellulose, a starch, a polysaccharide, a hydroxypropylcellulose, a hypromellose, a carboxymethylcellulose, a methylcellulose, a hydroxypropylmethylcellulose, a polyvinylpyrrolidone, a polyvinyl acrylate, a hydroxypropylcellulose, a hypromellose, a carboxymethylcellulose, a methylcellulose, a hydroxypropylmethylcellulose, or a combination thereof.

147. The pharmaceutical composition of claim 143, wherein the ductile filler comprises a microcrystalline cellulose.

148. The pharmaceutical composition of claim 147, wherein the microcrystalline cellulose comprises a silicified microcrystalline cellulose.

149. The pharmaceutical composition of claim 143, wherein the disintegrant comprises sodium starch glycolate, a crospovidone, croscarmellose sodium, or a combination thereof.

150. The pharmaceutical composition of claim 143, wherein the disintegrant comprises croscarmellose sodium.

151. The pharmaceutical composition of claim 143, wherein the glidant comprises colloidal silicon dioxide, talc, kaolin, bentonite, or a combination thereof.

152. The pharmaceutical composition of claim 143, wherein the glidant comprises colloidal silicon dioxide.

153. The pharmaceutical composition of claim 143, wherein the comprises sodium stearyl fumarate, magnesium stearate, stearic acid, glyceryl behenate, or a combination thereof.

154. The pharmaceutical composition of claim 143, wherein the comprises sodium stearyl fumarate.

155. The pharmaceutical composition of claim 143, wherein the crystalline form of the compound of formula (I) comprises crystalline Form A, wherein crystalline Form A exhibits an X-ray powder diffraction pattern comprising characteristic peaks at the following diffraction angles (2θ): 9.5°±0.2°, 10.8°±0.2°, 13.2°±0.2°, 14.9°±0.2°, 16.0°±0.2°, 18.3°±0.2°, 18.9°±0.2°, 21.1°±0.2°, 21.6°±0.2°, and 23.5°±0.2°.

156. The pharmaceutical composition of claim 143, wherein the crystalline form of the compound of formula (I) comprises crystalline Form C, wherein crystalline Form C exhibits an X-ray powder diffraction pattern comprising characteristic peaks at the following diffraction angles (2θ): 9.9°±0.2°, 11.8°±0.2°, 13.4°±0.2°, 14.4°±0.2°, 14.8°±0.2°, 17.0°±0.2°, 20.7°±0.2°, 21.5°±0.2°, and 22.6°±0.2°.

157. The pharmaceutical composition of claim 143, wherein the crystalline form of the compound of formula (I) comprises crystalline Form C, wherein crystalline Form C exhibits an X-ray powder diffraction pattern comprising characteristic peaks at the following diffraction angles (2θ): 9.9°±0.2°, 14.8°±0.2°, 17.0°±0.2°, 20.7°±0.2°, and 21.5°±0.2°.

158. A pharmaceutical composition comprising:
(i) about 20 mg of a crystalline form of the compound of formula (I) 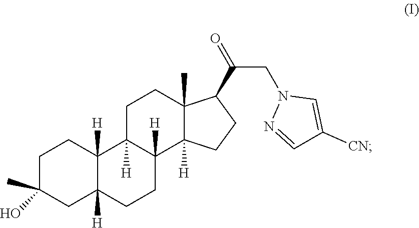
(ii) about 105.9 mg of mannitol;
(iii) about 26.2 mg of silicified microcrystalline cellulose;
(iv) about 10 mg of croscarmellose sodium;
(v) about 1.7 mg of colloidal silicon dioxide; and
(vi) about 2.9 mg of sodium stearyl fumarate.

159. A pharmaceutical composition comprising:
(i) about 25 mg of a crystalline form of the compound of formula (I) 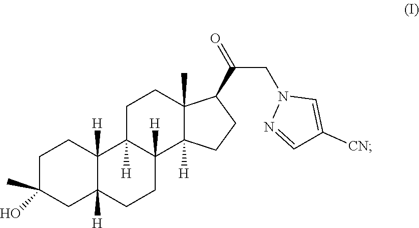
(ii) about 132 mg of mannitol;
(iii) about 32.7 mg of the silicified microcrystalline cellulose;
(iv) about 12.5 mg of croscarmellose sodium;
(v) about 2.1 mg of colloidal silicon dioxide; and
(vi) about 3.6 mg of sodium stearyl fumarate.

160. A pharmaceutical composition comprising:
(i) about 30 mg of a crystalline form of the compound of formula (I) 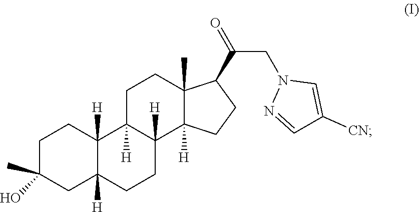
(ii) about 159 mg of mannitol;
(iii) about 39.3 mg of the silicified microcrystalline cellulose;
(iv) about 15 mg of croscarmellose sodium;
(v) about 2.5 mg of colloidal silicon dioxide; and
(vi) about 4.4 mg of sodium stearyl fumarate.

161. A dosage form intended for oral administration comprising a pharmaceutical composition of claim 143.

162. The dosage form of claim 161, wherein the dosage form is selected from the group consisting of a powder, a sachet, a stickpack, a capsule, a minitab, and a tablet.

163. The dosage form of claim 161, wherein the dosage form is a capsule.

164. The dosage form of claim 163, wherein the size of the capsule is selected from the group consisting of 000, 00, 0, 1, 2, 3, 4, and 5.

165. The dosage form of claim 164, wherein the size of the capsule is 1.

166. A method of treating a CNS-related disorder, the method comprising administering to a subject in need thereof a pharmaceutical composition of claim 143.

167. The method of claim 166, wherein the CNS-related disorder comprises a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, or status epilepticus.

168. A method of treating major depressive disorder, the method comprising administering to a subject in need thereof a pharmaceutical composition of claim 143.

169. A method of treating postpartum depression, the method comprising administering to a subject in need thereof a pharmaceutical composition of claim 143.
